(19) United States
(12) Patent Application Publication
(10) Pub. No.: US 2013/0029961 A1
(43) Pub. Date: Jan. 31, 2013

(54) Substituted Heterocyclic Aza Compounds

(71) Applicants: Robert FRANK, Aachen (DE); Thomas Christoph, Aachen (DE); Bernhard Lesch, Aachen (DE); Jeewoo Lee, Seoul (KR)

(72) Inventors: Robert FRANK, Aachen (DE); Thomas Christoph, Aachen (DE); Bernhard Lesch, Aachen (DE); Jeewoo Lee, Seoul (KR)

(73) Assignee: Gruenenthal GmbH, Aachen (DE)

(21) Appl. No.: 13/557,773

(22) Filed: Jul. 25, 2012

Related U.S. Application Data
(60) Provisional application No. 61/511,731, filed on Jul. 26, 2011.

(30) Foreign Application Priority Data
Jul. 26, 2011 (EP) 11 006 115.7

(51) Int. Cl. A61K 31/444; C07D 401/14; A61K 31/4545; A61K 31/506; A61P 25/00; A61P 25/06; A61P 25/24; A61P 25/28; A61P 25/08; A61P 11/00; A61P 11/14; A61P 13/00; A61P 13/10; A61P 1/00; A61P 1/04; A61P 27/02; A61P 17/00; A61P 37/08; A61P 17/06; A61P 31/22; A61P 29/00; A61P 1/12; A61P 17/04; A61P 19/10; A61P 19/02; A61P 25/30; A61P 25/32; A61P 7/10; A61P 9/00; A61P 17/02; A61P 23/02; C07D 401/12

(52) U.S. Cl. 514/210.2; 546/265; 514/332; 546/193; 514/318; 544/333; 514/256

(57) ABSTRACT

Heterocyclic aza compounds as vanilloid receptor ligands, pharmaceutical compositions containing these compounds and also methods of using these compounds for the treatment and/or inhibition of pain and further diseases and/or disorders.

### BACKGROUND OF THE INVENTION

The invention relates to substituted heterocyclic aza derivatives as vanilloid receptor ligands, to pharmaceutical compositions containing these compounds and also to these compounds for use in the treatment and/or inhibition of pain and further diseases and/or disorders.

The treatment of pain, in particular of neuropathic pain, is very important in medicine. There is a worldwide demand for effective pain therapies. The urgent need for action for a patient-focused and target-oriented treatment of chronic and non-chronic states of pain, this being understood to mean the successful and satisfactory treatment of pain for the patient, is also documented in the large number of scientific studies which have recently appeared in the field of applied analgesics or basic research on nociception.

The subtype 1 vanilloid receptor (VR1/TRPV1), which is often also referred to as the capsaicin receptor, is a suitable starting point for the treatment of pain, in particular of pain selected from the group consisting of acute pain, chronic pain, neuropathic pain and visceral pain. This receptor is stimulated inter alia by vanilloids such as capsaicin, heat and protons and plays a central role in the formation of pain. In addition, it is important for a large number of further physiological and pathophysiological processes and is a suitable target for the therapy of a large number of further disorders such as, for example, migraine, depression, neurodegenerative diseases, cognitive disorders, states of anxiety, epilepsy, coughs, diarrhoea, pruritus, inflammations, disorders of the cardiovascular system, eating disorders, medication dependency, misuse of medication and urinary incontinence.

There is a demand for further compounds having comparable or better properties, not only with regard to affinity to vanilloid receptors 1 (VR1/TRPV1 receptors) per se (potency, efficacy).

Thus, it may be advantageous to improve the metabolic stability, the solubility in aqueous media or the permeability of the compounds. These factors can have a beneficial effect on oral bioavailability or can alter the PK/PD (pharmacokinetic/pharmacodynamic) profile; this can lead to a more beneficial period of effectiveness, for example.

### SUMMARY OF THE INVENTION

It was therefore an object of the invention to provide novel compounds, preferably having advantages over the prior-art compounds. The compounds should be suitable in particular as pharmacological active ingredients in pharmaceutical compositions, preferably in pharmaceutical compositions for the treatment and/or inhibition of disorders or diseases which are at least partially mediated by vanilloid receptors 1 (VR1/TRPV1 receptors).

This object is achieved by the subject matter of the claims and the subject-matter as described herein.

It has surprisingly been found that the substituted compounds of general formula (I), as given below, display outstanding affinity to the subtype 1 vanilloid receptor (VR1/TRPV1 receptor) and are therefore particularly suitable for the inhibition and/or treatment of disorders or diseases which are at least partially mediated by vanilloid receptors 1 (VR1/TRPV1).

The present invention therefore relates to substituted compounds of general formula (I),

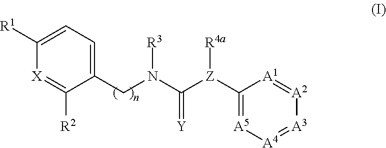

wherein
n represents 0, 1, 2, 3 or 4; preferably represents 1, 2, 3 or 4;
X represents N or CH;
Y represents O, S, or N—CN;
Z represents N or C—R^{4b};
A¹ represents N or CR⁵;
A² represents N or CR⁶;
A³ represents N or CR⁷;
A⁴ represents N or CR⁸;
A⁵ represents N or CR⁹;
with the proviso that 1, 2 or 3 of variables A¹, A², A³, A⁴ and A⁵ represent a nitrogen atom;
R⁰ represents a C₁₋₁₀ aliphatic residue, unsubstituted or mono- or polysubstituted; a C₃₋₁₀ cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; aryl or heteroaryl, in each case unsubstituted or mono- or polysubstituted and in each case optionally bridged via a C₁₋₈ aliphatic group, which in turn may be unsubstituted or mono- or polysubstituted;
R¹ represents a C₁₋₄ aliphatic residue, unsubstituted or mono- or polysubstituted, a C₃₋₆ cycloaliphatic residue or a 3 to 6 membered heterocycloaliphatic residue, in each case unsubstituted or mono- or polysubstituted;
R² represents R⁰; OR⁰; SR⁰; NH₂; NHR⁰ or N(R⁰)₂;
R³ represents H or a C₁₋₄ aliphatic residue, unsubstituted or mono- or polysubstituted;
R^{4a} represents H; a C₁₋₄ aliphatic residue, unsubstituted or mono- or polysubstituted; a C₃₋₆ cycloaliphatic residue, unsubstituted or mono- or polysubstituted; or aryl, unsubstituted or mono- or polysubstituted;
R^{4b} represents H; or a C₁₋₄ aliphatic residue, unsubstituted, mono- or polysubstituted;
or R^{4a} and R^{4b} together with the carbon atom connecting them form a C₃₋₆ cycloaliphatic residue, unsubstituted or mono- or polysubstituted;
R⁵, R⁶, R⁷, R⁸, and R⁹ each independently of one another represent H; F; Cl; Br; I; CN; CF₃; CF₂H; CFH₂; CF₂Cl; CFCl₂; NO₂; R⁰; C(═O)—H; C(═O)—R⁰; C(═O)—OH; C(═O)—OR⁰; C(═O)—NH₂; C(═O)—NHR⁰; C(═O)—N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NHR⁰; O—C(═O)—N(R⁰)₂; O—S(═O)₂—R⁰; O—S(═O)₂—OH; O—S(═O)₂—OR⁰; O—S(═O)₂—NH₂; O—S(═O)₂—NHR⁰; O—S(═O)₂—N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NHR⁰; NR⁰—C(═O)—N(R⁰)₂; NH—S(═O)₂—OH; NH—S(═O)₂—R⁰; NH—S(═O)₂—OR⁰; NH—S(═O)₂—NH₂; NH—S(═O)₂—NHR⁰; NH—S(═O)₂—N(R⁰)₂; NR⁰—S(═O)₂—OH; NR⁰—S(═O)₂—R⁰; NR⁰—S(═O)₂—OR⁰; NR⁰—S(═O)₂—NH₂; NR⁰—S(═O)₂—NHR⁰; NR⁰—S(═O)₂—N(R⁰)₂; SH; SCF₃; SCF₂H; SCFH₂; SCF₂Cl; SCFCl₂; SW; S(═O)—R⁰; S(═O)₂—R⁰; S(═O)₂—OH; S(═O)₂—OR⁰; S(═O)₂—NH₂; S(═O)₂—NHR⁰; or S(═O)₂—N(R⁰)₂;
in which an “aliphatic group” and an “aliphatic residue” can in each case, independently of one another, be branched or unbranched, saturated or unsaturated;
in which a “cycloaliphatic residue” and a “heterocycloaliphatic residue” can in each case, independently of one another, be saturated or unsaturated;
in which “mono- or polysubstituted” with respect to an “aliphatic group”, an “aliphatic residue”, a “cycloaliphatic residue” and a “heterocycloaliphatic residue” relates in each case independently of one another, with respect to the corresponding residues or groups, to the substitution of one or more hydrogen atoms each independently of one another by at least one substituent selected from the group consisting of F; Cl; Br; I; NO₂; CN; ═O; ═NH; ═N(OH); ═C(NH₂)₂; CF₃; CF₂H; CFH₂; CF₂Cl; CFCl₂; R⁰; C(═O)—H; C(═O)—R⁰; C(═O)—OH; C(═O)—OR⁰; CO—NH₂; C(═O)—NHR⁰; C(═O)—N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂—OH; O—S(═O)₂—OR⁰; O—S(═O)₂—NH₂; O—S(═O)₂—NHR⁰; O—S(═O)₂—N(R⁰)₂; NH₂; NH—R⁰; N(R⁰)₂; NH—C(═O)—R⁰; NH—C(═O)—O—R⁰; NH—C(═O)—NH₂; NH—C(═O)—NHR⁰; NH—C(═O)—N(R⁰)₂; NR⁰—C(═O)—R⁰; NR⁰—C(═O)—O—R⁰; NR⁰—C(═O)—NH₂; NR⁰—C(═O)—NHR⁰; NR⁰—C(═O)—N(R⁰)₂; NH—S(═O)₂—OH; NH—S(═O)₂—R⁰; NH—S(═O)₂—OR⁰; NH—S(═O)₂—NH₂; NH—S(═O)₂—NHR⁰; NH—S(═O)₂—N(R⁰)₂; NR⁰—S(═O)₂—OH; NR⁰—S(═O)₂—R⁰; NR⁰—S(═O)₂—OR⁰; NR⁰—S(═O)₂—NH₂; NR⁰—S(═O)₂—NHR⁰; NR⁰—S(═O)₂—N(R⁰)₂; SH; SCF₃; SCF₂H; SCFH₂; SCF₂Cl; SCFCl₂; SW; S(═O)—R⁰; S(═O)₂—R⁰; S(═O)₂—OH; S(═O)₂—OR⁰; S(═O)₂—NH₂; S(═O)₂—NHR⁰; and S(═O)₂—N(R⁰)₂;
in which “mono- or polysubstituted” with respect to “aryl” and a “heteroaryl” relates, with respect to the corresponding residues, in each case independently of one another, to the substitution of one or more hydrogen atoms each independently of one another by at least one substituent selected from the group consisting of F; Cl; Br; I; NO₂; CN; CF₃; CF₂H; CFH₂; CF₂Cl; CFCl₂; R⁰; C(═O)—H; C(═O)—R⁰; C(═O)—OH; C(═O)—OR⁰; CO—NH₂; C(═O)—NHR⁰; C(═O)—N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂—OH; O—S(═O)₂—OR⁰; O—S(═O)₂—NH₂; O—S(═O)₂—NHR⁰; O—S(═O)₂—N(R⁰)₂; NH₂; NHR⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂—OH; NH—S(═O)₂—R⁰; NH—S(═O)₂—OR⁰; NH—S(═O)₂—NH₂; NH—S(═O)₂—NHR⁰; NH—S(═O)₂—N(R⁰)₂; NR⁰—S(═O)₂—OH; NR⁰—S(═O)₂R⁰; NR⁰—S(═O)₂—OR⁰; NR⁰—S(═O)₂—NH₂; NR⁰—S(═O)₂—NHR⁰; NR⁰—S(═O)₂—N(R⁰)₂; SH; SCF₃; SCF₂H; SCFH₂; SCF₂Cl; SCFCl₂; SR⁰; S(═O)—R⁰; S(═O)₂—R⁰; S(═O)₂—OH; S(═O)₂—OR⁰; S(═O)₂—NH₂; S(═O)₂—NHR⁰; and S(═O)₂—N(R⁰)₂;
optionally in the form of a single stereoisomer or a mixture of stereoisomers, in the form of the free compound and/or a physiologically acceptable salt thereof.

### DETAILED DESCRIPTION

The term “single stereoisomer” comprises in the sense of this invention an individual enantiomer or diastereomer. The term “mixture of stereoisomers” comprises in the sense of this invention the racemate and mixtures of enantiomers and/or diastereomers in any mixing ratio.

The term “physiologically acceptable salt” comprises in the sense of this invention a salt of at least one compound according to the present invention and at least one physiologically acceptable acid or base.

The terms “C₁₋₁₀ aliphatic residue”, “C₁₋₈ aliphatic residue”, and “C₁₋₄ aliphatic residue” comprise in the sense of this invention acyclic saturated or unsaturated aliphatic hydrocarbon residues, which can be branched or unbranched and also unsubstituted or mono- or polysubstituted, which contain 1 to 10, or 1 to 8, or 1 to 4 carbon atoms, respectively, i.e. C₁₋₁₀ alkanyls (C₁₋₁₀ alkyls), C₂₋₁₀ alkenyls and C₂₋₁₀ alkynyls as well as C₁₋₈ alkanyls (C₁₋₈ alkyls), C₂₋₈ alkenyls and C₂₋₈ alkynyls as well as C₁₋₄ alkanyls (C₁₋₄ alkyls), C₂₋₄ alkenyls and C₂₋₄ alkynyls, respectively. Alkenyls comprise at least one C—C double bond (a C═C-bond) and alkynyls comprise at least one C—C triple bond (a C≡C-bond). Preferably, aliphatic residues are selected from the group consisting of alkanyl (alkyl) and alkenyl residues, more preferably are alkanyl (alkyl) residues. Preferred C₁₋₁₀ alkanyl residues are selected from the group consisting of methyl, ethyl, n-propyl, 2-propyl, n-butyl, isobutyl, sec.-butyl, tert.-butyl, n-pentyl, isopentyl, neopentyl, n-hexyl, n-heptyl, n-octyl, n-nonyl and n-decyl. Preferred C₁₋₈ alkanyl residues are selected from the group consisting of methyl, ethyl, n-propyl, 2-propyl, n-butyl, isobutyl, sec.-butyl, tert.-butyl, n-pentyl, isopentyl, neopentyl, n-hexyl, n-heptyl and n-octyl. Preferred C₁₋₄ alkanyl residues are selected from the group consisting of methyl, ethyl, n-propyl, 2-propyl, n-butyl, isobutyl, sec.-butyl and tert.-butyl. Preferred C₂₋₁₀ alkenyl residues are selected from the group consisting of ethenyl (vinyl), propenyl (—CH₂CH═CH₂, —CH═CH—CH₃, —C(═CH₂)—CH₃), butenyl, pentenyl, hexenyl heptenyl, octenyl, nonenyl and decenyl. Preferred C₂₋₈ alkenyl residues are selected from the group consisting of ethenyl (vinyl), propenyl (—CH₂CH═CH₂, —CH═CH—CH₃, —C(═CH₂)—CH₃), butenyl, pentenyl, hexenyl heptenyl and octenyl. Preferred C₂₋₄ alkenyl residues are selected from the group consisting of ethenyl (vinyl), propenyl (—CH₂CH═CH₂, —CH═CH—CH₃, —C(═CH₂)—CH₃) and butenyl. Preferred C₂₋₁₀ alkynyl residues are selected from the group consisting of ethynyl, propynyl (—CH₂—C≡CH, —C≡C—CH₃), butynyl, pentynyl, hexynyl, heptynyl, octynyl, nonynyl and decynyl. Preferred C₂₋₈ alkynyl residues are selected from the group consisting of ethynyl, propynyl (—CH₂—C≡CH, —C≡C—CH₃), butynyl, pentynyl, hexynyl, heptynyl and octynyl. Preferred C₂₋₄ alkynyl residues are selected from the group consisting of ethynyl, propynyl (—CH₂—C≡CH, —C≡C—CH₃) and butynyl.

The terms “C₃₋₆ cycloaliphatic residue” and “C₃₋₁₀ cycloaliphatic residue” mean for the purposes of this invention cyclic aliphatic hydrocarbons containing 3, 4, 5 or 6 carbon atoms and 3, 4, 5, 6, 7, 8, 9 or 10 carbon atoms, respectively, wherein the hydrocarbons in each case can be saturated or unsaturated (but not aromatic), unsubstituted or mono- or polysubstituted. The cycloaliphatic residues can be bound to the respective superordinate general structure via any desired and possible ring member of the cycloaliphatic residue. The cycloaliphatic residues can also be condensed with further saturated, (partially) unsaturated, (hetero)cyclic, aromatic or heteroaromatic ring systems, i.e. with cycloaliphatic, heterocycloaliphatic, aryl or heteroaryl residues, which in each case can in turn be unsubstituted or mono- or polysubstituted. C₃₋₁₀ cycloaliphatic residue can furthermore be singly or multiply bridged such as, for example, in the case of adamantyl, bicyclo[2.2.1]heptyl or bicyclo[2.2.2]octyl. Preferred C₃₋₁₀ cycloaliphatic residues are selected from the group consisting of cyclopropyl, cyclobutyl, cyclopentyl, cyclohexyl, cycloheptyl, cyclooctyl, cyclononyl, cyclodecyl, adamantyl,

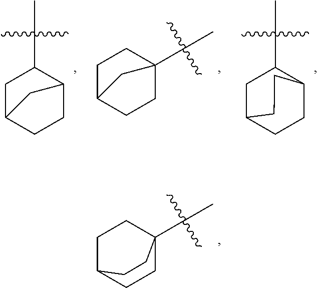

cyclopentenyl, cyclohexenyl, cycloheptenyl and cyclooctenyl. Preferred C₃₋₆ cycloaliphatic residues are selected from the group consisting of cyclopropyl, cyclobutyl, cyclopentyl, cyclohexyl, cyclopentenyl and cyclohexenyl. Particularly preferred C₃₋₁₀ cycloaliphatic and C₃₋₆ cycloaliphatic residues are C₆₋₆ cycloaliphatic residues such as cyclopentyl, cyclohexyl, cyclopentenyl and cyclohexenyl.

The terms “3-6-membered heterocycloaliphatic residue”, and “3-10-membered heterocycloaliphatic residue” mean for the purposes of this invention heterocycloaliphatic saturated or unsaturated (but not aromatic) residues having 3-6, i.e. 3, 4, 5 or 6 ring members, and 3-10, i.e. 3, 4, 5, 6, 7, 8, 9 or 10 ring members, respectively, in which in each case at least one, if appropriate also two or three carbon atoms are replaced by a heteroatom or a heteroatom group each selected independently of one another from the group consisting of O, S, S(═O)₂, N, NH and N(C₁₋₈ alkyl) such as N(CH₃), preferably are replaced by a heteroatom or a heteroatom group each selected independently of one another from the group consisting of O, S, N, NH and N(C₁₋₈ alkyl) such as N(CH₃), wherein the ring members can be unsubstituted or mono- or polysubstituted. The heterocycloaliphatic residue can be bound to the superordinate general structure via any desired and possible ring member of the heterocycloaliphatic residue if not indicated otherwise. The heterocycloaliphatic residues can also be condensed with further saturated, (partially) unsaturated (hetero)cycloaliphatic or aromatic or heteroaromatic ring systems, i.e. with cycloaliphatic, heterocycloaliphatic, aryl or heteroaryl residues, which can in turn be unsubstituted or mono- or polysubstituted. Preferred heterocycloaliphatic residues are selected from the group consisting of azetidinyl, aziridinyl, azepanyl, azocanyl, diazepanyl, dithiolanyl, dihydroquinolinyl, dihydropyrrolyl, dioxanyl, dioxolanyl, dioxepanyl, dihydroindenyl, dihydropyridinyl, dihydrofuranyl, dihydroisoquinolinyl, dihydroindolinyl, dihydroisoindolyl, imidazolidinyl, isoxazolidinyl, morpholinyl, oxiranyl, oxetanyl, oxazepanyl, pyrrolidinyl, piperazinyl, 4-methylpiperazinyl, piperidinyl, pyrazolidinyl, pyranyl, tetrahydropyrrolyl, tetrahydropyranyl, tetrahydro-2H-pyran-4-yl, tetrahydroquinolinyl, tetrahydroisoquinolinyl, tetrahydroindolinyl, tetrahydrofuranyl, tetrahydropyridinyl, tetrahydrothiophenyl, tetrahydropyridoindolyl, tetrahydronaphthyl, tetrahydrocarbolinyl, tetrahydroisoxazololyl, tetrahydropyridinyl, thiazolidinyl and thiomorpholinyl.

The term “aryl” means for the purpose of this invention aromatic hydrocarbons having 6 to 14, i.e. 6, 7, 8, 9, 10, 11, 12, 13 or 14 ring members, preferably having 6 to 10, i.e. 6, 7, 8, 9 or 10 ring members, including phenyls and naphthyls. Each aryl residue can be unsubstituted or mono- or polysubstituted, wherein the aryl substituents can be the same or different and in any desired and possible position of the aryl. The aryl can be bound to the superordinate general structure via any desired and possible ring member of the aryl residue. The aryl residues can also be condensed with further saturated, (partially) unsaturated, (hetero)cycloaliphatic, aromatic or heteroaromatic ring systems, i.e. with a cycloaliphatic, heterocycloaliphatic, aryl or heteroaryl residue, which can in turn be unsubstituted or mono- or polysubstituted. Examples of condensed aryl residues are benzodioxolanyl and benzodioxanyl. Preferably, aryl is selected from the group consisting of phenyl, 1-naphthyl, 2-naphthyl, fluorenyl and anthracenyl, each of which can be respectively unsubstituted or mono- or polysubstituted. A particularly preferred aryl is phenyl, unsubstituted or mono- or polysubstituted.

The term “heteroaryl” for the purpose of this invention represents a 5 or 6-membered cyclic aromatic residue containing at least 1, if appropriate also 2, 3, 4 or 5 heteroatoms, wherein the heteroatoms are each selected independently of one another from the group S, N and O and the heteroaryl residue can be unsubstituted or mono- or polysubstituted; in the case of substitution on the heteroaryl, the substituents can be the same or different and be in any desired and possible position of the heteroaryl. The binding to the superordinate general structure can be carried out via any desired and possible ring member of the heteroaryl residue if not indicated otherwise. The heteroaryl can also be part of a bi- or polycyclic system having up to 14 ring members, wherein the ring system can be formed with further saturated, (partially) unsaturated, (hetero)cycloaliphatic or aromatic or heteroaromatic rings, i.e. with a cycloaliphatic, heterocycloaliphatic, aryl or heteroaryl residue, which can in turn be unsubstituted or mono- or polysubstituted. It is preferable for the heteroaryl residue to be selected from the group consisting of benzofuranyl, benzoimidazolyl, benzothienyl, benzothiadiazolyl, benzothiazolyl, benzotriazolyl, benzooxazolyl, benzooxadiazolyl, quinazolinyl, quinoxalinyl, carbazolyl, quinolinyl, dibenzofuranyl, dibenzothienyl, furyl (furanyl), imidazolyl, imidazothiazolyl, indazolyl, indolizinyl, indolyl, isoquinolinyl, isoxazoyl, isothiazolyl, indolyl, naphthyridinyl, oxazolyl, oxadiazolyl, phenazinyl, phenothiazinyl, phthalazinyl, pyrazolyl, pyridyl (2-pyridyl, 3-pyridyl, 4-pyridyl), pyrrolyl, pyridazinyl, pyrimidinyl, pyrazinyl, purinyl, phenazinyl, thienyl (thiophenyl), triazolyl, tetrazolyl, thiazolyl, thiadiazolyl and triazinyl.

The term “bridged via a C₁₋₄ aliphatic group or via a C₁₋₈ aliphatic group” with respect to residues such as aryl, heteroaryl, a heterocycloaliphatic residue and a cycloaliphatic residue mean for the purpose of the invention that these residues have the above-defined meanings and that each of these residues is bound to the respective superordinate general structure via a C₁₋₄ aliphatic group or via a C₁₋₈ aliphatic group, respectively. The C₁₋₄ aliphatic group and the C₁₋₈-aliphatic group can in all cases be branched or unbranched, unsubstituted or mono- or polysubstituted. The C₁₋₄ aliphatic group can in all cases be furthermore saturated or unsaturated, i.e. can be a C₁₋₄ alkylene group, a C₂₋₄ alkenylene group or a C₂₋₄ alkynylene group. The same applies to a C₁₋₈-aliphatic group, i.e. a C₁₋₈-aliphatic group can in all cases be furthermore saturated or unsaturated, i.e. can be a C₁₋₈ alkylene group, a C₂₋₈ alkenylene group or a C₂₋₈ alkynylene group. Preferably, the C₁₋₄-aliphatic group is a C₁₋₄ alkylene group or a C₂₋₄ alkenylene group, more preferably a C₁₋₄ alkylene group. Preferably, the C₁₋₈-aliphatic group is a C₁₋₈ alkylene group or a C₂₋₈ alkenylene group, more preferably a C₁₋₈ alkylene group. Preferred C₁₋₄ alkylene groups are selected from the group consisting of —CH₂—, —CH₂—CH₂—, —CH(CH₃)—, —CH₂—CH₂—CH₂—, —CH(CH₃)—CH₂—, —CH(CH₂CH₃)—, —CH₂—(CH₂)₂—CH₂—, —CH(CH₃)—CH₂—CH₂—, —CH₂—CH(CH₃)—CH₂—, —CH(CH₃)—CH(CH₃)—, —CH(CH₂CH₃)—CH₂—, —C(CH₃)₂—CH₂—, —CH(CH₂CH₂CH₃)— and —C(CH₃)(CH₂CH₃)—. Preferred C₂₋₄ alkenylene groups are selected from the group consisting of —CH═CH—, —CH═CH—CH₂—, —C(CH₃)═CH₂—, —CH═CH—CH₂—CH₂—, —CH₂—CH═CH—CH₂—, —CH═CH—CH═CH—, —C(CH₃)═CH—CH₂—, —CH═C(CH₃)—CH₂—, —C(CH₃)═C(CH₃)— and —C(CH₂CH₃)═CH—. Preferred C₂₋₄ alkynylene groups are selected from the group consisting of —C≡C—, —C≡C—CH₂—, —C≡C—CH₂—CH₂—, —C≡C—CH(CH₃)—, —CH₂—C≡C—CH₂— and —C≡C—C≡C—. Preferred C₁₋₈ alkylene groups are selected from the group consisting of —CH₂—, —CH₂—CH₂—, —CH(CH₃)—, —CH₂—CH₂—CH₂—, —CH(CH₃)—CH₂—, —CH(CH₂CH₃)—, —CH₂—(CH₂)₂—CH₂—, —CH(CH₃)—CH₂—CH₂—, —CH₂—CH(CH₃)—CH₂—, —CH(CH₃)—CH(CH₃)—, —CH(CH₂CH₃)—CH₂—, —C(CH₃)₂—CH₂—, —CH(CH₂CH₂CH₃)—, —C(CH₃)(CH₂CH₃)—, —CH₂—(CH₂)₃CH₂—, —CH(CH₃)—CH₂—CH₂—CH₂—, —CH₂—CH(CH₃)—CH₂—CH₂—, —CH(CH₃)—CH₂—CH(CH₃)—, —CH(CH₃)—CH(CH₃)—CH₂—, —C(CH₃)₂—CH₂—CH₂—, —CH₂—C(CH₃)₂—CH₂—, —CH(CH₂CH₃)—CH₂—CH₂—, —CH₂—CH(CH₂CH₃)—CH₂—, —C(CH₃)₂—CH(CH₃)—, —CH(CH₂CH₃)—CH(CH₃)—, —C(CH₃)(CH₂CH₃)—CH₂—, —CH(CH₂CH₂CH₃)—CH₂—, —C(CH₂CH₂CH₃)—CH₂—, —CH(CH₂CH₂CH₂CH₃)—, —C(CH₃)(CH₂CH₂CH₃)—, —C(CH₂CH₃)₂— and —CH₂—(CH₂)₄—CH₂—. Preferred C₂₋₈ alkenylene groups are selected from the group consisting of —CH═CH—, —CH═CH—CH₂—, —C(CH₃)═CH₂—, —CH═CH—CH₂—CH₂—, —CH₂—CH═CH—CH₂—, —CH═CH—CH═CH—, —C(CH₃)═CH—CH₂—, —CH═C(CH₃)—CH₂—, —C(CH₃)═C(CH₃)—, —C(CH₂CH₃)═CH—, —CH═CH—CH₂—CH₂—CH₂—, —CH₂—CH═CH₂—CH₂—CH₂—, —CH═CH═CH—CH₂—CH₂— and —CH═CH₂—CH—CH═CH₂—. Preferred C₂₋₈ alkynylene groups are selected from the group consisting of —C≡C—, —C≡C—CH₂—, —C≡C—CH₂—CH₂—, —C≡C—CH(CH₃)—, —CH₂—C≡C—CH₂—, —C≡C—C≡C—, —C≡C—C(CH₃)₂—, —C≡C—CH₂—CH₂—CH₂—, —CH₂—C≡C—CH₂—CH₂—, —C≡C—C≡C—CH₂— and —C≡C—CH₂—C≡C.

In relation to the terms “aliphatic residue”, “aliphatic group”, “cycloaliphatic residue” and “heterocycloaliphatic residue”, the term “mono- or polysubstituted” refers in the sense of this invention, with respect to the corresponding residues or groups, to the single substitution or multiple substitution, e.g. disubstitution, trisubstitution, tetrasubstitution, or pentasubstitution, of one or more hydrogen atoms each independently of one another by at least one substituent selected from the group consisting of F; Cl; Br; I; NO₂; CN; ═O; ═NH; ═N(OH); ═C(NH₂)₂; CF₃; CF₂H; CFH₂; CF₂Cl; CFCl₂; R⁰; C(═O)—H; C(═O)—R⁰; C(═O)—OH; C(═O)—OR⁰; CO—NH₂; C(═O)—NHR⁰; C(═O)—N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂—OH; O—S(═O)₂—OR⁰; O—S(═O)₂—NH₂; O—S(═O)₂—NHR⁰; O—S(═O)₂—N(R⁰)₂; NH₂; NH—R⁰; N(R⁰)₂; NH—C(═O)—R⁰; NH—C(═O)—O—R⁰; NH—C(═O)—NH₂; NH—C(═O)—NHR⁰; NH—C(═O)—N(R⁰)₂; NR⁰—C(═O)—R⁰; NR⁰—C(═O)—O—R⁰; NR⁰—C(═O)—NH₂; NR⁰—C(═O)—NHR⁰; NR⁰—C(═O)—N(R⁰)₂; NH—S(═O)₂—OH; NH—S(═O)₂—R⁰; NH—S(═O)₂—OR⁰; NH—S(═O)₂—NH₂; NH—S(═O)₂—NHR⁰; NH—S(═O)₂—N(R⁰)₂; NR⁰—S(═O)₂—OH; NR⁰—S(═O)₂—R⁰; NR⁰—S(═O)₂—OR⁰; NR⁰—S(═O)₂—NH₂; NR⁰—S(═O)₂—NHR⁰; NR⁰—S(═O)₂—N(R⁰)₂; SH; SCF₃; SCF₂H; SCFH₂; SCF₂Cl; SCFCl₂; SR⁰; S(═O)—R⁰; S(═O)₂—R⁰; S(═O)₂—OH; S(═O)₂—OR⁰; S(═O)₂—NH₂; S(═O)₂—NHR⁰; and S(═O)₂—N(R⁰)₂. The term “polysubstituted” with respect to polysubstituted residues and groups includes the polysubstitution of these residues and groups either on different or on the same atoms, for example trisubstituted on the same carbon atom, as in the case of CF₃, CH₂CF₃ or 1,1-difluorocyclohexyl, or at various points, as in the case of CH(OH)—CH═CH—CHCl₂ or 1-chloro-3-fluorocyclohexyl. A substituent can if appropriate for its part in turn be mono- or polysubstituted. The multiple substitution can be carried out using the same or using different substituents.

Preferred substituents of “aliphatic residue” and “aliphatic group” are selected from the group consisting of F; Cl; Br; I; NO₂; CF₃; CN; ═O; ═NH; R⁰; (C₁₋₈ alkylene)-OH; C(═O)(R⁰ or H); C(═O)O(R⁰ or H); C(═O)N(R⁰ or H)₂; OH; OR⁰; O—C(═O)—R⁰; O—(C₁₋₈ alkyl)-OH; O—(C₁₋₈ alkyl)-O—C₁₋₈ alkyl; OCF₃; N(R⁰ or H)₂; N(R⁰ or H)—C(═O)—R⁰; N(R⁰ or H)—S(═O)₂—R⁰; N(R⁰ or H)—C(═O)—N(R⁰ or H)₂; SH; SCF₃; SW; S(═O)₂R⁰; S(═O)₂O(R⁰ or H) and S(═O)₂—N(R⁰ or H)₂.

Particularly preferred substituents of “aliphatic residue” and “aliphatic group” are selected from the group consisting of F; Cl; Br; I; NO₂; CF₃; CN; ═O; C₁₋₈ aliphatic residue; aryl; heteroaryl; C₃₋₆ cycloaliphatic residue; 3 to 6 membered heterocycloaliphatic residue; aryl, heteroaryl, C₃₋₆ cycloaliphatic residue or 3 to 6 membered heterocycloaliphatic bridged via a C₁ aliphatic group; CHO; C(═O)—C₁₋₈ aliphatic residue; C(═O)aryl; C(═O)heteroaryl; CO₂H; C(═O)O—C₁₋₈ aliphatic residue; C(═O)O-aryl; C(═O)O-heteroaryl; C(═O)—NH₂; C(═O)NH—C₁₋₈ aliphatic residue; C(═O)N(C₁₋₈ aliphatic residue)₂; C(═O)NH-aryl; C(═O)N(aryl)₂; C(═O)NH-heteroaryl; C(═O)N(heteroaryl)₂; C(═O)N(C₁₋₈ aliphatic residue)(aryl); C(═O)N(C₁₋₈ aliphatic residue)(heteroaryl); C(═O)N(heteroaryl)(aryl); OH; O—C₁₋₈ aliphatic residue; OCF₃; O—(C₁₋₈ aliphatic residue)-OH; O—(C₁₋₈ aliphatic residue)-O—C₁₋₈ aliphatic residue; O-benzyl; O-aryl; O-heteroaryl; O—C(═O)—C₁₋₈ aliphatic residue; O—C(═O)aryl; O—C(═O)heteroaryl; NH₂; NH—C₁₋₈ aliphatic residue; NH—(C₁₋₈ aliphatic group)-OH; N(C₁₋₈ aliphatic residue)[(C₁₋₈ aliphatic group)-OH]; N(C₁₋₈ aliphatic residue)₂; NH—C(═O)—C₁₋₈ aliphatic residue; NH—S(═O)₂—C₁₋₈ aliphatic residue; N(C₁₋₈ aliphatic residue)[S(═O)₂—C₁₋₈ aliphatic residue]; NH—S(═O)₂—NH₂; NH—C(═O)-aryl; NH—C(═O)-heteroaryl; SH; S—C₁₋₈ aliphatic residue; SCF₃; S-benzyl; S-aryl; S-heteroaryl; S(═O)₂—C₁₋₈ aliphatic residue; S(═O)₂ aryl; S(═O)₂ heteroaryl; S(═O)₂OH; S(═O)₂O—C₁₋₈ aliphatic residue; S(═O)₂O-aryl; S(═O)₂O-heteroaryl; S(═O)₂—NH—C₁₋₈ aliphatic residue; S(═O)₂—NH-aryl; and S(═O)₂—NH-heteroaryl.

Most preferred substituents of “aliphatic residue” and “aliphatic group” are selected from the group consisting of F; Cl; Br; I; CF₃; C(═O)—NH₂; C(═O)NH—C₁₋₈ aliphatic residue; C(═O)N(C₁₋₈ aliphatic residue)₂; OH; O—C₁₋₈ aliphatic residue; O—(C₁₋₈ aliphatic residue)-OH; O—(C₁₋₈ aliphatic group)-O—C₁₋₈ aliphatic residue; NH₂; NH—C₁₋₈ aliphatic residue; N(C₁₋₈ aliphatic residue)₂; NH—(C₁₋₈ aliphatic group)-OH; N(C₁₋₈ aliphatic residue)[(C₁₋₈ aliphatic group)-OH]; NH—C(═O)—C₁₋₈ aliphatic residue; NH—S(═O)₂—C₁₋₈ aliphatic residue; N(C₁₋₈ aliphatic residue)[S(═O)₂—C₁₋₈ aliphatic residue]; NH—S(═O)₂—NH₂; SH; S—C₁₋₈ aliphatic residue; S(═O)₂—C₁₋₈ aliphatic residue; and S(═O)₂—NH—C₁₋₈ aliphatic residue.

Preferred substituents of “cycloaliphatic residue” and “heterocycloaliphatic residue” are selected from the group consisting of F; Cl; Br; I; NO₂; CF₃; CN; ═O; ═NH; R⁰; C(═O)(R⁰ or H); C(═O)O(R⁰ or H); C(═O)N(R⁰ or H)₂; OH; OR⁰; O—C(═O)—R⁰; O—(C₁₋₈ alkyl)-OH; O—(C₁₋₈ alkyl)-O—C₁₋₈ alkyl; OCF₃; N(R⁰ or H)₂; N(R⁰ or H)—C(═O)—R⁰; N(R⁰ or H)—S(═O)₂—R⁰; N(R⁰ or H)—C(═O)—N(R⁰ or H)₂; SH; SCF₃; SW; S(═O)₂R⁰; S(═O)₂O(R⁰ or H) and S(═O)₂—N(R⁰ or H)₂.

Particularly preferred substituents of “cycloaliphatic residue” and “heterocycloaliphatic residue” are selected from the group consisting of F; Cl; Br; I; NO₂; CF₃; CN; ═O; C₁₋₈ aliphatic residue; aryl; heteroaryl; C₃₋₆ cycloaliphatic residue; 3 to 6 membered heterocycloaliphatic residue; aryl, heteroaryl, C₃₋₆ cycloaliphatic residue or 3 to 6 membered heterocycloaliphatic bridged via a C₁ aliphatic group; CHO; C(═O)—C₁₋₈ aliphatic residue; C(═O)aryl; C(═O)heteroaryl; CO₂H; C(═O)O—C₁₋₈ aliphatic residue; C(═O)O-aryl; C(═O)O-heteroaryl; CONH₂; C(═O)NH—C₁₋₈ aliphatic residue; C(═O)N(C₁₋₈ aliphatic residue)₂; C(═O)NH-aryl; C(═O)N(aryl)₂; C(═O)NH-heteroaryl; C(═O)N(heteroaryl)₂; C(═O)N(C₁₋₈ aliphatic residue)(aryl); C(═O)N(C₁₋₈ aliphatic residue)(heteroaryl); C(═O)N(heteroaryl)(aryl); OH; O—C₁₋₈ aliphatic residue; OCF₃; O—(C₁₋₈ aliphatic residue)-OH; O—(C₁₋₈ aliphatic residue)-O—C₁₋₈ aliphatic residue; O-benzyl; O-aryl; O-heteroaryl; O—C(═O)—C₁₋₈ aliphatic residue; O—C(═O)aryl; O—C(═O)heteroaryl; NH₂, NH—C₁₋₈ aliphatic residue; N(C₁₋₈ aliphatic residue)₂; NH—C(═O)—C₁₋₈ aliphatic residue; NH—C(═O)-aryl; NH—C(═O)-heteroaryl; SH; S—C₁₋₈ aliphatic residue; SCF₃; S-benzyl; S-aryl; S-heteroaryl; S(═O)₂—C₁₋₈ aliphatic residue; S(═O)₂ aryl; S(═O)₂ heteroaryl; S(═O)₂OH; S(═O)₂O—C₁₋₈ aliphatic residue; S(═O)₂O-aryl; S(═O)₂O-heteroaryl; S(═O)₂—NH—C₁₋₈ aliphatic residue; S(═O)₂—NH-aryl; and S(═O)₂—NH-heteroaryl.

In relation to the terms “aryl” and “heteroaryl”, the term “mono- or polysubstituted” refers in the sense of this invention, with respect to the corresponding residues or groups, to the single substitution or multiple substitution, e.g. disubstitution, trisubstitution, tetrasubstitution, or pentasubstitution, of one or more hydrogen atoms each independently of one another by at least one substituent selected from the group consisting of F; Cl; Br; I; NO₂; CN; CF₃; CF₂H; CFH₂; CF₂Cl; CFCl₂; C(═O)—H; C(═O)—R⁰; C(═O)—OH; C(═O)—OR⁰; CO—NH₂; C(═O)—NHR⁰; C(═O)—N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂—OH; O—S(═O)₂—OR⁰; O—S(═O)₂—NH₂; O—S(═O)₂—NHR⁰; O—S(═O)₂—N(R⁰)₂; NH₂; NHR⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂—OH; NH—S(═O)₂—R⁰; NH—S(═O)₂—OR⁰; NH—S(═O)₂—NH₂; NH—S(═O)₂—NHR⁰; NH—S(═O)₂—N(R⁰)₂; NR⁰—S(═O)₂—OH; NR⁰—S(═O)₂R⁰; NR⁰—S(═O)₂—OR⁰; NR⁰—S(═O)₂—NH₂; NR⁰—S(═O)₂—NHR⁰; NR⁰—S(═O)₂—N(R⁰)₂; SH; SCF₃; SCF₂H; SCFH₂; SCF₂Cl; SCFCl₂; SR⁰; S(═O)—R⁰; S(═O)₂—R⁰; S(═O)₂—OH; S(═O)₂—OR⁰; S(═O)₂—NH₂; S(═O)₂—NHR⁰; and S(═O)₂—N(R⁰)₂;

Preferred substituents of “aryl” and “heteroaryl” are selected from the group consisting of F; Cl; Br; I; NO₂; CF₃; CN; R⁰; C(═O)(R⁰ or H); C(═O)O(R⁰ or H); C(═O)N(R⁰ or H)₂; OH; OR⁰; O—C(═O)—R⁰; O—(C₁₋₈ alkyl)-O—C₁₋₈ alkyl; OCF₃; N(R⁰ or H)₂; N(R⁰ or H)—C(═O)—R⁰; N(R⁰ or Hy S(═O)₂—R⁰; N(R⁰ or H)—C(═O)—N(R⁰ or H)₂; SH; SCF₃; SR⁰; S(═O)₂R⁰; S(═O)₂O(R⁰ or H) and S(═O)₂—N(R⁰ or H)₂.

Particularly preferred substituents of “aryl” and “heteroaryl” are selected from the group consisting of F; Cl; Br; I; NO₂; CF₃; CN; C₁₋₈ aliphatic residue; aryl; heteroaryl; C₃₋₆ cycloaliphatic residue; 3 to 6 membered heterocycloaliphatic residue; aryl, heteroaryl, C₃₋₆ cycloaliphatic residue or 3 to 6 membered heterocycloaliphatic bridged via a C₁₋₄ aliphatic group; CHO; C(═O)—C₁₋₈ aliphatic residue; C(═O)aryl; C(═O)heteroaryl; CO₂H; C(═O)O—C₁₋₈ aliphatic residue; C(═O)O-aryl; C(═O)O-heteroaryl; CONH₂; C(═O)NH—C₁₋₈ aliphatic residue; C(═O)N(C₁₋₈ aliphatic residue)₂; C(═O)NH-aryl; C(═O)N(aryl)₂; C(═O)NH-heteroaryl; C(═O)N(heteroaryl)₂; C(═O)N(C₁₋₈ aliphatic residue)(aryl); C(═O)N(C₁₋₈ aliphatic residue)(heteroaryl); C(═O)N(heteroaryl)(aryl); OH; O—C₁₋₈ aliphatic residue; OCF₃; O—(C₁₋₈ aliphatic residue)-OH; O—(C₁₋₈ aliphatic residue)-O—C₁₋₈ aliphatic residue; O-benzyl; O-aryl; O-heteroaryl; O—C(═O)—C₁₋₈ aliphatic residue; O—C(═O)aryl; O—C(═O)heteroaryl; NH₂, NH—C₁₋₈ aliphatic residue; N(C₁₋₈ aliphatic residue)₂; NH—C(═O)—C₁₋₈ aliphatic residue; NH—C(═O)-aryl; NH—C(═O)-heteroaryl; SH; S—C₁₋₈ aliphatic residue; SCF₃; S-benzyl; S-aryl; S-heteroaryl; S(═O)₂—C₁₋₈ aliphatic residue; S(═O)₂ aryl; S(═O)₂ heteroaryl; S(═O)₂OH; S(═O)₂O—C₁₋₈ aliphatic residue; S(═O)₂O-aryl; S(═O)₂O-heteroaryl; S(═O)₂—NH—C₁₋₈ aliphatic residue; S(═O)₂—NH-aryl; and S(═O)₂—NH-heteroaryl.

The compounds according to the invention are defined by substituents, for example by R¹, R² and R³ (1^{st} generation substituents) which are for their part if appropriate themselves substituted (2^{nd} generation substituents). Depending on the definition, these substituents of the substituents can for their part be resubstituted (3^{rd} generation substituents). If, for example, R¹=a C₁₋₄ aliphatic residue (1^{st} generation substituent), then the C₁₋₄ aliphatic residue can for its part be substituted, for example with a NH—C₁₋₄ aliphatic residue (2^{nd} generation substituent). This produces the functional group R¹═(C₁₋₄ aliphatic residue-NH—C₁₋₄ aliphatic residue). The NH—C₁₋₄ aliphatic residue can then for its part be resubstituted, for example with Cl (3^{rd} generation substituent). Overall, this produces the functional group R¹═C₁₋₄ aliphatic residue-NH—C₁₋₄ aliphatic residue, wherein the C₁₋₄ aliphatic residue of the NH—C₁₋₄ aliphatic residue is substituted by Cl.

However, in a preferred embodiment, the 3^{rd} generation substituents may not be resubstituted, i.e. there are then no 4^{th} generation substituents.

In another preferred embodiment, the 2^{nd} generation substituents may not be resubstituted, i.e. there are then not even any 3^{rd} generation substituents. In other words, in this embodiment, in the case of general formula (I), for example, the functional groups for R¹ to R⁹ can each if appropriate be substituted; however, the respective substituents may then for their part not be resubstituted.

In some cases, the compounds according to the invention are defined by substituents which are or carry an aryl or heteroaryl residue, respectively unsubstituted or mono- or polysubstituted, or which form together with the carbon atom(s) or heteroatom(s) connecting them, as the ring member or as the ring members, a ring, for example an aryl or heteroaryl, in each case unsubstituted or mono- or polysubstituted. Both these aryl or heteroaryl residues and the (hetero)aromatic ring systems formed in this way can if appropriate be condensed with a cycloaliphatic, preferably a C₃₋₆ cycloaliphatic residue, or heterocycloaliphatic residue, preferably a 3 to 6 membered heterocycloaliphatic residue, or with aryl or heteroaryl, e.g. with a C₃₋₆ cycloaliphatic residue such as cyclopentyl, or a 3 to 6 membered heterocycloaliphatic residue such as morpholinyl, or an aryl such as phenyl, or a heteroaryl such as pyridyl, wherein the cycloaliphatic or heterocycloaliphatic residues, aryl or heteroaryl residues condensed in this way can for their part be respectively unsubstituted or mono- or polysubstituted.

In some cases, the compounds according to the invention are defined by substituents which are or carry a cycloaliphatic residue or a heterocycloaliphatic residue, respectively, in each case unsubstituted or mono- or polysubstituted, or which form together with the carbon atom(s) or heteroatom(s) connecting them, as the ring member or as the ring members, a ring, for example a cycloaliphatic or a heterocycloaliphatic ring system. Both these cycloaliphatic or heterocycloaliphatic ring systems and the (hetero)cycloaliphatic ring systems formed in this manner can if appropriate be condensed with aryl or heteroaryl, preferably selected from the group consisting of phenyl, pyridyl and thienyl, or with a cycloaliphatic residue, preferably a C₃₋₆ cycloaliphatic residue, or a heterocycloaliphatic residue, preferably a 3 to 6 membered heterocycloaliphatic residue, e.g. with an aryl such as phenyl, or a heteroaryl such as pyridyl, or a cycloaliphatic residue such as cyclohexyl, or a heterocycloaliphatic residue such as morpholinyl, wherein the aryl or heteroaryl residues or cycloaliphatic or heterocycloaliphatic residues condensed in this way can for their part be respectively unsubstituted or mono- or polysubstituted.

Within the scope of the present invention, the symbol

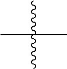

used in the formulae denotes a link of a corresponding residue to the respective superordinate general structure.

If a residue occurs multiply within a molecule, then this residue can have respectively different meanings for various substituents: if, for example, both R¹ and R² denote a 3 to 6 membered heterocycloaliphatic residue, then the 3 to 6 membered heterocycloaliphatic residue can e.g. represent morpholinyl for R¹ and can represent piperazinyl for R².

If a residue occurs multiply within a molecule, such as for example the residue R⁰, then this residue can have respectively different meanings for various substituents.

The term “(R⁰ or H)” within a residue means that R⁰ and H can occur within this residue in any possible combination. Thus, for example, the residue “N(R⁰ or H)₂” can represent “NH₂”, “NHR⁰” and “N(R⁰)₂”. If, as in the case of)“N(R⁰)₂”, R⁰ occurs multiply within a residue, then R⁰ can respectively have the same or different meanings: in the present example of)“N(R⁰)₂”, R⁰ can for example represent aryl twice, thus producing the functional group “N(aryl)₂”, or R⁰ can represent once aryl and once a C₁₋₁₀ aliphatic residue, thus producing the functional group “N(aryl)(C₁₋₁₀ aliphatic residue)”.

The term “inhibition” in the sense of this invention means to retard or lessen.

The terms “salt formed with a physiologically compatible acid” or “salt of physiologically acceptable acids” refers in the sense of this invention to salts of the respective active ingredient with inorganic or organic acids which are physiologically compatible—in particular when used in human beings and/or other mammals. Examples of physiologically acceptable acids are: hydrochloric acid, hydrobromic acid, sulphuric acid, methanesulphonic acid, p-toluenesulphonic acid, carbonic acid, formic acid, acetic acid, oxalic acid, succinic acid, tartaric acid, mandelic acid, fumaric acid, maleic acid, lactic acid, citric acid, glutamic acid, saccharic acid, monomethylsebacic acid, 5-oxoproline, hexane-1-sulphonic acid, nicotinic acid, 2, 3 or 4-aminobenzoic acid, 2,4,6-trimethylbenzoic acid, α-lipoic acid, acetyl glycine, hippuric acid, phosphoric acid, aspartic acid. Citric acid and hydrochloric acid are particularly preferred.

The terms “salt formed with a physiologically compatible base” or “salt of physiologically acceptable bases” refers in the sense of this invention to salts of the respective compound according to the invention—as an anion, e.g. upon deprotonation of a suitable functional group—with at least one cation or base—preferably with at least one inorganic cation—which are physiologically acceptable—in particular when used in human beings and/or other mammals. Particularly preferred are the salts of the alkali and alkaline earth metals, in particular (mono-) or (di)sodium, (mono-) or (di)potassium, magnesium or calcium salts, but also ammonium salts [NHₓR₄₋ₓ]⁺, in which x=0, 1, 2, 3 or 4 and R represents a branched or unbranched C₁₋₄ aliphatic residue.

In one embodiment of the compounds of formula (I) according to the present invention 1 or 2 of variables A¹, A², A³, A⁴ and A⁵ represent a nitrogen atom.

In another embodiment of the compounds of formula (I) according to the present invention 1 of variables A¹, A², A³, A⁴ and A⁵ represents a nitrogen atom.

In still another embodiment of the compounds of formula (I) according to the present invention A² represents a nitrogen atom, A¹ denotes C—R⁵, A³ denotes C—R⁷, A⁴ denotes C—R⁸ and A⁵ denotes C—R⁹

In a preferred embodiment of the compounds according to the present invention of general formula (I), n represents 1, 2, 3 or 4, preferably 1, 2 or 3, particularly preferably 1 or 2, most particularly preferably 1.

In the compounds according to the present invention Y preferably represents O or S, more preferably O.

In a further preferred embodiment of the compounds according to the present invention of general formula (I), X represents N.

In another further preferred embodiment of the compounds according to the present invention of general formula (I), X represents CH.

In another preferred embodiment of the compounds of general formula (I) according to the present invention
R¹ represents a C₁ aliphatic residue, unsubstituted or mono- or polysubstituted with one or more substituents each selected independently of one another from the group consisting of F, Cl, Br, I, NO₂, CN, OH, ═O, O—C₁₋₄ alkyl, OCF₃, C(═O)—OH, CF₃, NH₂, NH(C₁₋₄ alkyl), N(C₁₋₄ alkyl)₂, SH, S—C₁₋₄ alkyl, SCF₃ S(═O)₂OH, benzyl, phenyl, pyridyl and thienyl, wherein benzyl, phenyl, pyridyl, thienyl can be respectively unsubstituted or mono- or polysubstituted with one or more substituents selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH, or represents
   a C₃₋₆ cycloaliphatic residue or a 3 to 6 membered heterocycloaliphatic residue, in each case unsubstituted or mono- or polysubstituted with one or more substituents selected independently of one another from the group consisting of F, Cl, Br, I, OH, ═O, C₁₋₄ alkyl, O—C₁₋₄ alkyl, OCF₃, C(═O)—OH and CF₃.

Preferably,
R¹ represents a C₁ aliphatic residue, unsubstituted or mono- or polysubstituted with one or more substituents each selected independently of one another from the group consisting of F, Cl, Br, I, CN, OH, ═O, O—C₁₋₄ alkyl, OCF₃, CF₃, NH₂, NH(C₁₋₄ alkyl), N(C₁₋₄ alkyl)₂, SH, S—C₁₋₄ alkyl, SCF₃, or represents
   a C₃₋₆ cycloaliphatic residue or a 3 to 6 membered heterocycloaliphatic residue, in each case unsubstituted or mono- or polysubstituted with one or more substituents selected independently of one another from the group consisting of F, Cl, Br, I, OH, ═O, C₁₋₄ alkyl, O—C₁₋₄ alkyl, OCF₃ and CF₃.

More preferably
R¹ represents a C₁ aliphatic residue, unsubstituted or mono- or polysubstituted with one or more substituents each selected independently of one another from the group consisting of F, Cl, Br, I, and OH, or represents
   a C₃₋₆ cycloaliphatic residue or a 3 to 6 membered heterocycloaliphatic residue, in each case unsubstituted or mono- or polysubstituted with one or more substituents selected independently of one another from the group consisting of F, Cl, Br, I, and OH.

Even more preferably
R¹ represents a C₁₋₄ aliphatic residue, unsubstituted or mono- or polysubstituted with one or more substituents each selected independently of one another from the group consisting of F, Cl, Br, I, or represents
   a C₃₋₆ cycloaliphatic residue or a 3 to 6 membered heterocycloaliphatic residue, in each case unsubstituted.

Still more preferably
R¹ is selected from the group consisting of CF₃, methyl, ethyl, n-propyl, isopropyl, n-butyl, sec.-butyl, and tert.-butyl, or
   is selected from the group consisting of cyclopropyl, cyclobutyl, cyclopentyl, and cyclohexyl.

Particularly preferably,

R¹ is selected from the group consisting of tert-Butyl, CF₃, cyclopropyl, cyclobutyl, cyclopentyl, and cyclohexyl, preferably from the group consisting of tert-Butyl, CF₃ and cyclopropyl, more preferably from the group consisting of tert-Butyl and CF₃.

In yet another preferred embodiment of the compound of general formula (I) according to the present invention
R² represents a C₁₋₁₀ aliphatic residue, a O—C₁₋₁₀ aliphatic residue, a S—C₁₋₁₀ aliphatic residue, a NH—C₁₋₁₀ aliphatic residue, a N(C₁₋₁₀ aliphatic residue)₂, wherein in each case independently of one another the C₁₋₁₀ aliphatic residue can be unsubstituted or mono- or polysubstituted with one or more substituents each selected independently of one another from the group consisting of F, Cl, Br, I, NO₂, CN, OH, ═O, O—C₁₋₄ alkyl, OCF₃, CF₃, NH₂, NH(C₁₋₄ alkyl), N(C₁₋₄ alkyl)₂, SH, S—C₁₋₄ alkyl, SCF₃, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH;
   wherein each of the aforementioned residues can in each case be optionally bridged via a C₁₋₈ aliphatic group, which in turn may be unsubstituted or mono- or polysubstituted with one or more substituents each selected independently of one another from the group consisting of F, Cl, Br, I, NO₂, CN, OH, ═O, O—C₁₋₄ alkyl, OCF₃, CF₃, NH₂, NH(C₁₋₄ alkyl), N(C₁₋₄ alkyl)₂, SH, S—C₁₋₄ alkyl and SCF₃,
   or represents a C₃₋₁₀ cycloaliphatic residue, a O—C₃₋₁₀ cycloaliphatic residue, a O—(C₁₋₈ aliphatic group)-C₃₋₁₀ cycloaliphatic residue, a S—C₃₋₁₀ cycloaliphatic residue, a S—(C₁₋₈ aliphatic group)-C₃₋₁₀ cycloaliphatic residue, a NH—C₃₋₁₀ cycloaliphatic residue, a NH—(C₁₋₈ aliphatic group)-C₃₋₁₀ cycloaliphatic residue, a N(C₁₋₁₀ aliphatic residue)(C₃₋₁₀ cycloaliphatic residue), a 3 to 10 membered heterocycloaliphatic residue, O-(3 to 10 membered heterocycloaliphatic residue), O—(C₁₋₈ aliphatic group)-(3 to 10 membered heterocycloaliphatic residue), S-(3 to 10 membered heterocycloaliphatic residue), S—(C₁₋₈ aliphatic group)-(3 to 10 membered heterocycloaliphatic residue), NH-(3 to 10 membered heterocycloaliphatic residue), NH—(C₁₋₈ aliphatic group)-(3 to 10 membered heterocycloaliphatic residue), N(C₁₋₁₀ aliphatic residue)(3 to 10 membered heterocycloaliphatic residue), wherein in each case independently of one another the C₁₋₁₀ aliphatic residue, the C₁₋₈ aliphatic group, the C₃₋₁₀ cycloaliphatic residue and the 3 to 10 membered heterocycloaliphatic residue, respectively, can be unsubstituted or mono- or polysubstituted with one or more substituents each selected independently of one another from the group consisting of F, Cl, Br, I, NO₂, CN, OH, ═O, O—C₁₋₄ alkyl, OCF₃, C₁₋₄ alkyl, CF₃, SH, S—C₁₋₄ alkyl, SCF₃, NH₂, NH(C₁₋₄ alkyl), N(C₁₋₄ alkyl)₂, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH,
   wherein each of the aforementioned residues can in each case be optionally bridged via a C₁₋₈ aliphatic group, which in turn may be unsubstituted or mono- or polysubstituted with one or more substituents each selected independently of one another from the group consisting of F, Cl, Br, I, NO₂, CN, OH, ═O, O—C₁₋₄ alkyl, OCF₃, CF₃, NH₂, NH(C₁₋₄ alkyl), N(C₁₋₄ alkyl)₂, SH, S—C₁₋₄ alkyl and SCF₃,
   or represents aryl, O-aryl, a O—(C₁₋₈ aliphatic group)-aryl, S-aryl, a S—(C₁₋₈ aliphatic group)-aryl, a NH-aryl, a NH—(C₁₋₈ aliphatic group)-aryl, a N(C₁₋₁₀ aliphatic residue)(aryl), heteroaryl, O-heteroaryl, O—(C₁₋₈ aliphatic group)-heteroaryl, S-(heteroaryl), S—(C₁₋₈ aliphatic group)-(heteroaryl), NH-(heteroaryl), NH—(C₁₋₈ aliphatic group)-(heteroaryl), N(C₁₋₁₀ aliphatic residue)(heteroaryl), wherein in each case independently of one another the C₁₋₁₀ aliphatic residue, the C₁₋₈ aliphatic group, aryl and heteroaryl, respectively, can be unsubstituted or mono- or polysubstituted with one or more substituents each selected independently of one another from the group consisting of F, Cl, Br, I, NO₂, CN, OH, ═O, O—C₁₋₄ alkyl, OCF₃, C₁₋₄ alkyl, CF₃, SH, S—C₁₋₄ alkyl, SCF₃, NH₂, NH(C₁₋₄ alkyl), N(C₁₋₄ alkyl)₂, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH, wherein each of the aforementioned residues can in each case be optionally bridged via a C₁₋₈ aliphatic group, which in turn may be unsubstituted or mono- or polysubstituted with one or more substituents each selected independently of one another from the group consisting of F, Cl, Br, I, NO₂, CN, OH, ═O, O—C₁₋₄ alkyl, OCF₃, CF₃, NH₂, NH(C₁₋₄ alkyl), N(C₁₋₄ alkyl)₂, SH, S—C₁₋₄ alkyl and SCF₃.

In a further preferred embodiment of the compound of general formula (I) according to the present invention
R² represents substructure (T1)

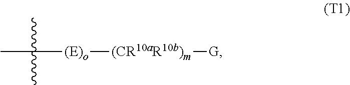

in which
E represents O, S, or NR¹¹,
   wherein R¹¹ represents H or a C₁₋₄ aliphatic residue, unsubstituted or mono- or polysubstituted with one or more substituents each selected independently of one another from the group consisting of F, Cl, Br, I, OH, O—C₁₋₄ alkyl, OCF₃, NH₂, NH—C₁₋₄ alkyl and N(C₁₋₄ alkyl)₂;
o represents 0 or 1;
R^{10a} and R^{10b} each independently of one another represent H; F; Cl; Br; I; or a C₁₋₄ aliphatic residue, unsubstituted or mono- or polysubstituted with one or more substituents each selected independently of one another from the group consisting of F, Cl, Br, I, OH, O—C₁₋₄ alkyl, OCF₃, NH₂, NH—C₁₋₄ alkyl and N(C₁₋₄ alkyl)₂;
m represents 0, 1, 2, 3 or 4, preferably 0, 1 or 2, more preferably 0 or 1;
G represents a C₁₋₄ aliphatic residue, unsubstituted or mono- or polysubstituted with one or more substituents each selected independently of one another from the group consisting of F, Cl, Br, I, NO₂, CN, OH, ═O, O—C₁₋₄ alkyl, O—C₁₋₄ alkylen-O—C₁₋₄ alkyl, OCF₃, CF₃, NH₂, NH(C₁₋₄ alkyl), N(C₁₋₄ alkyl)₂, SH, S—C₁₋₄ alkyl, SCF₃, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH;
   or represents a C₃₋₁₀ cycloaliphatic residue or a 3 to 10 membered heterocyclo-aliphatic residue, in each case unsubstituted or mono- or polysubstituted with one or more substituents each selected independently of one another from the group consisting of F, Cl, Br, I, NO₂, CN, OH, ═O, O—C₁₋₄ alkyl, OCF₃, C₁₋₄ alkyl, CF₃, SH, S—C₁₋₄ alkyl, SCF₃, NH₂, NH(C₁₋₄ alkyl), N(C₁₋₄ alkyl)₂, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH;
   or represents an aryl or heteroaryl, unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, CF₃, SH, S—C₁₋₄ alkyl, SCF₃, NH₂, NH(C₁₋₄ alkyl), N(C₁₋₄ alkyl)₂, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH.

In a particularly preferred embodiment of the compound according to the invention of general formula (I), the residue
R¹ represents substructure (T1), wherein o denotes 0.

Preferably, the residue
R² represents substructure (T1) in which
E represents O, S, or NR¹¹,
   wherein R¹¹ represents H or an unsubstituted C₁₋₄ aliphatic residue, preferably selected from the group consisting of methyl, ethyl, n-propyl, isopropyl, n-butyl, sec.-butyl and tert.-butyl;
o represents 0 or 1;
R^{10a} and R^{10b} each independently of one another represent H, F, Cl, Br, I or an unsubstituted C₁₋₄ aliphatic residue, preferably selected from the group consisting of methyl, ethyl, n-propyl, isopropyl, n-butyl, sec.-butyl, tert.-butyl;
m represents 0, 1 or 2, more preferably 0 or 1;
G represents a C₁₋₄ aliphatic residue, unsubstituted or mono- or polysubstituted with one or more substituents each selected independently of one another from the group consisting of F, Cl, Br, I, OH, O—C₁₋₄ alkyl, O—C₁₋₄ alkylen-O—C₁₋₄ alkyl, OCF₃, CF₃, NH₂, NH(C₁₋₄ alkyl), N(C₁₋₄ alkyl)₂, SH, S—C₁₋₄ alkyl, and SCF₃;
   or represents a C₃₋₁₀ cycloaliphatic residue or a 3 to 10 membered heterocyclo-aliphatic residue, in each case unsubstituted or mono- or polysubstituted with one or more substituents each selected independently of one another from the group consisting of F, Cl, Br, I, OH, O—C₁₋₄ alkyl, OCF₃, C₁₋₄ alkyl, CF₃, SH, S—C₁₋₄ alkyl, SCF₃, NH₂, NH(C₁₋₄ alkyl), N(C₁₋₄ alkyl)₂, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH;
   or represents an aryl or heteroaryl, unsubstituted or mono- or polysubstituted with one or more substituents each selected independently of one another from the group consisting of F, Cl, Br, I, NO₂, CN, OH, O—C₁₋₄ alkyl, OCF₃, CF₃, SH, S—C₁₋₄ alkyl, C₁₋₄ alkyl, SCF₃, NH₂, NH(C₁₋₄ alkyl), N(C₁₋₄ alkyl)₂, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH.

More preferably, the residue
R² represents substructure (T1) in which
E represents O, S, or NR¹¹,
   wherein R¹¹ represents H or is selected from the group consisting of methyl, ethyl, n-propyl, isopropyl, n-butyl, sec.-butyl and tert.-butyl;
o represents 0 or 1;
R^{10a} and R^{10b} are independently of one another selected from the group consisting of H, methyl, ethyl, n-propyl, isopropyl, n-butyl, sec.-butyl, tert.-butyl;
m represents 0, 1 or 2, more preferably 0 or 1;
G represents methyl, ethyl, n-propyl, isopropyl, n-butyl, sec.-butyl, or tert.-butyl, in each case unsubstituted or mono- or polysubstituted with one or more substituents each selected independently of one another from the group consisting of F, Cl, Br, I, OH, O—C₁₋₄ alkyl and O—C₁₋₄ alkylen-O—C₁₋₄ alkyl;
   or represents a C₃₋₆ cycloaliphatic residue, preferably selected from the group consisting of cyclopropyl, cyclobutyl, cyclopentyl and cyclohexyl, or a 3 to 6 membered heterocycloaliphatic residue, preferably selected from the group consisting of pyrrolidinyl, piperazinyl, 4-methylpiperazinyl, piperidinyl, morpholinyl, tetrahydropyrrolyl, tetrahydropyranyl, tetrahydro-2H-pyran-4-yl, tetrahydroquinolinyl, tetrahydroisoquinolinyl, dihydroquinolinyl, dihydropyrrolyl, dihydropyridinyl, dihydroisoquinolinyl, tetrahydropyridinyl and thiomorpholinyl, in each case unsubstituted or mono- or polysubstituted with one or more substituents each selected independently of one another from the group consisting of F, Cl, Br, I, OH, O—C₁₋₄ alkyl, OCF₃, C₁₋₄ alkyl, CF₃, NH₂, NH(C₁₋₄ alkyl), N(C₁₋₄ alkyl)₂, and phenyl, wherein phenyl can be unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, CF₃, and SCF₃;
   or represents an aryl or heteroaryl, preferably phenyl or pyridyl, in each case unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, CF₃, SH, S—C₁₋₄ alkyl, SCF₃, NH₂, NH(C₁₋₄ alkyl), N(C₁₋₄ alkyl)₂, and phenyl wherein phenyl can be unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, CF₃, and SCF₃.

Even more preferably, the residue
R² represents substructure (T1) in which
E represents O, S, or NR¹¹;
   wherein R¹¹ represents H or is selected from the group consisting of methyl, ethyl, n-propyl, isopropyl, n-butyl, sec.-butyl and tert.-butyl
o represents 0 or 1;
R^{10a} and R^{10b} are independently of one another selected from the group consisting of H, methyl and ethyl,
m represents 0, 1 or 2, more preferably 0 or 1;
G represents methyl, ethyl, n-propyl, isopropyl, n-butyl, sec.-butyl, or tert.-butyl, in each case unsubstituted;
   or is selected from the group consisting of cyclopropyl, cyclobutyl, cyclopentyl and cyclohexyl, or is selected from the group consisting of pyrrolidinyl, piperazinyl, 4-methylpiperazinyl, piperidinyl, tetrahydropyranyl, tetrahydro-2H-pyran-4-yl, morpholinyl and thiomorpholinyl, in each case unsubstituted or mono- or polysubstituted with one or more substituents each selected independently of one another from the group consisting of F, Cl, Br, I, OH, C₁₋₄ alkyl, O—C₁₋₄ alkyl, OCF₃, CF₃, NH₂, NH(C₁₋₄ alkyl), N(C₁₋₄ alkyl)₂, and phenyl, wherein phenyl can be unsubstituted or mono- or polysubstituted with one or more substituents each selected independently of one another from the group consisting of F, Cl, Br, I, CN, OH, O—C₁₋₄ alkyl, OCF₃, C₁₋₄ alkyl, CF₃, and SCF₃;
   or represents an aryl or heteroaryl, preferably phenyl or pyridyl, in each case unsubstituted or mono- or polysubstituted with one or more substituents each selected independently of one another from the group consisting of F, Cl, Br, I, CN, OH, O—C₁₋₄ alkyl, OCF₃, C₁₋₄ alkyl, CF₃, SCF₃, NH₂, NH(C₁₋₄ alkyl), N(C₁₋₄ alkyl)₂, and phenyl wherein phenyl can be unsubstituted or mono- or polysubstituted with one or more substituents each selected independently of one another from the group consisting of F, Cl, Br, I, CN, OH, O—C₁₋₄ alkyl, OCF₃, C₁₋₄ alkyl, CF₃, and SCF₃.

Most preferred,
R² represents phenyl, unsubstituted or mono- or polysubstituted with one or more substituents each selected independently of one another from the group consisting of F, Cl, Br, I, OH, O—CH₃, CH₃, CH(CH₃)₂, N(CH₃)₂, tert.-butyl and CF₃, preferably phenyl mono- or disubstituted with one or two substituents each selected independently of one another from the group consisting of F, Cl, Br, I, O—CH₃, CH₃, CH(CH₃)₂, N(CH₃)₂, tert.-butyl and CF₃, more preferably phenyl mono-substituted in meta position with one substituent selected from the group consisting of F, Cl, CH₃, OCH₃, CH(CH₃)₂ and N(CH₃)₂.

In yet another preferred embodiment of the compound of general formula (I) according to the present invention
R³ represents H or a C₁₋₄ aliphatic residue, unsubstituted or mono- or polysubstituted with one or more substituents each selected independently of one another from the group consisting of F, Cl, Br, I, CN, OH, ═O, O—C₁₋₄ alkyl, OCF₃, CF₃, NH₂, NH(C₁₋₄ alkyl), N(C₁₋₄ alkyl)₂, SH, S—C₁₋₄ alkyl and SCF₃.

Preferably,
R³ represents H or a C₁₋₄ aliphatic residue, unsubstituted or mono- or polysubstituted with one or more substituents each selected independently of one another from the group consisting of F, Cl, Br, I and OH.

More preferably,
R³ represents H or an unsubstituted C₁₋₄ aliphatic residue, preferably selected from the group consisting of methyl, ethyl, n-propyl, isopropyl, n-butyl, sec.-butyl, and tert.-butyl.

In particular,
R³ is selected from the group consisting of H, methyl and ethyl, preferably denotes H or methyl, more preferably represents H.

Preferred is also an embodiment of the compound of general formula (I) according to the present invention, wherein
R^{4a} represents H or a C₁₋₄ aliphatic residue, unsubstituted or mono- or polysubstituted with at least one substituent selected from the group consisting of F, Cl, Br, I, NO₂, CN, OH, ═O, O—C₁₋₄ alkyl, OCF₃, C(═O)—OH, CF₃, NH₂, NH(C₁₋₄ alkyl), N(C₁₋₄ alkyl)₂, SH, S—C₁₋₄ alkyl, SCF₃ S(═O)₂OH, benzyl, phenyl, pyridyl and thienyl, wherein benzyl, phenyl, pyridyl, thienyl can be respectively unsubstituted or mono- or polysubstituted with one or more substituents selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH,
   or represents a C₃₋₆ cycloaliphatic residue, unsubstituted or mono- or polysubstituted with at least one substituent selected from the group consisting of F, Cl, Br, I, NO₂, CN, OH, ═O, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ S(═O)₂OH, benzyl, phenyl, pyridyl and thienyl, wherein benzyl, phenyl, pyridyl, thienyl can be respectively unsubstituted or mono- or polysubstituted with one or more substituents selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH,
   or denotes an aryl, unsubstituted or mono- or polysubstituted with at least one substituent selected from the group consisting of F, Cl, Br, I, NO₂, CN, OH, O—C₁₋₄ alkyl, OCF₃, C₁₋₄ alkyl, C(═O)—OH, CF₃, CF₂H, CFH₂, CF₂Cl, CFCl₂, NH₂, NH(C₁₋₄ alkyl), N(C₁₋₄ alkyl)₂, SH, S—C₁₋₄ alkyl, SCF₃, S(═O)₂OH and NH—S(═O)₂—C₁₋₄ alkyl,
R^{4b} represents H or a C₁₋₄ aliphatic residue, unsubstituted or mono- or polysubstituted with at least one substituent selected from the group consisting of F, Cl, Br, I, NO₂, CN, OH, ═O, O—C₁₋₄ alkyl, OCF₃, C(═O)—OH, CF₃, NH₂, NH(C₁₋₄ alkyl), N(C₁₋₄ alkyl)₂, SH, S—C₁₋₄ alkyl, SCF₃ S(═O)₂OH, benzyl, phenyl, pyridyl and thienyl, wherein benzyl, phenyl, pyridyl, thienyl can be respectively unsubstituted or mono- or polysubstituted with one or more substituents selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH
or
R^{4a} and R^{4b} together with the carbon atom connecting them form a C₃₋₆ cycloaliphatic residue, unsubstituted or mono- or polysubstituted with at least one substituent selected from the group consisting of F, Cl, Br, I, NO₂, CN, OH, ═O, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ S(═O)₂OH, benzyl, phenyl, pyridyl and thienyl, wherein benzyl, phenyl, pyridyl, thienyl can be respectively unsubstituted or mono- or polysubstituted with one or more substituents selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH.

Preferably,
R^{4a} represents H or a C₁₋₄ aliphatic residue, unsubstituted or mono- or polysubstituted with at least one substituent selected from the group consisting of F, Cl, Br, I, NO₂, CN, OH, ═O, O—C₁₋₄ alkyl, OCF₃, C(═O)—OH, CF₃, NH₂, NH(C₁₋₄ alkyl), N(C₁₋₄ alkyl)₂, SH, S—C₁₋₄ alkyl, SCF₃ and S(═O)₂OH,
   or represents a C₃₋₆ cycloaliphatic residue, unsubstituted or mono- or polysubstituted with at least one substituent selected from the group consisting of F, Cl, Br, I, NO₂, CN, OH, ═O, O—C₁₋₄ alkyl, OCF₃, C(═O)—OH, C₁₋₄ alkyl, CF₃, NH₂, NH(C₁₋₄ alkyl), N(C₁₋₄ alkyl)₂, SH, S—C₁₋₄ alkyl, SCF₃ and S(═O)₂OH,
   or denotes an aryl, unsubstituted or mono- or polysubstituted with at least one substituent selected from the group consisting of F, Cl, Br, I, NO₂, CN, OH, O—C₁₋₄ alkyl, OCF₃, C₁₋₄ alkyl, C(═O)—OH, CF₃, CF₂H, CFH₂, CF₂Cl, CFCl₂, NH₂, NH(C₁₋₄ alkyl), N(C₁₋₄ alkyl)₂, SH, S—C₁₋₄ alkyl, SCF₃, S(═O)₂OH and NH—S(═O)₂—C₁₋₄ alkyl,
R^{4b} represents H or a C₁₋₄ aliphatic residue, unsubstituted or mono- or polysubstituted with at least one substituent selected from the group consisting of F, Cl, Br, I, NO₂, CN, OH, ═O, O—C₁₋₄ alkyl, OCF₃, C(═O)—OH, CF₃, NH₂, NH(C₁₋₄ alkyl), N(C₁₋₄ alkyl)₂, SH, S—C₁₋₄ alkyl, SCF₃ and S(═O)₂OH,
or
R^{4a} and R^{4b} together with the carbon atom connecting them form a C₃₋₆ cycloaliphatic residue, unsubstituted or mono- or polysubstituted with at least one substituent selected from the group consisting of F, Cl, Br, I, NO₂, CN, OH, ═O, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH.

More preferably,
R^{4a} represents H or a C₁₋₄ aliphatic residue, unsubstituted or mono- or polysubstituted with at least one substituent selected from the group consisting of F, Cl, Br, I, OH, ═O, O—C₁₋₄ alkyl, OCF₃, CF₃, and SCF₃,
   or represents a C₃₋₆ cycloaliphatic residue, unsubstituted or mono- or polysubstituted with at least one substituent selected from the group consisting of F, Cl, Br, I, OH, ═O, O—C₁₋₄ alkyl, OCF₃, C₁₋₄ alkyl, CF₃, and SCF₃,
   or denotes an aryl, preferably a phenyl, unsubstituted or mono- or polysubstituted with at least one substituent selected from the group consisting of F, Cl, Br, I, NO₂, CN, OH, O—C₁₋₄ alkyl, OCF₃, C₁₋₄ alkyl, C(═O)—OH, CF₃, CF₂H, CFH₂, CF₂Cl, CFCl₂, NH₂, NH(C₁₋₄ alkyl), N(C₁₋₄ alkyl)₂, SH, S—C₁₋₄ alkyl, SCF₃, S(═O)₂OH and NH—S(═O)₂—C₁₋₄ alkyl,
R^{4b} represents H or a C₁₋₄ aliphatic residue, unsubstituted or mono- or polysubstituted with at least one substituent selected from the group consisting of F, Cl, Br, I, OH, ═O, O—C₁₋₄ alkyl, OCF₃, CF₃, and SCF₃,
or
R^{4a} and R^{4b} together with the carbon atom connecting them form a C₃₋₆ cycloaliphatic residue, unsubstituted or mono- or polysubstituted with at least one substituent selected from the group consisting of F, Cl, Br, I, OH, ═O, O—C₁₋₄ alkyl, OCF₃, C₁₋₄ alkyl, CF₃, and SCF₃.

Even more preferably,
R^{4a} represents H or an unsubstituted C₁₋₄ aliphatic residue, preferably denotes H or is selected from the group consisting of methyl, ethyl, n-propyl, isopropyl, n-butyl, sec.-butyl, and tert.-butyl,
   or represents an unsubstituted C₃₋₆ cycloaliphatic residue, preferably selected from the group consisting of cyclopropyl, cyclobutyl, cyclopentyl and cyclohexyl,
   or denotes a phenyl, unsubstituted or mono- or polysubstituted with at least one substituent selected from the group consisting of F, Cl, Br, I, NO₂, CN, OH, O—C₁₋₄ alkyl, OCF₃, C₁₋₄ alkyl, C(═O)—OH, CF₃, CF₂H, CFH₂, CF₂Cl, CFCl₂, NH₂, NH(C₁₋₄ alkyl), N(C₁₋₄ alkyl)₂, SH, S—C₁₋₄ alkyl, SCF₃, S(═O)₂OH and NH—S(═O)₂—C₁₋₄ alkyl,
R^{4b} represents H or a C₁₋₄ aliphatic residue, unsubstituted or mono- or polysubstituted with at least one substituent selected from the group consisting of F, Cl, Br, I, OH, ═O, O—C₁₋₄ alkyl, OCF₃, CF₃, and SCF₃,
or
R^{4a} and R^{4b} together with the carbon atom connecting them form a C₃₋₆ cycloaliphatic residue, preferably selected from the group consisting of cyclopropyl, cyclobutyl, cyclopentyl and cyclohexyl, unsubstituted or mono- or polysubstituted with at least one substituent selected from the group consisting of F, Cl, Br, I, OH, ═O, O—C₁₋₄ alkyl, OCF₃, C₁₋₄ alkyl, CF₃, and SCF₃.

Still more preferably,
R^{4a} represents H; methyl, ethyl, cyclopropyl, cyclobutyl, cyclopentyl, cyclohexyl, or phenyl, wherein phenyl is unsubstituted or substituted with 1, 2, 3, 4 or 5 substituents independently selected from the group consisting of F, Cl, Br, I, NO₂, CN, CF₃, CF₂H, CFH₂, CF₂Cl, CFCl₂, OH, NH₂, NH(C₁₋₄ alkyl) and N(C₁₋₄ alkyl)(C₁₋₄ alkyl), C₁₋₄ alkyl, and O—C₁₋₄-alkyl;
R^{4b} represents H, methyl, or ethyl,
or R^{4a} and R^{4b} together with the carbon atom connecting them form a cyclopropyl, cyclobutyl, cyclopentyl, or cyclohexyl ring.

Particularly preferred is a compound of general formula (I) according to the present invention, wherein
R^{4a} represents H, methyl, ethyl, cyclopropyl, cyclobutyl, cyclopentyl, cyclohexyl, or phenyl, wherein phenyl is unsubstituted or substituted with 1, 2 or 3 substituents independently selected from the group consisting of F, Cl, Br, CF₃, methyl and methoxy;
R^{4b} represents H, methyl, or ethyl,
or R^{4a} and R^{4b} together with the carbon atom connecting them form cyclopropyl, cyclobutyl, cyclopentyl, or cyclohexyl ring.

Even more particularly preferred is a compound of general formula (I) according to the present invention, wherein
R^{4a} represents H, methyl, or ethyl,
R^{4b} represents H, methyl, or ethyl, preferably H or methyl, more preferably H,
or R^{4a} and R^{4b} together with the carbon atom connecting them form cyclopropyl, cyclobutyl, cyclopentyl, or cyclohexyl ring.

Most preferred is a compound of general formula (I) according to the present invention, wherein
R^{4a} represents H, methyl, or ethyl, more preferably H or methyl
R^{4b} represents H, methyl, or ethyl, preferably H or methyl,

In another preferred embodiment of the compounds according to the present invention the part structure

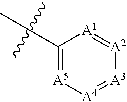

represents a moiety selected from the group consisting of

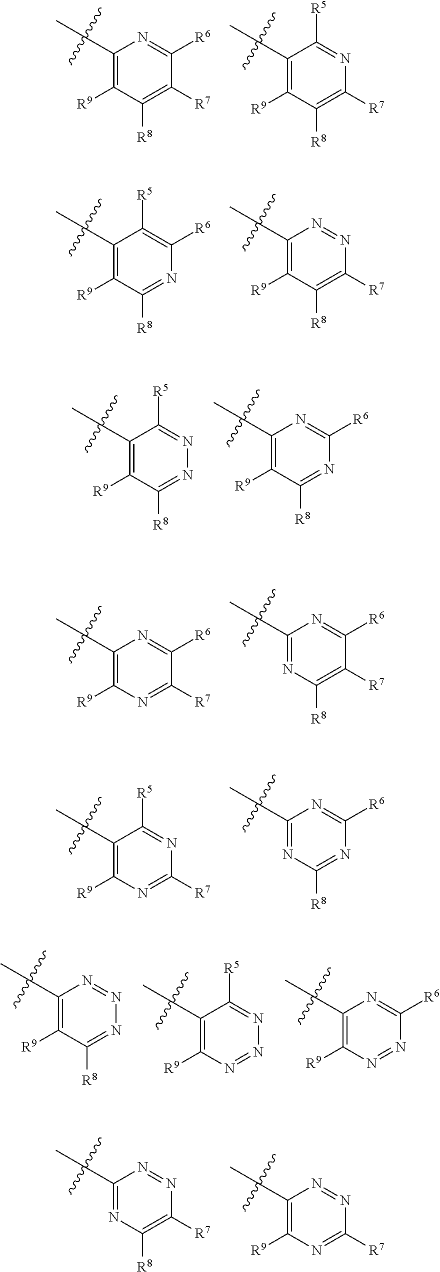

wherein substitutents R⁵, R⁶, R⁷, R⁸ and R⁹ have the meaning as described herein in connection with the compounds according to the invention and preferred embodiments thereof.

A particularly preferred part structure is

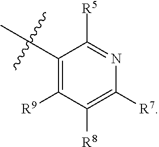

Another particularly preferred part structure is

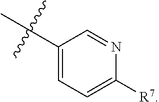

In yet another preferred embodiment of the compound according to the invention of general formula (I),
R⁵, R⁶, R⁷, R⁸ and R⁹ are each independently of one another selected from the group consisting of
   H; F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; NH₂; C(═O)—NH₂; C(═O)—H; C(═O)—OH; S(═O)₂—OH; S(═O)₂—NH₂;
   a C₁₋₁₀ aliphatic residue, (C₁₀ aliphatic group)-OH, (C₁₀ aliphatic group)-O—C₁₋₁₀ aliphatic residue, (C₁₀ aliphatic group)-O—(C₁₋₈ aliphatic group)-OH, (C₁₀ aliphatic group)-O—(C₁₋₈ aliphatic group)-O—C₁₋₁₀ aliphatic residue, a (C₁₋₈ aliphatic group)-NH—C₁₋₁₀ aliphatic residue, a (C₁₋₈ aliphatic group)-NH—(C₁₋₈ aliphatic residue)-OH, a (C₁₋₈ aliphatic group)-N(C₁₋₁₀ aliphatic residue)-(C₁₋₈ aliphatic residue)-OH, a (C₁₋₈ aliphatic group)-NH—S(═O)₂—C₁₋₁₀ aliphatic residue, a (C₁₋₈ aliphatic group)-NH—S(═O)₂—NH₂, a (C₁₋₈ aliphatic group)-S(═O)₂—C₁₋₁₀ aliphatic residue, a C(═O)—C₁₋₁₀ aliphatic residue, a C(═O)—NH—C₁₋₁₀ aliphatic residue,
   a O—C₁₋₁₀ aliphatic residue, a O—(C₁₋₈ aliphatic group)-O—C₁₋₁₀ aliphatic residue, O—(C₁₋₈ aliphatic group)-OH,
   a NH—C₁₋₁₀ aliphatic residue, a N(C₁₋₁₀ aliphatic residue)₂, a NH—[(C₁₋₈ aliphatic group)-O—C₁₋₁₀ aliphatic residue], a NH—[(C₁₋₈ aliphatic group)-OH], a N(C₁₋₁₀ aliphatic residue)[(C₁₋₈ aliphatic group)-OH], a N(C₁₋₁₀ aliphatic residue)[(C₁₋₈ aliphatic group)-O—C₁₋₁₀ aliphatic residue], a NH—C(═O)—C₁₋₁₀ aliphatic residue, a N(C₁₋₁₀ aliphatic residue)[(C(═O)—C₁₋₁₀ aliphatic residue)], a N(C₁₋₁₀ aliphatic residue)[(C₁₋₈ aliphatic group)-O—C₁₋₁₀ aliphatic residue], a N(C₁₋₁₀ aliphatic residue)[(C₁₋₈ aliphatic group)-OH], a NH—S(═O)₂—C₁₋₁₀ aliphatic residue, a N(C₁₋₁₀ aliphatic residue)[S(═O)₂—C₁₋₁₀ aliphatic residue],
   a S(═O)₂—C₁₋₁₀ aliphatic residue, a S(═O)₂—NH—C₁₋₁₀ aliphatic residue, a S(═O)₂—N(C₁₋₁₀ aliphatic residue)₂, a S—C₁₋₁₀ aliphatic residue,
      wherein each of the aforementioned C₁₋₁₀ aliphatic residue and C₁₋₈ aliphatic groups can in each case be unsubstituted or mono- or polysubstituted with one or more substituents each selected independently of one another from the group consisting of F, Cl, Br, I, NO₂, CN, OH, ═O, O—C₁₋₄ alkyl, OCF₃, CF₃, NH₂, NH(C₁₋₄ alkyl), N(C₁₋₄ alkyl)₂, SH, S—C₁₋₄alkyl, SCF₃, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄alkyl), N(C₁₋₄alkyl)₂, SH, S—C₁₋₄ alkyl, SCF₃ and S(═O)₂OH,
   a C₃₋₁₀ cycloaliphatic residue, a C(═O)—C₃₋₁₀ cycloaliphatic residue, a C(═O)NH—C₃₋₁₀ cycloaliphatic residue a O—C₃₋₁₀ cycloaliphatic residue, a O—(C₁₋₈ aliphatic group)-C₃₋₁₀ cycloaliphatic residue, a S—C₃₋₁₀ cycloaliphatic residue, a S—(C₁₋₈ aliphatic group)-C₃₋₁₀ cycloaliphatic residue, a NH—C₃₋₁₀ cycloaliphatic residue, a NH—C(═O)—C₃₋₁₀ cycloaliphatic residue, a NH—(C₁₋₈ aliphatic group)-C₃₋₁₀ cycloaliphatic residue, a N(C₁₋₁₀ aliphatic residue)(C₃₋₁₀ cycloaliphatic residue), a 3 to 10 membered heterocycloaliphatic residue, a C(═O)-(3 to 10 membered heterocycloaliphatic residue), a C(═O)—NH-(3 to 10 membered heterocycloaliphatic residue), a O-(3 to 10 membered heterocycloaliphatic residue), a O—(C₁₋₈ aliphatic group)-(3 to 10 membered heterocycloaliphatic residue), a S-(3 to 10 membered heterocycloaliphatic residue), a S—(C₁₋₈ aliphatic group)-(3 to 10 membered heterocyclo-aliphatic residue), a NH-(3 to 10 membered heterocycloaliphatic residue), a NH—C(═O)-(3 to 10 membered heterocycloaliphatic residue), NH—(C₁₋₈ aliphatic group)-(3 to 10 membered heterocycloaliphatic residue), a N(C₁₋₁₀ aliphatic residue)(3 to 10 membered heterocycloaliphatic residue),
      wherein each of the aforementioned residues can in each case be optionally bridged via a C₁₋₈ aliphatic group,
      wherein in each case independently of one another the C₁₋₁₀ aliphatic residue, the C₁₋₈ aliphatic group, the C₃₋₁₀ cycloaliphatic residue and the 3 to 10 membered heterocycloaliphatic residue, respectively, can be unsubstituted or mono- or polysubstituted with one or more substituents each selected independently of one another from the group consisting of F, Cl, Br, I, C₁₋₄ alkyl, C₁₋₄ alkylene-OH, C₁₋₄ alkylene-O—C₁₋₄ alkyl, CF₃, C(═O)—C₁₋₄ alkyl, O—C₁₋₄ alkyl, O—C₁₋₄ alkylene-OH, O—C₁₋₄ alkylene-O—C₁₋₄ alkyl, ═O, OCF₃, OH, SH, S—C₁₋₄ alkyl, SCF₃, SO₂—C₁₋₄ alkyl, NH₂, ═NH, ═N(OH), NH—C₁₋₄ alkyl, N(C₁₋₄ alkyl)₂, NH—SO₂—C₁₋₄ alkyl, NH—C(═O)—C₁₋₄ alkyl, phenyl and pyridyl, wherein phenyl and pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH,
   aryl, C(═O)-aryl, C(═O)—NH-aryl, O-aryl, a O—(C₁₋₈ aliphatic group)-aryl, S-aryl, a S—(C₁₋₈ aliphatic group)-aryl, a NH-aryl, NH—C(═O)-aryl, NH—S(═O)₂-aryl a NH—(C₁₋₈ aliphatic group)-aryl, a N(C₁₋₁₀ aliphatic residue)(aryl), heteroaryl, C(═O)-heteroaryl, C(═O)—NH-heteroaryl, O-heteroaryl, O—(C₁₋₈ aliphatic group)-heteroaryl, S-(heteroaryl), S—(C₁₋₈ aliphatic group)-(heteroaryl), NH-(heteroaryl), NH—C(═O)-heteroaryl, NH—S(═O)₂-heteroaryl, NH—(C₁₋₈ aliphatic group)(heteroaryl), N(C₁₋₁₀ aliphatic residue)(heteroaryl),
      wherein each of the aforementioned residues can in each case be optionally bridged via a C₁₋₈ aliphatic group,
      wherein in each case independently of one another the aryl and heteroaryl of the aforementioned residues, respectively, can be unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl, O—C₁₋₄ alkylene-OH, OCF₃, C₁₋₄ alkyl, C₁₋₄ alkylene-O—C₁₋₄-alkyl, C₁₋₄ alkylene-OH, C(═O)—C₁₋₄ alkyl, CF₃, CF₂H, CHF₂, SH, S—C₁₋₄ alkyl, SCF₃, SO₂—C₁₋₄ alkyl, NH₂, NH(C₁₋₄ alkyl), N(C₁₋₄ alkyl)₂, NH—SO₂—C₁₋₄ alkyl, NH—C(═O)—C₁₋₄ alkyl, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl OCF₃, C₁₋₄ alkyl, C₁₋₄ alkylene-O—C₁₋₄-alkyl, C(═O)—OH, CF₃, CF₂H, CHF₂, NH₂, NH(C₁₋₄ alkyl), N(C₁₋₄ alkyl)₂, SH, S—C₁₋₄ alkyl, SCF₃ and S(═O)₂OH,
      wherein in each case independently of one another the C₁₋₁₀ aliphatic residues and the C₁₋₈ aliphatic groups of the aforementioned residues, respectively, can be unsubstituted or mono- or polysubstituted with one or more substituents each selected independently of one another from the group consisting of F, Cl, Br, I, NO₂, CN, OH, ═O, O—C₁₋₄ alkyl, OCF₃, C₁₋₄ alkyl, CF₃, SH, S—C₁₋₄ alkyl, SCF₃, NH₂, NH(C₁₋₄ alkyl), N(C₁₋₄ alkyl)₂, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH.

Preferably,
R⁵, R⁶, R⁷, R⁸ and R⁹ are each independently of one another selected from the group consisting of
   H; F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; NH₂; C(═O)—NH₂; C(═O)—H; C(═O)—OH; S(═O)₂—OH; S(═O)₂—NH₂;
   a C₁₋₁₀ aliphatic residue, (C₁₋₈ aliphatic group)-OH, (C₁₋₈ aliphatic group)-O—C₁₋₁₀ aliphatic residue, (C₁₋₈ aliphatic group)-O—(C₁₋₈ aliphatic group)-OH, (C₁₋₈ aliphatic group)-O—(C₁₋₈ aliphatic group)-O—C₁₋₁₀ aliphatic residue, a (C₁₋₈ aliphatic group)-NH—C₁₋₁₀ aliphatic residue, a (C₁₋₈ aliphatic group)-NH—(C₁₋₈ aliphatic residue)-OH, a (C₁₋₈ aliphatic group)-N(C₁₋₁₀ aliphatic residue)-(C₁₋₈ aliphatic residue)-OH, a (C₁₋₈ aliphatic group)-NH—S(═O)₂—C₁₋₁₀ aliphatic residue, a (C₁₋₈ aliphatic group)-NH—S(═O)₂—NH₂, a (C₁₋₈ aliphatic group)-S(═O)₂—C₁₋₁₀ aliphatic residue, a C(═O)—C₁₋₁₀ aliphatic residue, a C(═O)—NH—C₁₋₁₀ aliphatic residue,
   a O—C₁₋₁₀ aliphatic residue, a O—(C₁₋₈ aliphatic group)-O—C₁₋₁₀ aliphatic residue, O—(C₁₋₈ aliphatic group)-OH,
   a NH—C₁₋₁₀ aliphatic residue, a N(C₁₋₁₀ aliphatic residue)₂, a NH—(C₁₋₈ aliphatic group)-O—C₁₋₁₀ aliphatic residue, a NH—(C₁₋₈ aliphatic group)-OH, a N(C₁₋₁₀ aliphatic residue)[(C₁₋₈ aliphatic group)-OH], a N(C₁₋₁₀ aliphatic residue)[(C₁₋₈ aliphatic group)-O—C₁₋₁₀ aliphatic residue], a NH—C(═O)—C₁₋₁₀ aliphatic residue, a N(C₁₋₁₀ aliphatic residue)[(C(═O)—C₁₋₁₀ aliphatic residue)], a N(C₁₋₁₀ aliphatic residue)[(C₁₋₈ aliphatic group)-O—C₁₋₁₀ aliphatic residue], a N(C₁₋₁₀ aliphatic residue)[(C₁₋₈ aliphatic group)-OH], a NH—S(═O)₂—C₁₋₁₀ aliphatic residue, a N(C₁₋₁₀ aliphatic residue)[S(═O)₂—C₁₋₁₀ aliphatic residue],
   a S(═O)₂—C₁₋₁₀ aliphatic residue, a S(═O)₂—NH—C₁₋₁₀ aliphatic residue, a S(═O)₂—N(C₁₋₁₀ aliphatic residue)₂, a S—C₁₋₁₀ aliphatic residue,
      wherein each of the aforementioned C₁₋₁₀ aliphatic residue and C₁₋₈ aliphatic groups can in each case be unsubstituted or monosubstituted with OH;
   a C₃₋₁₀ cycloaliphatic residue, a C(═O)—C₃₋₁₀ cycloaliphatic residue, a C(═O)NH—C₃₋₁₀ cycloaliphatic residue a O—C₃₋₁₀ cycloaliphatic residue, a O—(C₁₋₈ aliphatic group)-C₃₋₁₀ cycloaliphatic residue, a S—C₃₋₁₀ cycloaliphatic residue, a S—(C₁₋₈ aliphatic group)-C₃₋₁₀ cycloaliphatic residue, a NH—C₃₋₁₀ cycloaliphatic residue, a NH—C(═O)—C₃₋₁₀ cycloaliphatic residue, a NH—(C₁₋₈ aliphatic group)-C₃₋₁₀ cycloaliphatic residue, a N(C₁₋₁₀ aliphatic residue)(C₃₋₁₀ cycloaliphatic residue), a 3 to 10 membered heterocycloaliphatic residue, a C(═O)-(3 to 10 membered heterocycloaliphatic residue), a C(═O)—NH-(3 to 10 membered heterocycloaliphatic residue), a O-(3 to 10 membered heterocycloaliphatic residue), a O—(C₁₋₈ aliphatic group)-(3 to 10 membered heterocycloaliphatic residue), a S-(3 to 10 membered heterocycloaliphatic residue), a S—(C₁₋₈ aliphatic group)-(3 to 10 membered heterocyclo-aliphatic residue), a NH-(3 to 10 membered heterocycloaliphatic residue), a NH—C(═O)-(3 to 10 membered heterocycloaliphatic residue), NH—(C₁₋₈ aliphatic group)-(3 to 10 membered heterocycloaliphatic residue), a N(C₁₋₁₀ aliphatic residue)(3 to 10 membered heterocycloaliphatic residue),
      wherein each of the aforementioned residues can in each case be optionally bridged via an C₁₋₈ aliphatic group,
      wherein in each case independently of one another the C₁₋₁₀ aliphatic residue and the C₁₋₈ aliphatic group can be unsubstituted or monosubstituted with OH,
      wherein in each case independently of one another, the C₃₋₁₀ cycloaliphatic residue and the 3 to 10 membered heterocycloaliphatic residue, respectively, can be unsubstituted or mono- or polysubstituted with one or more substituents each selected independently of one another from the group consisting of F, Cl, Br, I, C₁₋₄ alkyl, C₁₋₄ alkylene-OH, C₁₋₄ alkylene-O—C₁₋₄ alkyl, CF₃, C(═O)—C₁₋₄ alkyl, O—C₁₋₄ alkyl, O—C₁₋₄ alkylene-OH, O—C₁₋₄ alkylene-O—C₁₋₄ alkyl, ═O, OCF₃, OH, SH, S—C₁₋₄ alkyl, SCF₃, SO₂—C₁₋₄ alkyl, NH₂, ═NH, ═N(OH), NH—C₁₋₄ alkyl, N(C₁₋₄ alkyl)₂, NH—SO₂—C₁₋₄ alkyl, NH—C(═O)—C₁₋₄ alkyl,
   aryl, C(═O)-aryl, C(═O)—NH-aryl, O-aryl, a O—(C₁₋₈ aliphatic group)-aryl, S-aryl, a S—(C₁₋₈ aliphatic group)-aryl, a NH-aryl, NH—C(═O)-aryl, NH—S(═O)₂-aryl a NH—(C₁₋₈ aliphatic group)-aryl, a N(C₁₋₁₀ aliphatic residue)(aryl), heteroaryl, C(═O)-heteroaryl, C(═O)—NH-heteroaryl, O-heteroaryl, O—(C₁₋₈ aliphatic group)-heteroaryl, S-(heteroaryl), S—(C₁₋₈ aliphatic group)-(heteroaryl), NH-(heteroaryl), NH—C(═O)-heteroaryl, NH—S(═O)₂-heteroaryl, NH—(C₁₋₈ aliphatic group)(heteroaryl), N(C₁₋₁₀ aliphatic residue)(heteroaryl),
      wherein each of the aforementioned residues can in each case be optionally bridged via a C₁₋₈ aliphatic group,
      wherein in each case independently of one another the aryl and heteroaryl of the aforementioned residues, respectively, can be unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl, O—C₁₋₄ alkylene-OH, OCF₃, C₁₋₄ alkyl, C₁₋₄ alkylene-O—C₁₋₄-alkyl, C₁₋₄ alkylene-OH, C(═O)—C₁₋₄ alkyl, CF₃, CF₂H, CHF₂, SH, S—C₁₋₄ alkyl, SCF₃, SO₂—C₁₋₄ alkyl, NH₂, NH(C₁₋₄ alkyl), N(C₁₋₄ alkyl)₂, NH—SO₂—C₁₋₄ alkyl, NH—C(═O)—C₁₋₄ alkyl, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl OCF₃, C₁₋₄ alkyl, C₁₋₄ alkylene-O—C₁₋₄-alkyl, C(═O)—OH, CF₃, CF₂H, CHF₂, NH₂, NH(C₁₋₄ alkyl), N(C₁₋₄ alkyl)₂, SH, S—C₁₋₄ alkyl, SCF₃ and S(═O)₂OH,
      wherein in each case the C₁₋₁₀ aliphatic residues and the C₁₋₈ aliphatic groups of the aforementioned residues can be unsubstituted or monosubstituted with OH.

More preferably,
R⁵, R⁶, R⁷, R⁸ and R⁹ are each independently of one another selected from the group consisting of
   H; F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; NH₂; C(═O)—NH₂; C(═O)—H; C(═O)—OH; S(═O)₂—OH; S(═O)₂—NH₂;
   a C₁₋₁₀ aliphatic residue, (C₁₋₈ aliphatic group)-OH, (C₁₋₈ aliphatic group)-O—C₁₋₁₀ aliphatic residue, (C₁₋₈ aliphatic group)-O—(C₁₋₈ aliphatic group)-OH, (C₁₋₈ aliphatic group)-O—(C₁₋₈ aliphatic group)-O—C₁₋₁₀ aliphatic residue, a (C₁₋₈ aliphatic group)-NH—C₁₋₁₀ aliphatic residue, a (C₁₋₈ aliphatic group)-NH—(C₁₋₈ aliphatic residue)-OH, a (C₁₋₈ aliphatic group)-N(C₁₋₁₀ aliphatic residue)-(C₁₋₈ aliphatic residue)-OH, a (C₁₋₈ aliphatic group)-NH—S(═O)₂—C₁₋₁₀ aliphatic residue, a (C₁₋₈ aliphatic group)-NH—S(═O)₂—NH₂, a (C₁₋₈ aliphatic group)-S(═O)₂—C₁₋₁₀ aliphatic residue,
   a O—C₁₋₁₀ aliphatic residue, a O—(C₁₋₈ aliphatic group)-O—C₁₋₁₀ aliphatic residue, O—(C₁₋₈ aliphatic group)-OH,
   a NH—C₁₋₁₀ aliphatic residue, a N(C₁₋₁₀ aliphatic residue)₂, a NH—(C₁₋₈ aliphatic group)-O—C₁₋₁₀ aliphatic residue, a NH—(C₁₋₈ aliphatic group)-OH, a N(C₁₋₁₀ aliphatic residue)[(C₁₋₈ aliphatic group)-O—C₁₋₁₀ aliphatic residue], a N(C₁₋₁₀ aliphatic residue)[(C₁₋₈ aliphatic group)-OH], a NH—S(═O)₂—C₁₋₁₀ aliphatic residue,
      wherein each of the aforementioned C₁₋₁₀ aliphatic residue and C₁₋₈ aliphatic groups can in each case be unsubstituted or monosubstituted with OH;
   a C₃₋₁₀ cycloaliphatic residue, a C(═O)—C₃₋₁₀ cycloaliphatic residue, a C(═O)NH—C₃₋₁₀ cycloaliphatic residue, a O—C₃₋₁₀ cycloaliphatic residue, a NH—C₃₋₁₀ cycloaliphatic residue, a NH—C(═O)—C₃₋₁₀ cycloaliphatic residue, a 3 to 10 membered heterocycloaliphatic residue, a C(═O)-(3 to 10 membered heterocycloaliphatic residue), a C(═O)—NH-(3 to 10 membered heterocycloaliphatic residue), a O-(3 to 10 membered heterocycloaliphatic residue), a NH-(3 to 10 membered heterocycloaliphatic residue), a NH—C(═O)-(3 to 10 membered heterocycloaliphatic residue),
      wherein in each case independently of one another, the C₃₋₁₀ cycloaliphatic residue and the 3 to 10 membered heterocycloaliphatic residue, respectively, can be unsubstituted or mono- or polysubstituted with one or more substituents each selected independently of one another from the group consisting of F, Cl, Br, I, C₁₋₄ alkyl, C₁₋₄ alkylene-OH, C₁₋₄ alkylene-O—C₁₋₄ alkyl, CF₃, C(═O)—C₁₋₄ alkyl, O—C₁₋₄ alkyl, O—C₁₋₄ alkylene-OH, O—C₁₋₄ alkylene-O—C₁₋₄ alkyl, OCF₃, OH, SH, S—C₁₋₄ alkyl, SCF₃, SO₂—C₁₋₄ alkyl, NH₂, NH—C₁₋₄ alkyl, N(C₁₋₄ alkyl)₂, NH—SO₂—C₁₋₄ alkyl, NH—C(═O)—C₁₋₄ alkyl;
   aryl, C(═O)-aryl, C(═O)—NH-aryl, NH—C(═O)-aryl, heteroaryl, C(═O)-heteroaryl, C(═O)—NH-heteroaryl, NH—C(═O)-heteroaryl,
      wherein in each case independently of one another the aryl and heteroaryl of the aforementioned residues, respectively, can be unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl, O—C₁₋₄ alkylene-OH, OCF₃, C₁₋₄ alkyl, C₁₋₄ alkylene-O—C₁₋₄-alkyl, C₁₋₄ alkylene-OH, C(═O)—C₁₋₄ alkyl, CF₃, CF₂H, CHF₂, SH, S—C₁₋₄ alkyl, SCF₃, SO₂—C₁₋₄ alkyl, NH₂, NH(C₁₋₄ alkyl), N(C₁₋₄ alkyl)₂, NH—SO₂—C₁₋₄ alkyl, NH—C(═O)—C₁₋₄ alkyl, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl OCF₃, C₁₋₄ alkyl, C₁₋₄ alkylene-O—C₁₋₄-alkyl, C(═O)—OH, CF₃, CF₂H, CHF₂, NH₂, NH(C₁₋₄ alkyl), N(C₁₋₄ alkyl)₂, SH, S—C₁₋₄ alkyl, SCF₃ and S(═O)₂OH.

Even more preferably,
R⁵, R⁶, R⁷, R⁸ and R⁹ are each independently of one another selected from the group consisting of
   H; F; Cl; Br; I; CN; CF₃; CF₂H; CFH₂; OH; OCF₃; SH; SCF₃; NH₂; C(═O)—NH₂; S(═O)₂—OH; S(═O)₂—NH₂;
   a C₁₋₄ aliphatic residue, (C₁₋₄ aliphatic group)-OH, (C₁₋₄ aliphatic group)-O—C₁₋₄ aliphatic residue, (C₁₋₄ aliphatic group)-O—(C₁₋₄ aliphatic group)-OH, (C₁₋₄ aliphatic group)-O—(C₁₋₄ aliphatic group)-O—C₁₋₄ aliphatic residue, a (C₁₋₄ aliphatic group)-NH—C₁₋₄ aliphatic residue, a (C₁₋₄ aliphatic group)-NH—(C₁₋₄ aliphatic residue)-OH, a (C₁₋₄ aliphatic group)-N(C₁₋₄ aliphatic residue)-(C₁₋₄ aliphatic residue)-OH, a (C₁₋₄ aliphatic group)-NH—S(═O)₂—C₁₋₄ aliphatic residue, a (C₁₋₄ aliphatic group)-NH—S(═O)₂—NH₂, a (C₁₋₄ aliphatic group)-S(═O)₂—C₁₋₄ aliphatic residue,
   a O—C₁₋₄ aliphatic residue, a O—(C₁₋₄ aliphatic group)-O—C₁₋₄ aliphatic residue, O—(C₁₋₄ aliphatic group)-OH,
   a NH—C₁₋₄ aliphatic residue, a N(C₁₋₄ aliphatic residue)₂, a NH—(C₁₋₄ aliphatic group)-O—C₁₋₄ aliphatic residue, a NH—(C₁₋₄ aliphatic group)-OH, a N(C₁₋₄ aliphatic residue)[(C₁₋₄ aliphatic group)-O—C₁₋₄ aliphatic residue], a N(C₁₋₄ aliphatic residue)[(C₁₋₄ aliphatic group)-OH], a NH—S(═O)₂—C₁₋₄ aliphatic residue,
      wherein each of the aforementioned C₁₋₄ aliphatic residues and C₁₋₄ aliphatic groups can in each case be unsubstituted or monosubstituted with OH;
   a C₃₋₆ cycloaliphatic residue, O—C₃₋₆ cycloaliphatic residue, a 3 to 6 membered heterocycloaliphatic residue, O-(3 to 6 membered heterocycloaliphatic residue),
      wherein in each case independently of one another, the C₃₋₆ cycloaliphatic residue and the 3 to 6 membered heterocycloaliphatic residue, respectively, can be unsubstituted or mono- or polysubstituted with one or more substituents each selected independently of one another from the group consisting of F, Cl, Br, I, C₁₋₄ alkyl, C₁₋₄ alkylene-OH, C₁₋₄ alkylene-O—C₁₋₄ alkyl, CF₃, C(═O)—C₁₋₄ alkyl, O—C₁₋₄ alkyl, O—C₁₋₄ alkylene-OH, O—C₁₋₄ alkylene-O—C₁₋₄ alkyl, OH, SH, S—C₁₋₄ alkyl, SO₂—C₁₋₄ alkyl, NH₂, NH—C₁₋₄ alkyl, N(C₁₋₄ alkyl)₂, NH—SO₂—C₁₋₄ alkyl, and NH—C(═O)—C₁₋₄ alkyl,
   aryl, C(═O)—NH-aryl, NH—C(═O)-aryl, heteroaryl, C(═O)—NH-heteroaryl, NH—C(═O)-heteroaryl,
      wherein in each case independently of one another the aryl and heteroaryl of the aforementioned residues, respectively, can be unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl, O—C₁₋₄ alkylene-OH, OCF₃, C₁₋₄ alkyl, C₁₋₄ alkylene-O—C₁₋₄-alkyl, C₁₋₄ alkylene-OH, C(═O)—C₁₋₄ alkyl, CF₃, CF₂H, CHF₂, SH, S—C₁₋₄ alkyl, SCF₃, SO₂—C₁₋₄ alkyl, NH₂, NH(C₁₋₄ alkyl), N(C₁₋₄ alkyl)₂, NH—SO₂—C₁₋₄ alkyl, and NH—C(═O)—C₁₋₄ alkyl.

Still more preferably,
R⁵, R⁶, R⁷, R⁸ and R⁹ are each independently of one another selected from the group consisting of
   H; F; Cl; Br; I; CN; CF₃; CF₂H; CFH₂; OH; OCF₃; SH; SCF₃; NH₂; C(═O)—NH₂; S(═O)₂—OH; S(═O)₂—NH₂;
   C₁₋₄ alkyl, C₁₋₄ alkylene-OH, C₁₋₄ alkylene-O—C₁₋₄ alkyl, C₁₋₄ alkylene-O—C₁₋₄ alkylene-OH, C₁₋₄ alkylene-O—C₁₋₄ alkylene-O—C₁₋₄ alkyl, C₁₋₄ alkylene-S(═O)₂—C₁₋₄ alkyl, C₁₋₄ alkylene-NH—S(═O)₂—C₁₋₄ alkyl, C₁₋₄ alkylene-NH—S(═O)₂—NH₂, C₁₋₄ alkylene-NH—C₁₋₄ alkylene-OH, C₁₋₄ alkylene-NH—C₁₋₄ alkylene-O—C₁₋₄ alkyl, C₁₋₄ alkylene-N(C₁₋₄ alkyl)-C₁₋₄ alkylene-OH, C₁₋₄ alkylene-N(C₁₋₄alkyl)-C₁₋₄ alkylene-O—C₁₋₄ alkyl, O—C₁₋₄ alkyl, O—C₁₋₄ alkylene-OH, O—C₁₋₄ alkylene-O—C₁₋₄ alkyl, NH—C₁₋₄ alkyl, N(C₁₋₄ alkyl)₂, NH—C₁₋₄ alkylene-OH, NH—C₁₋₄ alkylene-O—C₁₋₄ alkyl, N(C₁₋₄ alkyl)-[C₁₋₄ alkylene-OH], N(C₁₋₄ alkyl)-[C₁₋₄ alkylene-O—C₁₋₄ alkyl], NH—S(═O)₂—C₁₋₄ alkyl,
      wherein C₁₋₄ alkylene can in each case be unsubstituted or monosubstituted with OH,
   a C₃₋₆ cycloaliphatic residue, O—C₃₋₆ cycloaliphatic residue, a 3 to 6 membered heterocycloaliphatic residue,
      wherein the C₃₋₆ cycloaliphatic residue is preferably selected from the group consisting of cyclopropyl, cyclobutyl, cyclopentyl, cyclohexyl, and
      wherein the 3 to 6 membered heterocycloaliphatic residue is preferably selected from the group consisting of tetrahydropyranyl, preferably tetrahydro-2H-pyran-4-yl, azetidinyl, piperidinyl, morpholinyl and pyrrolidinyl,
      wherein the C₃₋₆ cycloaliphatic residue and the 3 to 6 membered heterocycloaliphatic residue, respectively, can be unsubstituted or mono- or polysubstituted with one or more substituents each selected independently of one another from the group consisting of F, Cl, Br, I, OH, O—C₁₋₄ alkyl, NH₂, NH(C₁₋₄ alkyl), and N(C₁₋₄ alkyl)₂, and C₁₋₄ alkyl,
   phenyl, C(═O)—NH-phenyl, NH—C(═O)-phenyl, heteroaryl, C(═O)—NH-heteroaryl, NH—C(═O)-heteroaryl, preferably phenyl, C(═O)—NH-phenyl and NH—C(═O)-phenyl,
      wherein heteroaryl is preferably selected from the group consisting of pyrdiyl, furyl and thienyl;
      wherein in each case independently of one another phenyl and heteroaryl of the aforementioned residues, respectively, can be unsubstituted or mono- or polysubstituted with one or more substituents each selected independently of one another from the group consisting of F, Cl, Br, I, OH, O—C₁₋₄ alkyl, C₁₋₄ alkyl, and CF₃.

In yet another preferred embodiment of the compound according to the invention of general formula (I),
R⁵, R⁶, R⁸ and R⁹ are each independently of one another selected from the group consisting of
   H; F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; NH₂; C(═O)—NH₂; C(═O)—H; C(═O)—OH; S(═O)₂—OH; S(═O)₂—NH₂; a C₁₋₁₀ aliphatic residue, a NH—C₁₋₁₀ aliphatic residue, a N(C₁₋₁₀ aliphatic residue)₂ and a O—C₁₋₁₀ aliphatic residue, wherein the C₁₋₁₀ aliphatic residue can in each case be unsubstituted or mono- or disubstituted with OH;
and R⁷ is selected from the group consisting of
   H; F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; NH₂; C(═O)—NH₂; C(═O)—H; C(═O)—OH; S(═O)₂—OH; S(═O)₂—NH₂;
   a C₁₋₁₀ aliphatic residue, (C₁₋₈ aliphatic group)-OH, (C₁₋₈ aliphatic group)-O—C₁₋₁₀ aliphatic residue, (C₁₋₈ aliphatic group)-O—(C₁₋₈ aliphatic group)-OH, (C₁₋₈ aliphatic group)-O—(C₁₋₈ aliphatic group)-O—C₁₋₁₀ aliphatic residue, a (C₁₋₈ aliphatic group)-NH—C₁₋₁₀ aliphatic residue, a (C₁₋₈ is aliphatic group)-NH—(C₁₋₈ aliphatic residue)-OH, a (C₁₋₈ aliphatic group)-N(C₁₋₁₀ aliphatic residue)-(C₁₋₈ aliphatic residue)-OH, a (C₁₋₈ is aliphatic group)-NH—S(═O)₂—C₁₋₁₀ aliphatic residue, a (C₁₋₈ aliphatic group)-NH—S(═O)₂—NH₂, a (C₁₋₈ aliphatic group)-S(═O)₂—C₁₋₁₀ aliphatic residue, a C(═O)—C₁₋₁₀ aliphatic residue, a C(═O)—NH—C₁₋₁₀ aliphatic residue,
   a O—C₁₋₁₀ aliphatic residue, a O—(C₁₋₈ aliphatic group)-O—C₁₋₁₀ aliphatic residue, O—(C₁₋₈ aliphatic group)-OH,
   a NH—C₁₋₁₀ aliphatic residue, a N(C₁₋₁₀ aliphatic residue)₂, a NH—[(C₁₋₈ aliphatic group)-O—C₁₋₁₀ aliphatic residue], a NH—[(C₁₋₈ aliphatic group)-OH], a N(C₁₋₁₀ aliphatic residue)[(C₁₋₈ aliphatic group)-OH], a N(C₁₋₁₀ aliphatic residue)[(C₁₋₈ aliphatic group)-O—C₁₋₁₀ aliphatic residue], a NH—C(═O)—C₁₋₁₀ aliphatic residue, a N(C₁₋₁₀ aliphatic residue)[(C(═O)—C₁₋₁₀ aliphatic residue)], a N(C₁₋₁₀ aliphatic residue)[(C₁₋₈ aliphatic group)-O—C₁₋₁₀ aliphatic residue], a N(C₁₋₁₀ aliphatic residue)[(C₁₋₈ aliphatic group)-OH], a NH—S(═O)₂—C₁₋₁₀ aliphatic residue, a N(C₁₋₁₀ aliphatic residue)[S(═O)₂—C₁₋₁₀ aliphatic residue],
   a S(═O)₂—C₁₋₁₀ aliphatic residue, a S(═O)₂—NH—C₁₋₁₀ aliphatic residue, a S(═O)₂—N(C₁₋₁₀ aliphatic residue)₂, a S—C₁₋₁₀ aliphatic residue,
      wherein each of the aforementioned C₁₋₁₀ aliphatic residue and C₁₋₈ aliphatic groups can in each case be unsubstituted or mono- or polysubstituted with one or more substituents each selected independently of one another from the group consisting of F, Cl, Br, I, NO₂, CN, OH, ═O, O—C₁₋₄ alkyl, OCF₃, CF₃, NH₂, NH(C₁₋₄alkyl), N(C₁₋₄alkyl)₂, SH, S—C₁₋₄alkyl, SCF₃, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄alkyl)₂, SH, S—C₁₋₄ alkyl, SCF₃ and S(═O)₂OH,
   a C₃₋₁₀ cycloaliphatic residue, a C(═O)—C₃₋₁₀ cycloaliphatic residue, a C(═O)NH—C₃₋₁₀ cycloaliphatic residue a O—C₃₋₁₀ cycloaliphatic residue, a O—(C₁₋₈ aliphatic group)-C₃₋₁₀ cycloaliphatic residue, a S—C₃₋₁₀ cycloaliphatic residue, a S—(C₁₋₈ aliphatic group)-C₃₋₁₀ cycloaliphatic residue, a NH—C₃₋₁₀ cycloaliphatic residue, a NH—C(═O)—C₃₋₁₀ cycloaliphatic residue, a NH—(C₁₋₈ aliphatic group)-C₃₋₁₀ cycloaliphatic residue, a N(C₁₋₁₀ aliphatic residue)(C₃₋₁₀ cycloaliphatic residue), a 3 to 10 membered heterocycloaliphatic residue, a C(═O)-(3 to 10 membered heterocycloaliphatic residue), a C(═O)—NH-(3 to 10 membered heterocycloaliphatic residue), a O-(3 to 10 membered heterocycloaliphatic residue), a O—(C₁₋₈ aliphatic group)-(3 to 10 membered heterocycloaliphatic residue), a S-(3 to 10 membered heterocycloaliphatic residue), a S—(C₁₋₈ aliphatic group)-(3 to 10 membered heterocyclo-aliphatic residue), a NH-(3 to 10 membered heterocycloaliphatic residue), a NH—C(═O)-(3 to 10 membered heterocycloaliphatic residue), NH—(C₁₋₈ aliphatic group)-(3 to 10 membered heterocycloaliphatic residue), a N(C₁₋₁₀ aliphatic residue)(3 to 10 membered heterocycloaliphatic residue),
      wherein each of the aforementioned residues can in each case be optionally bridged via a C₁₋₈ aliphatic group,
      wherein in each case independently of one another the C₁₋₁₀ aliphatic residue, the C₁₋₈ aliphatic group, the C₃₋₁₀ cycloaliphatic residue and the 3 to 10 membered heterocycloaliphatic residue, respectively, can be unsubstituted or mono- or polysubstituted with one or more substituents each selected independently of one another from the group consisting of F, Cl, Br, I, C₁₋₄ alkyl, C₁₋₄ alkylene-OH, C₁₋₄ alkylene-O—C₁₋₄ alkyl, CF₃, C(═O)—C₁₋₄ alkyl, O—C₁₋₄ alkyl, O—C₁₋₄ alkylene-OH, O—C₁₋₄ alkylene-O—C₁₋₄ alkyl, ═O, OCF₃, OH, SH, S—C₁₋₄ alkyl, SCF₃, SO₂—C₁₋₄ alkyl, NH₂, ═NH, ═N(OH), NH—C₁₋₄ alkyl, N(C₁₋₄ alkyl)₂, NH—SO₂—C₁₋₄ alkyl, NH—C(═O)—C₁₋₄ alkyl, phenyl and pyridyl, wherein phenyl and pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH,
   aryl, C(═O)-aryl, C(═O)—NH-aryl, O-aryl, a O—(C₁₋₈ aliphatic group)-aryl, S-aryl, a S—(C₁₋₈ aliphatic group)-aryl, a NH-aryl, NH—C(═O)-aryl, NH—S(═O)₂-aryl a NH—(C₁₋₈ aliphatic group)-aryl, a N(C₁₋₁₀ aliphatic residue)(aryl), heteroaryl, C(═O)-heteroaryl, C(═O)—NH-heteroaryl, O-heteroaryl, O—(C₁₋₈ aliphatic group)-heteroaryl, S-(heteroaryl), S—(C₁₋₈ aliphatic group)-(heteroaryl), NH-(heteroaryl), NH—C(═O)-heteroaryl, NH—S(═O)₂-heteroaryl, NH—(C₁₋₈ aliphatic group)(heteroaryl), N(C₁₋₁₀ aliphatic residue)(heteroaryl),
      wherein each of the aforementioned residues can in each case be optionally bridged via a C₁₋₈ aliphatic group,
      wherein in each case independently of one another the aryl and heteroaryl of the aforementioned residues, respectively, can be unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl, O—C₁₋₄ alkylene-OH, OCF₃, C₁₋₄ alkyl, C₁₋₄ alkylene-O—C₁₋₄-alkyl, C₁₋₄ alkylene-OH, C(═O)—C₁₋₄ alkyl, CF₃, CF₂H, CHF₂, SH, S—C₁₋₄ alkyl, SCF₃, SO₂—C₁₋₄ alkyl, NH₂, NH(C₁₋₄ alkyl), N(C₁₋₄ alkyl)₂, NH—SO₂—C₁₋₄ alkyl, NH—C(═O)—C₁₋₄ alkyl, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl OCF₃, C₁₋₄ alkyl, C₁₋₄ alkylene-O—C₁₋₄-alkyl, C(═O)—OH, CF₃, CF₂H, CHF₂, NH₂, NH(C₁₋₄ alkyl), N(C₁₋₄ alkyl)₂, SH, S—C₁₋₄ alkyl, SCF₃ and S(═O)₂OH,
      wherein in each case independently of one another the C₁₋₁₀ aliphatic residues and the C₁₋₈ aliphatic groups of the aforementioned residues, respectively, can be unsubstituted or mono- or polysubstituted with one or more substituents each selected independently of one another from the group consisting of F, Cl, Br, I, NO₂, CN, OH, ═O, O—C₁₋₄ alkyl, OCF₃, C₁₋₄ alkyl, CF₃, SH, S—C₁₋₄ alkyl, SCF₃, NH₂, NH(C₁₋₄ alkyl), N(C₁₋₄ alkyl)₂, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH.

Preferably,
R⁵, R⁶, R⁸ and R⁹ are each independently of one another selected from the group consisting of
   H; F; Cl; Br; I; CN; NO₂; CF₃; CF₂H; CFH₂; OH; OCF₃; OCF₂Cl; OCFCl₂; SH; SCF₃; NH₂; C(═O)—NH₂; CH₂ OH; methyl; ethyl; tert.-butyl; O-methyl; NH-methyl; N(methyl)₂; preferably F; Cl; Br; I; CN; NO₂; CF₃; CF₂H; CFH₂; OH; OCF₃; OCF₂Cl; OCFCl₂; SH; SCF₃; NH₂; C(═O)—NH₂; methyl; ethyl; tert.-butyl; O-methyl; NH-methyl; N(methyl)₂;
and R⁷ is selected from the group consisting of
   H; F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; NH₂; C(═O)—NH₂; C(═O)—H; C(═O)—OH; S(═O)₂—OH; S(═O)₂—NH₂;
   a C₁₋₁₀ aliphatic residue, (C₁₋₈ aliphatic group)-OH, (C₁₋₈ aliphatic group)-O—C₁₋₁₀ aliphatic residue, (C₁₋₈ aliphatic group)-O—(C₁₋₈ aliphatic group)-OH, (C₁₋₈ aliphatic group)-O—(C₁₋₈ aliphatic group)-O—C₁₋₁₀ aliphatic residue, a (C₁₋₈ aliphatic group)-NH—C₁₋₁₀ aliphatic residue, a (C₁₋₈ aliphatic group)-NH—(C₁₋₈ aliphatic residue)-OH, a (C₁₋₈ aliphatic group)-N(C₁₋₁₀ aliphatic residue)-(C₁₋₈ aliphatic residue)-OH, a (C₁₋₈ aliphatic group)-NH—S(═O)₂—C₁₋₁₀ aliphatic residue, a (C₁₋₈ aliphatic group)-NH—S(═O)₂—NH₂, a (C₁₋₈ aliphatic group)-S(═O)₂—C₁₋₁₀ aliphatic residue, a C(═O)—C₁₋₁₀ aliphatic residue, a C(═O)—NH—C₁₋₁₀ aliphatic residue,
   a O—C₁₋₁₀ aliphatic residue, a O—(C₁₋₈ aliphatic group)-O—C₁₋₁₀ aliphatic residue, O—(C₁₋₈ aliphatic group)-OH,
   a NH—C₁₋₁₀ aliphatic residue, a N(C₁₋₁₀ aliphatic residue)₂, a NH—(C₁₋₈ aliphatic group)-O—C₁₋₁₀ aliphatic residue, a NH—(C₁₋₈ aliphatic group)-OH, a N(C₁₋₁₀ aliphatic residue)[(C₁₋₈ aliphatic group)-OH], a N(C₁₋₁₀ aliphatic residue)[(C₁₋₈ aliphatic group)-O—C₁₋₁₀ aliphatic residue], a NH—C(═O)—C₁₋₁₀ aliphatic residue, a N(C₁₋₁₀ aliphatic residue)[(C(═O)—C₁₋₁₀ aliphatic residue)], a N(C₁₋₁₀ aliphatic residue)[(C₁₋₈ aliphatic group)-O—C₁₋₁₀ aliphatic residue], a N(C₁₋₁₀ aliphatic residue)[(C₁₋₈ aliphatic group)-OH], a NH—S(═O)₂—C₁₋₁₀ aliphatic residue, a N(C₁₋₁₀ aliphatic residue)[S(═O)₂—C₁₋₁₀ aliphatic residue],
   a S(═O)₂—C₁₋₁₀ aliphatic residue, a S(═O)₂—NH—C₁₋₁₀ aliphatic residue, a S(═O)₂—N(C₁₋₁₀ aliphatic residue)₂, a S—C₁₋₁₀ aliphatic residue,
      wherein each of the aforementioned C₁₋₁₀ aliphatic residue and C₁₋₈ aliphatic groups can in each case be unsubstituted or monosubstituted with OH;
   a C₃₋₁₀ cycloaliphatic residue, a C(═O)—C₃₋₁₀ cycloaliphatic residue, a C(═O)NH—C₃₋₁₀ cycloaliphatic residue a O—C₃₋₁₀ cycloaliphatic residue, a O—(C₁₋₈ aliphatic group)-C₃₋₁₀ cycloaliphatic residue, a S—C₃₋₁₀ cycloaliphatic residue, a S—(C₁₋₈ aliphatic group)-C₃₋₁₀ cycloaliphatic residue, a NH—C₃₋₁₀ cycloaliphatic residue, a NH—C(═O)—C₃₋₁₀ cycloaliphatic residue, a NH—(C₁₋₈ aliphatic group)-C₃₋₁₀ cycloaliphatic residue, a N(C₁₋₁₀ aliphatic residue)(C₃₋₁₀ cycloaliphatic residue), a 3 to 10 membered heterocycloaliphatic residue, a C(═O)-(3 to 10 membered heterocycloaliphatic residue), a C(═O)—NH-(3 to 10 membered heterocycloaliphatic residue), a O-(3 to 10 membered heterocycloaliphatic residue), a O—(C₁₋₈ aliphatic group)-(3 to 10 membered heterocycloaliphatic residue), a S-(3 to 10 membered heterocycloaliphatic residue), a S—(C₁₋₈ aliphatic group)-(3 to 10 membered heterocyclo-aliphatic residue), a NH-(3 to 10 membered heterocycloaliphatic residue), a NH—C(═O)-(3 to 10 membered heterocycloaliphatic residue), NH—(C₁₋₈ aliphatic group)-(3 to 10 membered heterocycloaliphatic residue), a N(C₁₋₁₀ aliphatic residue)(3 to 10 membered heterocycloaliphatic residue),
      wherein each of the aforementioned residues can in each case be optionally bridged via an C₁₋₈ aliphatic group,
      wherein in each case independently of one another the C₁₋₁₀ aliphatic residue and the C₁₋₈ aliphatic group can be unsubstituted or monosubstituted with OH,
      wherein in each case independently of one another, the C₃₋₁₀ cycloaliphatic residue and the 3 to 10 membered heterocycloaliphatic residue, respectively, can be unsubstituted or mono- or polysubstituted with one or more substituents each selected independently of one another from the group consisting of F, Cl, Br, I, C₁₋₄ alkyl, C₁₋₄ alkylene-OH, C₁₋₄ alkylene-O—C₁₋₄ alkyl, CF₃, C(═O)—C₁₋₄ alkyl, O—C₁₋₄ alkyl, O—C₁₋₄ alkylene-OH, O—C₁₋₄ alkylene-O—C₁₋₄ alkyl, ═O, OCF₃, OH, SH, S—C₁₋₄ alkyl, SCF₃, SO₂—C₁₋₄ alkyl, NH₂, ═NH, ═N(OH), NH—C₁₋₄ alkyl, N(C₁₋₄ alkyl)₂, NH—SO₂—C₁₋₄ alkyl, NH—C(═O)—C₁₋₄ alkyl,
   aryl, C(═O)-aryl, C(═O)—NH-aryl, O-aryl, a O—(C₁₋₈ aliphatic group)-aryl, S-aryl, a S—(C₁₋₈ aliphatic group)-aryl, a NH-aryl, NH—C(═O)-aryl, NH—S(═O)₂-aryl a NH—(C₁₋₈ aliphatic group)-aryl, a N(C₁₋₁₀ aliphatic residue)(aryl), heteroaryl, C(═O)-heteroaryl, C(═O)—NH-heteroaryl, O-heteroaryl, O—(C₁₋₈ aliphatic group)-heteroaryl, S-(heteroaryl), S—(C₁₋₈ aliphatic group)-(heteroaryl), NH-(heteroaryl), NH—C(═O)-heteroaryl, NH—S(═O)₂-heteroaryl, NH—(C₁₋₈ aliphatic group)(heteroaryl), N(C₁₋₁₀ aliphatic residue)(heteroaryl),
      wherein each of the aforementioned residues can in each case be optionally bridged via a C₁₋₈ aliphatic group,
      wherein in each case independently of one another the aryl and heteroaryl of the aforementioned residues, respectively, can be unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl, O—C₁₋₄ alkylene-OH, OCF₃, C₁₋₄ alkyl, C₁₋₄ alkylene-O—C₁₋₄-alkyl, C₁₋₄ alkylene-OH, C(═O)—C₁₋₄ alkyl, CF₃, CF₂H, CHF₂, SH, S—C₁₋₄ alkyl, SCF₃, SO₂—C₁₋₄ alkyl, NH₂, NH(C₁₋₄ alkyl), N(C₁₋₄ alkyl)₂, NH—SO₂—C₁₋₄ alkyl, NH—C(═O)—C₁₋₄ alkyl, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl OCF₃, C₁₋₄ alkyl, C₁₋₄ alkylene-O—C₁₋₄-alkyl, C(═O)—OH, CF₃, CF₂H, CHF₂, NH₂, NH(C₁₋₄ alkyl), N(C₁₋₄ alkyl)₂, SH, S—C₁₋₄ alkyl, SCF₃ and S(═O)₂OH,
      wherein in each case the C₁₋₁₀ aliphatic residues and the C₁₋₈ aliphatic groups of the aforementioned residues can be unsubstituted or monosubstituted with OH.

More preferably,
R⁵, R⁶, R⁸ and R⁹ are each independently of one another selected from the group consisting of
   H; F; Cl; Br; I; CF₃; CF₂H; CFH₂; OH; CH₂OH; methyl; and O-methyl; preferably H; F; Cl; Br; I; CF₃; CF₂H; CFH₂; OH; methyl; and O-methyl;
and R⁷ is selected from the group consisting of
   H; F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; NH₂; C(═O)—NH₂; C(═O)—H; C(═O)—OH; S(═O)₂—OH; S(═O)₂—NH₂;
   a C₁₋₁₀ aliphatic residue, (C₁₋₈ aliphatic group)-OH, (C₁₋₈ aliphatic group)-O—C₁₋₁₀ aliphatic residue, (C₁₋₈ aliphatic group)-O—(C₁₋₈ aliphatic group)-OH, (C₁₋₈ aliphatic group)-O—(C₁₋₈ aliphatic group)-O—C₁₋₁₀ aliphatic residue, a (C₁₋₈ aliphatic group)-NH—C₁₋₁₀ aliphatic residue, a (C₁₋₈ aliphatic group)-NH—(C₁₋₈ aliphatic residue)-OH, a (C₁₋₈ aliphatic group)-N(C₁₋₁₀ aliphatic residue)-(C₁₋₈ aliphatic residue)-OH, a (C₁₋₈ aliphatic group)-NH—S(═O)₂—C₁₋₁₀ aliphatic residue, a (C₁₋₈ aliphatic group)-NH—S(═O)₂—NH₂, a (C₁₋₈ aliphatic group)-S(═O)₂—C₁₋₁₀ aliphatic residue,
   a O—C₁₋₁₀ aliphatic residue, a O—(C₁₋₈ aliphatic group)-O—C₁₋₁₀ aliphatic residue, O—(C₁₋₈ aliphatic group)-OH,
   a NH—C₁₋₁₀ aliphatic residue, a N(C₁₋₁₀ aliphatic residue)₂, a NH—(C₁₋₈ aliphatic group)-O—C₁₋₁₀ aliphatic residue, a NH—(C₁₋₈ aliphatic group)-OH, a N(C₁₋₁₀ aliphatic residue)[(C₁₋₈ aliphatic group)-O—C₁₋₁₀ aliphatic residue], a N(C₁₋₁₀ aliphatic residue)[(C₁₋₈ aliphatic group)-OH], a NH—S(═O)₂—C₁₋₁₀ aliphatic residue,
      wherein each of the aforementioned C₁₋₁₀ aliphatic residue and C₁₋₈ aliphatic groups can in each case be unsubstituted or monosubstituted with OH;
   a C₃₋₁₀ cycloaliphatic residue, a C(═O)—C₃₋₁₀ cycloaliphatic residue, a C(═O)NH—C₃₋₁₀ cycloaliphatic residue, a O—C₃₋₁₀ cycloaliphatic residue, a NH—C₃₋₁₀ cycloaliphatic residue, a NH—C(═O)—C₃₋₁₀ cycloaliphatic residue, a 3 to 10 membered heterocycloaliphatic residue, a C(═O)-(3 to 10 membered heterocycloaliphatic residue), a C(═O)—NH-(3 to 10 membered heterocycloaliphatic residue), a O-(3 to 10 membered heterocycloaliphatic residue), a NH-(3 to 10 membered heterocycloaliphatic residue), a NH—C(═O)-(3 to 10 membered heterocycloaliphatic residue),
      wherein in each case independently of one another, the C₃₋₁₀ cycloaliphatic residue and the 3 to 10 membered heterocycloaliphatic residue, respectively, can be unsubstituted or mono- or polysubstituted with one or more substituents each selected independently of one another from the group consisting of F, Cl, Br, I, C₁₋₄ alkyl, C₁₋₄ alkylene-OH, C₁₋₄ alkylene-O—C₁₋₄ alkyl, CF₃, C(═O)—C₁₋₄ alkyl, O—C₁₋₄ alkyl, O—C₁₋₄ alkylene-OH, O—C₁₋₄ alkylene-O—C₁₋₄ alkyl, OCF₃, OH, SH, S—C₁₋₄ alkyl, SCF₃, SO₂—C₁₋₄ alkyl, NH₂, NH—C₁₋₄ alkyl, N(C₁₋₄ alkyl)₂, NH—SO₂—C₁₋₄ alkyl, NH—C(═O)—C₁₋₄ alkyl;
   aryl, C(═O)-aryl, C(═O)—NH-aryl, NH—C(═O)-aryl, heteroaryl, C(═O)-heteroaryl, C(═O)—NH-heteroaryl, NH—C(═O)-heteroaryl,
      wherein in each case independently of one another the aryl and heteroaryl of the aforementioned residues, respectively, can be unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl, O—C₁₋₄ alkylene-OH, OCF₃, C₁₋₄ alkyl, C₁₋₄ alkylene-O—C₁₋₄-alkyl, C₁₋₄ alkylene-OH, C(═O)—C₁₋₄ alkyl, CF₃, CF₂H, CHF₂, SH, S—C₁₋₄ alkyl, SCF₃, SO₂—C₁₋₄ alkyl, NH₂, NH(C₁₋₄ alkyl), N(C₁₋₄ alkyl)₂, NH—SO₂—C₁₋₄ alkyl, NH—C(═O)—C₁₋₄ alkyl, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl OCF₃, C₁₋₄ alkyl, C₁₋₄ alkylene-O—C₁₋₄-alkyl, C(═O)—OH, CF₃, CF₂H, CHF₂, NH₂, NH(C₁₋₄ alkyl), N(C₁₋₄ alkyl)₂, SH, S—C₁₋₄ alkyl, SCF₃ and S(═O)₂OH.

Even more preferably,
R⁵, R⁶, R⁸ and R⁹ are each independently of one another selected from the group consisting of
   H; F; Cl; Br; I; CF₃; OH; CH₂OH; methyl; and O-methyl; preferably H; F; Cl; Br; I; CF₃; OH; methyl; and O-methyl;
and R⁷ is selected from the group consisting of
   H; F; Cl; Br; I; CN; CF₃; CF₂H; CFH₂; OH; OCF₃; SH; SCF₃; NH₂; C(═O)—NH₂; S(═O)₂—OH; S(═O)₂—NH₂;
   a C₁₋₄ aliphatic residue, (C₁₋₄ aliphatic group)-OH, (C₁₋₄ aliphatic group)-O—C₁₋₄ aliphatic residue, (C₁₋₄ aliphatic group)-O—(C₁₋₄ aliphatic group)-OH, (C₁₋₄ aliphatic group)-O—(C₁₋₄ aliphatic group)-O—C₁₋₄ aliphatic residue, a (C₁₋₄ aliphatic group)-NH—C₁₋₄ aliphatic residue, a (C₁₋₄ aliphatic group)-NH—(C₁₋₄ aliphatic residue)-OH, a (C₁₋₄ aliphatic group)-N(C₁₋₄ aliphatic residue)-(C₁₋₄ aliphatic residue)-OH, a (C₁₋₄ aliphatic group)-NH—S(═O)₂—C₁₋₄ aliphatic residue, a (C₁₋₄ aliphatic group)-NH—S(═O)₂—NH₂, a (C₁₋₄ aliphatic group)-S(═O)₂—C₁₋₄ aliphatic residue,
   a O—C₁₋₄ aliphatic residue, a O—(C₁₋₄ aliphatic group)-O—C₁₋₄ aliphatic residue, O—(C₁₋₄ aliphatic group)-OH,
   a NH—C₁₋₄ aliphatic residue, a N(C₁₋₄ aliphatic residue)₂, a NH—(C₁₋₄ aliphatic group)-O—C₁₋₄ aliphatic residue, a NH—(C₁₋₄ aliphatic group)-OH, a N(C₁₋₄ aliphatic residue)[(C₁₋₄ aliphatic group)-O—C₁₋₄ aliphatic residue], a N(C₁₋₄ aliphatic residue)[(C₁₋₄ aliphatic group)-OH], a NH—S(═O)₂—C₁₋₄ aliphatic residue,
      wherein each of the aforementioned C₁₋₄ aliphatic residues and C₁₋₄ aliphatic groups can in each case be unsubstituted or monosubstituted with OH;
   a C₃₋₆ cycloaliphatic residue, O—C₃₋₆ cycloaliphatic residue, a 3 to 6 membered heterocycloaliphatic residue, O-(3 to 6 membered heterocycloaliphatic residue),
      wherein in each case independently of one another, the C₃₋₆ cycloaliphatic residue and the 3 to 6 membered heterocycloaliphatic residue, respectively, can be unsubstituted or mono- or polysubstituted with one or more substituents each selected independently of one another from the group consisting of F, Cl, Br, I, C₁₋₄ alkyl, C₁₋₄ alkylene-OH, C₁₋₄ alkylene-O—C₁₋₄ alkyl, CF₃, C(═O)—C₁₋₄ alkyl, O—C₁₋₄ alkyl, O—C₁₋₄ alkylene-OH, O—C₁₋₄ alkylene-O—C₁₋₄ alkyl, OH, SH, S—C₁₋₄ alkyl, SO₂—C₁₋₄ alkyl, NH₂, NH—C₁₋₄ alkyl, N(C₁₋₄ alkyl)₂, NH—SO₂—C₁₋₄ alkyl, and NH—C(═O)—C₁₋₄ alkyl,
   aryl, C(═O)—NH-aryl, NH—C(═O)-aryl, heteroaryl, C(═O)—NH-heteroaryl, NH—C(═O)-heteroaryl,
      wherein in each case independently of one another the aryl and heteroaryl of the aforementioned residues, respectively, can be unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl, O—C₁₋₄ alkylene-OH, OCF₃, C₁₋₄ alkyl, C₁₋₄ alkylene-O—C₁₋₄-alkyl, C₁₋₄ alkylene-OH, C(═O)—C₁₋₄ alkyl, CF₃, CF₂H, CHF₂, SH, S—C₁₋₄ alkyl, SCF₃, SO₂—C₁₋₄ alkyl, NH₂, NH(C₁₋₄ alkyl), N(C₁₋₄ alkyl)₂, NH—SO₂—C₁₋₄ alkyl, and NH—C(═O)—C₁₋₄ alkyl.

Still more preferably,
R⁵, R⁶, R⁸ and R⁹ are each independently of one another selected from the group consisting of
   H; F; Cl; Br; I; CF₃; OH; CH₂OH; methyl; O-methyl; preferably H; F; Cl; Br; I; CF₃; OH; methyl; O-methyl;
and R⁷ is selected from the group consisting of
   H; F; Cl; Br; I; CN; CF₃; CF₂H; CFH₂; OH; OCF₃; SH; SCF₃; NH₂; C(═O)—NH₂; S(═O)₂—OH; S(═O)₂—NH₂;
   C₁₋₄ alkyl, C₁₋₄ alkylene-OH, C₁₋₄ alkylene-O—C₁₋₄ alkyl, C₁₋₄ alkylene-O—C₁₋₄ alkylene-OH, C₁₋₄ alkylene-O—C₁₋₄ alkylene-O—C₁₋₄ alkyl, C₁₋₄ alkylene-S(═O)₂—C₁₋₄ alkyl, C₁₋₄ alkylene-NH—S(═O)₂—C₁₋₄ alkyl, C₁₋₄ alkylene-NH—S(═O)₂—NH₂, C₁₋₄ alkylene-NH—C₁₋₄ alkylene-OH, C₁₋₄ alkylene-NH—C₁₋₄ alkylene-O—C₁₋₄ alkyl, C₁₋₄ alkylene-N(C₁₋₄ alkyl)-C₁₋₄ alkylene-OH, C₁₋₄ alkylene-N(C₁₋₄ alkyl)-C₁₋₄ alkylene-O—C₁₋₄ alkyl, O—C₁₋₄ alkyl, O—C₁₋₄ alkylene-OH, O—C₁₋₄ alkylene-O—C₁₋₄ alkyl, NH—C₁₋₄ alkyl, N(C₁₋₄ alkyl)₂, NH—C₁₋₄ alkylene-OH, NH—C₁₋₄ alkylene-O—C₁₋₄ alkyl, N(C₁₋₄ alkyl)-[C₁₋₄ alkylene-OH], N(C₁₋₄ alkyl)-[C₁₋₄ alkylene-O—C₁₋₄ alkyl], NH—S(═O)₂—C₁₋₄ alkyl,
      wherein C₁₋₄ alkylene can in each case be unsubstituted or monosubstituted with OH,
   a C₃₋₆ cycloaliphatic residue, O—C₃₋₆ cycloaliphatic residue, a 3 to 6 membered heterocycloaliphatic residue,
      wherein the C₃₋₆ cycloaliphatic residue is preferably selected from the group consisting of cyclopropyl, cyclobutyl, cyclopentyl, cyclohexyl, and
      wherein the 3 to 6 membered heterocycloaliphatic residue is preferably selected from the group consisting of tetrahydropyranyl, preferably tetrahydro-2H-pyran-4-yl, azetidinyl, piperidinyl, morpholinyl and pyrrolidinyl,
      wherein the C₃₋₆ cycloaliphatic residue and the 3 to 6 membered heterocycloaliphatic residue, respectively, can be unsubstituted or mono- or polysubstituted with one or more substituents each selected independently of one another from the group consisting of F, Cl, Br, I, OH, O—C₁₋₄ alkyl, NH₂, NH(C₁₋₄ alkyl), and N(C₁₋₄ alkyl)₂, and C₁₋₄ alkyl,
   phenyl, C(═O)—NH-phenyl, NH—C(═O)-phenyl, heteroaryl, C(═O)—NH-heteroaryl, NH—C(═O)-heteroaryl, preferably phenyl, C(═O)—NH-phenyl and NH—C(═O)-phenyl,
      wherein heteroaryl is preferably selected from the group consisting of pyrdiyl, furyl and thienyl;
      wherein in each case independently of one another phenyl and heteroaryl of the aforementioned residues, respectively, can be unsubstituted or mono- or polysubstituted with one or more substituents each selected independently of one another from the group consisting of F, Cl, Br, I, OH, O—C₁₋₄ alkyl, C₁₋₄ alkyl, and CF₃.

In a particularly preferred embodiment of the compound according to the invention of general formula (I),
R⁵ and R⁹ are each independently of one another selected from the group consisting of
   H; F; Cl; Br; I; CF₃; OH; CH₂OH; methyl; O-methyl; preferably H; F; Cl; Br; I; CF₃; OH; methyl; O-methyl; more preferably both denote H,
R⁶ and R⁸ are each independently of one another selected from the group consisting of
   H; F; Cl; Br; I; CF₃; OH; CH₂OH; methyl; O-methyl; preferably H; F; Cl; Br; I; CF₃; OH; methyl; O-methyl;
and R⁷ is selected from the group consisting of
   H; F; Cl; Br; I; CN; CF₃; CF₂H; CFH₂; OH; OCF₃; SH; SCF₃; NH₂; C(═O)—NH₂; S(═O)₂—OH; S(═O)₂—NH₂;
   C₁₋₄ alkyl, C₁₋₄ alkylene-OH, C₁₋₄ alkylene-O—C₁₋₄ alkyl, C₁₋₄ alkylene-O—C₁₋₄ alkylene-OH, C₁₋₄ alkylene-O—C₁₋₄ alkylene-O—C₁₋₄ alkyl, C₁₋₄ alkylene-S(═O)₂—C₁₋₄ alkyl, C₁₋₄ alkylene-NH—S(═O)₂—C₁₋₄ alkyl, C₁₋₄ alkylene-NH—S(═O)₂—NH₂, C₁₋₄ alkylene-NH—C₁₋₄-alkylene-OH, C₁₋₄ alkylene-NH—C₁₋₄ alkylene-O—C₁₋₄ alkyl, C₁₋₄ alkylene-N(C₁₋₄ alkyl)-C₁₋₄ alkylene-OH, C₁₋₄ alkylene-N(C₁₋₄alkyl)-C₁₋₄ alkylene-O—C₁₋₄ alkyl, O—C₁₋₄ alkyl, O—C₁₋₄ alkylene-OH, O—C₁₋₄ alkylene-O—C₁₋₄ alkyl, NH—C₁₋₄ alkyl, N(C₁₋₄ alkyl)₂, NH—C₁₋₄ alkylene-OH, NH—C₁₋₄ alkylene-O—C₁₋₄ alkyl, N(C₁₋₄ alkyl)-[C₁₋₄ alkylene-OH], N(C₁₋₄ alkyl)-[C₁₋₄ alkylene-O—C₁₋₄ alkyl], NH—S(═O)₂—C₁₋₄ alkyl,
      wherein C₁₋₄ alkylene can in each case be unsubstituted or monosubstituted with OH,
   a C₃₋₆ cycloaliphatic residue, O—C₃₋₆ cycloaliphatic residue, a 3 to 6 membered heterocycloaliphatic residue,
      wherein the C₃₋₆ cycloaliphatic residue is preferably selected from the group consisting of cyclopropyl, cyclobutyl, cyclopentyl, cyclohexyl, and
      wherein the 3 to 6 membered heterocycloaliphatic residue is preferably selected from the group consisting of tetrahydropyranyl, preferably tetrahydro-2H-pyran-4-yl, azetidinyl, piperidinyl, morpholinyl and pyrrolidinyl,
      wherein the C₃₋₆ cycloaliphatic residue and the 3 to 6 membered heterocycloaliphatic residue, respectively, can be unsubstituted or mono- or polysubstituted with one or more substituents each selected independently of one another from the group consisting of F, Cl, Br, I, OH, O—C₁₋₄ alkyl, NH₂, NH(C₁₋₄ alkyl), and N(C₁₋₄ alkyl)₂, and C₁₋₄ alkyl,
   phenyl, C(═O)—NH-phenyl, NH—C(═O)-phenyl, heteroaryl, C(═O)—NH-heteroaryl, NH—C(═O)-heteroaryl, preferably phenyl, C(═O)—NH-phenyl and NH—C(═O)-phenyl,
      wherein heteroaryl is preferably selected from the group consisting of pyrdiyl, furyl and thienyl;
      wherein in each case independently of one another phenyl and heteroaryl of the aforementioned residues, respectively, can be unsubstituted or mono- or polysubstituted with one or more substituents each selected independently of one another from the group consisting of F, Cl, Br, I, OH, O—C₁₋₄ alkyl, C₁₋₄ alkyl, and CF₃.

In another preferred embodiment of the compound according to the invention of general formula (I),
at least one of R⁵ and R⁹, preferably both R⁵ and R⁹, denote(s) H.

In a further preferred embodiment of the compound according to the invention of general formula (I),
at least one, preferably one, of R⁶ and R⁸ denotes H.

In another preferred embodiment of the compound according to the invention of general formula (I),
both of R⁶ and R⁸ denote H.

In yet another preferred embodiment of the compound according to the invention of general formula (I),
at least one of R⁵ and R⁹, preferably both R⁵ and R⁹, denote(s) H and
at least one, preferably one, of R⁶ and R⁸ denotes H
or both of R⁶ and R⁸ denote H.

In another particularly preferred embodiment of the compound according to the invention of general formula (I),
R⁵ and R⁹ both denote H,
or one of R⁵ and R⁹ denotes H and the remaining residue of R⁵ and R⁹ denotes CH₂OH; more preferably R⁵ and R⁹ both denote H,
R⁶ and R⁸ are each independently of one another selected from the group consisting of
   H; F; Cl; Br; I; CF₃; OH; CH₂OH; methyl; O-methyl; preferably H; F; Cl; Br; I; CF₃; OH; methyl; O-methyl;
and R⁷ is selected from the group consisting of
   H, F, Cl, Br, I, CN, CF₃, CF₂H, CFH₂, OH, OCF₃, SH, SCF₃, NH₂, C(═O)—NH₂, S(═O)₂—OH, S(═O)₂—NH₂,
   CH₃, C₂H₅, CH₂—OH, C₂H₄—OH, CH(OH)—CH₂OH, CH₂—CH(OH)—CH₂—OH, CH₂—O—CH₃, C₂H₄—O—CH₃, CH₂—O—CH₂—OH, CH₂—O—C₂H₄—OH, CH₂—O—CH₂—O—CH₃, CH₂—O—C₂H₄—O—CH₃, CH₂—S(═O)₂—CH₃, C₂H₄—S(═O)₂—CH₃, CH₂—NH—S(═O)₂—CH₃, CH₂—NH—S(═O)₂—NH₂, CH₂—NH—CH₂—OH, CH₂—NH—C₂H₄—OH, CH₂—NH—C₂H₄—O—CH₃, CH₂—N(CH₃)—C₂H₄—OH, CH₂—N(CH₃)—C₂H₄—O—CH₃, O—CH₃, O—C₂H₄—OH, O—C₂H₄—O—CH₃, NH—CH₃, N(CH₃)₂, NH—C₂H₄—OH, NH—C₂H₄—O—CH₃, N(CH₃)—[C₂H₄—OH], N(CH₃)—[C₂H₄—O—CH₃], NH—S(═O)₂—CH₃,
   cyclopropyl, cyclobutyl, cyclopentyl, cyclohexyl, O-cyclopropyl, tetrahydropyranyl, preferably tetrahydro-2H-pyran-4-yl, azetidinyl, piperidinyl, morpholinyl or pyrrolidinyl, in each case independently of one another unsubstituted or mono- or polysubstituted with one or more substituents each selected independently of one another from the group consisting of F, Cl, Br, I, OH, O—CH₃, NH₂, N(CH₃)₂, CH₃, C₂H₅ and tert.-butyl,
   phenyl, C(═O)—NH-phenyl, or NH—C(═O)-phenyl, wherein in each case independently of one another phenyl can be unsubstituted or mono- or polysubstituted with one or more substituents each selected independently of one another from the group consisting of F, Cl, Br, I, OH, O—CH₃, CH₃, C₂H₅, and CF₃.

Particularly preferred residues for R⁷ are selected from the group consisting of
H, F, Cl, Br, I, CN, CF₃, CF₂H, CFH₂, OH, OCF₃, SH, SCF₃, NH₂, C(═O)—NH₂, S(═O)₂—OH, S(═O)₂—NH₂,
CH₃, C₂H₅, CH₂—OH, C₂H₄—OH, CH(OH)—CH₂OH, CH₂—CH(OH)—CH₂—OH, CH₂—O—CH₃, C₂H₄—O—CH₃, CH₂—O—CH₂—OH, CH₂—O—C₂H₄—OH, CH₂—O—CH₂—O—CH₃, CH₂—O—C₂H₄O—CH₃, CH₂—S(═O)₂—CH₃, C₂H₄—S(═O)₂—CH₃, CH₂—NH—S(═O)₂—CH₃, CH₂—NH—S(═O)₂—NH₂, CH₂—NH—CH₂—OH, CH₂—NH—C₂H₄—OH, CH₂—NH—C₂H₄—O—CH₃, CH₂—N(CH₃)—C₂H₄—OH, CH₂—N(CH₃)—C₂H₄—O—CH₃, O—CH₃, O—C₂H₄—OH, O—C₂H₄—O—CH₃, NH—CH₃, N(CH₃)₂, NH—C₂H₄—OH, NH—C₂H₄—O—CH₃, N(CH₃)—[C₂H₄—OH], N(CH₃)—[C₂H₄—O—CH₃], NH—S(═O)₂—CH₃,
cyclopropyl, cyclobutyl, cyclopentyl, cyclohexyl, O-cyclopropyl, tetrahydropyranyl, preferably tetrahydro-2H-pyran-4-yl, azetidinyl, piperidinyl, morpholinyl or pyrrolidinyl, in each case independently of one another unsubstituted or mono- or polysubstituted with one or more substituents each selected independently of one another from the group consisting of F, Cl, Br, I, OH, O—CH₃, NH₂, N(CH₃)₂, CH₃, C₂H₅ and tert.-butyl,
phenyl, C(═O)—NH-phenyl, or NH—C(═O)-phenyl, wherein in each case independently of one another phenyl can be unsubstituted or mono- or polysubstituted with one or more substituents each selected independently of one another from the group consisting of F, Cl, Br, I, OH, O—CH₃, CH₃, C₂H₅, and CF₃.

Preferred are also compounds of formula (I) according to the present invention, wherein
n represents 1;
X represents N or CH;
Y represents O;
Z represents N or C—R^{4b};
A¹ represents N or CR⁵;
A² represents N or CR⁶;
A³ represents N or CR⁷;
A⁴ represents N or CR⁸;
A⁵ represents N or CR⁹;
with the proviso that 1, 2 or 3 of variables A¹, A², A³, A⁴ and A⁵ represent a nitrogen atom;
R¹ is selected from the group consisting of tert-Butyl, CF₃, cyclopropyl, cyclobutyl, cyclopentyl, and cyclohexyl;
R² represents substructure (T1)

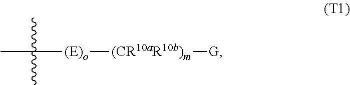

in which
E represents O, S, or NR¹¹,
wherein R¹¹ represents H or a C₁₋₄ aliphatic residue, unsubstituted or mono- or polysubstituted with one or more substituents each selected independently of one another from the group consisting of F, Cl, Br, I, OH, O—C₁₋₄ alkyl, OCF₃, NH₂, NH—C₁₋₄ alkyl and N(C₁₋₄ alkyl)₂;
o represents 0 or 1; preferably denotes 0,
R^{10a} and R^{10b} each independently of one another represent H; F; Cl; Br; I; or a C₁₋₄ aliphatic residue, unsubstituted or mono- or polysubstituted with one or more substituents each selected independently of one another from the group consisting of F, Cl, Br, I, OH, O—C₁₋₄ alkyl, OCF₃, NH₂, NH—C₁₋₄ alkyl and N(C₁₋₄ alkyl)₂;
m represents 0, 1, 2, 3 or 4, preferably 0, 1 or 2, more preferably 0 or 1;
G represents a C₁₋₄ aliphatic residue, unsubstituted or mono- or polysubstituted with one or more substituents each selected independently of one another from the group consisting of F, Cl, Br, I, NO₂, CN, OH, ═O, O—C₁₋₄ alkyl, O—C₁₋₄ alkylen-O—C₁₋₄ alkyl, OCF₃, CF₃, NH₂, NH(C₁₋₄ alkyl), N(C₁₋₄ alkyl)₂, SH, S—C₁₋₄ alkyl, SCF₃, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH;
or represents a C₃₋₁₀ cycloaliphatic residue or a 3 to 10 membered heterocyclo-aliphatic residue, in each case unsubstituted or mono- or polysubstituted with one or more substituents each selected independently of one another from the group consisting of F, Cl, Br, I, NO₂, CN, OH, ═O, O—C₁₋₄ alkyl, OCF₃, C₁₋₄ alkyl, CF₃, SH, S—C₁₋₄ alkyl, SCF₃, NH₂, NH(C₁₋₄ alkyl), N(C₁₋₄ alkyl)₂, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH;
or represents an aryl or heteroaryl, unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, CF₃, SH, S—C₁₋₄ alkyl, SCF₃, NH₂, NH(C₁₋₄ alkyl), N(C₁₋₄ alkyl)₂, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH;
R³ is selected from the group consisting of H, methyl, and ethyl.
R^{4a} represents H; methyl, ethyl, cyclopropyl, cyclobutyl, cyclopentyl, cyclohexyl, or phenyl, wherein phenyl is unsubstituted or substituted with 1, 2, 3, 4 or 5 substituents independently selected from the group consisting of F; Cl; Br; I; NO₂; CN; CF₃; CF₂H; CFH₂; CF₂Cl; CFCl₂; OH, NH₂, NH(C₁₋₄ alkyl) and N(C₁₋₄ alkyl)(C₁₋₄ alkyl), C₁₋₄ alkyl, and O—C₁₋₄-alkyl;
R^{4b} represents H; methyl, or ethyl,
or R^{4a} and R^{4b} together with the carbon atom connecting them form cyclopropyl, cyclobutyl, cyclopentyl, or cyclohexyl ring;
R⁵, R⁶, R⁷, R⁸ and R⁹ are each independently of one another selected from the group consisting of
   H; F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; NH₂; C(═O)—NH₂; C(═O)—H; C(═O)—OH; S(═O)₂—OH; S(═O)₂—NH₂;
   a C₁₋₁₀ aliphatic residue, (C₁₋₈ aliphatic group)-OH, (C₁₋₈ aliphatic group)-O—C₁₋₁₀ aliphatic residue, (C₁₋₈ aliphatic group)-O—(C₁₋₈ aliphatic group)-OH, (C₁₋₈ aliphatic group)-O—(C₁₋₈ aliphatic group)-O—C₁₋₁₀ aliphatic residue, a (C₁₋₈ aliphatic group)-NH—C₁₋₁₀ aliphatic residue, a (C₁₋₈ aliphatic group)-NH—(C₁₋₈ aliphatic residue)-OH, a (C₁₋₈ aliphatic group)-N(C₁₋₁₀ aliphatic residue)-(C₁₋₈ aliphatic residue)-OH, a (C₁₋₈ aliphatic group)-NH—S(═O)₂—C₁₋₁₀ aliphatic residue, a (C₁₋₈ aliphatic group)-NH—S(═O)₂—NH₂, a (C₁₋₈ aliphatic group)-S(═O)₂—C₁₋₁₀ aliphatic residue,
   a O—C₁₋₁₀ aliphatic residue, a O—(C₁₋₈ aliphatic group)-O—C₁₋₁₀ aliphatic residue, O—(C₁₋₈ aliphatic group)-OH,
   a NH—C₁₋₁₀ aliphatic residue, a N(C₁₋₁₀ aliphatic residue)₂, a NH—(C₁₋₈ aliphatic group)-O—C₁₋₁₀ aliphatic residue, a NH—(C₁₋₈ aliphatic group)-OH, a N(C₁₋₁₀ aliphatic residue)[(C₁₋₈ aliphatic group)-O—C₁₋₁₀ aliphatic residue], a N(C₁₋₁₀ aliphatic residue)[(C₁₋₈ aliphatic group)-OH], a NH—S(═O)₂—C₁₋₁₀ aliphatic residue,
      wherein each of the aforementioned C₁₋₁₀ aliphatic residue and C₁₋₈ aliphatic groups can in each case be unsubstituted or monosubstituted with OH;
   a C₃₋₁₀ cycloaliphatic residue, a C(═O)—C₃₋₁₀ cycloaliphatic residue, a C(═O)NH—C₃₋₁₀ cycloaliphatic residue, a O—C₃₋₁₀ cycloaliphatic residue, a NH—C₃₋₁₀ cycloaliphatic residue, a NH—C(═O)—C₃₋₁₀ cycloaliphatic residue, a 3 to 10 membered heterocycloaliphatic residue, a C(═O)-(3 to 10 membered heterocycloaliphatic residue), a C(═O)—NH-(3 to 10 membered heterocycloaliphatic residue), a O-(3 to 10 membered heterocycloaliphatic residue), a NH-(3 to 10 membered heterocycloaliphatic residue), a NH—C(═O)-(3 to 10 membered heterocycloaliphatic residue),
      wherein in each case independently of one another, the C₃₋₁₀ cycloaliphatic residue and the 3 to 10 membered heterocycloaliphatic residue, respectively, can be unsubstituted or mono- or polysubstituted with one or more substituents each selected independently of one another from the group consisting of F, Cl, Br, I, C₁₋₄ alkyl, C₁₋₄ alkylene-OH, C₁₋₄ alkylene-O—C₁₋₄ alkyl, CF₃, C(═O)—C₁₋₄ alkyl, O—C₁₋₄ alkyl, O—C₁₋₄ alkylene-OH, O—C₁₋₄ alkylene-O—C₁₋₄ alkyl, OCF₃, OH, SH, S—C₁₋₄ alkyl, SCF₃, SO₂—C₁₋₄ alkyl, NH₂, NH—C₁₋₄ alkyl, N(C₁₋₄ alkyl)₂, NH—SO₂—C₁₋₄ alkyl, NH—C(═O)—C₁₋₄ alkyl;
   aryl, C(═O)-aryl, C(═O)—NH-aryl, NH—C(═O)-aryl, heteroaryl, C(═O)-heteroaryl, C(═O)—NH-heteroaryl, NH—C(═O)-heteroaryl,
      wherein in each case independently of one another the aryl and heteroaryl of the aforementioned residues, respectively, can be unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl, O—C₁₋₄ alkylene-OH, OCF₃, C₁₋₄ alkyl, C₁₋₄-alkylene-O—C₁₋₄-alkyl, C₁₋₄ alkylene-OH, C(═O)—C₁₋₄ alkyl, CF₃, CF₂H, CHF₂, SH, S—C₁₋₄ alkyl, SCF₃, SO₂—C₁₋₄ alkyl, NH₂, NH(C₁₋₄ alkyl), N(C₁₋₄ alkyl)₂, NH—SO₂—C₁₋₄ alkyl, NH—C(═O)—C₁₋₄ alkyl, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl OCF₃, C₁₋₄ alkyl, C₁₋₄ alkylene-O—C₁₋₄-alkyl, C(═O)—OH, CF₃, CF₂H, CHF₂, NH₂, NH(C₁₋₄ alkyl), N(C₁₋₄ alkyl)₂, SH, S—C₁₋₄ alkyl, SCF₃ and S(═O)₂OH.

Another preferred embodiment of the present invention is the compound according to the general formula (I), wherein
R¹ is selected from the group consisting of CF₃, tert.-butyl, and cyclopropyl,
R² represents phenyl, unsubstituted or mono- or polysubstituted with one or more substituents each selected independently of one another from the group consisting of F, Cl, Br, I, OH, O—CH₃, CH₃, CH(CH₃)₂, N(CH₃)₂, tert.-butyl and CF₃, preferably phenyl mono- or disubstituted with one or two substituents each selected independently of one another from the group consisting of F, Cl, Br, I, O—CH₃, CH₃, CH(CH₃)₂, N(CH₃)₂, tert.-butyl and CF₃, more preferably phenyl mono-substituted in meta position with one substituent selected from the group consisting of F, Cl, CH₃, OCH₃, CH(CH₃)₂ and N(CH₃)₂,
   or represents 4-methylpiperidinyl,
R³ represents H,
n represents 1,
X represents CH or N, preferably N,
R^{4a} represents H, or methyl,
Y denotes O,
Z represents N or CR^{4b},
   preferably represents N when R^{4a} denotes H or
   preferably represents CR^{4b} when R^{4a} and R^{4b} each represent H or
   preferably represents CR^{4b} when R^{4a} represents methyl and R^{4b} represents H,
R^{4b} represents H or methyl,
A¹ represents C—R⁵,
A² represents N,
A³ represents C—R⁷,
A⁴ represents N or C—R⁸, preferably CR⁸,
A⁵ represents C—R⁹,
R⁵ and R⁹ both denote H,
or one of R⁵ and R⁹ denotes H and the remaining residue of R⁵ and R⁹ denotes CH₂OH; more preferably R⁵ and R⁹ both denote H,
R⁶ and R⁸ are each independently of one another selected from the group consisting of
   H; F; Cl; Br; I; CF₃; OH; CH₂OH; methyl; O-methyl; preferably H; F; Cl; Br; I; CF₃; OH; methyl; O-methyl;
and R⁷ is selected from the group consisting of
   H, F, Cl, Br, I, CN, CF₃, CF₂H, CFH₂, OH, OCF₃, SH, SCF₃, NH₂, C(═O)—NH₂, S(═O)₂—OH, S(═O)₂—NH₂,
   CH₃, C₂H₅, CH₂—OH, C₂H₄—OH, CH(OH)—CH₂OH, CH₂—O—CH₃, C₂H₄—O—CH₃, CH₂—O—CH₂—OH, CH₂—O—C₂H₄—OH, CH₂—O—CH₂—O—CH₃, CH₂—O—C₂H₄—O—CH₃, CH₂—S(═O)₂—CH₃, C₂H₄—S(═O)₂—CH₃, CH₂—NH—S(═O)₂—CH₃, CH₂—NH—S(═O)₂—NH₂, CH₂—NH—CH₂—OH, CH₂—NH—C₂H₄—OH, CH₂—NH—C₂H₄—O—CH₃, CH₂—N(CH₃)—C₂H₄—OH, CH₂—N(CH₃)—C₂H₄—O—CH₃, O—CH₃, O—C₂H₄—OH, O—C₂H₄—O—CH₃, NH—CH₃, N(CH₃)₂, NH—C₂H₄—OH, NH—C₂H₄—O—CH₃, N(CH₃)—[C₂H₄—OH], N(CH₃)—[C₂H₄—O—CH₃], NH—S(═O)₂—CH₃,
   cyclopropyl, cyclobutyl, cyclopentyl, cyclohexyl, O-cyclopropyl, tetrahydropyranyl, preferably tetrahydro-2H-pyran-4-yl, azetidinyl, piperidinyl, morpholinyl or pyrrolidinyl,
   in each case independently of one another unsubstituted or mono- or polysubstituted with one or more substituents each selected independently of one another from the group consisting of F, Cl, Br, I, OH, O—CH₃, NH₂, N(CH₃)₂, CH₃, C₂H₅ and tert.-butyl,
   phenyl, C(═O)—NH-phenyl, or NH—C(═O)-phenyl, wherein in each case independently of one another phenyl can be unsubstituted or mono- or polysubstituted with one or more substituents each selected independently of one another from the group consisting of F, Cl, Br, I, OH, O—CH₃, CH₃, C₂H₅, and CF₃,
   preferably R⁷ is selected from the group consisting of C₂H₄—S(═O)₂—CH₃, CH₂—O—C₂H₄—OH, CH₂—OH, CH₂—NH—S(═O)₂—CH₃, CH(OH)—CH₂OH, and C₂H₄—OH, more preferably selected from the group consisting of C₂H₄—S(═O)₂—CH₃, CH₂—O—C₂H₄—OH, CH₂—OH, CH₂—NH—S(═O)₂—CH₃ and C₂H₄—OH.

Further embodiments of the compounds according to the invention are those which are represented by the general formulae A1-A14 shown in the following:

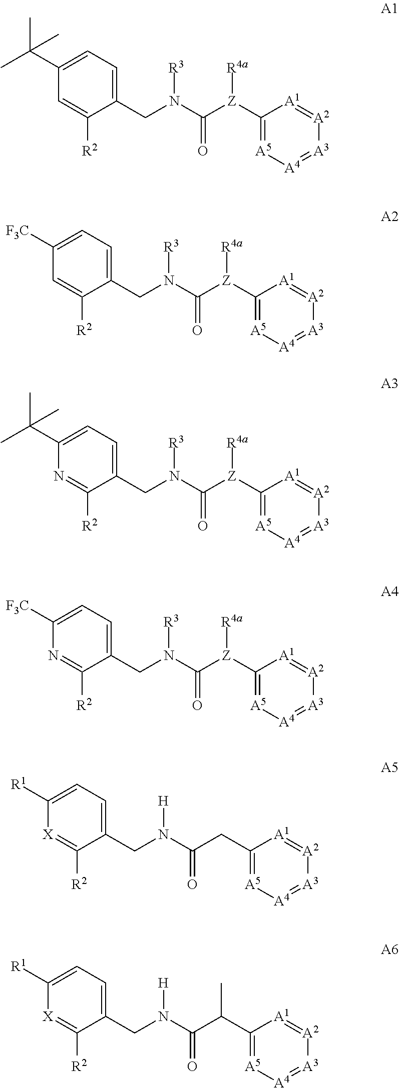

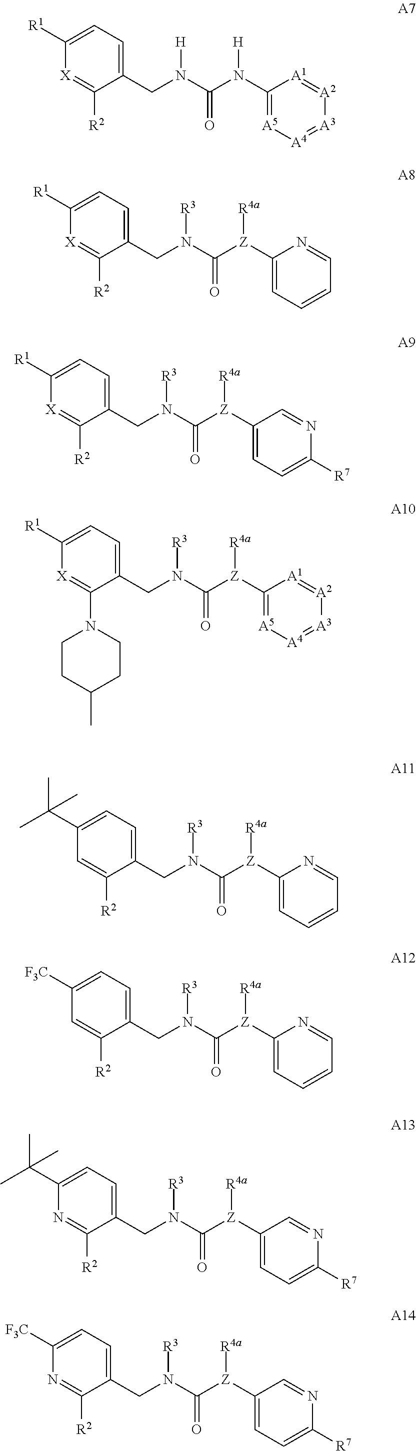

wherein the particular radicals, variables and indices have the meanings described herein in connection with the compounds according to the invention and preferred embodiments thereof.

Particularly preferred are compounds according to the invention selected from the group consisting of:
1. N-((2-Pentyl-6-(trifluoromethyl)pyridin-3-yl)methyl)-2-(pyridin-2-yl)acetamide;
2. N-((2-Cyclopentyl-6-(trifluoromethyl)pyridin-3-yl)methyl)-2-(pyridin-2-yl)acetamide;
3. 1-(Pyridin-2-yl)-3-((2-(tetrahydro-2H-pyran-4-yl)-6-(trifluoromethyl)pyridin-3-yl)methyl)urea;
4. N-((2-(Cyclohexyl methyl)-6-(trifluoromethyl)pyridin-3-yl)methyl)-2-(pyridin-2-yl)acetamide;
5. N-((2-(3-Chlorophenyl)-6-(trifluoromethyl)pyridin-3-yl)methyl)-2-(pyridin-2-yl)acetamide
6. N-((2-(3-Chloro-4-fluorophenyl)-6-(trifluoromethyl)pyridin-3-yl)methyl)-2-(pyridin-2-yl)acetamide;
7. 2-(Pyridin-2-yl)-N-((2-m-tolyl-6-(trifluoromethyl)pyridin-3-yl)methyl)acetamide
8. N-((2-(3-Methoxyphenyl)-6-(trifluoromethyl)pyridin-3-yl)methyl)-2-(pyridin-2-yl)acetamide;
9. N-((2-(Butylamino)-6-(trifluoromethyl)pyridin-3-yl)methyl)-2-(pyridin-2-yl)acetamide;
10. 2-(Pyridin-2-yl)-N-((2-(pyrrolidin-1-yl)-6-(trifluoromethyl)pyridin-3-yl)methyl)acetamide;
11. N-(2-(4-Methylpiperidin-1-yl)-4-(trifluoromethyl)benzyl)-2-(pyridin-2-yl)acetamide;
12. N-((6-tert-Butyl-2-(4-methylpiperidin-1-yl)pyridin-3-yl)methyl)-2-(pyridin-2-yl)acetamide;
13. N-((2-(4-Methylpiperidin-1-yl)-6-(trifluoromethyl)pyridin-3-yl)methyl)-2-(pyridin-2-yl)acetamide;
14. N-((2-(4-Methylpiperidin-1-yl)-6-(trifluoromethyl)pyridin-3-yl)methyl)-2-(pyridin-2-yl)propanamide;
15. 2-Methyl-N-((2-(4-methylpiperidin-1-yl)-6-(trifluoromethyl)pyridin-3-yl)methyl)-2-(pyridin-2-yl)propanamide;
16. N-((2-(4-Methylpiperidin-1-yl)-6-(trifluoromethyl)pyridin-3-yl)methyl)-1-(pyridin-2-yl)cyclopropanecarboxamide;
17. 2-Cyclohexyl-N-((2-(4-methylpiperidin-1-yl)-6-(trifluoromethyl)pyridin-3-yl)methyl)-2-(pyridin-2-yl)acetamide;
18. N-((2-(4-Methylpiperidin-1-yl)-6-(trifluoromethyl)pyridin-3-yl)methyl)-2-(pyridin-2-yl)-2-m-tolylacetamide;
19. 1-((2-(4-Methylpiperidin-1-yl)-6-(trifluoromethyl)pyridin-3-yl)methyl)-3-(pyridin-2-yl)urea;
20. 1-Methyl-3-((2-(4-methylpiperidin-1-yl)-6-(trifluoromethyl)pyridin-3-yl)methyl)-1-(pyridin-2-yl)urea;
21. 1-Methyl-1-((2-(4-methylpiperidin-1-yl)-6-(trifluoromethyl)pyridin-3-yl)methyl)-3-(pyridin-2-yl)urea;
22. N-((2-Morpholino-6-(trifluoromethyl)pyridin-3-yl)methyl)-2-(pyridin-2-yl)acetamide;
23. 1-((2-(4-(Dimethylamino)piperidin-1-yl)-6-(trifluoromethyl)pyridin-3-yl)methyl)-3-(pyridin-2-yl)urea;
24. N-((2-((2-Methoxyethoxy)methyl)-6-(trifluoromethyl)pyridin-3-yl)methyl)-2-(pyridin-2-yl)acetamide;
25. N-((2-Butoxy-6-(trifluoromethyl)pyridin-3-yl)methyl)-2-(pyridin-2-yl)acetamide;
26. N-((2-(Cyclobutylmethoxy)-6-(trifluoromethyl)pyridin-3-yl)methyl)-2-(pyridin-2-yl)acetamide;
27. N-((2-(Cyclohexyloxy)-6-(trifluoromethyl)pyridin-3-yl)methyl)-2-(pyridin-2-yl)acetamide;
28. N-(4-tert-Butyl-2-(cyclohexylthio)benzyl)-2-(pyridin-2-yl)acetamide;
29. N-(2-(Cyclohexylthio)-4-(trifluoromethyl)benzyl)-2-(pyridin-2-yl)acetamide;
30. N-((6-Cyclopropyl-2-(4-methylpiperidin-1-yl)pyridin-3-yl)methyl)-2-(pyridin-2-yl)acetamide;
31. N-((2-(4-Methylpiperidin-1-yl)-6-(trifluoromethyl)pyridin-3-yl)methyl)-2-(pyridin-3-yl)acetamide;
32. N-((2-(4-Methylpiperidin-1-yl)-6-(trifluoromethyl)pyridin-3-yl)methyl)-2-(pyridin-3-yl)propanamide;
33. N-(4-tert-Butyl-2-(4-methylpiperidin-1-yl)benzyl)-2-(pyridin-3-yl)acetamide;
34. N-((2-(Cyclohexylthio)-6-(trifluoromethyl)pyridin-3-yl)methyl)-2-(pyridin-3-yl)acetamide;
35. 1-((2-(3-Chlorophenyl)-6-(trifluoromethyl)pyridin-3-yl)methyl)-3-(pyridin-3-yl)urea;
36. 1-(Pyridin-3-yl)-3-((2-m-tolyl-6-(trifluoromethyl)pyridin-3-yl)methyl)urea;
37. 1-((2-(3-Methoxyphenyl)-6-(trifluoromethyl)pyridin-3-yl)methyl)-3-(pyridin-3-yl)urea;
38. N-((2-(4-Methylpiperidin-1-yl)-6-(trifluoromethyl)pyridin-3-yl)methyl)-2-(pyridin-4-yl)acetamide;
39. N-((2-(4-Methylpiperidin-1-yl)-6-(trifluoromethyl)pyridin-3-yl)methyl)-2-(pyrimidin-4-yl)acetamide;
40. N-((2-(4-Methylpiperidin-1-yl)-6-(trifluoromethyl)pyridin-3-yl)methyl)-2-(pyrazin-2-yl)acetamide;
41. N-((2-(4-Methylpiperidin-1-yl)-6-(trifluoromethyl)pyridin-3-yl)methyl)-2-(pyrimidin-2-yl)acetamide;
42. 1-((2-(4-Methylpiperidin-1-yl)-6-(trifluoromethyl)pyridin-3-yl)methyl)-3-(pyridazin-4-yl)urea;
43. 1-(2-(4-Methylpiperidin-1-yl)-4-(trifluoromethyl)benzyl)-3-(pyridazin-4-yl)urea;
44. 1-(4-tert-Butyl-2-(cyclohexylthio)benzyl)-3-(pyridazin-4-yl)urea;
45. 1-((2-(3-Fluorophenyl)-6-(trifluoromethyl)pyridin-3-yl)methyl)-3-(pyridazin-4-yl)urea;
46. 1-((2-(3-Chloro-4-fluorophenyl)-6-(trifluoromethyl)pyridin-3-yl)methyl)-3-(pyridazin-4-yl)urea;
47. N-((2-(4-Methylpiperidin-1-yl)-6-(trifluoromethyl)pyridin-3-yl)methyl)-2-(pyrimidin-5-yl)acetamide;
48. 1-((2-(4-Methylpiperidin-1-yl)-6-(trifluoromethyl)pyridin-3-yl)methyl)-3-(1,3,5-triazin-2-yl)urea;
49. 2-(6-Chloropyridin-3-yl)-N-((2-(4-methylpiperidin-1-yl)-6-(trifluoromethyl)pyridin-3-yl)methyl)propanamide;
50. 2-(5-Fluoropyridin-3-yl)-N-((2-(4-methylpiperidin-1-yl)-6-(trifluoromethyl)pyridin-3-yl)methyl)acetamide;
51. 1-(5-Fluoropyridin-3-yl)-3-((2-(4-methylpiperidin-1-yl)-6-(trifluoromethyl)pyridin-3-yl)methyl)urea;
52. 1-((2-(4-Methylpiperidin-1-yl)-6-(trifluoromethyl)pyridin-3-yl)methyl)-3-(2-methylpyrimidin-5-yl)urea;
53. 2-(6-(Hydroxymethyl)pyridin-3-yl)-N-((2-(4-methylpiperidin-1-yl)-6-(trifluoromethyl)pyridin-3-yl)methyl)propanamide;
54. N-((2-(3-Fluorophenyl)-6-(trifluoromethyl)pyridin-3-yl)methyl)-2-(6-(hydroxymethyl)pyridin-3-yl)propanamide;
55. 1-(6-(Hydroxymethyl)pyridin-3-yl)-3-((2-(4-methylpiperidin-1-yl)-6-(trifluoromethyl)pyridin-3-yl)methyl)urea;
56. 1-(6-(Hydroxymethyl)pyridin-3-yl)-3-((2-pentyl-6-(trifluoromethyl)pyridin-3-yl)methyl)urea;
57. 1-((2-(3-Fluorphenyl)-6-(trifluormethyl)pyridin-3-yl)methyl)-3-(6-(hydroxymethyl)pyridin-3-yl)urea;
58. 1-(6-(Hydroxymethyl)pyridin-3-yl)-3-((2-m-tolyl-6-(trifluoromethyl)pyridin-3-yl)methyl)urea;
59. 1-(6-(Hydroxymethyl)pyridin-3-yl)-3-((2-(3-isopropylphenyl)-6-(trifluoromethyl)pyridin-3-yl)methyl)urea;
60. 1-((2-(3-(Dimethylamino)phenyl)-6-(trifluoromethyl)pyridin-3-yl)methyl)-3-(6-(hydroxymethyl)pyridin-3-yl)urea;
61. 1-(5-Fluoro-6-(hydroxymethyl)pyridin-3-yl)-3-((2-(4-methylpiperidin-1-yl)-6-(trifluoromethyl)pyridin-3-yl)methyl)urea;
62. 2-(6-(2-Hydroxyethyl)pyridin-3-yl)-N-((2-(4-methylpiperidin-1-yl)-6-(trifluoromethyl)pyridin-3-yl)methyl)propanamide;
63. 1-(6-(2-Hydroxyethyl)pyridin-3-yl)-3-((2-(4-methylpiperidin-1-yl)-6-(trifluoromethyl)pyridin-3-yl)methyl)urea;
64. 2-(6-((2-Hydroxyethoxy)methyl)pyridin-3-yl)-N-((2-(4-methylpiperidin-1-yl)-6-(trifluoromethyl)pyridin-3-yl)methyl)propanamide;
65. 1-((2-(4-Methylpiperidin-1-yl)-6-(trifluoromethyl)pyridin-3-yl)methyl)-3-(6-(methylsulfonylmethyl)pyridin-3-yl)urea;
66. 1-((2-(4-Methylpiperidin-1-yl)-6-(trifluoromethyl)pyridin-3-yl)methyl)-3-(6-(2-(methylsulfonyl)ethyl)pyridin-3-yl)urea;
67. 1-(5-Fluoro-6-(2-(methylsulfonyl)ethyl)pyridin-3-yl)-3-((2-(4-methylpiperidin-1-yl)-6-(trifluoromethyl)pyridin-3-yl)methyl)urea;
68. 1-((6-Cyclopropyl-2-(4-methylpiperidin-1-yl)pyridin-3-yl)methyl)-3-(5-fluoro-6-(2-(methylsulfonyl)ethyl)pyridin-3-yl)urea;
69. 1-(5-Fluoro-6-(2-(methylsulfonyl)ethyl)pyridin-3-yl)-3-((2-(3-fluorophenyl)-6-(trifluoromethyl)pyridin-3-yl)methyl)urea;
70. N-((5-(3-((2-(4-Methylpiperidin-1-yl)-6-(trifluoromethyl)pyridin-3-yl)methyl)ureido)pyridin-2-yl)methyl)methanesulfonamide;
71. N-((5-(3-((2-(4-Methylpiperidin-1-yl)-6-(trifluoromethyl)pyridin-3-yl)methyl)ureido)pyridin-2-yl)methyl)sulfuric diamide;
72. N-((5-(3-(2-(Cyclohexyloxy)-4-(trifluoromethyl)benzyl)ureido)pyridin-2-yl)methyl)sulfuric diamide;
73. N-((5-(3-((2-m-Tolyl-6-(trifluoromethyl)pyridin-3-yl)methyl)ureido)pyridin-2-yl)methyl)sulfuric diamide;
74. 5-(1-((2-(4-Methylpiperidin-1-yl)-6-(trifluoromethyl)pyridin-3-yl)methylamino)-1-oxopropan-2-yl)picolinamide;
75. 5-(1-((2-(4-Methylpiperidin-1-yl)-6-(trifluoromethyl)pyridin-3-yl)methylamino)-1-oxopropan-2-yl)-N-phenylpicolinamide;
76. 5-(1-((2-(4-Methylpiperidin-1-yl)-6-(trifluoromethyl)pyridin-3-yl)methylamino)-1-oxopropan-2-yl)-N-phenylpyrimidine-2-carboxamide;
77. 5-(1-((2-(Ethylamino)-6-(trifluoromethyl)pyridin-3-yl)methylamino)-1-oxopropan-2-yl)-N-(4-fluorophenyl)pyrimidine-2-carboxamide;
78. N-(4-Fluorophenyl)-5-(1-oxo-1-((2-(pyrrolidin-1-yl)-6-(trifluoromethyl)pyridin-3-yl)methylamino)propan-2-yl)pyrimidine-2-carboxamide;
79. N-(4-Fluorophenyl)-5-(1-oxo-1-((2-(piperidin-1-yl)-6-(trifluoromethyl)pyridin-3-yl)methylamino)propan-2-yl)pyrimidine-2-carboxamide;
80. N-(4-Fluorophenyl)-5-(1-((2-morpholino-6-(trifluoromethyl)pyridin-3-yl)methylamino)-1-oxopropan-2-yl)pyrimidine-2-carboxamide;
81. N-(4-Fluorophenyl)-5-(1-oxo-1-((2-m-tolyl-6-(trifluoromethyl)pyridin-3-yl)methylamino)propan-2-yl)pyrimidine-2-carboxamide;
82. 5-(1-oxo-1-((2-(piperidin-1-ylmethyl)-6-(trifluoromethyl)pyridin-3-yl)methylamino)propan-2-yl)-N-(4-(trifluoromethyl)phenyl)pyrimidine-2-carboxamide;
83. 1-((2-(4-Methylpiperidin-1-yl)-6-(trifluoromethyl)pyridin-3-yl)methyl)-3-(6-(tetrahydro-2H-pyran-4-yl)pyridin-3-yl)urea;
84. 2-(5-Amino-6-bromopyridin-2-yl)-N-((2-(4-methylpiperidin-1-yl)-6-(trifluoromethyl)pyridin-3-yl)methyl)propanamide;
85. 2-(6-(2-Hydroxyethylamino)pyridin-3-yl)-N-((2-(4-methylpiperidin-1-yl)-6-(trifluoromethyl)pyridin-3-yl)methyl)propanamide;
86. 1-(6-(2-Hydroxyethylamino)pyridin-3-yl)-3-((2-(4-methylpiperidin-1-yl)-6-(trifluoromethyl)pyridin-3-yl)methyl)urea;
87. 2-(6-(2-Methoxyethylamino)pyridin-3-yl)-N-((2-(4-methylpiperidin-1-yl)-6-(trifluoromethyl)pyridin-3-yl)methyl)propanamide;
88. 1-(6-(2-Methoxyethylamino)pyridin-3-yl)-3-((2-(4-methylpiperidin-1-yl)-6-(trifluoromethyl)pyridin-3-yl)methyl)urea;
89. 2-(6-((2-Hydroxyethyl)(methyl)amino)pyridin-3-yl)-N-((2-(4-methylpiperidin-1-yl)-6-(trifluoromethyl)pyridin-3-yl)methyl)propanamide;
90. 1-(6-((2-Hydroxyethyl)(methyl)amino)pyridin-3-yl)-3-((2-(4-methylpiperidin-1-yl)-6-(trifluoromethyl)pyridin-3-yl)methyl)urea;
91. 1-(6-((2-Methoxyethyl)(methyl)amino)pyridin-3-yl)-3-((2-(4-methylpiperidin-1-yl)-6-(trifluoromethyl)pyridin-3-yl)methyl)urea;
92. N-((2-(4-Methylpiperidin-1-yl)-6-(trifluormethyl)pyridin-3-yl)methyl)-2-(6-(methylsulfonamido)pyridin-3-yl)propanamide;
93. N-(5-(3-((2-(4-Methylpiperidin-1-yl)-6-(trifluoromethyl)pyridin-3-yl)methyl)ureido)pyridin-2-yl)methanesulfonamide;
94. N-(5-(3-((6-Cyclopropyl-2-(4-methylpiperidin-1-yl)pyridin-3-yl)methyl)ureido)pyridin-2-yl)methanesulfonamide;
95. 2-(6-(Methylsulfonamido)pyridin-3-yl)-N-((2-morpholino-6-(trifluoromethyl)pyridin-3-yl)methyl)propanamide;
96. 2-(5-Fluoro-6-(methylsulfonamido)pyridin-3-yl)-N-((2-(4-methylpiperidin-1-yl)-6-(trifluoromethyl)pyridin-3-yl)methyl)propanamide;
97. 2-(5-Methoxy-6-(methylsulfonamido)pyridin-3-yl)-N-((2-(4-methylpiperidin-1-yl)-6-(trifluoromethyl)pyridin-3-yl)methyl)propanamide;
98. N-(5-(1-((2-(4-Methylpiperidin-1-yl)-6-(trifluoromethyl)pyridin-3-yl)methylamino)-1-oxopropan-2-yl)pyridin-2-yl)benzamide;
99. 4-Chloro-N-(5-(1-((2-(4-methylpiperidin-1-yl)-6-(trifluoromethyl)pyridin-3-yl)methylamino)-1-oxopropan-2-yl)pyridin-2-yl)benzamide;
100. 4-Chloro-N-(5-(1-(2-(4-methylpiperidin-1-yl)-4-(trifluoromethyl)benzylamino)-1-oxopropan-2-yl)pyridin-2-yl)benzamide;
101. 4-Chloro-N-(5-(1-(2-(cyclohexylthio)-4-(trifluoromethyl)benzylamino)-1-oxopropan-2-yl)pyridin-2-yl)benzamide;
102. N-(5-(1-(4-tert-Butyl-2-(cyclohexylthio)benzylamino)-1-oxopropan-2-yl)pyridin-2-yl)-4-chlorobenzamide;
103. 4-Chloro-N-(5-(1-(2-(cyclopentyloxy)-4-(trifluoromethyl)benzylamino)-1-oxopropan-2-yl)pyridin-2-yl)benzamide;
104. 1-(6-(Dimethylamino)-5-(trifluoromethyl)pyridin-3-yl)-3-((2-(4-methylpiperidin-1-yl)-6-(trifluoromethyl)pyridin-3-yl)methyl)urea;
105. 1-(6-(Azetidin-1-yl)pyridin-3-yl)-3-((2-(4-methylpiperidin-1-yl)-6-(trifluoromethyl)pyridin-3-yl)methyl)urea;
106. 1-(6-(Azetidin-1-yl)-5-fluoropyridin-3-yl)-3-((2-(4-methylpiperidin-1-yl)-6-(trifluoromethyl)pyridin-3-yl)methyl)urea;
107. 1-(6-(Azetidin-1-yl)-5-methoxypyridin-3-yl)-3-((2-(4-methylpiperidin-1-yl)-6-(trifluoromethyl)pyridin-3-yl)methyl)urea;
108. 1-(6-(3-Hydroxyazetidin-1-yl)pyridin-3-yl)-3-((2-(4-methylpiperidin-1-yl)-6-(trifluoromethyl)pyridin-3-yl)methyl)urea;
109. 1-(6-(3-Hydroxyazetidin-1-yl)pyridin-3-yl)-3-((2-pentyl-6-(trifluoromethyl)pyridin-3-yl)methyl)urea;
110. 1-(6-(3-Hydroxyazetidin-1-yl)pyridin-3-yl)-3-((2-m-tolyl-6-(trifluoromethyl)pyridin-3-yl)methyl)urea;
111. 1-(6-(3-Hydroxyazetidin-1-yl)pyridin-3-yl)-3-((2-methoxy-6-(trifluoromethyl)pyridin-3-yl)methyl)urea;
112. 1-(6-(3-Hydroxyazetidin-1-yl)pyridin-3-yl)-3-((2-isobutoxy-6-(trifluoromethyl)pyridin-3-yl)methyl)urea;
113. 1-((2-(Cyclobutylmethoxy)-6-(trifluoromethyl)pyridin-3-yl)methyl)-3-(6-(3-hydroxyazetidin-1-yl)pyridin-3-yl)urea;
114. 1-((2-(4-Methylpiperidin-1-yl)-6-(trifluoromethyl)pyridin-3-yl)methyl)-3-(6-(pyrrolidin-1-yl)pyridin-3-yl)urea;
115. 1-(5-Fluoro-6-(pyrrolidin-1-yl)pyridin-3-yl)-3-((2-(4-methylpiperidin-1-yl)-6-(trifluoromethyl)pyridin-3-yl)methyl)urea;
116. 1-(5-Methoxy-6-(pyrrolidin-1-yl)pyridin-3-yl)-3-((2-(4-methylpiperidin-1-yl)-6-(trifluoromethyl)pyridin-3-yl)methyl)urea;
117. (S)-1-(6-(3-Hydroxypyrrolidin-1-yl)pyridin-3-yl)-3-((2-(4-methylpiperidin-1-yl)-6-(trifluoromethyl)pyridin-3-yl)methyl)urea;
118. (R)-1-(6-(3-Hydroxypyrrolidin-1-yl)pyridin-3-yl)-3-((2-(4-methylpiperidin-1-yl)-6-(trifluoromethyl)pyridin-3-yl)methyl)urea;
119. 1-(6-Hydroxypyridin-3-yl)-3-((2-(4-methylpiperidin-1-yl)-6-(trifluoromethyl)pyridin-3-yl)methyl)urea;
120. 2-(6-Methoxypyridin-3-yl)-N-((2-(4-methylpiperidin-1-yl)-6-(trifluoromethyl)pyridin-3-yl)methyl)propanamide;
121. 1-(2-Methoxypyrimidin-5-yl)-3-((2-(4-methylpiperidin-1-yl)-6-(trifluoromethyl)pyridin-3-yl)methyl)urea;
122. 1-(2-Cyclobutoxypyrimidin-5-yl)-3-((2-(4-methylpiperidin-1-yl)-6-(trifluoromethyl)pyridin-3-yl)methyl)urea;
123. 1-(6-(2-Hydroxyethoxy)pyridin-3-yl)-3-((2-(4-methylpiperidin-1-yl)-6-(trifluoromethyl)pyridin-3-yl)methyl)urea;
124. 1-(6-(2-Methoxyethoxy)pyridin-3-yl)-3-((2-(4-methylpiperidin-1-yl)-6-(trifluoromethyl)pyridin-3-yl)methyl)urea;
125. 1-(6-(2-Hydroxyethoxy)pyridin-3-yl)-3-((2-m-tolyl-6-(trifluoromethyl)pyridin-3-yl)methyl)urea;
126. 1-(5-(Hydroxymethyl)pyridin-3-yl)-3-((2-(4-methylpiperidin-1-yl)-6-(trifluoromethyl)pyridin-3-yl)methyl)urea;
127. 1-(5-(Hydroxymethyl)pyridin-2-yl)-3-((2-(4-methylpiperidin-1-yl)-6-(trifluoromethyl)pyridin-3-yl)methyl)urea;
128. 1-(3-(Hydroxymethyl)pyridin-4-yl)-3-((2-(4-methylpiperidin-1-yl)-6-(trifluoromethyl)pyridin-3-yl)methyl)urea;
129. 1-(6-(1,2-Dihydroxyethyl)pyridin-3-yl)-3-((2-(4-methylpiperidin-1-yl)-6-(trifluoromethyl)pyridin-3-yl)methyl)urea;
130. 1-((2-(3-Fluorophenyl)-6-(trifluoromethyl)pyridin-3-yl)methyl)-3-(6-(2-hydroxyethylamino)pyridin-3-yl)urea. and
131. 1-((5′-Chloro-6-(trifluoromethyl)-2,3′-bipyridin-3-yl)methyl)-3-(6-(2-hydroxyethylamino)pyridin-3-yl)urea,
   optionally in the form of a single stereoisomer or a mixture of stereoisomers, in the form of the free compound and/or a physiologically acceptable salt thereof.

Furthermore, preference may be given to compounds according to the invention that cause a 50 percent displacement of capsaicin, which is present at a concentration of 100 nM, in a FLIPR assay with CHO K1 cells which were transfected with the human VR1 gene at a concentration of less than 2,000 nM, preferably less than 1,000 nM, particularly preferably less than 300 nM, most particularly preferably less than 100 nM, even more preferably less than 75 nM, additionally preferably less than 50 nM, most preferably less than 10 nM.

In the process, the Ca²⁺ influx is quantified in the FLIPR assay with the aid of a Ca²⁺-sensitive dye (type Fluo-4, Molecular Probes Europe BV, Leiden, the Netherlands) in a fluorescent imaging plate reader (FLIPR, Molecular Devices, Sunnyvale, USA), as described hereinafter.

The substituted compounds according to the invention of the aforementioned general formula (I) and corresponding stereoisomers and also the respective corresponding acids, bases, salts and solvates are toxicologically safe and are therefore suitable as pharmaceutical active ingredients in pharmaceutical compositions.

The present invention therefore further relates to a pharmaceutical composition containing at least one compound according to the invention of the above-indicated formula (I), in each case if appropriate in the form of one of its pure stereoisomers, in particular enantiomers or diastereomers, its racemates or in the form of a mixture of stereoisomers, in particular the enantiomers and/or diastereomers, in any desired mixing ratio, or respectively in the form of a corresponding salt, or respectively in the form of a corresponding solvate, and also if appropriate one or more pharmaceutically compatible auxiliaries.

These pharmaceutical compositions according to the invention are suitable in particular for vanilloid receptor 1-(VR1/TRPV1) regulation, preferably for vanilloid receptor 1-(VR1/TRPV1) inhibition and/or for vanilloid receptor 1-(VR1/TRPV1) stimulation, i.e. they exert an agonistic or antagonistic effect.

Likewise, the pharmaceutical compositions according to the invention are preferably suitable for the inhibition and/or treatment of disorders or diseases which are mediated, at least in part, by vanilloid receptors 1.

The pharmaceutical composition according to the invention is suitable for administration to adults and children, including toddlers and babies.

The pharmaceutical composition according to the invention may be found as a liquid, semisolid or solid pharmaceutical form, for example in the form of injection solutions, drops, juices, syrups, sprays, suspensions, tablets, patches, capsules, plasters, suppositories, ointments, creams, lotions, gels, emulsions, aerosols or in multiparticulate form, for example in the form of pellets or granules, if appropriate pressed into tablets, decanted in capsules or suspended in a liquid, and also be administered as much.

In addition to at least one substituted compound of the above-indicated formula (I), if appropriate in the form of one of its pure stereoisomers, in particular enantiomers or diastereomers, its racemate or in the form of mixtures of the stereoisomers, in particular the enantiomers or diastereomers, in any desired mixing ratio, or if appropriate in the form of a corresponding salt or respectively in the form of a corresponding solvate, the pharmaceutical composition according to the invention conventionally contains further physiologically compatible pharmaceutical auxiliaries which can for example be selected from the group consisting of excipients, fillers, solvents, diluents, surface-active substances, dyes, preservatives, blasting agents, slip additives, lubricants, aromas and binders.

The selection of the physiologically compatible auxiliaries and also the amounts thereof to be used depend on whether the pharmaceutical composition is to be applied orally, subcutaneously, parenterally, intravenously, intraperitoneally, intradermally, intramuscularly, intranasally, buccally, rectally or locally, for example to infections of the skin, the mucous membranes and of the eyes. Preparations in the form of tablets, dragees, capsules, granules, pellets, drops, juices and syrups are preferably suitable for oral application; solutions, suspensions, easily reconstitutable dry preparations and also sprays are preferably suitable for parenteral, topical and inhalative application. The substituted compounds according to the invention used in the pharmaceutical composition according to the invention in a repository in dissolved form or in a plaster, agents promoting skin penetration being added if appropriate, are suitable percutaneous application preparations. Orally or percutaneously applicable preparation forms can release the respective substituted compound according to the invention also in a delayed manner.

The pharmaceutical compositions according to the invention are prepared with the aid of conventional means, devices, methods and process known in the art, such as are described for example in “Remington's Pharmaceutical Sciences”, A. R. Gennaro (Editor), 17^{th} edition, Mack Publishing Company, Easton, Pa., 1985, in particular in Part 8, Chapters 76 to 93. The corresponding description is introduced herewith by way of reference and forms part of the disclosure. The amount to be administered to the patient of the respective substituted compounds according to the invention of the above-indicated general formula I may vary and is for example dependent on the patient's weight or age and also on the type of application, the indication and the severity of the disorder. Conventionally 0.001 to 100 mg/kg, preferably 0.05 to 75 mg/kg, particularly preferably 0.05 to 50 mg of at least one such compound according to the invention are applied per kg of the patient's body weight.

The pharmaceutical composition according to the invention is preferably suitable for the treatment and/or inhibition of one or more disorders and/or diseases selected from the group consisting of pain, preferably pain selected from the group consisting of acute pain, chronic pain, neuropathic pain, visceral pain and joint pain; hyperalgesia; allodynia; causalgia; migraine; depression; nervous affection; axonal injuries; neurodegenerative diseases, preferably selected from the group consisting of multiple sclerosis, Alzheimer's disease, Parkinson's disease and Huntington's disease; cognitive dysfunctions, preferably cognitive deficiency states, particularly preferably memory disorders; epilepsy; respiratory diseases, preferably selected from the group consisting of asthma, bronchitis and pulmonary inflammation; coughs; urinary incontinence; overactive bladder (OAB); disorders and/or injuries of the gastrointestinal tract; duodenal ulcers; gastric ulcers; irritable bowel syndrome; strokes; eye irritations; skin irritations; neurotic skin diseases; allergic skin diseases; psoriasis; vitiligo; herpes simplex; inflammations, preferably inflammations of the intestine, the eyes, the bladder, the skin or the nasal mucous membrane; diarrhoea; pruritus; osteoporosis; arthritis; osteoarthritis; rheumatic diseases; eating disorders, preferably selected from the group consisting of bulimia, cachexia, anorexia and obesity; medication dependency; misuse of medication; withdrawal symptoms in medication dependency; development of tolerance to medication, preferably to natural or synthetic opioids; drug dependency; misuse of drugs; withdrawal symptoms in drug dependency; alcohol dependency; misuse of alcohol and withdrawal symptoms in alcohol dependency; for diuresis; for antinatriuresis; for influencing the cardiovascular system; for increasing vigilance; for the treatment of wounds and/or burns; for the treatment of severed nerves; for increasing libido; for modulating movement activity; for anxiolysis; for local anaesthesia and/or for inhibiting undesirable side effects, preferably selected from the group consisting of hyperthermia, hypertension and bronchoconstriction, triggered by the administration of vanilloid receptor 1 (VR1/TRPV1 receptor) agonists, preferably selected from the group consisting of capsaicin, resiniferatoxin, olvanil, arvanil, SDZ-249665, SDZ-249482, nuvanil and capsavanil.

Particularly preferably, the pharmaceutical composition according to the invention is suitable for the treatment and/or inhibition of one or more disorders and/or diseases selected from the group consisting of pain, preferably of pain selected from the group consisting of acute pain, chronic pain, neuropathic pain, visceral pain and joint pain; migraine; depression; neurodegenerative diseases, preferably selected from the group consisting of multiple sclerosis, Alzheimer's disease, Parkinson's disease and Huntington's disease; cognitive dysfunctions, preferably cognitive deficiency states, particularly preferably memory disorders; inflammations, preferably inflammations of the intestine, the eyes, the bladder, the skin or the nasal mucous membrane; urinary incontinence; overactive bladder (OAB); medication dependency; misuse of medication; withdrawal symptoms in medication dependency; development of tolerance to medication, preferably development of tolerance to natural or synthetic opioids; drug dependency; misuse of drugs; withdrawal symptoms in drug dependency; alcohol dependency; misuse of alcohol and withdrawal symptoms in alcohol dependency.

Most particularly preferably, the pharmaceutical composition according to the invention is suitable for the treatment and/or inhibition of pain, preferably of pain selected from the group consisting of acute pain, chronic pain, neuropathic pain and visceral pain.

The present invention further relates to a substituted compound according to general formula (I) and also if appropriate to a substituted compound according to general formula (I) and one or more pharmaceutically acceptable auxiliaries for use in vanilloid receptor 1-(VR1/TRPV1) regulation, preferably for use in vanilloid receptor 1-(VR1/TRPV1) inhibition and/or vanilloid receptor 1-(VR1/TRPV1) stimulation.

The present invention therefore further relates to a substituted compound according to general formula (I) and also if appropriate to a substituted compound according to general formula (I) and one or more pharmaceutically acceptable auxiliaries for use in the inhibition and/or treatment of disorders and/or diseases which are mediated, at least in part, by vanilloid receptors 1.

In particular, the present invention therefore further relates to a substituted compound according to general formula (I) and also if appropriate to a substituted compound according to general formula (I) and one or more pharmaceutically acceptable auxiliaries for use in the inhibition and/or treatment of disorders and/or diseases selected from the group consisting of pain, preferably pain selected from the group consisting of acute pain, chronic pain, neuropathic pain, visceral pain and joint pain; hyperalgesia; allodynia; causalgia; migraine; depression; nervous affection; axonal injuries; neurodegenerative diseases, preferably selected from the group consisting of multiple sclerosis, Alzheimer's disease, Parkinson's disease and Huntington's disease; cognitive dysfunctions, preferably cognitive deficiency states, particularly preferably memory disorders; epilepsy; respiratory diseases, preferably selected from the group consisting of asthma, bronchitis and pulmonary inflammation; coughs; urinary incontinence; overactive bladder (OAB); disorders and/or injuries of the gastrointestinal tract; duodenal ulcers; gastric ulcers; irritable bowel syndrome; strokes; eye irritations; skin irritations; neurotic skin diseases; allergic skin diseases; psoriasis; vitiligo; herpes simplex; inflammations, preferably inflammations of the intestine, the eyes, the bladder, the skin or the nasal mucous membrane; diarrhoea; pruritus; osteoporosis; arthritis; osteoarthritis; rheumatic diseases; eating disorders, preferably selected from the group consisting of bulimia, cachexia, anorexia and obesity; medication dependency; misuse of medication; withdrawal symptoms in medication dependency; development of tolerance to medication, preferably to natural or synthetic opioids; drug dependency; misuse of drugs; withdrawal symptoms in drug dependency; alcohol dependency; misuse of alcohol and withdrawal symptoms in alcohol dependency; for diuresis; for antinatriuresis; for influencing the cardiovascular system; for increasing vigilance; for the treatment of wounds and/or burns; for the treatment of severed nerves; for increasing libido; for modulating movement activity; for anxiolysis; for local anaesthesia and/or for inhibiting undesirable side effects, preferably selected from the group consisting of hyperthermia, hypertension and bronchoconstriction, triggered by the administration of vanilloid receptor 1 (VR1/TRPV1 receptor) agonists, preferably selected from the group consisting of capsaicin, resiniferatoxin, olvanil, arvanil, SDZ-249665, SDZ-249482, nuvanil and capsavanil.

Most particularly preferred is a substituted compound according to general formula (I) and also if appropriate to a substituted compound according to general formula (I) and one or more pharmaceutically acceptable auxiliaries for use in the inhibition and/or treatment of pain, preferably of pain selected from the group consisting of acute pain, chronic pain, neuropathic pain and visceral pain.

The present invention further relates to the use of at least one compound according to general formula (I) and also if appropriate of one or more pharmaceutically acceptable auxiliaries for the preparation of a pharmaceutical composition for vanilloid receptor 1-(VR1/TRPV1) regulation, preferably for vanilloid receptor 1-(VR1/TRPV1) inhibition and/or for vanilloid receptor 1-(VR1/TRPV1) stimulation, and, further for the inhibition and/or treatment of disorders and/or diseases which are mediated, at least in part, by vanilloid receptors 1, such as e.g. disorders and/or diseases selected from the group consisting of pain, preferably pain selected from the group consisting of acute pain, chronic pain, neuropathic pain, visceral pain and joint pain; hyperalgesia; allodynia; causalgia; migraine; depression; nervous affection; axonal injuries; neurodegenerative diseases, preferably selected from the group consisting of multiple sclerosis, Alzheimer's disease, Parkinson's disease and Huntington's disease; cognitive dysfunctions, preferably cognitive deficiency states, particularly preferably memory disorders; epilepsy; respiratory diseases, preferably selected from the group consisting of asthma, bronchitis and pulmonary inflammation; coughs; urinary incontinence; overactive bladder (OAB); disorders and/or injuries of the gastrointestinal tract; duodenal ulcers; gastric ulcers; irritable bowel syndrome; strokes; eye irritations; skin irritations; neurotic skin diseases; allergic skin diseases; psoriasis; vitiligo; herpes simplex; inflammations, preferably inflammations of the intestine, the eyes, the bladder, the skin or the nasal mucous membrane; diarrhoea; pruritus; osteoporosis; arthritis; osteoarthritis; rheumatic diseases; eating disorders, preferably selected from the group consisting of bulimia, cachexia, anorexia and obesity; medication dependency; misuse of medication; withdrawal symptoms in medication dependency; development of tolerance to medication, preferably to natural or synthetic opioids; drug dependency; misuse of drugs; withdrawal symptoms in drug dependency; alcohol dependency; misuse of alcohol and withdrawal symptoms in alcohol dependency; for diuresis; for antinatriuresis; for influencing the cardiovascular system; for increasing vigilance; for the treatment of wounds and/or burns; for the treatment of severed nerves; for increasing libido; for modulating movement activity; for anxiolysis; for local anaesthesia and/or for inhibiting undesirable side effects, preferably selected from the group consisting of hyperthermia, hypertension and bronchoconstriction, triggered by the administration of vanilloid receptor 1 (VR1/TRPV1 receptor) agonists, preferably selected from the group consisting of capsaicin, resiniferatoxin, olvanil, arvanil, SDZ-249665, SDZ-249482, nuvanil and capsavanil.

Another aspect of the present invention is a method for vanilloid receptor 1-(VR1/TRPV1) regulation, preferably for vanilloid receptor 1-(VR1/TRPV1) inhibition and/or for vanilloid receptor 1-(VR1/TRPV1) stimulation, and, further, a method of treatment and/or inhibition of disorders and/or diseases, which are mediated, at least in part, by vanilloid receptors 1, in a mammal, preferably of disorders and/or diseases selected from the group consisting of pain, preferably pain selected from the group consisting of acute pain, chronic pain, neuropathic pain, visceral pain and joint pain; hyperalgesia; allodynia; causalgia; migraine; depression; nervous affection; axonal injuries; neurodegenerative diseases, preferably selected from the group consisting of multiple sclerosis, Alzheimer's disease, Parkinson's disease and Huntington's disease; cognitive dysfunctions, preferably cognitive deficiency states, particularly preferably memory disorders; epilepsy; respiratory diseases, preferably selected from the group consisting of asthma, bronchitis and pulmonary inflammation; coughs; urinary incontinence; overactive bladder (OAB); disorders and/or injuries of the gastrointestinal tract; duodenal ulcers; gastric ulcers; irritable bowel syndrome; strokes; eye irritations; skin irritations; neurotic skin diseases; allergic skin diseases; psoriasis; vitiligo; herpes simplex; inflammations, preferably inflammations of the intestine, the eyes, the bladder, the skin or the nasal mucous membrane; diarrhoea; pruritus; osteoporosis; arthritis; osteoarthritis; rheumatic diseases; eating disorders, preferably selected from the group consisting of bulimia, cachexia, anorexia and obesity; medication dependency; misuse of medication; withdrawal symptoms in medication dependency; development of tolerance to medication, preferably to natural or synthetic opioids; drug dependency; misuse of drugs; withdrawal symptoms in drug dependency; alcohol dependency; misuse of alcohol and withdrawal symptoms in alcohol dependency; for diuresis; for antinatriuresis; for influencing the cardiovascular system; for increasing vigilance; for the treatment of wounds and/or burns; for the treatment of severed nerves; for increasing libido; for modulating movement activity; for anxiolysis; for local anaesthesia and/or for inhibiting undesirable side effects, preferably selected from the group consisting of hyperthermia, hypertension and bronchoconstriction, triggered by the administration of vanilloid receptor 1 (VR1/TRPV1 receptor) agonists, preferably selected from the group consisting of capsaicin, resiniferatoxin, olvanil, arvanil, SDZ-249665, SDZ-249482, nuvanil and capsavanil, which comprises administering an effective amount of at least one compound of general formula (I) to the mammal.

The effectiveness against pain can be shown, for example, in the Bennett or Chung model (Bennett, G. J. and Xie, Y. K., A peripheral mononeuropathy in rat that produces disorders of pain sensation like those seen in man, Pain 1988, 33(1), 87-107; Kim, S. H. and Chung, J. M., An experimental model for peripheral neuropathy produced by segmental spinal nerve ligation in the rat, Pain 1992, 50(3), 355-363), by tail flick experiments (e.g. according to D'Amour and Smith (J. Pharm. Exp. Ther. 72, 74 79 (1941)) or by the formalin test (e.g. according to D. Dubuisson et al., Pain 1977, 4, 161-174).

The present invention further relates to processes for preparing inventive compounds of the above-indicated general formula (I).

In particular, the compounds according to the present invention of general formula (I) can be prepared by a process according to which at least one compound of general formula (II),

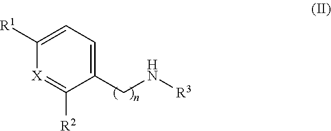

in which X, R¹, R², R³ and n have one of the foregoing meanings, is reacted in a reaction medium, if appropriate in the presence of at least one suitable coupling reagent, if appropriate in the presence of at least one base, with a compound of general formula (III) with D=OH or Hal,

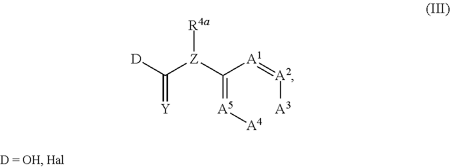

in which Hal represents a halogen, preferably Br or Cl, and R^{4a}, Y, A¹, A², A³, A⁴ and A⁵ each have one of the foregoing meanings and Z denotes C—R^{4b}, wherein R^{4b} has one of the foregoing meanings, in a reaction medium, if appropriate in the presence of at least one suitable coupling reagent, if appropriate in the presence of at least one base, to form a compound of general formula (I),

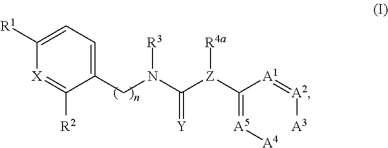

in which Z represents CR^{4b} and X, R¹, R², R³, R^{4a}, R^{4b}, Y, A¹, A², A³, A⁴ and A⁵ and n have one of the foregoing meanings;
or in that at least one compound of general formula (II),

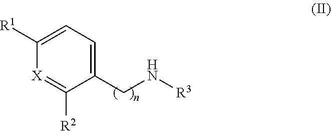

in which X, R¹, R², R³ and n have one of the foregoing meanings, is reacted to form a compound of general formula (IV)

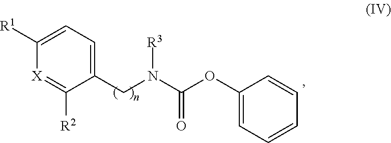

in which X, R¹, R², R³ and n have one of the foregoing meanings, in a reaction medium, in the presence of phenyl chloroformate, if appropriate in the presence of at least one base and/or at least one coupling reagent, and said compound is if appropriate purified and/or isolated, and a compound of general formula (IV) is reacted with a compound of general formula (V),

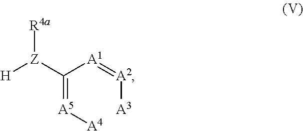

in which R^{4a}, A¹, A², A³, A⁴ and A⁵ have one of the foregoing meanings, and Z denotes N, in a reaction medium, if appropriate in the presence of at least one suitable coupling reagent, if appropriate in the presence of at least one base, to form a compound of general formula (I),

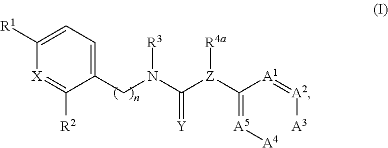

in which Z represents N and X, R¹, R², R³, R^{4a}, Y, A¹, A², A³, A⁴, Y, A¹, A², A³, A⁴ and A⁵ and n have one of the foregoing meanings.

The reaction of compounds of the above-indicated general formulae (II) and (V) with carboxylic acids of the above-indicated general formula (III), particularly with D=OH, to form compounds of the above-indicated general formula (I) is carried out preferably in a reaction medium selected from the group consisting of diethyl ether, tetrahydrofuran, acetonitrile, methanol, ethanol, (1,2)-dichloroethane, dimethylformamide, dichloromethane and corresponding mixtures, if appropriate in the presence of at least one coupling reagent, preferably selected from the group consisting of 1-benzotriazolyloxy-tris-(dimethylamino)-phosphonium hexafluorophosphate (BOP), dicyclohexylcarbodiimide (DCC), N′-(3-dimethylaminopropyl)-N-ethylcarbodiimide (EDCI), diisopropylcarbodiimide, 1,1′-carbonyldiimidazole (CDI), N-[(dimethylamino)-1H-1,2,3-triazolo[4,5-b]pyridino-1-yl-methylene]-N-methylmethanaminium hexafluorophosphate N-oxide (HATU), 0-(benzotriazol-1-yl)-N,N,N′,N′-tetramethyluronium hexafluorophosphate (HBTU), 0-(benzotriazol-1-yl)-N,N,N′,N′-tetramethyluronium tetrafluoroborate (TBTU), N-hydroxybenzotriazole (HOBt) and 1-hydroxy-7-azabenzotriazole (HOAt), if appropriate in the presence of at least one organic base, preferably selected from the group consisting of triethylamine, pyridine, dimethylaminopyridine, N-methylmorpholine and diisopropylethylamine, preferably at temperatures of from −70° C. to 100° C.

Alternatively, the reaction of compounds of the above-indicated general formulae (II) and (V) with carboxylic acid halides of the above-indicated general formula (III) with D=Hal, in which Hal represents a halogen as the leaving group, preferably a chlorine or bromine atom, to form compounds of the above-indicated general formula (I) is carried out in a reaction medium preferably selected from the group consisting of diethyl ether, tetrahydrofuran, acetonitrile, methanol, ethanol, dimethylformamide, dichloromethane and corresponding mixtures, if appropriate in the presence of an organic or inorganic base, preferably selected from the group consisting of triethylamine, dimethylaminopyridine, pyridine and diisopropylamine, at temperatures of from −70° C. to 100° C.

The compounds of the above-indicated formulae (II), (III), (IV), and (V) are each commercially available and/or can be prepared using conventional processes known to the person skilled in the art. In particular, processes to prepare these compounds are e.g. disclosed in WO 2007/045462-A2, WO 2008/125342-A2 and WO 2008/125337-A2. The corresponding parts of these references are hereby deemed to be part of the disclosure.

All reactions which can be applied for synthesizing the compounds according to the present invention can each be carried out under the conventional conditions with which the person skilled in the art is familiar, for example with regard to pressure or the order in which the components are added. If appropriate, the person skilled in the art can determine the optimum procedure under the respective conditions by carrying out simple preliminary tests. The intermediate and end products obtained using the reactions described hereinbefore can each be purified and/or isolated, if desired and/or required, using conventional methods known to the person skilled in the art. Suitable purifying processes are for example extraction processes and chromatographic processes such as column chromatography or preparative chromatography. All of the process steps of the reaction sequences which can be applied for synthesizing the compounds according to the present invention as well as the respective purification and/or isolation of intermediate or end products, can be carried out partly or completely under an inert gas atmosphere, preferably under a nitrogen atmosphere.

The substituted compounds according to the invention can be isolated both in the form of their free bases, their free acids and also in the form of corresponding salts, in particular physiologically compatible salts, i.e. physiologically acceptable salts.

The free bases of the respective substituted compounds according to the invention can be converted into the corresponding salts, preferably physiologically compatible salts, for example by reaction with an inorganic or organic acid, preferably with hydrochloric acid, hydrobromic acid, sulphuric acid, methanesulphonic acid, p-toluenesulphonic acid, carbonic acid, formic acid, acetic acid, oxalic acid, succinic acid, tartaric acid, mandelic acid, fumaric acid, maleic acid, lactic acid, citric acid, glutamic acid, saccharic acid, monomethylsebacic acid, 5-oxoproline, hexane-1-sulphonic acid, nicotinic acid, 2, 3 or 4-aminobenzoic acid, 2,4,6-trimethylbenzoic acid, α-lipoic acid, acetyl glycine, hippuric acid, phosphoric acid and/or aspartic acid. The free bases of the respective substituted compounds of the aforementioned general formula (I) and of corresponding stereoisomers can likewise be converted into the corresponding physiologically compatible salts using the free acid or a salt of a sugar additive, such as for example saccharin, cyclamate or acesulphame.

Accordingly, the free acids of the substituted compounds according to the invention can be converted into the corresponding physiologically compatible salts by reaction with a suitable base. Examples include the alkali metal salts, alkaline earth metals salts or ammonium salts [NHₓR₄₋ₓ]⁺, in which x=0, 1, 2, 3 or 4 and R represents a branched or unbranched C₁₋₄ aliphatic residue.

The substituted compounds according to the invention and of corresponding stereoisomers can if appropriate, like the corresponding acids, the corresponding bases or salts of these compounds, also be obtained in the form of their solvates, preferably in the form of their hydrates, using conventional methods known to the person skilled in the art.

If the substituted compounds according to the invention are obtained, after preparation thereof, in the form of a mixture of their stereoisomers, preferably in the form of their racemates or other mixtures of their various enantiomers and/or diastereomers, they can be separated and if appropriate isolated using conventional processes known to the person skilled in the art. Examples include chromatographic separating processes, in particular liquid chromatography processes under normal pressure or under elevated pressure, preferably MPLC and HPLC processes, and also fractional crystallisation processes. These processes allow individual enantiomers, for example diastereomeric salts formed by means of chiral stationary phase HPLC or by means of crystallisation with chiral acids, for example (+)-tartaric acid, (−)-tartaric acid or (+)-10-camphorsulphonic acid, to be separated from one another.

The chemicals and reaction components used in the reactions and schemes described below are available commercially or in each case can be prepared by conventional methods known to the person skilled in the art.

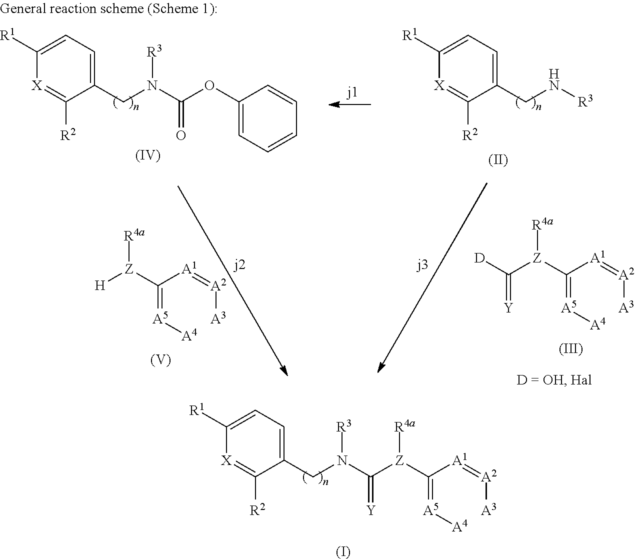

In step j1 the compound (II) can be converted into the compound (IV) by means of methods known to the person skilled in the art, such as using phenyl chloroformate, if appropriate in the presence of a coupling reagent and/or a base. In addition to the methods disclosed in the present document for preparing unsymmetrical ureas using phenyl chloroformate, there are further processes with which the person skilled in the art is familiar, based on the use of activated carbonic acid derivatives or isocyanates, if appropriate.

In step j2 the amine (V) can be converted into the urea compound (I) (wherein Z═N). This can be achieved by reaction with (IV) by means of methods with which the person skilled in the art is familiar, if appropriate in the presence of a base.

In step j3 the amine (II) can be converted into the amide (I) (wherein A=C—R^{4b}). This can for example be achieved by reaction with an acid halide, preferably a chloride, of formula (III) with D=Hal, by means of methods with which the person skilled in the art is familiar, if appropriate in the presence of a base or by reaction with an acid of formula (III) with D=OH, if appropriate in the presence of a suitable coupling reagent, for example HATU or CDI, if appropriate with the addition of a base. Further, the amine (II) may be converted into the amide (I) (wherein Z═C—R^{4b}) by reaction of a compound (IIIa)

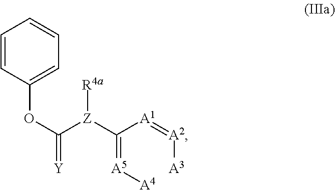

by means of methods with which the person skilled in the art is familiar, if appropriate in the presence of a base.

### General Reaction Scheme (Scheme 2):

The compounds according to general formula (I), wherein Z═N, may be further prepared by a reaction sequence according to general reaction scheme 2:

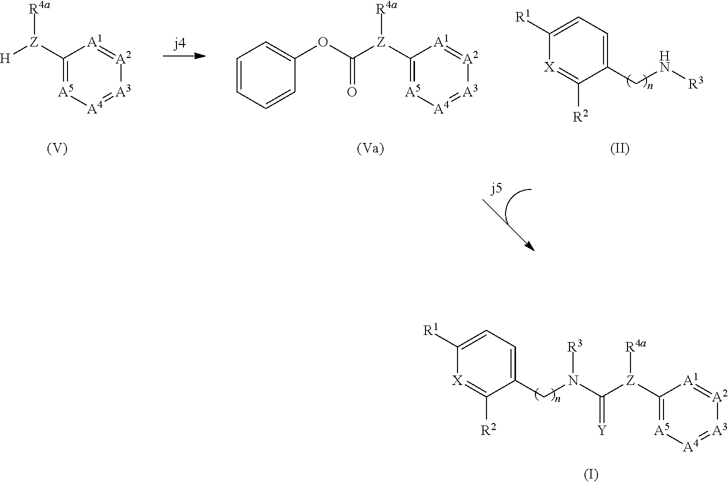

In step j4 the compound (V) can be converted into the compound (Va), wherein Z═N, by means of methods known to the person skilled in the art, such as using phenyl chloroformate, if appropriate in the presence of a coupling reagent and/or a base. In addition to the methods disclosed in the present document for preparing unsymmetrical ureas using phenyl chloroformate, there are further processes with which the person skilled in the art is familiar, based on the use of activated carbonic acid derivatives or isocyanates, if appropriate.

In step j5 the amine (II) can be converted into the urea compound (I) (wherein Z═N). This can be achieved by reaction with (Va) by means of methods with which the person skilled in the art is familiar, if appropriate in the presence of a base.

The methods with which the person skilled in the art is familiar for carrying out the reaction steps j1 to j5 may be inferred from the standard works on organic chemistry such as, for example, J. March, Advanced Organic Chemistry, Wiley & Sons, 6th edition, 2007; F. A. Carey, R. J. Sundberg, Advanced Organic Chemistry, Parts A and B, Springer, 5th edition, 2007; team of authors, Compendium of Organic Synthetic Methods, Wiley & Sons. In addition, further methods and also literature references can be issued by the common databases such as, for example, the Reaxys® database of Elsevier, Amsterdam, NL or the SciFinder® database of the American Chemical Society, Washington, US.

The invention will be described hereinafter with the aid of a number of examples. This description is intended merely by way of example and does not limit the general idea of the invention.

### EXAMPLES

The indication “equivalents” (“eq.”) means molar equivalents, “RT” means room temperature, “M” and “N” are indications of concentration in mol/l, “aq.” means aqueous, “sat.” means saturated, “sol.” means solution, “conc.” means concentrated.

Further abbreviations include:
AcOH acetic acid
d days
BOP 1-benzotriazolyloxy-tris-(dimethylamino)phosphonium hexafluorophosphate brine saturated sodium chloride solution (NaCl sol.)
bipy 2,2′-bipyridine/2,2′-bipyridyl
Boc tert-butyloxycarbonyl
n-BuLi n-butyllithium
DCC N,N′-dicyclohexylcarbodiimide
DCM dichloromethane
DIPEA N,N-diisopropylethylamine
DMF N,N-dimethylformamide
DMAP 4-dimethylaminopyridine
EDC N-(3-dimethylaminopropyl)-N′-ethylcarbodiimide
EDCI N-ethyl-W-(3-dimethylaminopropyl)carbodiimide hydrochloride
EE ethyl acetate
ether diethyl ether
EtOH ethanol
sat. saturated
h hour(s)
H₂O water
HOBt N-hydroxybenzotriazole
LAH lithium aluminium hydride
LG leaving group
m/z mass-to-charge ratio
MeOH methanol
min minutes
MS mass spectrometry
NA not available
NEt₃ triethylamine
Pd(dppf)Cl_{2 [}1,1′-bis(diphenylphosphino)ferrocene]dichloropalladium(II)
Pd(OAc)₂ palladium(II) acetate
Pd(PPh₃)₄ tetrakis(triphenylphosphine)palladium(0)
R_{f} retention factor
SC silica gel column chromatography
THF tetrahydrofuran
TFA trifluoroacetic acid
TLC thin layer chromatography
vv volume ratio

The yields of the compounds prepared were not optimized. All temperatures are uncorrected. All starting materials which are not explicitly described were either commercially available (the details of suppliers such as for example Acros, Avocado, Aldrich, Bachem, Fluka, Lancaster, Maybridge, Merck, Sigma, TCI, Oakwood, etc. can be found in the Symyx® Available Chemicals Database of MDL, San Ramon, US, for example) or the synthesis thereof has already been described precisely in the specialist literature (experimental guidelines can be looked up in the Reaxys® Database of Elsevier, Amsterdam, NL, for example) or can be prepared using the conventional methods known to the person skilled in the art.

The stationary phase used for the column chromatography was silica gel 60 (0.0-0-0.063 mm) from E. Merck, Darmstadt. The thin-layer chromatographic tests were carried out using HPTLC precoated plates, silica gel 60 F 254, from E. Merck, Darmstadt. The mixing ratios of solvents, mobile solvents or for chromatographic tests are respectively specified in volume/volume.

### SYNTHESIS OF THE EXAMPLE COMPOUNDS

The example compounds 5-10, 13, 14, 19, 22, 24, 31, 32, 38, 39-42, 47, 49, 55, 67, 74-81, 84-92, 95-99, 104-105, 107-108, 114, 116-118, 120, 123-124 and 126-131 were prepared by one of the methods described herein. The other exemplary compounds may be prepared by analogous methods. Those skilled in the art are aware which method and materials have to be employed to obtain a particular exemplary compound.

### Synthesis of Example 6

### N-((2-(3-Chloro-4-fluorophenyl)-6-(trifluoromethyl)pyridin-3-yl)methyl)-2-(pyridin-2-yl)acetamide

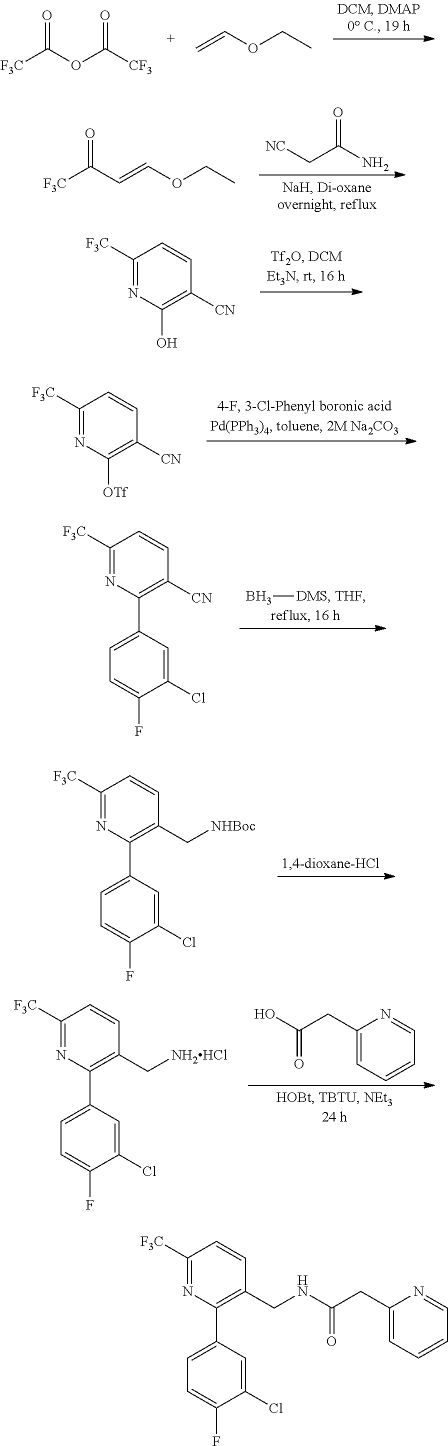

Step 1: To a stirred solution of 4-dimethylaminopyridine (0.1 g, 1.0 mmol) and trifluoro acetic anhydride (23.2 g, 1.1 mol) in dichloromethane (75 mL), ethyl vinyl ether (7.5 g, 1 mol) was added dropwise at −10° C. The reaction mixture was stirred at 0° C. for 16 h and then allowed to warm at 25-30° C. TLC showed complete consumption of starting material. The organic layer was then washed with water (2×60 mL), saturated sodium bicarbonate solution (2×25 mL) and finally with brine (1×30 mL). The washed organic layer was dried over anhydrous magnesium sulfate and concentrated under reduced pressure to get a dark brown oily residue. This residue was finally distilled out to afford a colorless liquid compound (14.5 g, 82%).

Step 2: To a solution of 1,4-dioxane (70 mL) and 2-cyanoacetamide (7.25 g, 0.086 mol), sodium hydride (4.12 g, 60%, 0.13 mol) was added portionwise at 10-15° C. It was allowed to stir for 30 min at ambient temperature after complete addition. A solution of (E)-4-ethoxy-1,1,1-trifluorobut-3-en-2-one (14.5 g, 0.086 mol) in 1,4-dioxane (70 mL) was added dropwise to this mixture. After complete addition the resulting solution was refluxed gently for 22 h. A solid was separated in the mixture. The mixture was cooled to ambient temperature and filtered through a sintered funnel. The residue was washed with 2 L of 1,4-dioxane. The washed solid was dissolved in water and acidified with 4N HCl (200 mL). The mixture was extracted with ethyl acetate (3×75 mL). The overall ethyl acetate layer was washed with brine (75 mL) and finally dried over magnesium sulfate. After removal of organic solvent under reduced pressure yellow solid was afforded (11 g, 68%).

Step 3: A stirred solution of 2-hydroxy-6-(trifluoromethyl)nicotinonitrile (10 g, 53.19 mmol) in dichloromethane (50 mL) was cooled to 0-5° C. To this solution, triethylamine (11 mL, 79.78 mmol) was added and allowed to stir for 30 min at 0-5° C. Triflic anhydride (19 mL, 106.38 mmol) was added dropwise at 0-5° C. to the mixture and the mixture was stirred for 16 h at room temperature. TLC showed complete consumption of starting material. The reaction mixture was diluted with dichloromethane and the organic part was washed with water (2×250 mL). The washed organic layer was dried over anhydrous magnesium sulfate and concentrated under reduced pressure to afford the crude product and the crude product was purified by column chromatography (silica gel:100-200; eluent: 10% ethyl acetate in n-hexane) to afford the pure 3-cyano-6-(trifluoromethyl)pyridin-2-yl trifluoromethanesulfonate (12.5 g, 73%).

Step 4: In a 500 mL round bottomed flask, 3-cyano-6-(trifluoromethyl)pyridin-2-yl trifluoromethanesulfonate (12 g, 37.48 mmol) was dissolved in toluene (70 mL) and to it 4-fluoro-3-chloro boronic acid (7.48 g, 44.97 mmol), aqueous sodium carbonate solution (2M, 75 mL) and Pd(PPh₃)₄ (2.16 g, 1.87 mmol) was added and finally the system was flushed with nitrogen. Reaction mixture was heated to 100° C. and stirred at that temperature for 4 h. TLC showed complete consumption of starting material. The reaction mixture was cooled and was diluted with water (300 mL) and extracted with 20% ethyl acetate in n-hexane (2×200 mL). The combined organic layer was washed with water (200 mL) and brine (200 mL). It was dried over anhydrous magnesium sulfate and concentrated under reduced pressure. This crude compound was purified by column chromatography (silica gel: 100-200 mesh, eluent: 5% ethyl acetate in n-hexane) to afford 2-(3-chloro-4-fluorophenyl)-6-(trifluoromethyl)nicotinonitrile (9.2 g, 82%).

Step 5: 2-(3-Chloro-4-fluorophenyl)-6-(trifluoromethyl)nicotinonitrile (7.1 g, 23.66 mmol) was dissolved in dry tetrahydrofuran (70 mL), cooled and borane-dimethyl sulphide (3.41 mL, 35.44 mmol) was added to it under nitrogen atmosphere at 0-5° C. The reaction mixture was then refluxed for 20 h. Excess borane dimethyl sulphide was quenched with methanol (6 mL) under cold condition and then di-tert-butyl dicarbonate (10.86 mL, 47.32 mmol) was added to it and stirred for one hour at ambient temperature. TLC showed complete conversion of starting material. The organic volatiles were concentrated to obtain the crude compound, which was purified by column chromatography (silica gel: 100-200 mesh, eluent: 5% ethyl acetate in n-hexane) to afford a white solid (5.27 g, 55%).

Step 6: To a stirred solution of tert-butyl (2-(3-chloro-4-fluorophenyl)-6-(trifluoromethyl)pyridin-3-yl)methylcarbamate (5.27 g, 13.04 mmol) in 1,4-dioxane (5 mL) was added with 1,4-dioxane.HCl (10 mL) under cooling and the reaction mixture was allowed to stir for 12 h. The reaction mixture was concentrated under reduced pressure and was co-distilled with methanol thrice and the solid obtained was filtered through sintered funnel and was washed with 10% ethyl acetate in n-hexane to afford pure (2-(3-chloro-4-fluorophenyl)-6-(trifluoromethyl)pyridin-3-yl)methanamine hydrochloride (4.14 g, 93%). ¹H NMR (DMSO-d₆, 400 MHz): δ 8.70 (s, 3H), 8.49 (d, 1H), 8.11 (d, 1H), 7.83 (d, 1H), 7.60 (t, 2H), 4.16 (s, 2H).

Step 7: To a stirred solution of (2-(3-chloro-4-fluorophenyl)-6-(trifluoromethyl)pyridin-3-yl)methanamine hydrochloride (0.1 g, 0.329 mmol) and 2-(pyridin-2-yl)acetic acid (0.057 g, 0.329 mmol) in tetrahydrofuran (2.5 mL) was added 1-hydroxybenzotriazolhydrate (0.0447 mL, 0.329 mmol), O-(1H-benzotriazol-1-yl)-N,N,N′,N′-tetramethyluronium tetrafluoroborate (0.106 g, 0.329 mmol) and N-ethyldiisopropylamine (0.124 mL, 0.658 mmol) and the reaction mixture was allowed to stir for 24 h. The reaction mixture was concentrated under reduced pressure and the solid obtained was purified by column chromatography (silica gel: 100-200 mesh, eluent: 10% methanol in ethyl acetate) to afford a white solid (81 mg, 58%).

Example compounds 7-10, 13, 22 and 24 were prepared in a similar manner and exemplary compounds 25-27 may be prepared analogously.

### Synthesis of Example 14

### N-((2-(4-Methylpiperidin-1-yl)-6-(trifluormethyl)pyridin-3-yl)methyl)-2-(pyridin-3-yl)propanamid

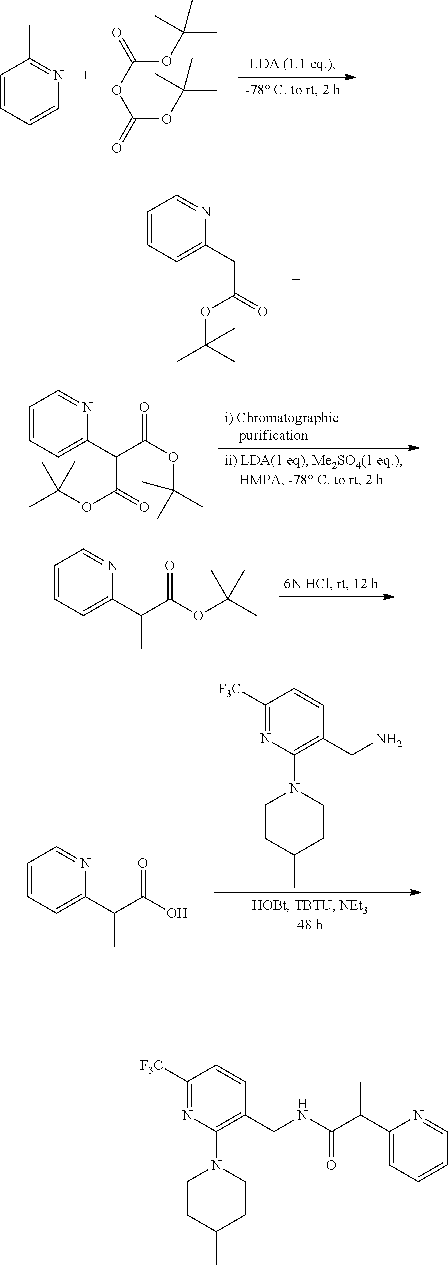

Step 1: To a stirred solution of diisopropylamine (10.8 g, 0.1 mol) in (20 mL) of dry tetrahydrofuran was added n-BuLi (49 mL, 2.04M, 0.10 mol) at −78° C. The reaction mixture was allowed to stir for 30 min. To this solution, 2-methylpyridine (10 g, 0.107 mol) in (20 mL) of dry tetrahydrofuran was added dropwise. The reaction mixture was allowed to stir for 1 h at −78° C. To this di-tert-butyl dicarbonate (24 g, 0.11 mol) was added at −78° C. and was allowed to attain room temperature in 2 h. The reaction mixture was quenched with saturated ammonium chloride solution (50 mL), diluted with water (60 mL) and extracted with ethyl acetate (3×80 mL). The total organic layer was washed with brine (50 mL). The final organic layer was dried over anhydrous magnesium sulfate and was concentrated under reduced pressure to obtain crude compound which was purified by column chromatography (silica gel: 100-200 mesh, eluent: 10% ethyl acetate in n-hexane) to afford tert-butyl 2-(pyridin-2-yl)acetate (6 g, 29%).

Step 2: To a stirred solution of diisopropylamine (1.56 g, 15.55 mmol) in dry tetrahydrofuran (5 mL) was added n-BuLi (7.6 mL, 2.04M, 15.55 mmol) at −78° C. The reaction mixture was allowed to stir for 30 min. To this solution, hexamethylphosphoramide (2.78 g, 15.55 mmol) and tert-butyl 2-(pyridin-2-yl)acetate (3 g, 15.55 mmol) dry tetrahydrofuran (5 mL) were added dropwise. The reaction mixture was allowed to stir for 1 h at −78° C. To this solution, dimethyl sulphate (1.95 g, 15.55 mol) in 5 mL of dry tetrahydrofuran was added at −78° C. and was allowed to attain ambient temperature in 2 h. The reaction mixture was quenched with saturated ammonium chloride solution (30 mL) and was diluted with water (50 mL) and was extracted with ethyl acetate (2×50 mL). The total organic layer was washed with brine (50 mL). The final organic layer was dried over anhydrous magnesium sulfate and was concentrated under reduced pressure to obtain crude compound which was purified by using column chromatography (silica gel:100-200 mesh, eluent: 5% ethyl acetate in n-hexane) to afford tert-butyl 2-(pyridin-2-yl)propanoate (1.8 g, 56%).

Step 3: To tert-butyl 2-(pyridin-2-yl)propanoate (2.5 g, 12.07 mmol), 6N HCl (65 mL) was added and was allowed to stir for 12 h. The reaction mixture was concentrated under reduced pressure to obtain crude compound which was co distilled with benzene (3×10 mL) to obtain 2-(pyridin-2-yl)propanoic acid (1.6 g).

¹H NMR (DMSO-d₆, 400 MHz): 1.54 (d, 3H), 4.27 (d, 1H), 7.78 (t, 1H), 7.80 (d, 1H), 8.38 (t, 1H), 8.76 (d, 1H)

Step 4: To a stirred solution of 2-(pyridin-2-yl)propanoic acid (0.093 g, 0.496 mmol) and (2-(4-methylpiperidin-1-yl)-6-(trifluoromethyl)pyridin-3-yl)methanamine (0.09 g, 0.331 mmol) in tetrahydrofuran (2.5 mL) was added 1-hydroxybenzotriazolhydrate (0.045 mL, 0.331 mmol), O-(1H-benzotriazol-1-yl)-N,N,N′,N′-tetramethyluronium tetrafluoroborate (0.107 g, 0.331 mmol) and N-ethyldiisopropylamine (0.128 mL, 0.993 mmol) to gave an suspension. After addition of N,N-dimethylformamide (0.1 mL) the reaction mixture was stirred for 48 h. The reaction mixture was concentrated under reduced pressure and the solid obtained was purified by column chromatography (silica gel: 100-200 mesh, eluent: ethyl acetate) to afford N-((2-(4-methylpiperidin-1-yl)-6-(trifluoromethyl)pyridin-3-yl)methyl)-2-(pyridin-2-yl)propanamide (35 mg, 26%).

### Synthesis of Example 19

### 1-((2-(4-Methylpiperidin-1-yl)-6-(trifluoromethyl)pyridin-3-yl)methyl)-3-(pyridin-2-yl)urea

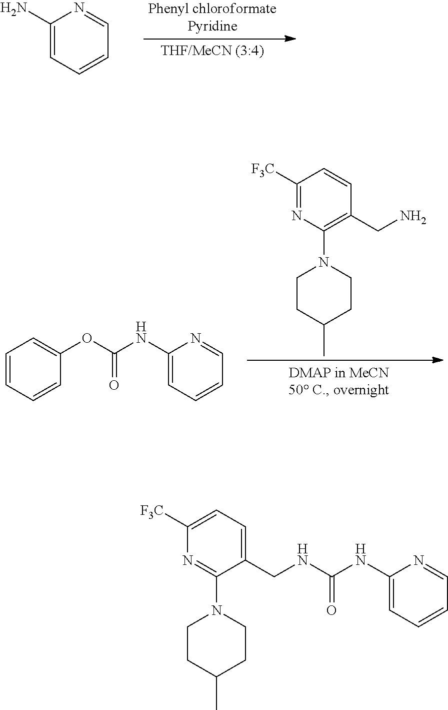 [SMILES: Nc1ccccn1 ; O=C(Nc1ccccn1)Oc1ccccc1 ; CC1CCN(c2nc(C(F)(F)F)ccc2CN)CC1 ; CC1CCN(c2nc(C(F)(F)F)ccc2CNC(=O)Nc2ccccn2)CC1]

Step 1: To a solution of 2-amino pyridine (400 mg, 4.25 mmol) in tetrahydrofuran and acetonitrile (50 mL, 3:4) was slowly added phenyl chloroformate (0.8 mL, 6.376 mmol) and pyridine (0.4 mL, 5.525 mmol) at room temperature. The reaction mixture was stirred for 3 h. TLC showed complete consumption of starting material. After adding water, the mixture was extracted with ethyl acetate. The extract was dried over MgSO₄ and concentrated under reduced pressure. The crude residue was purified by column chromatography (silica gel: 100-200 mesh, eluent: n-hexane—ethyl acetate, 4:1) to give the phenyl pyridin-2-ylcarbamate (710 mg, 78%).

Step 2: To a solution of phenyl pyridin-2-ylcarbamate (70 mg, 0.327 mmol) in acetonitrile (20 mL) was added DMAP (40 mg, 0.327 mmol, 1 equip) and (2-(4-methylpiperidin-1-yl)-6-(trifluoromethyl)pyridin-3-yl)methanamine (116 mg, 0.425 mmol, 1.3 equip) at room temperature. The reaction mixture was heated to 50° C. for 12 h. TLC showed complete consumption of starting material. The reaction mixture was diluted with water and extracted with ethyl acetate. The organic part was washed with water and brine. The organic layer was dried over MgSO₄ and concentrated under reduced pressure. The crude was purified by column chromatography (silica gel: 100-200 mesh, eluent: n-hexane—ethyl acetate, 1:1) to give 1-((2-(4-methylpiperidin-1-yl)-6-(trifluoromethyl)pyridin-3-yl)methyl)-3-(pyridin-2-yl)urea (58 mg, 45%).

¹H NMR (300 MHz, CDCl₃): δ 8.27 (S, 1H, Ar—NH), 8.12 (dd, 1H, J=4.05 Hz, Ar—H), 7.78 (d, 1H, J=7.5 Hz, Ar—H), 7.59 (M, 1H, Ar—H), 7.22 (d, 1H, J=7.68 Hz, Ar—H), 6.88 (m, 1H, Ar—H), 6.75 (d, 1H, J=8.22 Hz, Ar—H), 4.63 (d, 2H, J=5.85 Hz, Ar—CH₂), 3.47 (d, 2H, J=12.81 Hz, Piperidine-H), 2.90 (m, 2H, Piperidine-H), 1.76 (m, 2H, Piperidine-H), 1.40 (m, 2H, Piperidine-H), 1.00 (d, 3H, J=6.39 Hz, Piperidine-CH₃)

The exemplary compound 23 can be prepared in a similar manner and exemplary compounds 35-37, 43-46 and 48 can be prepared analogously. Exemplary compound 42 has been prepared analogously.

### Synthesis of Example 55

### 1-(6-(Hydroxymethyl)pyridin-3-yl)-3-((2-(4-methylpiperidin-1-yl)-6-(trifluoromethyl)pyridin-3-yl)methyl)urea

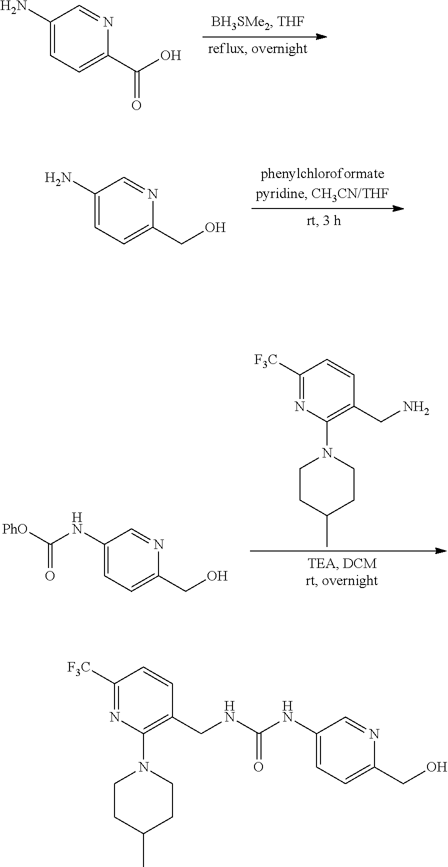 [SMILES: Nc1ccc(C(=O)O)nc1 ; Nc1ccc(CO)nc1 ; O=C(Nc1ccc(CO)nc1)Oc1ccccc1 ; CC1CCN(c2nc(C(F)(F)F)ccc2CN)CC1 ; CC1CCN(c2nc(C(F)(F)F)ccc2CNC(=O)Nc2ccc(CO)nc2)CC1]

Step 1: To a stirred solution of 5-aminopicolinic acid (400 mg, 2.90 mmol) in tetrahydrofuran were added BH₃SMe₂ (2 M in tetrahydrofuran) (4.34 mL, 8.69 mmol, 3 eq) at room temperature. The reaction mixture was refluxed for overnight. TLC showed complete consumption of starting material. The reaction mixture was quenched with water and extracted with ethylacetate. The organic part was washed with brine. The organic layer was dried over MgSO₄ and concentrated under reduced pressure to afford crude product which was purified by column chromatography to afford (5-aminopyridin-2-yl)methanol (136 mg, 36%).

Step 2: (5-Aminopyridin-2-yl)methanol (118 mg, 0.95 mmol) was dissolved in acetonitrile (3 mL) and tetrahydrofuran (4 mL). The reaction mixture was added pyridine (0.09 mL, 1.14 mmol, 1.2 eq) and phenyl chloroformate (0.12 mL, 0.98 mmol, 1.03 eq) and stirred at room temperature for 3 h under nitrogen atmosphere. TLC showed complete consumption of starting material. The reaction mixture was diluted with water and extracted with ethylacetate. The organic part was washed with water and brine. The organic layer was dried over MgSO₄ and concentrated under reduced pressure. The crude was purified by column chromatography to give phenyl 6-(hydroxymethyl)pyridin-3-ylcarbamate (191 mg, 82%).

Step 3: To a solution of phenyl 6-(hydroxymethyl)pyridin-3-ylcarbamate (63 mg, 0.26 mmol) in dichloromethane was added triethylamine (0.11 mL, 0.77 mmol, 3 equiv) and (2-(4-methylpiperidin-1-yl)-6-(trifluoromethyl)pyridin-3-yl)methanamine (70 mg, 0.26 mmol, 1 eq) at room temperature. The reaction mixture was stirred for overnight. TLC showed complete consumption of starting material. The reaction mixture was diluted with water and extracted with ethylacetate. The organic part was washed with water and brine. The organic layer was dried over MgSO₄ and concentrated under reduced pressure. The crude was purified by column chromatography to give 1-(6-(hydroxymethyl)pyridin-3-yl)-3-((2-(4-methylpiperidin-1-yl)-6-(trifluoromethyl)pyridin-3-yl)methyl)urea (73 mg, 67%).

Example compounds 56-60 can be prepared analogously.

### Synthesis of Example 67

### 1-(5-Fluoro-6-(2-(methylsulfonyl)ethyl)pyridin-3-yl)-3-((2-(4-methylpiperidin-1-yl)-6-(trifluoromethyl)pyridin-3-yl)methyl)urea

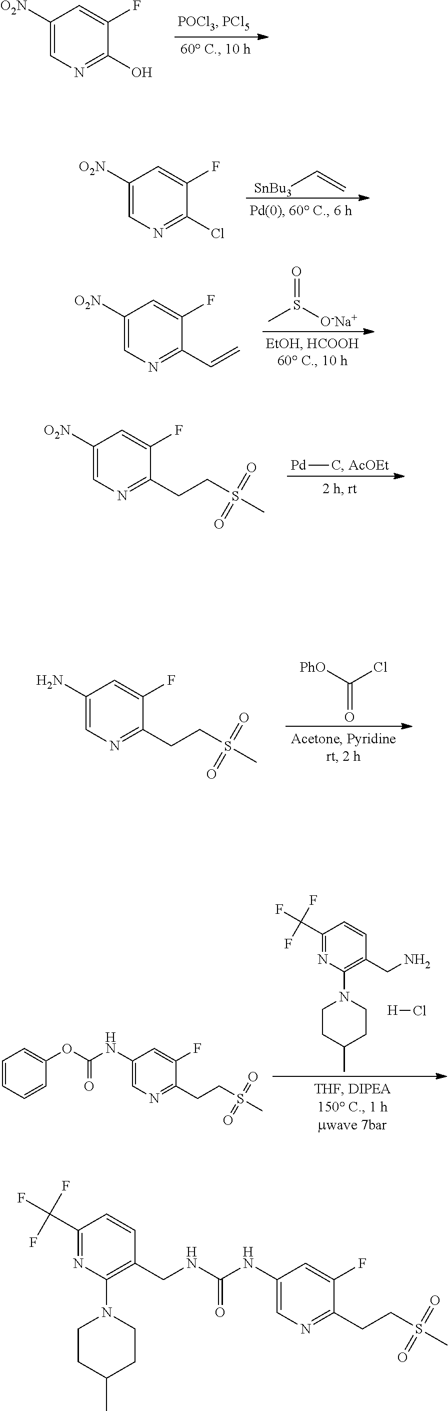

Step 1: To a stirred solution of 3-fluoro-5-nitropyridin-2-ol (1.5 g, 9.48 mmol) in phosphorous oxychloride (15 mL) was added phosphorous pentachloride (2.96 g, 14.22 mmol) at 60° C. The reaction mixture was allowed to stir for 10 h at the same temperature. The reaction mixture was cooled to ambient temperature and was poured into crushed ice and was extracted with ethyl acetate (3×20 mL). The total organic layer was washed with saturated sodium carbonate solution (25 mL). The washed organic layer was dried over anhydrous magnesium sulfate and was concentrated under reduced pressure to obtain crude compound which was purified by using silica gel column chromatography (100-200 mesh, 5% ethyl acetate in hexane) to afford 2-chloro-3-fluoro-5-nitropyridine (1.62 g, 97%).

Step 2: To a stirred solution of 2-chloro-3-fluoro-5-nitropyridine (1.6 g, 9.0 mmol) in tetrahydrofuran (16 mL) was added tributylvinyltin (3.42 g, 10.8 mmol) and Pd₂(dba)₃ (0.42 g, 0.45 mmol), trifuryl phosphene (0.2 g, 0.9 mmol) under nitrogen atmosphere. The reaction mixture was deoxygenated thoroughly and was heated to 60° C. for 6 h. The reaction mixture was diluted with water (20 mL) and was extracted with ethyl acetate (3×25 mL). The combined organic layer was washed with brine (25 mL) and dried over anhydrous magnesium sulfate and concentrated under reduced pressure to afford the crude compound. The crude compound was purified by column chromatography (silica gel: 100-200 mesh; eluent: 5% ethyl acetate in hexane) to afford 3-fluoro-5-nitro-2-vinylpyridine. (1.5 g, 96%).

Step 3: To a stirred solution 3-fluoro-5-nitro-2-vinylpyridine (1.5 g, 8.92 mmol) in ethanol (15 mL) was added sodium methane sulfinate (9.1 g, 89.3 mmol) and acetic acid (0.53 g, 8.92 mmol) at ambient temperature. The reaction mixture was heated to 60° C. for 10 h. The reaction mixture was cooled to ambient temperature and was concentrated under reduced pressure to obtain crude compound which was filtered and the solid obtained was washed with water (25 mL) to obtain 3-fluoro-2-(2-(methylsulfonyl)ethyl)-5-nitropyridine (0.81 g, 36%).

Step 4: 3-Fluoro-2-(2-(methylsulfonyl)ethyl)-5-nitropyridine (0.8 g, 3.22 mmol) was dissolved in ethyl acetate (8 mL), was added palladium on charcoal (80 mg) under argon atmosphere which was subjected to hydrogenated in Parr apparatus and the reaction was continued to stir for 2 h. The reaction mixture was filtered through celite bed and was washed thoroughly with ethyl acetate and was concentrated under reduced pressure to obtain 5-fluoro-6-(2-(methylsulfonyl)ethyl)pyridin-3-amine (0.62 g, 88%).

Step 5: 5-Fluoro-6-(2-(methylsulfonyl)ethyl)pyridin-3-amine (99 mg, 0.454 mmol) was dissolved in acetone/dimethylformamide (1.5 mL+0.63 mL). To the reaction mixture was added dropwise pyridine (0.11 mL, 1.36 mmol) followed by phenyl chloroformate (0.075 mL, 0.59 mmol) at 0° C. The mixture was stirred at room temperature for 2 h. The reaction mixture was concentrated under reduced pressure and diluted with dichloromethane and washed with sodium bicarbonate solution (1×15 mL). The aqueous layer was extracted with dichloromethane (2×20 mL). The organic layer was dried over MgSO₄ and concentrated under reduced pressure to give phenyl 5-fluoro-6-(2-(methylsulfonyl)ethyl)pyridin-3-ylcarbamate (249 mg).

Step 6: Phenyl 5-fluoro-6-(2-(methylsulfonyl)ethyl)pyridin-3-ylcarbamate (80 mg, 0.237 mmol) and (2-(4-methylpiperidin-1-yl)-6-(trifluoromethyl)pyridin-3-yl)methanamine hydrochloride (73 mg, 0.237 mmol) was dissolved in tetrahydrofuran (3.6 mL). Then N-ethyldiisopropylamine (0.157 mL, 0.924 mmol) was added to it. The mixture was stirred at 1 h at 150° C. in a microwave (at 7 bar). After completion, the mixture was concentrated under reduced pressure to get the crude compound. The crude compound was purified by column chromatography by using ethyl acetate-methanol (4:1) as eluent to afford 1-(5-fluoro-6-(2-(methylsulfonyl)ethyl)pyridin-3-yl)-3-((2-(4-methylpiperidin-1-yl)-6-(trifluoromethyl)pyridin-3-yl)methyl)urea (40 mg, 33%).

Example compounds 68 and 69 can be prepared analogously.

### Synthesis of Example 74

### 5-(1-((2-(4-Methylpiperidin-1-yl)-6-(trifluoromethyl)pyridin-3-yl)methylamino)-1-oxopropan-2-yl)picolinamide

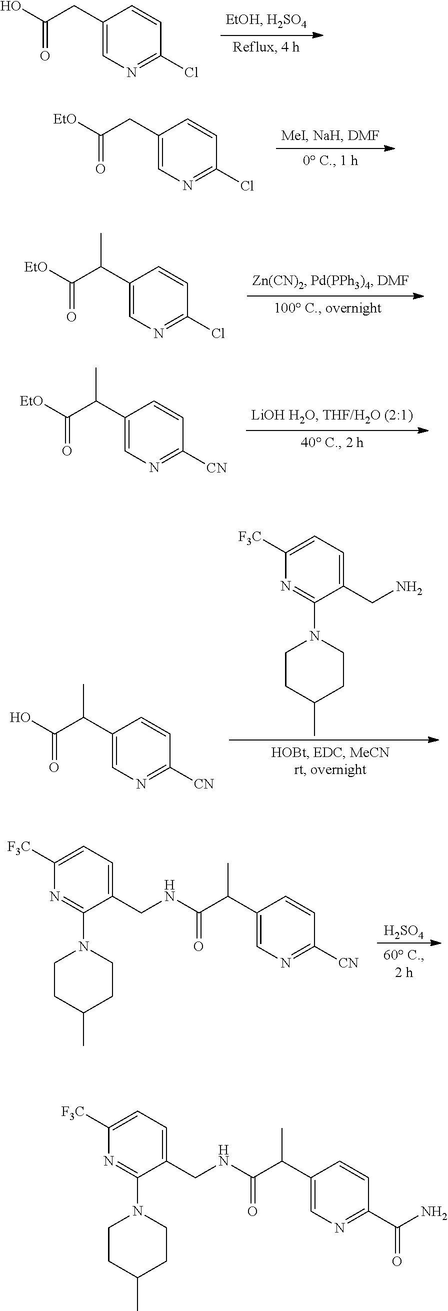

Step 1: To a solution of 6-chloro-3-pyridineacetic acid (1 g, 5.83 mmol) in ethanol was added sulfuric acid (1.6 mL). The mixture was refluxed for 4 h, then cooled to room temperature and concentrated. The residue was diluted with ethyl acetate and washed with a saturated sodium hydrogen carbonate solution. The resulting mixture was dried over MgSO₄ and concentrated under reduced pressure to afford crude which was purified by column chromatography to afford ethyl 2-(6-chloropyridin-3-yl)acetate (1.1 g, 95%).

Step 2: To a solution of ethyl 2-(6-chloropyridin-3-yl)acetate (1.1 g, 5.51 mmol) in dimethylformamide was added slowly sodium hydride (242 mg, 6.06 mmol) at 0° C., followed by iodomethane (821 mg, 5.79 mmol). The mixture was stirred at same degree for 1 hour, and then quenched with water. The resulting mixture was diluted with ethyl acetate and washed with water. The organic layer was dried over MgSO₄ and concentrated under reduced pressure to afford crude which was purified by column chromatography to afford ethyl 2-(6-chloropyridin-3-yl)propanoate (790 mg, 67%).

Step 3: To a solution of ethyl 2-(6-chloropyridin-3-yl)propanoate (790 mg, 3.7 mmol) in dimethylformamide was added Zn(CN)₂ (434 mg, 3.7 mmol) and Pd(PPh₃)₄ (1280 mg, 1.11 mmol). The reaction mixture was stirred for 12 h at 100° C. and then cooled to room temperature. The mixture was filtered through a plug of celite and concentrated. The residue was diluted with ethyl acetate and washed with 10% HCl. The organic layer was dried over MgSO₄ and concentrated under reduced pressure to afford crude which was purified by column chromatography to afford ethyl 2-(6-cyanopyridin-3-yl)propanoate (420 mg, 56%).

Step 4: To a solution of ethyl 2-(6-cyanopyridin-3-yl)propanoate (420 mg, 2.06 mmol) in tetrahydrofuran and water was added lithium hydroxide monohydrate (129 mg, 3.08 mmol). The reaction mixture was stirred for 2 h at 40° C. and then acidified with 10% HCl. The mixture was extracted with ethyl acetate. The organic layer dried over MgSO₄ and concentrated under reduced pressure to afford the desired 2-(6-cyanopyridin-3-yl)propanoic acid (330 mg, 94%).

Step 5: To a solution of 2-(6-cyanopyridin-3-yl)propanoic acid (330 mg, 1.87 mmol) in acetonitrile was added 1-hydroxybenzotriazole (380 mg, 2.81 mmol), 1-ethyl-3-(3-dimethylaminopropyl) carbodiimide (537 mg, 2.81 mmol) and (2-(4-methylpiperidin-1-yl)-6-(trifluoromethyl)pyridin-3-yl)methanamine (537 mg, 1.97 mmol). The reaction mixture was stirred for 12 h at room temperature. The reaction mixture was added water and extracted with ethyl acetate. The organic layer was dried over MgSO₄ and concentrated under reduced pressure. The crude was purified by column chromatography to give pure 2-(6-cyanopyridin-3-yl)-N-((2-(4-methylpiperidin-1-yl)-6-(trifluoromethyl)pyridin-3-yl)methyl)propanamide (500 mg, 62%).

Step 6: Starting material 2-(6-cyanopyridin-3-yl)-N-((2-(4-methylpiperidin-1-yl)-6-(trifluoromethyl)pyridin-3-yl)methyl)propanamide (140 mg, 0.33 mmol) was dissolved in sulfuric acid (1.7 mL). The reaction mixture was stirred for 2 h at 60° C. and then cooled to room temperature. The reaction mixture was diluted with ice water and neutralized (pH=7) with 2 M NaOH solution. The mixture was extracted with ethyl acetate. The organic layer was dried over MgSO₄ and concentrated under reduced pressure. The crude was purified by column chromatography to give pure 5-(1-((2-(4-methylpiperidin-1-yl)-6-(trifluoromethyl)pyridin-3-yl)methylamino)-1-oxopropan-2-yl)picolinamide (40 mg, 27%).

¹H NMR (300 MHz, CDCl₃) δ 8.52 (d, 1H, J=2.01 Hz, pyridine-H), 8.15 (d, 1H, J=8.14 Hz, pyridine-H), 7.85 (dd, 1H, J=8.09, 2.21 Hz, pyridine-H), 7.80 (br.s, NH), 7.50 (d, 1H, J=7.73 Hz), 7.21 (d, 1H, J=7.73 Hz, Ar—H), 6.55 (m, NH), 5.78 (br.s, NH), 4.50 (m, 2H, Ar—CH₂), 3.67 (quartet, 1H, J=6.96 Hz, amide-CH), 3.31 (m, 2H, piperidine-H), 2.82 (m, 2H, piperidine-H), 1.72 (m, 2H, piperidine-H), 1.56 (m, 4H, amide-CH₃, piperidine-H), 1.19 (m, 2H, piperidine-H), 0.97 (d, 3H, J=6.39 Hz, piperidine-CH₃).

### Synthesis of Example 75

### 5-(1-((2-(4-Methylpiperidin-1-yl)-6-(trifluormethyl)pyridin-3-yl)methylamino)-1-oxopropan-2-yl)-N-phenylpicolinamid

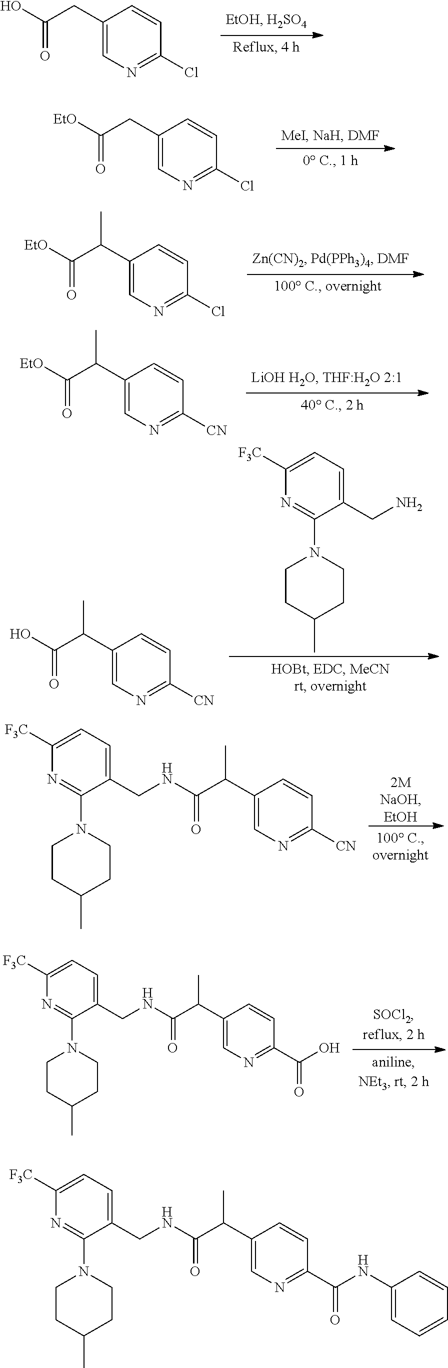

Step 1-5: as described for example 74.

Step 6: 2-(6-Cyanopyridin-3-yl)-N-((2-(4-methylpiperidin-1-yl)-6-(trifluoromethyl)pyridin-3-yl)methyl)propanamide (200 mg, 0.46 mmol) was suspended in ethanol, 2M NaOH (2.3 mL, 4.64 mmol) was added and the mixture was refluxed for 20 h. The mixture was cooled to room temperature and concentrated. The reaction mixture was diluted with ethyl acetate and acidified with 1M HCl solution. The mixture was extracted with ethyl acetate. The organic layer was dried over MgSO₄ and concentrated under reduced pressure. The crude was purified by column chromatography to give pure 5-(1-((2-(4-methylpiperidin-1-yl)-6-(trifluoromethyl)pyridin-3-yl)methylamino)-1-oxopropan-2-yl)picolinic acid (180 mg, 78%).

Step 7: To a solution of 5-(1-((2-(4-methylpiperidin-1-yl)-6-(trifluoromethyl)pyridin-3-yl)methylamino)-1-oxopropan-2-yl)picolinic acid (180 mg, 0.4 mmol) in chloromethane was added thionyl chloride (0.14 mL, 2 mmol). The reaction mixture was refluxes for 2 h and then thionyl chloride was removed under reduced pressure. The residue was dissolved in chloromethane and it was added to the solution aniline (0.037 mL, 0.4 mmol) and triethylamine (0.08 mL, 0.6 mmol) in chloromethane. The reaction mixture was stirred at room temperature for 2 h and then added water and extracted with chloromethane. The organic layer was dried over MgSO₄ and concentrated under reduced pressure. The crude was purified by column chromatography to give 5-(1-((2-(4-methylpiperidin-1-yl)-6-(trifluoromethyl)pyridin-3-yl)methylamino)-1-oxopropan-2-yl)-N-phenylpicolinamide (50 mg, 25%).

¹H NMR (300 MHz, CDCl₃) δ 9.94 (br.s, 1H, NH), 8.56 (d, 1H, J=2.01 Hz, pyridine-H), 8.26 (d, 1H, J=8.04 Hz, pyridine-H), 7.89 (dd, 1H, J=8.11, 2.04 Hz, pyridine-H), 7.76 (d, 2H, J=7.75 Hz, Ar—H), 7.51 (d, 1H, J=7.52 Hz, Ar—H), 7.40 (m, 2H, Ar—H), 7.18 (m, 2H, Ar—H), 6.51 (br.s, 1H, NH), 4.51 (m, 2H, Ar—CH₂), 3.68 (quartet, 1H, J=7.04 Hz, amide-CH), 3.32 (m, 2H, piperidine-H), 2.83 (m, 2H, piperidine-H), 1.71 (m, 2H, piperidine-H), 1.60 (m, 4H, amide-CH₃, piperidine-H), 1.23 (m, 2H, piperidine-H), 0.96 (d, 3H, J=6.41 Hz, piperidine-CH₃).

### Synthesis of Example 76

### 5-(1-((2-(4-methylpiperidin-1-yl)-6-(trifluoromethyl)pyridin-3-yl)methylamino)-1-oxopropan-2-yl)-N-phenylpyrimidine-2-carboxamide

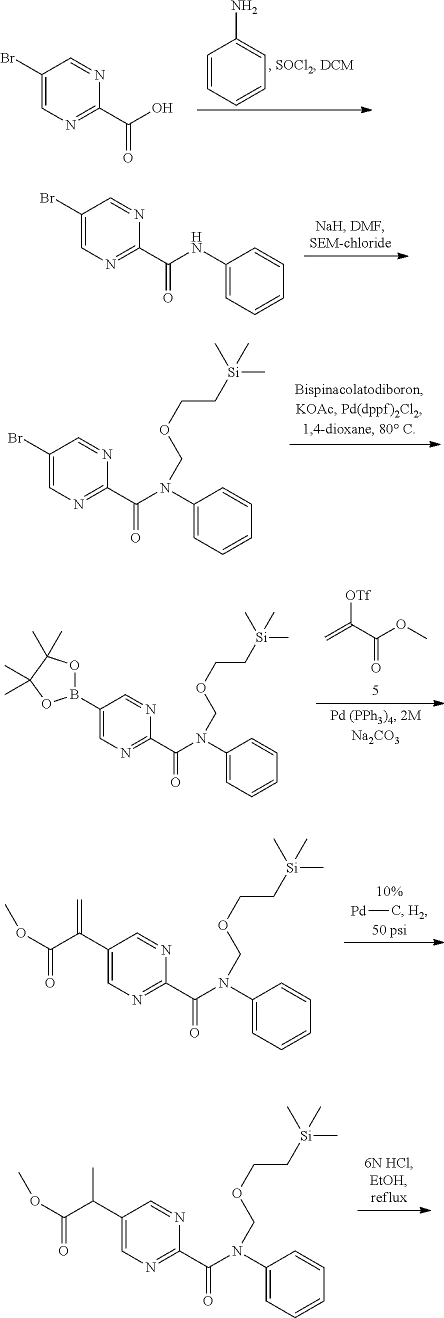

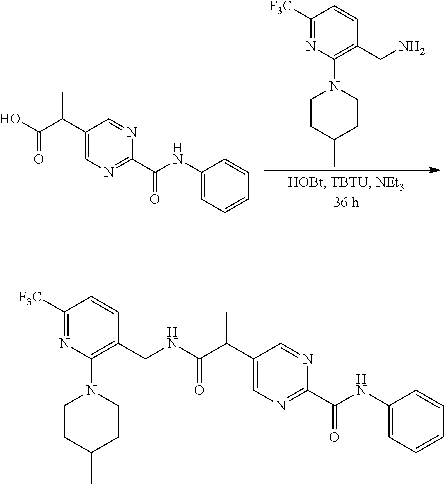 [SMILES: CC(C(=O)O)c1cnc(C(=O)Nc2ccccc2)nc1 ; CC1CCN(c2nc(C(F)(F)F)ccc2CN)CC1 ; CC1CCN(c2nc(C(F)(F)F)ccc2CNC(=O)C(C)c2cnc(C(=O)Nc3ccccc3)nc2)CC1]

Step 1: 5-Bromopyrimidine-2-carboxylic acid (5.22 g, 24.63 mmol) was dissolved in benzene (100 mL) and thionyl chloride (5.4 mL, 73.89 mmol) was added to it in a 250 mL round bottomed flask. The reaction mixture was refluxed for 2 h at 100° C. After that thionyl chloride and benzene was removed under reduced pressure. Water was removed by making azeotrope using benzene. The residue was dissolved in dichloromethane (100 mL) and it was added to the solution of aniline (2.27 g, 24.42 mmol) in dichloromethane (100 mL) under nitrogen atmosphere. The reaction mixture was stirred for 16 h at ambient temperature. After total consumption of starting material, the reaction mixture was diluted with dichloromethane (50 mL) and washed with water (2×100 mL) followed by sodium bicarbonate solution (2×100 mL) and brine (100 mL). The organic layer was dried over MgSO₄ and concentrated under reduced pressure. The crude compound was purified by column chromatography (silica gel: 100-200 mesh, eluent: 20% ethyl acetate in n-hexane) to get 5-bromo-N-phenylpyrimidine-2-carboxamide (5.5 g, 77%).

Step 2: Sodium hydride (950 mg, 23.91 mmol) was taken in a 250 mL round bottomed two-necked flask and dry dimethylformamide (20 mL) was added to it under nitrogen atmosphere. To the suspension of sodium hydride in dimethylformamide solution of 5-bromo-N-phenylpyrimidine-2-carboxamide (5.5 g, 19.92 mmol) in dry dimethylformamide (39.76 mL) was added at −5° C. The reaction mixture was stirred at the same temperature for 30 minutes. After that 2-(trimethylsilyl)ethoxymethyl chloride (4.98 g, 29.89 mmol) was added to it dropwise maintaining the temperature. The reaction mixture was stirred at ambient temperature for 2 h. After total consumption of starting material the reaction mixture was quenched with ammonium chloride solution (150 mL) and extracted with ethyl acetate (3×100 mL). The combined organic layer was dried over MgSO₄ and concentrated under reduced pressure. The crude compound was purified by column chromatography (silica gel: 100-200 mesh, eluent: 20% ethyl acetate in n-hexane) to afford the pure 5-bromo-N-phenyl-N-((2-(trimethylsilyl)ethoxy)methyl)pyrimidine-2-carboxamide (7.2 g, 90%).

Step 3: 5-Bromo-N-phenyl-N-((2-(trimethylsilyl)ethoxy)methyl)pyrimidine-2-carboxamide (6.5 g, 15.92 mmol) was dissolved in 1,4-dioxane (80 mL) and 4,4,4′,4′,5,5,5′,5′-octamethyl-2,2′-Bi-(1,3,2-dioxaborolane) (4.24 g, 16.7 mmol) was added to it followed by potassium acetate (4.68 g, 47.76 mmol) under nitrogen atmosphere. The reaction mixture was stirred for 5 minutes and Pd(dppf)Cl₂ (582 mg, 0.79 mmol) was added to it. The reaction mixture was refluxed for 16 h. After total consumption of starting material the reaction mixture was diluted with water and extracted with ethyl acetate (3×100 mL). The combined organic layer was dried over magnesium sulfate and concentrated under reduced pressure. The crude N-phenyl-5-(4,4,5,5-tetramethyl-1,3,2-dioxaborolan-2-yl)-N-((2-(trimethylsilyl)ethoxy)methyl)pyrimidine-2-carboxamide was used for next step without purification (8.0 g, crude).

Step 4: N-Phenyl-5-(4,4,5,5-tetramethyl-1,3,2-dioxaborolan-2-yl)-N-((2-(trimethylsilyl)ethoxy)-methyl)pyrimidine-2-carboxamide (7.3 g, 16.04 mmol) was dissolved in toluene (73 mL) and methyl 2-(trifluoromethylsulfonyloxy)acrylate (4.5 g, 19.25 mmol) was added to it followed by 2M sodium carbonate solution (32 mL) under nitrogen atmosphere. After that Pd(PPh₃)₄ (927 mg, 0.80 mmol) was added to it. The reaction mixture was refluxed for 16 h. After total consumption of starting material the reaction mixture was diluted with water and extracted with ethyl acetate (3×100 mL). The combined organic layer was dried over magnesium sulfate and concentrated under reduced pressure. The crude was purified by column chromatography (silica gel: 100-200 mesh, eluent: 10% ethyl acetate in n-hexane) to afford the pure methyl 2-(2-(phenyl((2-(trimethylsilyl)ethoxy)methyl)carbamoyl)pyrimidin-5-yl)acrylate (4.3 g, 65%).

Step 5: Methyl 2-(2-(phenyl((2-(trimethylsilyl)ethoxy)methyl)carbamoyl)pyrimidin-5-yl)acrylate (4.3 g) was dissolved in ethyl acetate (43 mL) in a 250 mL Parr vessel and palladium on activated charcoal (10% Pd, 430 mg) was added to it under nitrogen atmosphere. The vessel was equipped in Parr apparatus under 50 psi hydrogen pressure. After 2 h TLC showed the total consumption of starting material. The catalyst was filtered through celite bed and filtrate was concentrated under reduced pressure to afford methyl 2-(2-(phenyl((2-(trimethylsilyl)ethoxy)methyl)carbamoyl)pyrimidin-5-yl)propanoate (4.0 g, 93%)

Step 6: Methyl 2-(2-(phenyl((2-(trimethylsilyl)ethoxy)methyl)carbamoyl)pyrimidin-5-yl)propanoate (2.5 g, 6.0 mmol) was dissolved in ethanol (76 mL) and 6N HCl (76 mL) was added to it. The reaction mixture was refluxed for 2 h at 90° C. After complete conversion of starting material ethanol was evaporated under reduced pressure and residue was diluted with water and basified by sodium carbonate solution. The aqueous layer was washed with ethyl acetate. After that the aqueous layer was acidified with 6N HCl and extracted with ethyl acetate (3×50 mL). The combined organic layer was dried over magnesium sulphate and concentrated under reduced pressure to afford the pure 2-(2-(phenylcarbamoyl)pyrimidin-5-yl)propanoic acid (750 mg, 47%).

¹H NMR (DMSO-d₆, 400 MHz): δ 12.87 (1H, s), δ 10.70 (1H, s), δ 8.97 (2H, s), δ 7.86 (2H, d), δ 7.37 (2H, t), δ 7.13 (1H, t), δ 3.97 (1H, q), δ 1.52 (3H, d); LCMS (M+H): 272.0; HPLC: 95.02%

Step 7: To a stirred solution of (2-(4-methylpiperidin-1-yl)-6-(trifluoromethyl)pyridin-3-yl)methanamine (0.07 g, 0.256 mmol) and 2-(2-(phenylcarbamoyl)pyrimidin-5-yl)propanoic acid (0.069 g, 0.256 mmol) in tetrahydrofuran (2 mL) was added 1-hydroxybenzotriazolhydrate (0.034 mL, 0.256 mmol), O-(1H-benzotriazol-1-yl)-N,N,N′,N′-tetramethyluronium tetrafluoroborate (0.082 g, 0.256 mmol) and N-ethyldiisopropylamine (0.066 mL, 0.512 mmol) and the reaction mixture was allowed to stir for 36 h. The reaction mixture was concentrated under reduced pressure and the solid obtained was purified by column chromatography (silica gel: 100-200 mesh, eluent: ethyl acetate) to afford 5-(1-((2-(4-methylpiperidin-1-yl)-6-(trifluoromethyl)pyridin-3-yl)methylamino)-1-oxopropan-2-yl)-N-phenylpyrimidine-2-carboxamide (35 mg, 26%).

### Synthesis of Example 77

### 5-(1-((2-(ethylamino)-6-(trifluoromethyl)pyridin-3-yl)methylamino)-1-oxopropan-2-yl)-N-(4-fluorophenyl)pyrimidine-2-carboxamide

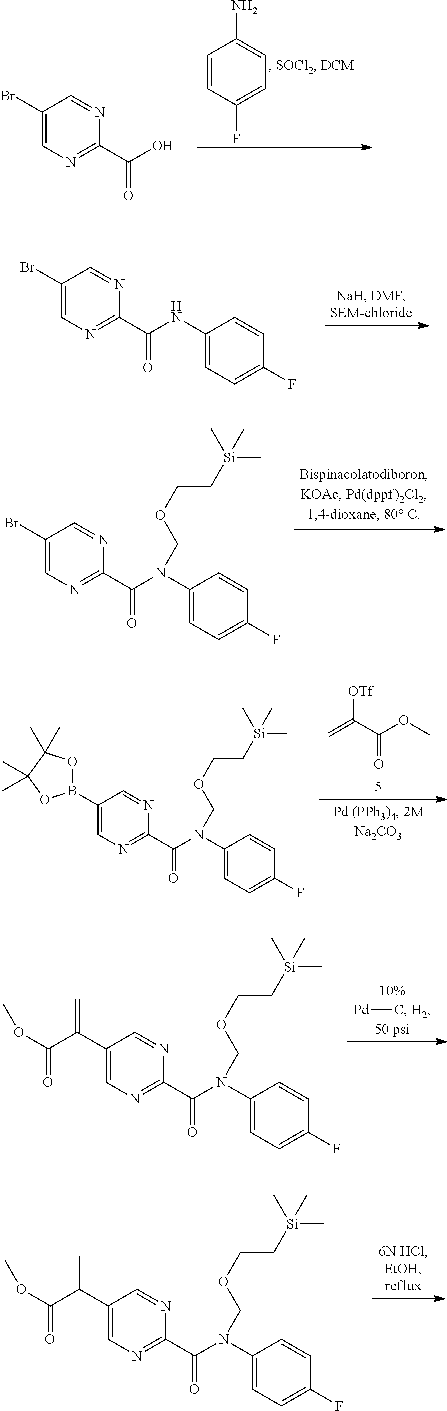

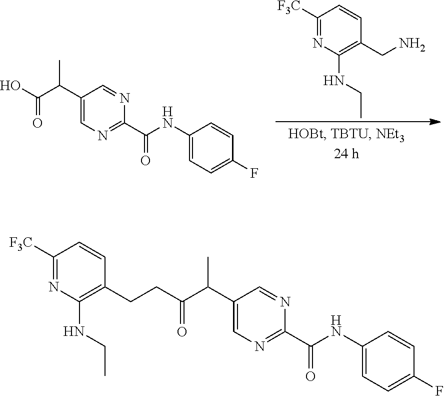 [SMILES: CC(C(=O)O)c1cnc(C(=O)Nc2ccc(F)cc2)nc1 ; CCNc1nc(C(F)(F)F)ccc1CN ; CCNc1nc(C(F)(F)F)ccc1CCC(=O)C(C)c1cnc(C(=O)Nc2ccc(F)cc2)nc1]

Step 1: 5-Bromopyrimidine-2-carboxylic acid (5 g, 24.63 mmol) was dissolved in benzene (50 mL) and thionyl chloride (5.63 mL, 73.89 mmol) was added to it in a 250 mL round bottomed flask. The reaction mixture was refluxed for 2 h at 100° C. After that thionyl chloride and benzene was removed under reduced pressure. Water was removed by making azeotrope using benzene. The residue was dissolved in dichloromethane (100 mL) and it was added to the solution of 4-fluoroaniline (2.68 g, 24.13 mmol) in dichloromethane (100 mL) under nitrogen atmosphere. The reaction mixture was stirred for 16 h at room temperature. After total consumption of starting material, the reaction mixture was diluted with dichloromethane (50 mL) and washed with water (2×100 mL) followed by sodium bicarbonate solution (2×100 mL) and brine (100 mL). The organic layer was dried over magnesium sulfate and concentrated under reduced pressure. The crude compound was purified by column chromatography (silica gel: 100-200 mesh, eluent: 20% ethyl acetate in n-hexane) to afford 5-bromo-N-(4-fluorophenyl)pyrimidine-2-carboxamide (5.6 g, 78%).

Step 2: Sodium hydride (60%, 872 mg, 21.81 mmol) was taken in a 250 mL round bottomed two-necked flask and dry dimethylformamide (25 mL) was added to it under nitrogen atmosphere. To the suspension of sodium hydride in dimethylformamide solution of 5-bromo-N-(4-fluorophenyl)pyrimidine-2-carboxamide (5.4 g, 18.24 mmol) in dry dimethylformamide (30 mL) was added at −5° C. The reaction mixture was stirred at same temperature for 30 minutes. After that 2-(trimethylsilyl)ethoxymethyl chloride (4.52 g, 27.36 mmol) was added to it drop wise maintaining the temperature. The reaction mixture was stirred at ambient temperature for 2 h. After total consumption of starting material the reaction mixture was quenched with ammonium chloride solution (150 mL) and extracted with ethyl acetate (3×100 mL). The combined organic layer was over MgSO₄ and concentrated under reduced pressure. The crude compound was purified by column chromatography (100-200 mesh silica gel, 20% ethyl acetate in n-hexane) to afford 5-bromo-N-(4-fluorophenyl)-N-((2-(trimethylsilyl)ethoxy)methyl)pyrimidine-2-carboxamide (6.5 g, 84%).

Step 3: 5-Bromo-N-(4-fluorophenyl)-N-((2-(trimethylsilyl)ethoxy)methyl)pyrimidine-2-carboxamide (7.5 g, 17.59 mmol) was dissolved in 1,4-dioxane (86 mL) and 4,4,4′,4′,5,5,5′,5′-octamethyl-2,2′-Bi-(1,3,2-dioxaborolane) (4.7 g, 18.47 mmol) was added to it followed by potassium acetate (5.2 g, 52.77 mmol) under nitrogen atmosphere. The reaction mixture was stirred for 5 minutes and Pd(dppf)₂Cl₂ (644 mg, 0.87 mmol) was added to it. The reaction mixture was refluxed for 16 h. After total consumption of starting material the reaction mixture was diluted with water and extracted with ethyl acetate (3×100 mL). The combined organic layer was dried over MgSO₄ and concentrated under reduced pressure. The crude N-(4-fluorophenyl)-5-(4,4,5,5-tetramethyl-1,3,2-dioxaborolan-2-yl)-N-((2-(trimethylsilyl)ethoxy)-methyl)pyrimidine-2-carboxamide was used for next step without purification (9.0 g, crude).

Step 4: N-(4-Fluorophenyl)-5-(4,4,5,5-tetramethyl-1,3,2-dioxaborolan-2-yl)-N-((2-(trimethylsilyl)-ethoxy)methyl)pyrimidine-2-carboxamide (8.3 g, 17.59 mmol) was dissolved in toluene (83 mL) and methyl 2-(trifluoromethylsulfonyloxy)acrylate (4.94 g, 21.12 mmol) was added to it followed by 2 M sodium carbonate solution (35.2 mL) under nitrogen atmosphere. After that Pd(PPh₃)₄ (1.02 g, 0.87 mmol) was added to it. The reaction mixture was refluxed for 16 h. After total consumption of starting material the reaction mixture was diluted with water and extracted with ethyl acetate (3×100 mL). The combined organic layer was dried over MgSO₄ and concentrated under reduced pressure. The crude was purified by column chromatography (silica gel: 100-200 mesh, eluent: 10% ethyl acetate in n-hexane) to afford methyl 2-(2-((4-fluorophenyl)((2-(trimethylsilyl)ethoxy)methyl)carbamoyl)pyrimidin-5-yl)acrylate(5 g, 67%).

Step 5: Methyl 2-(2-((4-fluorophenyl)((2-(trimethylsilyl)ethoxy)methyl)carbamoyl)pyrimidin-5-yl)acrylate (5.0 g) was dissolved in ethyl acetate (50 mL) in a 500 mL Parr vessel and palladium on activated charcoal (10% on Pd, 500 mg) was added to it under nitrogen atmosphere. The vessel was equipped in Parr apparatus under 50 psi hydrogen pressure. After two hours TLC showed the total consumption of starting material. The catalyst was filtered through celite bed and filtrate was concentrated under reduced pressure to afford methyl 2-(2-((4-fluorophenyl)((2-(trimethylsilyl)ethoxy)methyl)carbamoyl)pyrimidin-5-yl)propanoate (5 g, quantitative).

Step 6: Methyl 2-(2-((4-fluorophenyl)((2-(trimethylsilyl)ethoxy)methyl)carbamoyl)pyrimidin-5-yl)propanoate (3.0 g, 6.92 mmol) was dissolved in ethanol (87 mL) and 6N HCl (87 mL) was added to it. The reaction mixture was refluxed for 2 h at 90° C. After complete conversion of starting material ethanol was evaporated under reduced pressure and residue was diluted with water and basified by sodium carbonate solution. The aqueous layer was washed with ethyl acetate. After that the aqueous layer was acidified with 6N HCl and extracted with ethyl acetate (3×50 mL). The combined organic layer was dried over over MgSO₄ and concentrated under reduced pressure to afford the pure 2-(2-(4-fluorophenylcarbamoyl)pyrimidin-5-yl)propanoic acid (700 mg, 35%).

¹H NMR (DMSO-d₆, 400 MHz): δ 12.82 (1H, s), 10.80 (1H, s), 8.94 (2H, s), 7.91-7.88 (2H, m), 7.20 (2H, t), 3.96 (1H, q), 1.52 (3H, d); LCMS (M+H): 290; HPLC: 97.71%

Step 7: To a stirred solution of 3-(aminomethyl)-N-ethyl-6-(trifluoromethyl)pyridin-2-amine (0.055 g, 0.251 mmol) and 2-(2-(4-fluorophenylcarbamoyl)pyrimidin-5-yl)propanoic acid (0.072 g, 0.251 mmol) in tetrahydrofuran (2 mL) was added 1-hydroxybenzotriazolhydrate (0.034 mL, 0.251 mmol), 0-(1H-benzotriazol-1-yl)N,N,N′,N′-tetramethyluronium tetrafluoroborate (0.082 g, 0.251 mmol) and N-ethyldiisopropylamine (0.034 mL, 0.251 mmol) and the reaction mixture was allowed to stir for 24 h. The reaction mixture was concentrated under reduced pressure and the solid obtained was purified by column chromatography (silica gel: 100-200 mesh, eluent: 5% methanol in ethyl acetate) to afford 5-(5-(2-(ethylamino)-6-(trifluoromethyl)pyridin-3-yl)-3-oxopentan-2-yl)-N-(4-fluorophenyl)pyrimidine-2-carboxamide (74 mg, 60%).

The example compounds 78-81 were prepared in a similar manner.

### Synthesis of Example 84

### 2-(5-Amino-6-brompyridin-2-yl)-N-((2-(4-methylpiperidin-1-yl)-6-(trifluoromethyl)pyridin-3-yl)methyl)propanamid

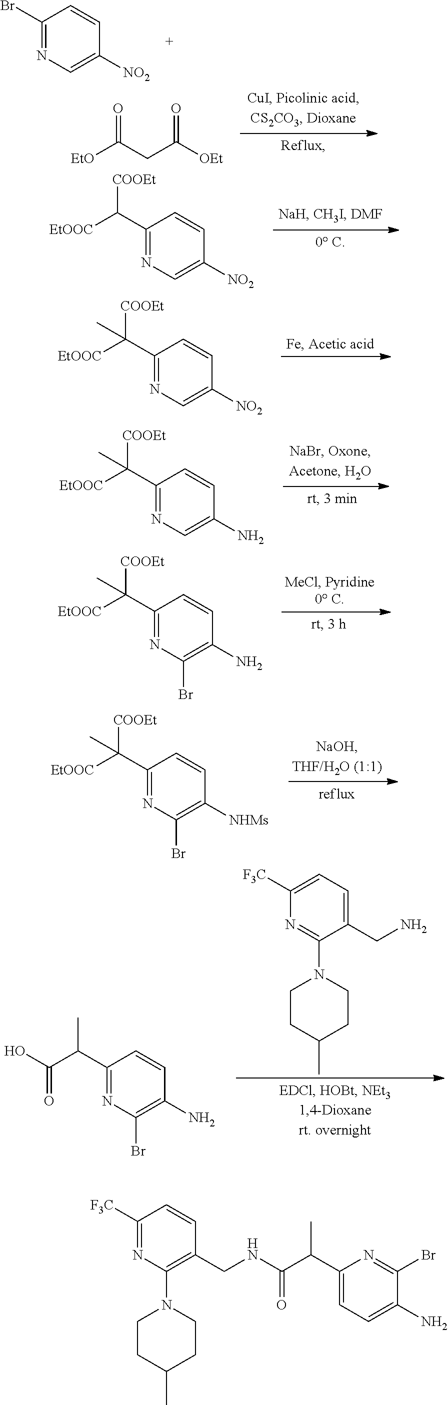

Step 1: To a solution of 2-bromo-5-nitropyridine (1.5 g, 7.4 mmol) and malonic acid diethyl ester in 1,4-dioxane was added CuI (0.28 g, 1.476 mmol), CS₂CO₃ (7 g, 22.2 mmol) and picolinic acid (0.182 g, 1.478 mmol). The mixture was refluxed. To the mixture was added water and extracted with ethyl acetate. The organic layer was dried over MgSO₄, filtered and concentrated. The residue was purified by column chromatography to yield diethyl 2-(5-nitropyridin-2-yl)malonate (2.9 g, 99%).

Step 2: To a solution of diethyl 2-(5-nitropyridin-2-yl)malonate (2.9 g, 10.27 mmol) in dimethylformamide was added sodium hydride (0.4 g, 15.4 mmol) and iodomethane (0.6 mL, 15.4 mmol) at 0° C. To the mixture was added water and extracted with ethyl acetate. The organic layer was dried over MgSO₄, filtered and concentrated. The residue was purified column chromatography, diethyl 2-methyl-2-(5-nitropyridin-2-yl)malonate (0.956 g, 32%) was obtained.

Step 3: To a solution of diethyl 2-methyl-2-(5-nitropyridin-2-yl)malonate (0.956 g, 3.23 mmol) in acetic acid was added Fe (0.901 g, 10.5 mmol). To the mixture was added water and extracted with ethyl acetate. The organic layer was dried over MgSO₄, filtered and concentrated. The residue was purified column chromatography, diethyl 2-(5-aminopyridin-2-yl)-2-methylmalonate (0.85 g, 99%) was obtained.

Step 4: To a solution of diethyl 2-(5-aminopyridin-2-yl)-2-methylmalonate (0.5 g, 1.9 mmol) in water and acetone was added sodium bromide (0.133 g, 1.9 mmol) and oxone (1.29 g, 1.9 mmol). The mixture was stirred for 3 min at room temperature. To the mixture was added water and extracted with ethyl acetate. The organic layer was dried over MgSO₄, filtered and concentrated. The residue was purified column chromatography, diethyl 2-(5-amino-6-bromopyridin-2-yl)-2-methylmalonate (0.36 g, 41%) was obtained.

Step 5: To a solution of diethyl 2-(5-amino-6-bromopyridin-2-yl)-2-methylmalonate in pyridine was added Methanesulfonyl chloride (0.1 mL, 1.8 mmol) at 0° C. The mixture was stirred for 30 min at 0° C. and then 3 h at room temperature. To the mixture was added water and extracted with ethyl acetate. The organic layer was dried over MgSO₄, filtered and concentrated. The residue was purified column chromatography. Diethyl 2-(6-bromo-5-(methylsulfonamido)pyridin-2-yl)-2-methylmalonate (0.37 g, 99%) was obtained.

Step 6: To a solution of diethyl 2-(6-bromo-5-(methylsulfonamido)pyridin-2-yl)-2-methylmalonate (0.215 g, 0.5 mmol) in tetrahydrofuran and water was added NaOH (0.042 g, 1 mmol). The mixture was refluxed and then added water and acidified with acetic acid. The mixture was extracted with dichloromethane. The organic layer was dried over MgSO₄, filtered and concentrated. The residue was purified column chromatography. 2-(5-amino-6-bromopyridin-2-yl)propanoic acid (0.238 g, 99%) was obtained.

Step 7: To a solution of 2-(5-amino-6-bromopyridin-2-yl)propanoic acid (0.238 g, 0.74 mmol) and (2-(4-methylpiperidin-1-yl)-6-(trifluoromethyl)pyridin-3-yl)methanamine (0.201 g, 0.74 mmol) in 1,4-dioxane was added 1-ethyl-3-(3-dimethylaminopropyl) carbodiimide) (0.226 g, 1.184 mmol), 1-hydroxybenzotriazole (0.16 g, 1.184 mmol) and triethylamine (0.008 g, 0.67 mmol) at room temperature. The reaction mixture was stirred for 15 h at room temperature and then added water and extracted with ethyl acetate. The organic layer was dried over MgSO₄, filtered and concentrated. The residue was purified column chromatography. 2-(5-amino-6-bromopyridin-2-yl)-N-((2-(4-methylpiperidin-1-yl)-6-(trifluoromethyl)pyridin-3-yl)methyl)propanamide (0.2 g, 54%) was obtained.

¹H NMR (300 MHz, CDCl3) δ 7.53 (d, 1H, J=7.68 Hz, Ar—H), 7.20 (d, 1H, J=7.71 Hz, Ar—H), 7.08 (d, 1H, J=8.04 Hz, Ar—H), 6.09 (m, 2H, Ar—H and CO—NH), 4.47 (m, 2H, Ar—CH₂) 4.10 (br.s, 2H, Ar—NH₂), 3.69 (q, 1H, J=7.3 Hz, Ar—CH), 3.37 (m, 2H, piperidine-H), 2.83 (m, 2H, piperidine-H), 1.72 (m, 2H, piperidine-H), 1.55 (d, 3H, J=7.14 Hz, ArCH—CH₃), 1.39 (m, 3H, piperidine-H and 2H), 0.96 (d, 3H, J=7.3 Hz, piperidine-CH₃).

### Synthesis of Example 85

### 2-(6-(2-Hydroxyethylamino)pyridin-3-yl)-N-((2-(4-methylpiperidin-1-yl)-6-(trifluoromethyl)pyridin-3-yl)methyl)propanamide

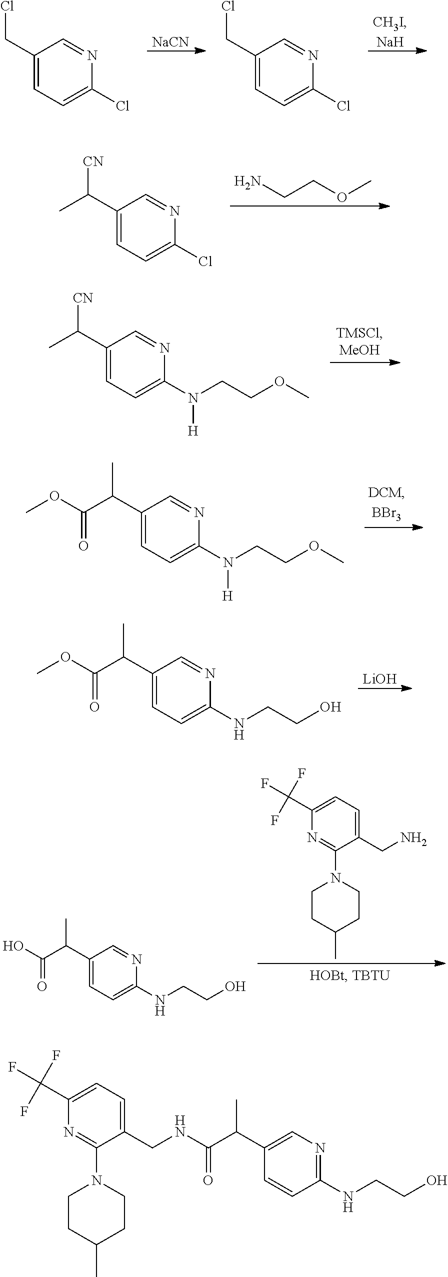

Step 1: To a stirred solution of 2-chloro-5-(chloromethyl)pyridine (1 g, 6.17 mmol, 1.0 equiv.) in ethanol (10 mL) was added the solution of NaCN (325 mg, 6.79 mmol, 1.1 eq) in H₂O (10 mL) dropwise at 0° C. and stirred for 3 h at 100° C. The reaction mixture was diluted with water (50 ml), extracted with ethyl acetate (70 mL×2) washed with brine (20 mL), dried over anhydrous Na₂SO₄ and evaporated under vacuum. The crude was purified by using silica gel chromatography (100-200 mesh) using ethyl acetate/petrol ether (3:7) to get 2-(6-chloropyridin-3-yl)acetonitrile (400 mg, 63%) as a yellow solid.

Step 2: To a stirred solution of 2-(6-chloropyridin-3-yl)acetonitrile (10 g, 65.7 mmol, 1.0 equiv.) in tetrahydrofuran (100 mL) cooled to 0° C. was added NaH (1.578 g, 65.7 mmol, 1.0 equiv.) as portion wise stirred for 10 min. CH₃I (4.02 mL, 65.7 mmol, 1.0 equiv.) was added at 0° C. The reaction mixture was diluted with water (150 ml), extracted with ethyl acetate (100 mL×2) and brine (100 mL) and dried over sodium sulfate and evaporated under vacuum. The crude was purified by silica gel chromatography (100-200 mesh) using ethyl acetate/petrol ether (1:4) to get 2-(6-chloropyridin-3-yl)propanenitrile (5 g, 46%) as solid.

Step 3: To a stirred solution of 2-(6-chloropyridin-3-yl)propanenitrile (2 g, 12.04 mmol, 1.0 equiv.) in DMSO (15 mL) was added TEA (3.34 mL, 24.09 mmol, 2.0 equiv.) and N(2-methoxy ethyl)methyl amine (1.8 g, 24.09 mmol, 2.0 equiv.) and heated to 100° C. for 16 h. The reaction mixture was diluted with water (50 mL), extracted with ethyl acetate (60 mL×2). The organic layer was washed with brine (50 mL), dried over sodium sulfate and evaporated under vacuum. The residue obtained was purified by neutral alumina using ethyl acetate/petrol ether (3:7) as eluent to get 2-(6-(2-methoxyethylamino)pyridin-3-yl)propanenitrile (500 mg, 40%) as white solid.

Step 4: To a stirred solution of TMSCl (4.6 mL, 20.4 mmol, 3.0 equiv.) in methanol (8 mL) was added 2-(6-(2-methoxyethylamino)pyridin-3-yl)propanenitrile (1.4 g, 6.8 mmol, 1.0 eq) and heated to 60° C. for 5 h. The reaction mixture was diluted with water (50 mL) and pH≈9 adjusted with NaHCO₃ (10 mL) extracted with ethyl acetate (2×100 mL). The organic layer was separated and washed with brine (50 mL), dried over Na₂SO₄ and evaporated under vacuum. The residue was purified by silica gel column (100-200 mesh) using ethyl acetate/petrol ether (1:1) as eluent to get methyl 2-(6-(2-methoxyethylamino)pyridin-3-yl)propanoate (1.2 g, 74%) as a pale yellow liquid.

Step 5: To a stirred solution of methyl 2-(6-(2-methoxyethylamino)pyridin-3-yl)propanoate (1.5 g, 6.3 mmol, 1.0 equiv.) in dichloromethane (20 mL) was added compound BBr₃ (9.4 mL, 9.4 mmol, 1.5 equiv.) at −78° C. and stirred at room temperature for 3 h. The pH of the reaction was adjusted to ˜8 with NaHCO₃, diluted with water (100 mL) and extracted with ethyl acetate (150 mL×2). The combined organic layer was separated, washed with brine (100 mL), dried over Na₂SO₄ and evaporated under vacuum. The residue was purified by silica gel column (100-200 mesh) using methanol/chloroform (1:9) as eluent to get methyl 2-(6-(2-hydroxyethylamino)pyridin-3-yl)propanoate (300 mg, 21%) as a pale yellow oil.

Step 6: To a stirred solution of 2-(6-(2-hydroxyethylamino)pyridin-3-yl)propanoate (324 mg, 1.45 mmol, 1.0 equiv.) in tetrahydrofuran/H₂O (9 mL/9 mL) was added LiOH.H₂O (100 mg, 4.33 mmol, 3.0 equiv.) at 60° C. and stirred for 16 h. tetrahydrofuran was distilled off, the reaction mixture was extracted with Et₂O (10 mL), acidified (pH 3-4) with 1N HCl, and the solvent was evaporated. The residue was suspended in methanol (10 mL) and sonicated for 15 min. The mixture was filtrated, dried over anhydrous Mg₂SO₄ and evaporated under vacuum to get 2-(6-(2-hydroxyethylamino)pyridin-3-yl)propanoic acid (662 mg), which was used without further purification.

Step 7: To a stirred solution of 2-(6-(2-hydroxyethylamino)pyridin-3-yl)propanoic acid (59 mg, 0.29 mmol, 1.0 equiv.) in tetrahydrofuran/DMF (2 mL/0.1 mL) was added Hünig's base (0.193 mL, 1.14 mmol. 4 equiv.), 1-hydroxybenzotriazole (39 mg, 0.29 mmol, 1 equiv) and TBTU (92 mg, 0.29 mmol, 1 equiv) was added (2-(4-methylpiperidin-1-yl)-6-(trifluoromethyl)pyridin-3-yl)methanamine (77 mg. 0.29 mmol, 1 equiv.) and the mixture was stirred at room temperature for 16 h. The solvent was evaporated, the residue was dissolved in 20 mL of ethyl acetate and extracted with 20 mL of water. The aqueous layer was extracted with 3×20 mL of ethyl acetate, the organic phases were dried over Mg₂SO₄, the solvent was evaporated and the residue was purified by column chromatography using a linear gradient (start: 100% ethyl acetate, end ethyl acetate/methanol 80/20, 15 column voluminous) as eluent to get 2-(6-(2-hydroxyethylamino)pyridin-3-yl)-N-((2-(4-methylpiperidin-1-yl)-6-(trifluoromethyl)pyridin-3-yl)methyl)propanamide (example compound 85, 30 mg; 23%) as a yellow oil.

### Synthesis of Example 86

### 1-(6-(2-Hydroxyethylamino)pyridin-3-yl)-3-((2-(4-methylpiperidin-1-yl)-6-(trifluoromethyl)pyridin-3-yl)methyl)urea

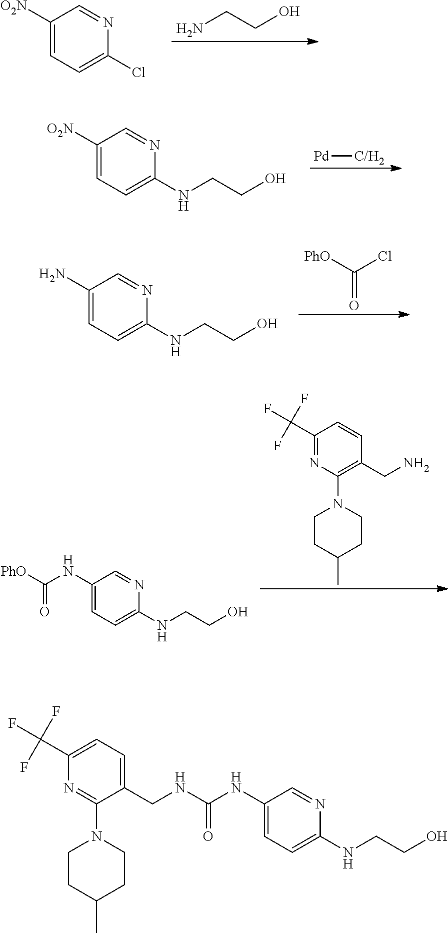

Step 1: 2-chloro-5-nitropyridine (4.0 g) was stirred with 2-aminoethanol (20 mL) at room temperature for 1 h. The reaction mixture was diluted with water (30 mL) and extracted with ethyl acetate (50 mL×2), washed with brine (20 mL), dried over Na₂SO₄ and evaporated under vacuum. The residue was washed with n-pentane (25 mL) to get 2-(5-nitropyridin-2-ylamino)ethanol (4.16 g, 91%, yellow solid). TLC system: methanol/chloroform (1:19), R_{f}: 0.2.

Step 2: To a stirred solution of 2-(5-nitropyridin-2-ylamino)ethanol (4.0 g, 21.85 mmol, 1 equiv.) in tetrahydrofuran (50 mL) was added 10% Pd—C (600 mg) and stirred at room temperature for 16 h under H₂ gas balloon pressure. The reaction mixture was passed through celite, evaporated and the residue obtained was washed with diethylether (20 mL) to get 2-(5-aminopyridin-2-ylamino)ethanol (3.02 g, 90%). TLC system: methanol/chloroform (3:17), R_{f}: 0.5.

Step 3: To a stirred acetone (35 mL) solution of 2-(5-aminopyridin-2-ylamino)ethanol (3.0 g, 19.60 mmol, 1 eq) pyridine (4.7 mL, 58.82 mmol, 3 equiv.) was added followed by phenyl chloroformate (2.7 mL, 21.56 mmol, 1.1 equiv.) at 0° C. and stirred room temperature for 1 h. The solvent was evaporated and the residue obtained was dissolved in ethyl acetate (150 mL) and washed with water (50 mL), brine (50 mL) dried (Na₂SO₄), evaporated and the residue was purified (neutral alumina, methanol/chloroform (1:49) as eluents) to get phenyl 6-(2-hydroxyethylamino)pyridin-3-ylcarbamate (0.80 g, 19%, pink solid). TLC system: methanol/chloroform (1:9), R_{f}: 0.5.

Step 4: To a stirred solution of (2-(4-methylpiperidin-1-yl)-6-(trifluoromethyl)pyridin-3-yl)methanamine (100 mg, 0.368 mmol, 1.0 equiv.) in acetonitrile (9 mL) was added triethylamine (0.204 mL, 1.47 mmol, 4.0 equiv.) followed by phenyl 6-(2-hydroxyethylamino)pyridin-3-ylcarbamate (102 mg, 0.375 mmol, 1.02 equiv.) and stirred for 16 h at reflux. The reaction mixture was concentrated under vacuum and the residue purified (column chromatography, silica gel, ethyl acetate/methanol (20:1) as eluent) to get 1-(6-(2-hydroxyethylamino)pyridin-3-yl)-3-((2-(4-methylpiperidin-1-yl)-6-(trifluoromethyl)pyridin-3-yl)methyl)urea (example compound 86 mg; 17%).

Example compounds 130 and 131 were prepared analogously.

### Synthesis of Example 87

### 2-(6-(2-Methoxyethylamino)pyridin-3-yl)-N-((2-(4-methylpiperidin-1-yl)-6-(trifluoromethyl)pyridin-3-yl)methyl)propanamide

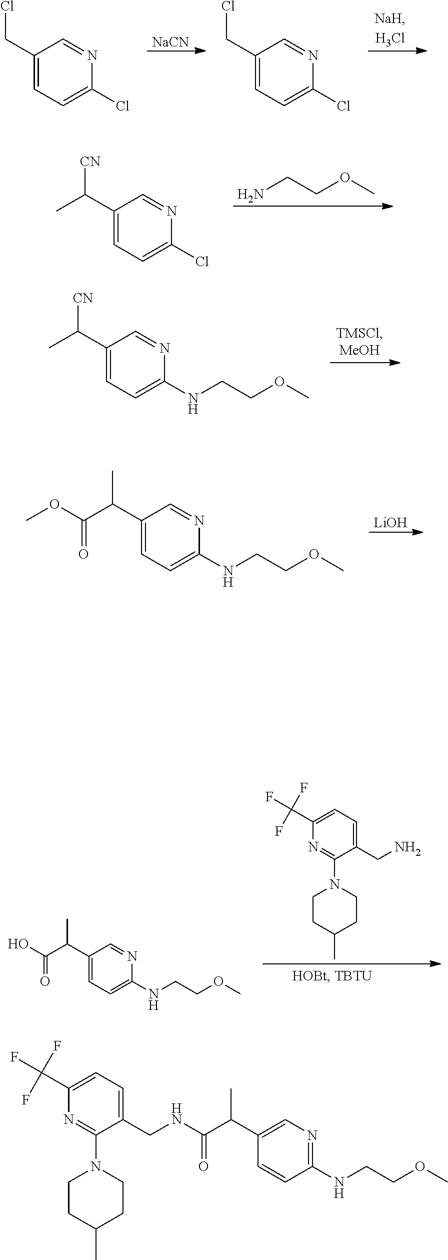

Step 1: To a stirred solution of 2-chloro-5-(chloromethyl)pyridine (1 g, 6.17 mmol, 1.0 equiv.) in ethanol (10 mL) was added the solution of NaCN (325 mg, 6.79 mmol, 1.1 eq) in H₂O (10 mL) dropwise at 0° C. and stirred for 3 h at 100° C. The reaction mixture was diluted with water (50 mL), extracted with ethyl acetate (70 mL×2) washed with brine (20 mL), dried over anhydrous Na₂SO₄ and evaporated under vacuum. The crude was purified by using silica gel chromatography (100-200 mesh) using ethyl acetate/petrol ether (3:7) to get 2-(6-chloropyridin-3-yl)acetonitrile (400 mg, 63%) as a yellow solid. TLC system: ethyl acetate/petrol ether (2:3), R_{f}: 0.30.

Step 2: To a stirred solution of 2-(6-chloropyridin-3-yl)acetonitrile (10 g, 65.7 mmol, 1.0 equiv.) in tetrahydrofuran (100 mL) cooled to 0° C. was added NaH (1.578 g, 65.7 mmol, 1.0 equiv.) as portion wise stirred for 10 min. CH₃I (4.02 mL, 65.7 mmol, 1.0 equiv.) was added at 0° C. The reaction mixture was diluted with water (150 mL), extracted with ethyl acetate (100 mL×2) and brine (100 mL) and dried over sodium sulfate and evaporated under vacuum. The crude was purified by silica gel chromatography (100-200 mesh) using ethyl acetate/petrol ether (1:4) to get 2-(6-chloropyridin-3-yl)propanenitrile (5 g, 46%) as solid. TLC system: ethyl acetate/petrol ether (3:7), R_{f}: 0.4.

Step 3: To a stirred solution of 2-(6-chloropyridin-3-yl)propanenitrile (2 g, 12.04 mmol, 1.0 equiv.) in DMSO (15 mL) was added TEA (3.34 mL, 24.09 mmol, 2.0 equiv.) and N(2-methoxy ethyl)methyl amine (1.8 g, 24.09 mmol, 2.0 equiv.) and heated to 100° C. for 16 h. The reaction mixture was diluted with water (50 mL), extracted with ethyl acetate (60 mL×2). The organic layer was washed with brine (50 mL), dried over sodium sulfate and evaporated under vacuum. The residue obtained was purified by neutral alumina using ethyl acetate/petrol ether (3:7) as eluent to get 2-(6-(2-methoxyethylamino)pyridin-3-yl)propanenitrile (500 mg, 40%) as white solid. TLC system: ethyl acetate/petrol ether (4:1), R_{f}: 0.2.

Step 4: To a stirred solution of TMSCl (4.6 mL, 20.4 mmol, 3.0 equiv.) in methanol (8 mL) was added 2-(6-(2-methoxyethylamino)pyridin-3-yl)propanenitrile (1.4 g, 6.8 mmol, 1.0 eq) and heated to 60° C. for 5 h. The reaction mixture was diluted with water (50 mL) and P^{H}≈9 adjusted with NaHCO₃ (10 mL) extracted with ethyl acetate (100 mL×2). The organic layer was separated and washed with brine (50 mL), dried over Na₂SO₄ and evaporated under vacuum. The residue was purified by silica gel column (100-200 mesh) using ethyl acetate/petrol ether (1:1) as eluent to get methyl 2-(6-(2-methoxyethylamino)pyridin-3-yl)propanoate (1.2 g, 74%) as a pale yellow liquid. TLC system: ethyl acetate/petrol ether (3:2), R_{f}: 0.3.

Step 5: To a stirred solution of methyl 2-(6-(2-methoxyethylamino)pyridin-3-yl)propanoate (83 mg, 0.35 mmol, 1.0 equiv.) in tetrahydrofuran/H₂O (2 mL+2 mL) was added LiOH.H₂O (24 mg, 1.0 mmol, 3.0 equiv.) at 60° C. and stirred for 16 h. The reaction mixture was diluted with water (1.5 mL), acidified (pH 3-4) with 1N HCl, and the solvent was evaporated. The residue was suspended in ethyl acetate/methanol (6 mL+6 mL) and sonicated for 15 min. The mixture was filtrated, dried over anhydrous Mg₂SO₄ and evaporated under vacuum to get 2-(6-(2-methoxyethylamino)pyridin-3-yl)propanoic acid (240 mg), which was used without further purification.

Step 6: To a stirred solution of 2-(6-(2-methoxyethylamino)pyridin-3-yl)propanoic acid (62 mg, 0.28 mmol, 1.0 equiv.) in tetrahydrofuran/DMF (2 mL/0.1 mL) was added Hünig's base (0.187 mL, 1.10 mmol. 4 equiv.), 1-hydroxybenzotriazole (37 mg, 0.28 mmol, 1 equiv) and TBTU (89 mg, 0.28 mmol, 1 equiv) was added (2-(4-methylpiperidin-1-yl)-6-(trifluoromethyl)pyridin-3-yl)methanamine (75 mg, 0.28 mmol, 1 equiv.) and the mixture was stirred at room temperature for 3 days. The solvent was evaporated, the residue was dissolved in 20 mL of ethyl acetate and extracted with 20 mL of water. The aqueous layer was extracted with 3×20 mL of ethyl acetate, the organic phases were dried over Mg₂SO₄, the solvent was evaporated and the residue was purified by column chromatography using ethyl acetate/cyclohexane (3:2) as eluent to get 2-(6-(2-methoxyethylamino)pyridin-3-yl)-N-((2-(4-methylpiperidin-1-yl)-6-(trifluoromethyl)pyridin-3-yl)methyl)propanamide (example compound 87, 42 mg; 32%) as a colorless oil.

### Synthesis of Example 88

### 1-(6-(2-Methoxyethylamino)pyridin-3-yl)-3-((2-(4-methylpiperidin-1-yl)-6-(trifluoromethyl)pyridin-3-yl)methyl)urea

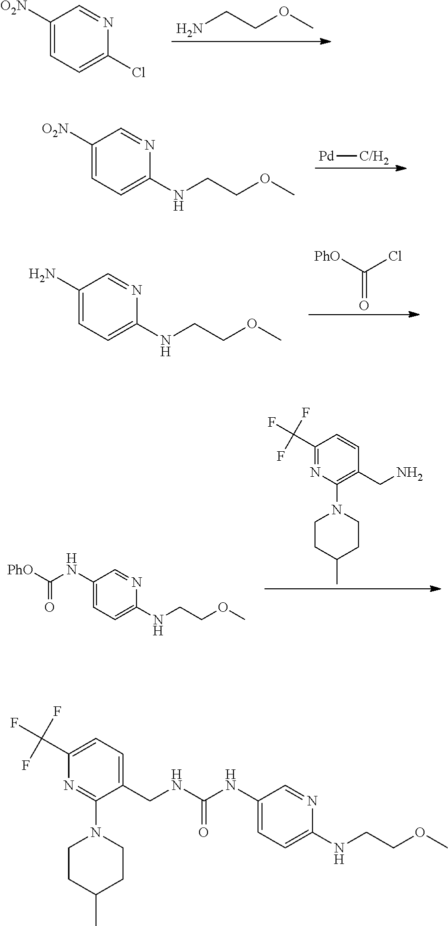

Step 1: 2-chloro-5-nitropyridine (4.0 g) was stirred with 2-methoxyethylamine (20 mL) at room temperature for 1 h. The reaction mixture was diluted with water (30 mL) and extracted with ethyl acetate (50 mL×2), washed with brine (20 mL), dried over Na₂SO₄ and evaporated under vacuum. The residue was washed with n-pentane (25 mL) to get N-(2-methoxyethyl)-5-nitropyridin-2-amine (4.8 g, 87%, yellow solid).

Step 2: To a stirred solution of N-(2-methoxyethyl)-5-nitropyridin-2-amine (4.8 g, 22.84 mmol, 1 equiv.) in ethyl acetate (50 mL) was added 10% Pd—C (550 mg) then allowed to stir room temperature for 16 h H₂ gas balloon. The reaction mixture was passed through celite and evaporated under reduced pressure. The residue thus obtained was washed with pentane (20 mL) to get N2-(2-methoxyethyl)pyridine-2,5-diamine (3.51 g, 87%).

Step 3: To a stirred solution of N2-(2-methoxyethyl)pyridine-2,5-diamine (3.8 g, 22.75 mmol, 1 eq) in acetone (35 mL) was added pyridine (5.5 mL, 68.25 mmol, 3 equiv.) followed by phenyl chloroformate (3.2 mL, 25.025 mmol, 1.1 equiv.) at 0° C. and stirred room temperature for 1 h. The solvent was evaporated and residue obtained was dissolved in ethyl acetate (150 mL) and washed with water (50 mL), brine (50 mL) dried (Na₂SO₄), evaporated and residue was purified (silica gel; 100-200 mesh; using methanol/chloroform (1:99) as eluent) to get phenyl 6-(2-methoxyethylamino)pyridin-3-ylcarbamate (3.1 g, 47%, white solid).

Step 4: To a stirred solution of (2-(4-methylpiperidin-1-yl)-6-(trifluoromethyl)pyridin-3-yl)methanamine (96 mg, 0.352 mmol, 1.0 equiv.) in acetonitrile (8 mL) was added triethylamine (0.195 mL, 1.41 mmol, 4.0 equiv.) followed by phenyl-6-(2-methoxyethylamino)pyridin-3-ylcarbamate (102 mg, 0.359 mmol, 1.02 equiv.) and stirred for 16 h at reflux. The reaction mixture was concentrated under vacuum and the residue purified (column chromatography, silica gel, ethyl acetate/methanol (10:1) as eluent) to get 1-(6-(2-hydroxyethylamino)pyridin-3-yl)-3-((2-(4-methylpiperidin-1-yl)-6-(trifluoromethyl)pyridin-3-yl)methyl)urea (example compound 89 mg; 44%).

### Synthesis of Example 89

### 2-(6-((2-Hydroxyethyl)(methyl)amino)pyridin-3-yl)-N-((2-(4-methylpiperidin-1-yl)-6-(trifluoromethyl)pyridin-3-yl)methyl)propanamide

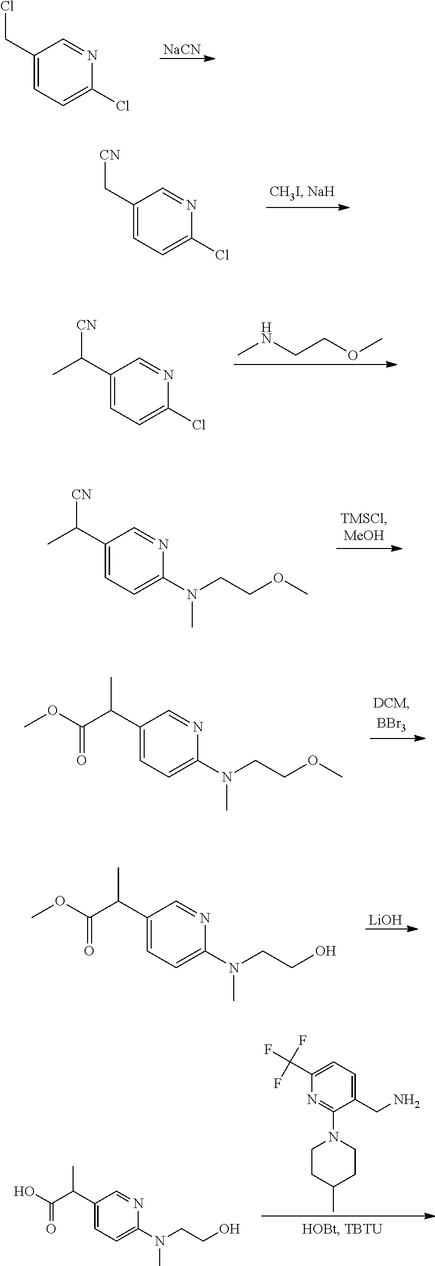

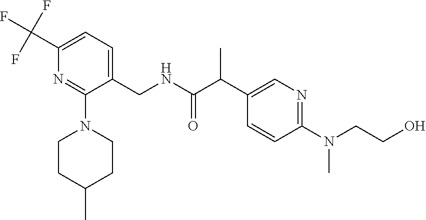 [SMILES: CC1CCN(c2nc(C(F)(F)F)ccc2CNC(=O)C(C)c2ccc(N(C)CCO)nc2)CC1]

Step 1: To a stirred solution of 2-chloro-5-(chloromethyl)pyridine (1 g, 6.17 mmol, 1.0 equiv.) in ethanol (10 mL) was added the solution of NaCN (325 mg, 6.79 mmol, 1.1 eq) in H₂O (10 mL) dropwise at 0° C. and then stirred for 3 h at 100° C. The reaction mixture was diluted with water (50 mL) and extracted with ethyl acetate (70 mL×2). The organic layer was dried over sodium sulfate and evaporated under vacuum. The crude was purified by silica gel chromatography (100-200 mesh) using ethyl acetate/petrol ether (3:7) to get 2-(6-chloropyridin-3-yl)acetonitrile (400 mg, 63%) as a yellow solid. TLC system: ethyl acetate/petrol ether (2:3), R_{f}: 0.30.

Step 2: To a stirred solution of 2-(6-chloropyridin-3-yl)acetonitrile (10 g, 65.7 mmol, 1.0 equiv.) in tetrahydrofuran (100 mL), was added NaH (1.578 g, 65.7 mmol, 1.0 equiv.) as portion wise at 0° C. and stirred for 10 min, then CH₃I (4.02 mL, 65.7 mmol, 1.0 equiv.) at 0° C. and stirred for 5 h at room temperature. The reaction mixture was diluted with water (150 mL), extracted with ethyl acetate (100 mL×2) and brine (100 mL) and dried over sodium sulfate and evaporated under vacuum. The crude was purified by silica gel chromatography (100-200 mesh) using ethyl acetate/petrol ether (1:4) as eluent to get 2-(6-chloropyridin-3-yl)propanenitrile (5 g, 46%) as a solid. TLC system: ethyl acetate/petrol ether (3:7), R_{f}: 0.4.

Step 3: To a stirred solution 2-(6-chloropyridin-3-yl)propanenitrile (1 g, 6.02 mmol, 1.0 equiv.) in DMSO (7 mL) was added TEA (1.67 mL, 12.04 mmol, 2.0 equiv.) followed by N (2-methoxy ethyl)methyl amine (1.07 g, 12.04 mmol, 2.0 equiv.). The mixture was heated to 100° C. for 16 h and diluted with water (50 mL), extracted with ethyl acetate (60 mL×2). The organic layer was washed with brine (50 mL), dried over sodium sulfate and evaporated under vacuum. The residue obtained was purified by neutral alumina using ethyl acetate/petrol ether (1:4) as eluent to get 2-(6-((2-methoxyethyl)(methyl)amino)pyridin-3-yl)propanenitrile (600 mg, 45%) as white solid. TLC system: ethyl acetate/petrol ether (2:3), R_{f}: 0.3.

Step 4: To a stirred solution of TMSCl (3.0 mL, 13.69 mmol, 3.0 equiv.) and methanol (0.73 mL, 22.8 mmol, 5.0 equiv.) was added 2-(6-((2-methoxyethyl)(methyl)amino)pyridin-3-yl)propanenitrile (1 g, 22.8 mmol, 5.0 equiv.) and heated to 60° C. for 5 h. The reaction mixture was diluted with water (50 mL) and pH≈9 adjusted with NaHCO₃ (10 mL) extracted with ethyl acetate (60 mL×2). The organic layer was separated and washed with brine (50 mL), dried over Na₂SO₄ and evaporated under vacuum. The residue was purified by silica gel column chromatography (100-200 mesh) using ethyl acetate/petrol ether (2:3) as eluent to get methyl 2-(6-((2-methoxyethyl)(methyl)amino)pyridin-3-yl)propanoate (700 mg, 61%) as a pale yellow oil. TLC system: ethyl acetate/petrol ether (2:3), R_{f}: 0.3.

Step 5: To a stirred solution of methyl 2-(6-((2-methoxyethyl)(methyl)amino)pyridin-3-yl)propanoate (2.0 g, 7.93 mmol, 1.0 equiv.) in dichloromethane (20 mL) was added compound BBr₃ (1.61 mL, 16.8 mmol, 2.0 equiv.) at −78° C. and stirred at room temperature for 3 h and pH≈8 was adjusted with NaHCO₃, diluted with water (100 mL). The aqueous layer was extracted with ethyl acetate (150 mL×2) and the combined organic layer was separated and washed with brine (100 mL), dried over Na₂SO₄ and evaporated under vacuum. The residue was purified by silica gel column (100-200 mesh) using ethyl acetate/petrol ether (7:3) as eluent to get methyl 2-(6-((2-hydroxyethyl)(methyl)amino)pyridin-3-yl)propanoate (800 mg, 42%) as a pale yellow oil. TLC system: ethyl acetate/petrol ether (4:1), R_{f}: 0.15.

Step 6: To a stirred solution of methyl 2-(6-((2-hydroxyethyl)(methyl)amino)pyridin-3-yl)propanoate (83 mg, 0.35 mmol, 1.0 equiv.) in tetrahydrofuran/H₂O (2 mL+2 mL) was added LiOH.H₂O (24 mg, 1.0 mmol, 3.0 equiv.) at 60° C. and stirred for 16 h. The reaction mixture was diluted with water (1.5 mL), acidified (pH 3-4) with 1N HCl, and the solvent was evaporated. The residue was suspended in ethyl acetate/methanol (6 mL+6 mL) and sonicated for 15 min. The mixture was filtrated, dried over anhydrous Mg₂SO₄ and evaporated under vacuum to get 2-(6-((2-hydroxyethyl)(methyl)amino)pyridin-3-yl)propanoic acid (138 mg), which was used without further purification.

Step 7: To a stirred solution of 2-(6-((2-hydroxyethyl)(methyl)amino)pyridin-3-yl)propanoic acid (61 mg, 0.28 mmol, 1.0 equiv.) in tetrahydrofuran/DMF (2 mL/0.1 mL) was added Hünig's base (0.186 mL, 1.10 mmol. 4 equiv.), 1-hydroxybenzotriazole (37 mg, 0.28 mmol, 1 equiv.) and TBTU (89 mg, 0.28 mmol, 1 equiv.) was added (2-(4-methylpiperidin-1-yl)-6-(trifluoromethyl)pyridin-3-yl)methanamine (74 mg. 0.28 mmol, 1 equiv.) and the mixture was stirred at room temperature for 16 h. The solvent was evaporated, the residue was dissolved in 20 mL of ethyl acetate and extracted with 20 mL of water. The aqueous layer was extracted with 3×20 mL of ethyl acetate, the organic phases were dried over Mg₂SO₄, the solvent was evaporated and the residue was purified by column chromatography using a linear gradient (start: 100% ethyl acetate, end ethyl acetate/ethanol 95/5, 10 column voluminous) as eluent to get 2-(6-((2-hydroxyethyl)(methyl)amino)pyridin-3-yl)-N-((2-(4-methylpiperidin-1-yl)-6-(trifluoromethyl)pyridin-3-yl)methyl)propanamide (example compound 89, 49 mg; 37%) as a yellow oil.

### Synthesis of Example 90

### 1-(6-((2-Hydroxyethyl)(methyl)amino)pyridin-3-yl)-3-((2-(4-methylpiperidin-1-yl)-6-(trifluoromethyl)pyridin-3-yl)methyl)urea

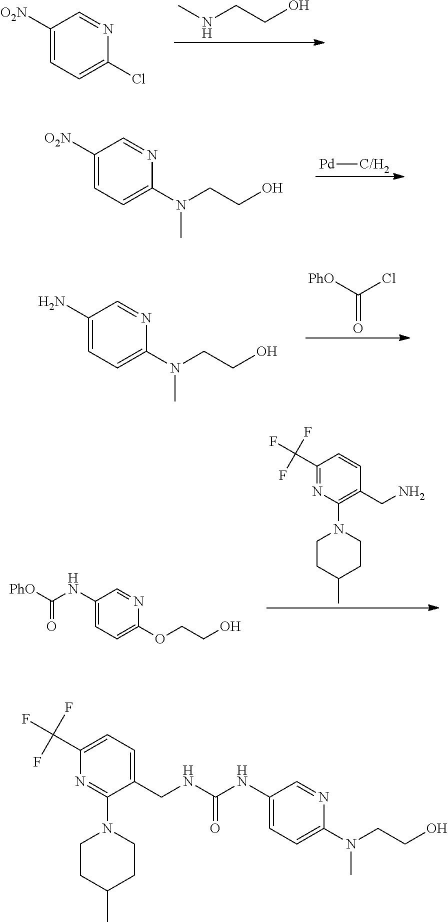

Step 1: 2-chloro-5-nitropyridine (4.0 g) was stirred with 2-methylaminoethanol (20 mL) at room temperature for 1 h. The reaction mixture was diluted with water (30 mL) and extracted with ethyl acetate (50 mL×2), washed with brine (20 mL), dried over Na₂SO₄ and evaporated under vacuum. The residue was washed with n-pentane (25 mL) to get 2-(methyl(5-nitropyridin-2-yl)amino)ethanol (4.5 g, 91%, yellow solid). TLC system: methanol/chloroform (1:19), R_{f}: 0.4.

Step 2: To a stirred ethyl acetate (50 mL) solution of 2-(methyl(5-nitropyridin-2-yl)amino)ethanol (4.8 g, 24.36 mmol, 1 equiv.) 10% Pd—C (550 mg) was added and stirred at room temperature for 16 h H₂ gas balloon. The reaction mixture was passed through celite and evaporated under reduced pressure. The obtained residue was washed with diethylether (20 mL) to get 2-((5-aminopyridin-2-yl)(methyl)amino)ethanol (3.3 g, 8%). TLC system: methanol/chloroform (1:9), R_{f}: 0.4.

Step 3: To a stirred solution of 2-((5-aminopyridin-2-yl)(methyl)amino)ethanol (3.3 g, 16.75 mmol, leg) in acetone (40 mL) pyridine (4.0 mL, 50.25 mmol, 3 equiv.) followed by phenyl chloroformate (2.3 mL, 18.425 mmol, 1.1 equiv.) were added at 0° C. and stirred room temperature for 1 h. The solvent was evaporated, the residue was dissolved in ethyl acetate (150 mL) and washed with water (50 mL), brine (50 mL) dried (Na₂SO₄), evaporated and residue was purified (silica gel; 100-200; methanol/chloroform (1:19) as eluent) to get phenyl 6-((2-hydroxyethyl)(methyl)amino)pyridin-3-ylcarbamate (1.2 g, 25%, green solid). TLC system: methanol/chloroform (1:19), R_{f}: 0.4.

Step 4: To a stirred solution of (2-(4-methylpiperidin-1-yl)-6-(trifluoromethyl)pyridin-3-yl)methanamine (95 mg, 0.35 mmol, 1.0 equiv.) in acetonitrile (8 mL) was added triethylamine (0.193 mL, 1.41 mmol, 4.0 equiv.) followed by phenyl 6-((2-hydroxyethyl)(methyl)amino)pyridin-3-ylcarbamate (102 mg, 0.355 mmol, 1.02 equiv.) and stirred for 16 h at reflux. The reaction mixture was concentrated under vacuum and the residue purified (column chromatography, silica gel, ethyl acetate/cyclohexane (9:1) as eluent) to get 1-(6-((2-hydroxyethyl)(methyl)amino)pyridin-3-yl)-3-((2-(4-methylpiperidin-1-yl)-6-(trifluoromethyl)pyridin-3-yl)methyl)urea (example compound 90, 59 mg; 36%).

### Synthesis of Example 91

### 1-(6-((2-Methoxyethyl)(methyl)amino)pyridin-3-yl)-3-((2-(4-methylpiperidin-1-yl)-6-(trifluoromethyl)pyridin-3-yl)methyl)urea

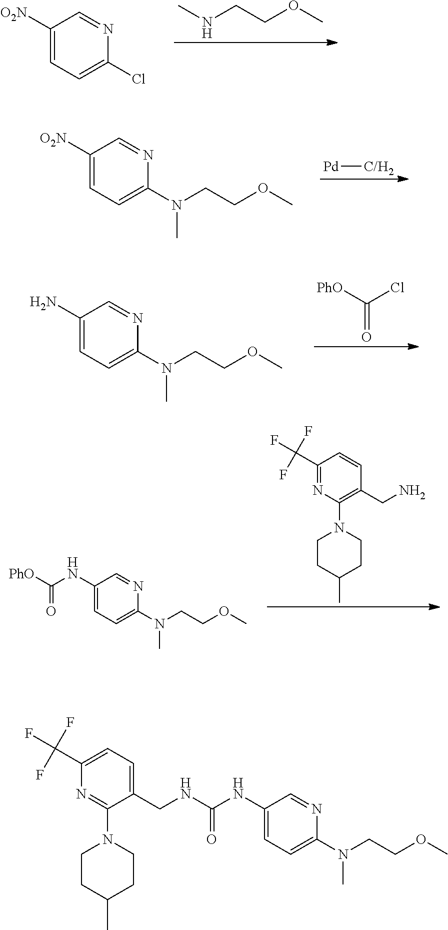

Step 1: 2-chloro-5-nitropyridine (3.0 g) was stirred with 2-methoxyethylmethylamine (10 mL) at room temperature for 1 h. The reaction mixture was diluted with water (50 mL), extracted with ethyl acetate (150 mL×2), washed with brine (50 mL), dried over Na₂SO₄ and concentrated to get N-(2-methoxyethyl)-N-methyl-5-nitropyridin-2-amine (3.3 g, 83%, yellow solid). TLC system: ethyl acetate/petrol ether (1:1), R_{f}: 0.40.

Step 2: To a stirred solution of N-(2-methoxyethyl)-N-methyl-5-nitropyridin-2-amine (3.3 g, 15.63 mmol, 1 equiv.) in ethyl acetate (35 mL) 10% Pd—C (450 mg) was added and stirred at room temperature for 16 h under H₂ gas balloon. The reaction mixture was then passed through celite and concentrated. The residue was washed with pentane (20 mL) to get N2-(2-methoxyethyl)-N2-methylpyridine-2,5-diamine (2.0 g, 73%). TLC system: methanol/chloroform (1:19), R_{f}: 0.6.

Step 3: To a stirred solution of N2-(2-methoxyethyl)-N2-methylpyridine-2,5-diamine (2.0 g, 11.04 mmol, 1 equiv.) in acetone (30 mL) pyridine (4.3 mL, 33.12 mmol, 3 equiv.) was added followed by phenyl chloroformate (2.46 mL, 12.144 mmol, 1.1 equiv.) at 0° C. and stirred room temperature for 1 h. The reaction mixture was and the residue was dissolved in ethyl acetate (150 mL), washed with water (50 mL), brine (50 mL), dried (Na₂SO₄), evaporated and the residue was purified (silica gel; 100-200 mesh; using ethyl acetate/petrol ether (2:3) as eluent) to get phenyl 6-((2-methoxyethyl)(methyl)amino)pyridin-3-ylcarbamate (2.56 g, 77%, white solid). TLC system: methanol/chloroform (1:49), R_{f}: 0.5.

Step 4: To a stirred solution of (2-(4-methylpiperidin-1-yl)-6-(trifluoromethyl)pyridin-3-yl)methanamine (130 mg, 0.476 mmol, 1.0 equiv.) in acetonitrile (9 mL) was added triethylamine (0.264 mL, 1.90 mmol, 4.0 equiv.) followed by phenyl 6-((2-methoxyethyl)(methyl)amino)pyridin-3-ylcarbamate (146 mg, 0.486 mmol, 1.02 equiv.) and stirred for 16 h at reflux. The reaction mixture was concentrated under vacuum and the residue purified (column chromatography, silica gel, ethyl acetate/cyclohexane (4:1) as eluent) to get 1-(6-((2-methoxyethyl)(methyl)amino)pyridin-3-yl)-3-((2-(4-methylpiperidin-1-yl)-6-(trifluoromethyl)pyridin-3-yl)methyl)urea (example compound 91, 89 mg; 39%).

### Synthesis of Example 96

### 2-(5-Fluoro-6-(methylsulfonamido)pyridin-3-yl)-N-((2-(4-methylpiperidin-1-yl)-6-(trifluoromethyl)pyridin-3-yl)methyl)propanamide

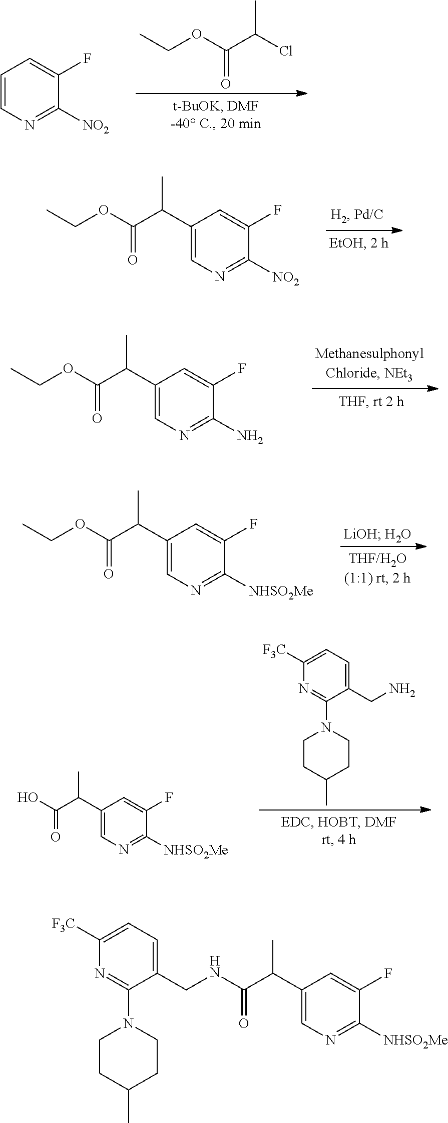

Step 1: In a round bottom flask potassium tertiary butoxide (0.473 g, 4221 mmol) was taken under nitrogen atmosphere, Anhydrous dimethylformamide (5 mL) was added and stirred at room temperature for 10 min. Then cooled to −20° C. and 3-fluoro-2-nitropyridine (200 mg, 1.407 mmol) was added followed by dropwise addition of 2-chloro-propionic acid ethyl ester (0.273 mL, 2.111 mol) and stirred for 20 min. Then diluted HCl was added and stirred at room temperature for 10 min. Extracted in ethyl acetate, washed with water, dried over MgSO₄, filtered and solvent was evaporated and finally purified by column chromatography to afford 2-(5-fluoro-6-nitro-pyridin-3-yl)-propionic acid ethyl ester (153 mg, 45%).

Step 2: In a round bottom flask 2-(5-fluoro-6-nitro-pyridin-3-yl)-propionic acid ethyl ester (100 mg) was taken followed by addition of ethanol and Pd/C (20 wt %) stirred at room temperature in presence of hydrogen for 2 h. Then celite filtration and solvent was evaporated to afford 2-(6-amino-5-fluoro-pyridin-3-yl)-propionic acid ethyl ester (69 mg, 79%).

Step 3: In a round bottom flask 2-(6-amino-5-fluoro-pyridin-3-yl)-propionic acid ethyl ester (1.525 g, 7.185 mmol) was taken under nitrogen atmosphere, anhydrous tetrahydrofuran (14 mL) was added and stirred. Then cooled to 0° C. and triethylamine (2.181 mL, 21.555 mmol) was added followed by addition methanesulphonylchloride (0.837 mL, 10.778 mmol) and stirred at room temperature for 2 h. Reaction mixture was extracted in ethyl acetate, washed with water, dried over MgSO₄, filtered and solvent was evaporated and finally purified by column chromatography to afford 2-(5-fluoro-6-methanesulfonylamino-pyridin-3-yl)-propionic acid ethyl ester (1.39 g, 67%).

Step 4: In a round bottom flask 2-(5-Fluoro-6-methanesulfonylamino-pyridin-3-yl)-propionic acid (110 mg, 0.378 mmol) ethyl ester was taken, then tetrahydrofuran (5 mL) was added and cooled to 0° C. and lithiumhydroxide monohydrate (0.039 g, 0.947 mmol) solution in water (5 mL) was added dropwise and stirred at room temperature for 2 h. Then reaction mixture was extracted in ethyl acetate, washed with water and aqueous layer was acidified by using diluted HCl and extracted again in ethyl acetate and washed with water, dried over MgSO₄, filtered and solvent was evaporated to afford 2-(5-fluoro-6-(methylsulfonamido)pyridin-3-yl)propanoic acid (59 mg, 60%).

Step 5: In a round bottom flask 2-(5-fluoro-6-(methylsulfonamido)pyridin-3-yl)propanoic acid (100 mg, 0.365 mmol) was taken under nitrogen atmosphere dimethylformamide (5 mL) was added. Followed by addition of 1-ethyl-3-(3-dimethylaminopropyl) carbodiimide) (104 mg, 0.547 mmol) and 1-hydroxybenzotriazole (74 mg, 0.547 mmol) stirred for 1 h. (2-(4-Methylpiperidin-1-yl)-6-(trifluoromethyl)pyridin-3-yl)methanamine (96 mg, 0.365 mmol) was added and stirred at room temperature for 4 h. The reaction mixture was extracted in ethyl acetate, washed with water and dried over MgSO₄, filtered and solvent was evaporated and finally purified by column chromatography to afford 2-(5-fluoro-6-(methylsulfonamido)pyridin-3-yl)-N-((2-(4-methylpiperidin-1-yl)-6-(trifluoromethyl)pyridin-3-yl)methyl)propanamide as a white solid (144 mg, 73%).

¹H NMR (300 MHz, CDCl₃) δ 8.04 (s, 1H, Ar—H), 7.53 (dd, 2H, Ar—H, J=2.01 Hz), 7.24 (d, 1H, Ar—H, J=7.68 Hz), 6.43 (s, 1H, R—NH), 4.51 (m, 2H, Ar—CH₂), 3.56 (q, 1H, J=6.6 Hz, Ar—CH), 3.47 (s, 1H, Ar-MS), 3.33 (t, 2H, J=11.34 Hz, Piperidine-H), 1.73 (br.s, 2H, Piperidine-H), 1.54 (d, 3H, J=7.14 Hz, ArCH—CH₃), 1.26 (m, 2H, Piperidine-H), 1.00 (d, 3H, J=6.6 Hz, Piperidine-CH₃).

### Synthesis of Example 97

### 2-(5-Methoxy-6-(methylsulfonamido)pyridin-3-yl)-N-((2-(4-methylpiperidin-1-yl)-6-(trifluoromethyl)pyridin-3-yl)methyl)propanamide

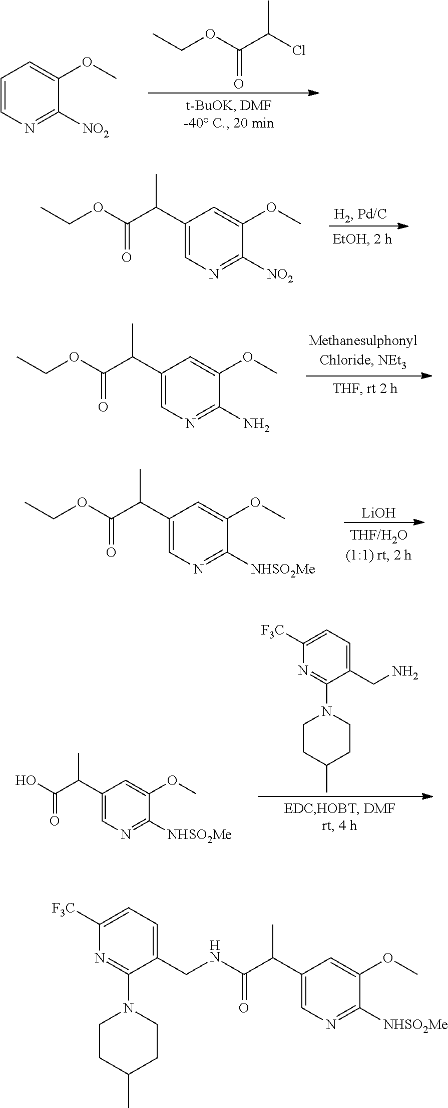

Step 1: In a round bottom flask potassium tertiary butoxide (146 mg, 1.297 mmol) was taken under nitrogen atmosphere, anhydrous dimethylformamide (3 mL) was added and stirred at room temperature for 10 min. Then cooled to −40° C. and 2-nitro-3-methoxypyridine(100 mg, 0.648 mmol) was added followed by dropwise addition of 2-chloro-propionic acid ethyl ester (0.0908 mL, 0.712 mmol) and stirred for 20 min. Then dilute HCl was added and stirred at room temperature for 10 min. Extracted in ethyl acetate, washed with water, dried over MgSO₄, filtered and solvent was evaporated and finally purified by column chromatography to afford 2-(5-methoxy-6-nitro-pyridin-3-yl)-propionic acid ethyl ester (82 mg, 50%).

Step 2: In a round bottom flask 2-(5-methoxy-6-nitro-pyridin-3-yl)-propionic acid ethyl (100 mg) ester was taken followed by addition of ethanol and Pd/C (20 wt %) then stirred at room temperature in presence of hydrogen for 2 h. Then celite filtration and solvent was evaporated to afford 2-(6-Amino-5-methoxy-pyridin-3-yl)-propionic acid ethyl ester (68 mg, 78%).

Step 3: In a round bottom flask 2-(6-amino-5-methoxy-pyridin-3-yl)-propionic acid ethyl ester (200 mg, 0.891 mmol) was taken under nitrogen atmosphere, anhydrous tetrahydrofuran was added and stirred Then cooled to 0° C. and triethylamine (0.137 mL, 0.981 mmol) was added. Followed by addition of methanesulphonylchloride (0.076 mL, 0.981 mmol) and stirred at room temperature for 2 h. Reaction mixture was extracted in ethyl acetate, washed with water, dried over MgSO₄, filtered and solvent was evaporated and finally purified by column chromatography to afford 2-(6-methanesulfonylamino-5-methoxy-pyridin-3-yl)-propionic acid ethyl ester (180 mg, 67%).

Step 4: In a round bottom flask 2-(5-methoxy-6-methanesulfonylamino-pyridin-3-yl)-propionic acid ethyl ester (1.6 g, 5.291 mmol) was taken, then tetrahydrofuran was added and cooled to 0° C. Lithiumhydroxide monohydrate (556 mg, 13.229 mmol) solution in water (10 mL) was added dropwise and stirred at room temperature for 2 h. Then reaction mixture was extracted in ethyl acetate, washed with water and aqueous layer was acidified by using diluted HCl and extracted in ethylacetate washed with water, dried over MgSO₄, filtered and solvent was evaporated to afford 2-(5-methoxy-6-(methylsulfonamido)pyridin-3-yl)propanoic acid (870 mg, 60%).

Step 5: In a round bottom flask 2-(5-methoxy-6-(methylsulfonamido)pyridin-3-yl)propanoic acid (77 mg, 0.282 mmol) was taken under nitrogen atmosphere dimethylformamide (5 mL) was added, Followed by addition of 1-ethyl-3-(3-dimethylaminopropyl) carbodiimide) (74 mg, 0.384 mmol) and 1-hydroxybenzotriazole (52 mg, 0.384 mmol) stirred for 1 h. (2-(4-Methylpiperidin-1-yl)-6-(trifluoromethyl)pyridin-3-yl)methanamine (70 mg, 0.256 mmol) was added and stirred at room temperature for 4 h. The reaction mixture was extracted in ethyl acetate, washed with water, dried over MgSO₄, filtered and solvent was evaporated and finally purified by column chromatography to afford 2-(5-methoxy-6-(methylsulfonamido)pyridin-3-yl)-N-((2-(4-methylpiperidin-1-yl)-6-(trifluoromethyl)pyridin-3-yl)methyl)propanamide as a white solid (115 mg, 78%).

¹H NMR (300 MHz, CDCl₃) δ 7.82 (s, 1H, Ar—H), 7.64 (d, 1H, J=7.53 Hz, Ar—H), 7.36 (s, 1H, Ar—H), 7.26 (d, 1H, J=3.6 Hz, Ar—H), 7.10 (s, 1H, Ar—H), 6.34 (s, 1H, R—NH), 4.50 (m, 2H, Ar—CH₂), 3.84 (s, 3H, Ar—OCH₃), 3.57 (m, 1H, Ar—CH), 3.34 (s, 3H, Ar-MS) 3.36 (t, 2H, J=14.82 Hz, Piperidine-H), 2.82 (t, 2H, J=12.63 Hz, Piperidine-H), 1.74 (br.s, 2H, Piperidine-H), 1.30 (d, 3H, J=8.43 Hz, ArCH—CH₃), 1.18 (m, 2H, piperidine-Hs), 1.01 (d, 3H, J=6.6 Hz, Piperidine-CH₃).

### Synthesis of Example 98

### N-(5-(1-((2-(4-Methylpiperidin-1-yl)-6-(trifluormethyl)pyridin-3-yl)methylamino)-1-oxopropan-2-yl)pyridin-2-yl)benzamid

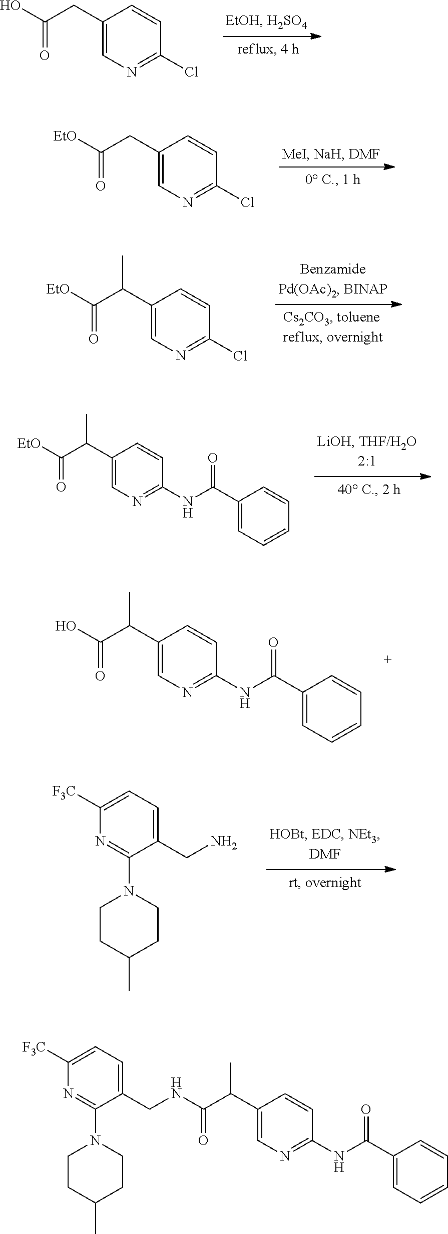

Step 1-2: as described for example 74.

Step 3: The round bottom flask was charged with Pd(OAc)₂ (78 mg, 0.35 mmol), BINAP (218 mg, 0.35 mmol) and toluene. The mixture was stirred under nitrogen flow for 15 min and then was added ethyl 2-(6-chloropyridin-3-yl)propanoate (370 mg, 1.73 mmol), benzamide (189 mg, 1.56 mmol) and Cs₂CO₃ (2258 mg, 6.93 mmol). The reaction mixture was refluxed overnight and then cooled to room temperature. The mixture was filtered through a plug of celite and concentrated. The residue was diluted with ethyl acetate and washed with 10% HCl solution. The organic layer was dried over MgSO₄ and concentrated under reduced pressure to afford crude which was purified by column chromatography to afford the pure ethyl 2-(6-benzamidopyridin-3-yl)propanoate (295 mg, 63%).

Step 4: To a solution ethyl 2-(6-benzamidopyridin-3-yl)propanoate (295 mg, 0.99 mmol) in tetrahydrofuran and water was added lithium hydroxide monohydrate (62 mg, 1.48 mmol). The reaction mixture was stirred for 2 h at 40° C. and then acidified with 10% HCl solution. The mixture was extracted with ethyl acetate. The organic layer dried over MgSO₄ and concentrated under reduced pressure to afford desired 2-(6-benzamidopyridin-3-yl)propanoic acid (250 mg, 94%).

Step 5: To a solution of 2-(6-benzamidopyridin-3-yl)propanoic acid (100 mg, 0.37 mmol) in dimethylformamide was added 1-hydroxybenzotriazole (75 mg, 0.55 mmol), 1-ethyl-3-(3-dimethylaminopropyl) carbodiimide) (106 mg, 0.55 mmol), triethylamine (0.1 mL, 0.74 mmol) and (2-(4-methylpiperidin-1-yl)-6-(trifluoromethyl)pyridin-3-yl)methanamine (106 mg, 0.39 mmol). The reaction mixture was stirred for 12 h at room temperature. The mixture was diluted with water and extracted with ethyl acetate. The organic layer was dried over MgSO₄ and concentrated under reduced pressure. The crude was purified by column chromatography to give pure N-(5-(1-((2-(4-methylpiperidin-1-yl)-6-(trifluoromethyl)pyridin-3-yl)methylamino)-1-oxopropan-2-yl)pyridin-2-yl)benzamide (100 mg, 51%).

¹H NMR (300 MHz, CDCl₃) δ 9.03 (br.s, NH), 8.35 (d, 1H, J=8.62 Hz, pyridine-H), 8.13 (d, 1H, J=2.02 Hz, pyridine-H), 7.90 (m, 2H, Ar—H), 7.75 (dd, 1H, J=8.63, 2.21 Hz, pyridine-H), 7.57 (m, 1H, Ar—H), 7.48 (m, 3H, Ar—H), 7.18 (d, 1H, J=7.71 Hz, Ar—H), 6.53 (t, NH, J=5.51 Hz), 4.46 (m, 2H, Ar—CH₂), 3.57 (quartet, 1H, J=7.14 Hz, amide-CH), 3.31 (m, 2H, piperidine-H), 2.80 (m, 2H, piperidine-H), 1.70 (m, 2H, piperidine-H), 1.55 (m, 4H, amide-CH₃, piperidine-H), 1.22 (m, 2H, piperidine-H), 0.95 (d, 3H, J=6.43 Hz, piperidine-CH₃).

Example compound 99 was prepared in a similar manner, exemplary compounds 100-103 can also be prepared in a similar manner.

### Synthesis of Example 104

### 1-(6-(Dimethylamino)-5-(trifluoromethyl)pyridin-3-yl)-3-((2-(4-methylpiperidin-1-yl)-6-(trifluoromethyl)pyridin-3-yl)methyl)urea

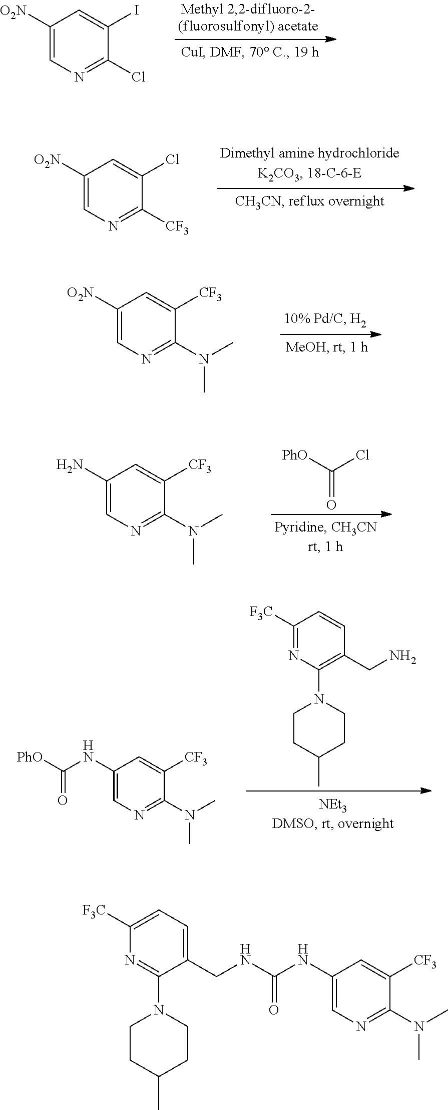

Step 1: In a 100 mL round bottom flask, a mixture of 2-chloro-3-iodo-5-nitropyridine (250 mg, 0.88 mmol), methyl 2,2-difluoro-2-(fluorosulfonyl)acetate (0.06 mL, 0.44 mmol) and Copper(I) iodide (25 mg, 0.13 mmol) in dimethylformamide was heated at 70° C. for 3 h under hydrogen atmosphere. Another 0.03 mL methyl 2,2-difluoro-2-(fluorosulfonyl)acetate was added and the mixture was heated at 70° C. for 16 h. The reaction mixture was cooled to room temperature, diluted with water and extracted with ethyl acetate. The organic layer was concentrated under reduced pressure to afford the crude which was purified by column chromatography to give 2-chloro-5-nitro-3-(trifluoromethyl)pyridine (41 mg, 21%).

Step 2: 2-Chloro-5-nitro-3-(trifluoromethyl)pyridine (41 mg, 0.18 mmol), dimethylamine hydrochloride (18 mg, 0.22 mmol), potassium carbonate (88 mg, 0.63 mmol) and 1,4,7,10,13,16-hexaoxacyclooctadecane (10 mg) was dissolved in acetonitrile. The reaction mixture was refluxed for 12 h. The reaction mixture was cooled to room temperature and then was concentrated under reduced pressure. Then the mixture was extracted with ethyl acetate and washed with water. The organic layer was concentrated under reduced pressure. The crude was purified by column chromatography to give N,N-dimethyl-5-nitro-3-(trifluoromethyl)pyridin-2-amine (36 mg, 84%).

Step 3: N,N-dimethyl-5-nitro-3-(trifluoromethyl)pyridin-2-amine (200 mg, 0.85 mmol) was dissolved in methanol. 10% Pd/C (40 mg) was added to it. The resulting mixture was stirred at room temperature under hydrogen atmosphere for 1 h. The mixture was filtered through celite bed and the filtrate was concentrated under reduced pressure to afford the N2,N2-dimethyl-3-(trifluoromethyl)pyridine-2,5-diamine (60 mg, 34%).

Step 4: N2,N2-dimethyl-3-(trifluoromethyl)pyridine-2,5-diamine (60 mg, 0.29 mmol) was dissolved in acetonitrile. The reaction mixture was added pyridine (0.03 mL, 0.35 mmol) and phenyl chloroformate (0.04 mL, 0.31 mmol), respectively and stirred at room temperature for 1 h. The reaction mixture was diluted with water and extracted with ethyl acetate. The organic layer was concentrated under reduced pressure. The crude was purified by column chromatography to give phenyl 6-(dimethylamino)-5-(trifluoromethyl)pyridin-3-ylcarbamate (47 mg, 49%).

Step 5: Phenyl 6-(dimethylamino)-5-(trifluoromethyl)pyridin-3-ylcarbamate (40 mg, 0.12 mmol) and (2-(4-methylpiperidin-1-yl)-6-(trifluoromethyl)pyridin-3-yl)methanamine (36 mg, 0.13 mmol) was dissolved in dimethyl sulfoxide. Then triethylamine (0.03 mL, 0.25 mmol) was added to it. The mixture was stirred at room temperature overnight. The reaction mixture was diluted with water and extracted with ethyl acetate. The organic layer was concentrated under reduced pressure. The crude was purified by column chromatography to give desired 1-(6-(dimethylamino)-5-(trifluoromethyl)pyridin-3-yl)-3-((2-(4-methylpiperidin-1-yl)-6-(trifluoromethyl)pyridin-3-yl)methyl)urea (45 mg, 73%).

¹H NMR (300 MHz, CD₃OD): δ 8.39 (d, 1H, J=2.73 Hz, Ar—H), 8.14 (d, 1H, J=2.76 Hz, Ar—H), 7.82 (d, 1H, J=7.5 Hz, Ar—H), 7.34 (d, 1H, J=7.5 Hz, Ar—H), 4.45 (s, 1H, Ar—CH₂), 3.47 (m, 2H, piperidine-CH₂), 2.91 (m, 8H, piperidine-CH₂ and Ar—N(CH₃)₂), 1.78 (m, 2H, piperidine-CH₂), 1.58 (m, 1H, piperidine-CH), 1.45 (m, 2H, piperidine-CH₂), 1.02 (d, 3H, J=6.42 Hz, piperidine-CH₃).

### Synthesis of Example 105

### 1-(6-(Azetidin-1-yl)pyridin-3-yl)-3-((2-(4-methylpiperidin-1-yl)-6-(trifluoromethyl)pyridin-3-yl)methyl)urea

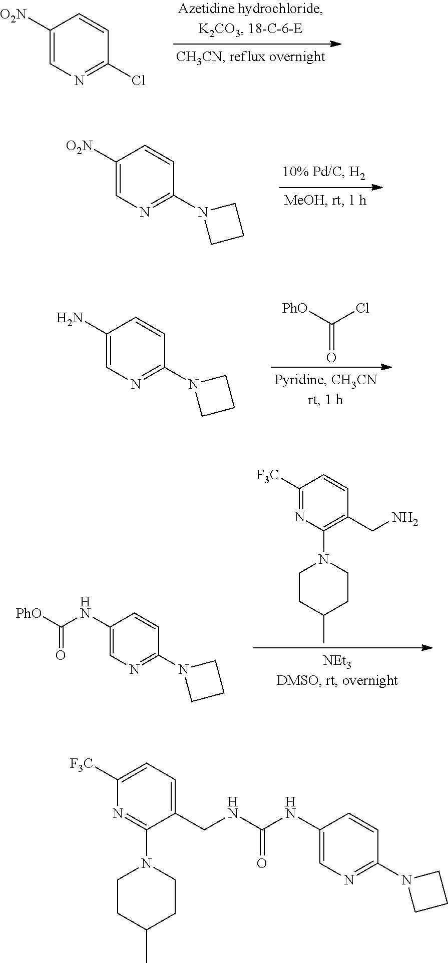

Step 1: 2-Chloro-5-nitropyridine (300 mg, 1.89 mmol), azetidine hydrochloride (212 mg, 2.27 mmol), potassium carbonate (915 mg, 6.62 mmol) and 1,4,7,10,13,16-hexaoxacyclooctadecane (60 mg) was dissolved in acetonitrile. The reaction mixture was refluxed overnight. The reaction mixture was cooled to room temperature and then was concentrated under reduced pressure. Then the mixture was extracted with ethyl acetate and washed with water. The organic layer was concentrated under reduced pressure. The crude was purified by column chromatography to give 2-(azetidin-1-yl)-5-nitropyridine (196 mg, 58%).

Step 2: 2-(Azetidin-1-yl)-5-nitropyridine (185 mg, 1.03 mmol) was dissolved in methanol. 10% Pd/C (37 mg) was added to it. The resulting mixture was stirred at room temperature under hydrogen atmosphere for 1 h. The mixture was filtered through celite bed and the filtrate was concentrated under reduced pressure to afford the 6-(azetidin-1-yl)pyridin-3-amine (154 mg, 99%).

Step 3: 6-(Azetidin-1-yl)pyridin-3-amine (154 mg, 1.03 mmol) was dissolved in acetonitrile.

To the reaction mixture was added pyridine (0.1 mL, 1.24 mmol) and phenyl chloroformate (0.14 mL, 1.08 mmol), respectively and stirred at room temperature for 1 h. The reaction mixture was diluted with water and extracted with ethyl acetate. The organic layer was concentrated under reduced pressure. The crude was purified by column chromatography to give phenyl 6-(azetidin-1-yl)pyridin-3-ylcarbamate (123 mg, 44%).

Step 4: Phenyl 6-(azetidin-1-yl)pyridin-3-ylcarbamate (70 mg, 0.26 mmol) and (2-(4-methylpiperidin-1-yl)-6-(trifluoromethyl)pyridin-3-yl)methanamine (75 mg, 0.27 mmol) was dissolved in dimethyl sulfoxide. Then triethylamine (0.07 mL, 0.52 mmol) was added to it. The mixture was stirred at room temperature overnight. The reaction mixture was diluted with water and extracted with ethyl acetate. The organic layer was concentrated under reduced pressure. The crude was purified by column chromatography to give desired compound I-(6-(azetidin-1-yl)pyridin-3-yl)-3-((2-(4-methylpiperidin-1-yl)-6-(trifluoromethyl)-pyridin-3-yl)methyl)urea (66 mg, 56%).

¹H NMR (300 MHz, CD₃OD): δ 7.99 (d, 1H, J=2.01 Hz, Ar—H), 7.81 (d, 1H, J=7.32 Hz, Ar—H), 7.61 (dd, 1H, J=8.79 Hz, 2.55 Hz, Ar—H), 7.34 (d, 1H, J=7.71 Hz, Ar—H), 6.39 (d, 1H, J=8.97 Hz, Ar—H), 4.43 (s, 1H, Ar—CH₂), 4.019 (m, 4H, azetidine-CH₂), 3.46 (m, 2H, piperidine-CH₂), 2.89 (m, 2H, piperidine-CH₂), 2.43 (m, 2H, azetidine-CH₂), 1.77 (m, 2H, piperidine-CH₂), 1.57 (m, 1H, piperidine-CH), 1.43 (m, 2H, piperidine-CH₂), 1.02 (d, 3H, J=6.39 Hz, piperidine-CH₃).

### Synthesis of Example 114

### 1-((2-(4-Methylpiperidin-1-yl)-6-(trifluoromethyl)pyridin-3-yl)methyl)-3-(6-(pyrrolidin-1-yl)pyridin-3-yl)urea

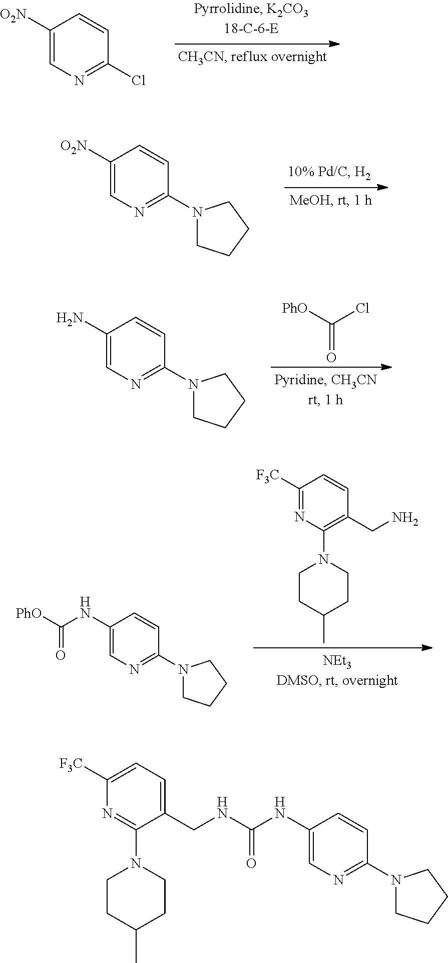

Step 1: 2-Chloro-5-nitropyridine (300 mg, 1.89 mmol), pyrrolidine (0.19 mL, 2.27 mmol), potassium carbonate (785 mg, 5.68 mmol) and 1,4,7,10,13,16-hexaoxacyclooctadecane (60 mg) was dissolved in acetonitrile. The reaction mixture was refluxed overnight. The reaction mixture was cooled to room temperature and then was concentrated under reduced pressure. Then the mixture was extracted with ethyl acetate and washed with water. The organic layer was concentrated under reduced pressure. The crude was purified by column chromatography to give 5-nitro-2-(pyrrolidin-1-yl)pyridine (317 mg, 87%).

Step 2: 5-Nitro-2-(pyrrolidin-1-yl)pyridine (317 mg, 1.65 mmol) was dissolved in methanol. 10% Pd/C (64 mg) was added to it. The resulting mixture was stirred at room temperature under hydrogen for 1 h. The mixture was filtered through celite bed and the filtrate was concentrated under reduced pressure to afford the 6-(pyrrolidin-1-yl)pyridin-3-amine (261 mg, 97%).

Step 3: 6-(Pyrrolidin-1-yl)pyridin-3-amine (261 mg, 1.6 mmol) was dissolved in acetonitrile.

To the reaction mixture was added pyridine (0.16 mL, 1.92 mmol) and phenyl chloroformate (0.21 mL, 1.68 mmol), respectively and stirred at room temperature for 1 h. The reaction mixture was diluted with water and extracted with ethyl acetate. The organic layer was concentrated under reduced pressure. The crude was purified by column chromatography to give phenyl 6-(pyrrolidin-1-yl)pyridin-3-ylcarbamate (218 mg, 48%).

Step 4: Phenyl 6-(pyrrolidin-1-yl)pyridin-3-ylcarbamate (70 mg, 0.25 mmol) and (2-(4-methylpiperidin-1-yl)-6-(trifluoromethyl)pyridin-3-yl)methanamine (71 mg, 0.26 mmol) was dissolved in dimethyl sulfoxide. Then triethylamine (0.07 mL, 0.49 mmol) was added to it. The mixture was stirred at room temperature overnight. The reaction mixture was diluted with water and extracted with ethyl acetate. The organic layer was concentrated under reduced pressure. The crude was purified by column chromatography to give the desired 1-((2-(4-methylpiperidin-1-yl)-6-(trifluoromethyl)pyridin-3-yl)methyl)-3-(6-(pyrrolidin-1-yl)pyridin-3-yl)urea (90 mg, 79%).

¹H NMR (300 MHz, CD₃OD): δ 7.97 (d, 1H, J=2.73 Hz, Ar—H), 7.82 (d, 1H, J=7.68 Hz, Ar—H), 7.57 (dd, 1H, J=8.97 Hz, 2.55 Hz, Ar—H), 7.34 (d, 1H, J=7.68 Hz, Ar—H), 6.49 (d, 1H, J=9.15 Hz, Ar—H), 4.43 (s, 2H, Ar—CH₂), 3.46 (m, 6H, pyrrolidine-CH₂ and piperidine-CH₂), 2.89 (m, 2H, piperidine-CH₂), 2.04 (m, 4H, pyrrolidine-CH₂), 1.77 (m, 2H, piperidine-CH₂), 1.57 (m, 1H, piperidine-CH), 1.43 (m, 2H, piperidine-CH₂), 1.01 (d, 3H, J=6.39 Hz, piperidine-CH₃).

### Synthesis of Example 123

### 1-(6-(2-hydroxyethoxy)pyridin-3-yl)-3-((2-(4-methylpiperidin-1-yl)-6-(trifluoromethyl)pyridin-3-yl)methyl)urea

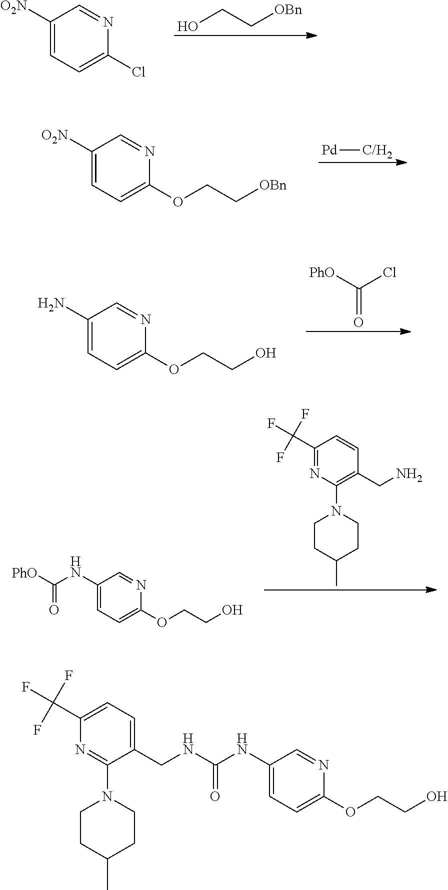

Step 1: 2-chloro-5-nitropyridine (1.51 g, 9.55 mmol, 1 equiv.) and 2-(benzyloxy)ethanol (1.53 g, 10.0 mmol, 1.05 equiv.) were dissolved in DMF (9 mL) and cooled to 0° C. Sodium hydride (60% w/w in mineral oil, 392 mg, 9.84 mmol, 1.03 equiv.) was added in portions and the mixture was allowed to warm to room temperature overnight. After the reaction was complete (TLC), acetic acid (1 mL) was added and the solvent was evaporated. The residue was suspended in Et₂O (20 mL) and filtered. The filter cake was washed with dichloromethane (2×2 mL), the filtrate was evaporated and purified by column chromatography (silica gel, ethyl acetate/n-hexane 1/4, v/v as eluent) to yield 2-(2-(benzyloxy)ethoxy)-5-nitropyridine (2.09 g, 80%) as a yellow solid.

Step 2: 2-(2-(benzyloxy)ethoxy)-5-nitropyridine (2.09 g, 7.61 mmol, 1 equiv) was dissolved in ethanol (90 m) and hydrogenated on an H-cube using 10% Pd on charcoal. The mixture was evaporated and the residue was purified by column chromatography to yield 2-(5-aminopyridin-2-yloxy)ethanol (silica gel, methyl tert-buthyl ether/methanol 9/1, v/v as eluent) to yield (209 mg, 18%) as a colorless solid.

Step 3: To a stirred solution of 2-(5-aminopyridin-2-yloxy)ethanol (209 mg, 1.36 mmol, 1 equiv.) in acetone (5 mL mL) pyridine (329 μL, 4.07 mmol, 3 equiv.) was added followed by phenyl chloroformate (276 μL, 1.76 mmol, 1.3 equiv.) at 0° C. and stirred at room temperature overnight The reaction mixture was evaporated and purified by column chromatography to yield 2-(5-aminopyridin-2-yloxy)ethanol (silica gel, methyl tert-buthyl ether/methanol 9/1, v/v as eluent) to yield phenyl 6-(2-hydroxyethoxy)pyridin-3-ylcarbamate (138 mg, 37%) as a colorless solid.

Step 4: To a stirred solution of (2-(4-methylpiperidin-1-yl)-6-(trifluoromethyl)pyridin-3-yl)methanamine (95 mg, 0.35 mmol, 1.0 eq) in acetonitrile (8 mL) was added triethylamine (0.193 mL, 1.39 mmol, 4.0 eq) followed by phenyl 6-(2-hydroxyethoxy)pyridin-3-ylcarbamate (97 mg, 0.36 mmol, 1.02 eq) and stirred for 16 h at reflux. The reaction mixture was concentrated under vacuum and the residue was purified (column chromatography, silica gel, ethyl acetate/cyclohexane, 9/1, v/v as eluent) to yield 1-(6-(2-hydroxyethoxy)pyridin-3-yl)-3-((2-(4-methylpiperidin-1-yl)-6-(trifluoromethyl)pyridin-3-yl)methyl)urea (example compound 92, 119 mg; 75%) as a colorless solid.

### Synthesis of Example 124

### 1-(6-(2-methoxyethoxy)pyridin-3-yl)-3-((2-(4-methylpiperidin-1-yl)-6-(trifluoromethyl)pyridin-3-yl)methyl)urea

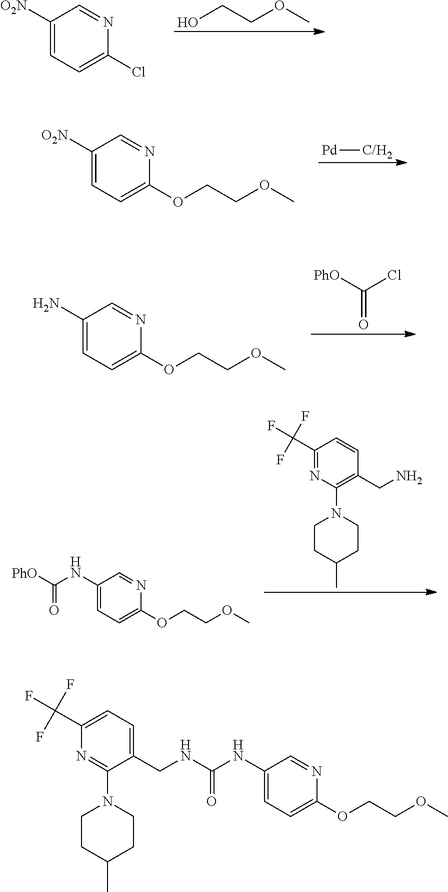

Step 1: 2-chloro-5-nitropyridine (5.00 g, 31.6 mmol, 1 equiv.) and 2-methoxyethanol (2.52 g, 33.1 mmol, 1.05 equiv.) were dissolved in DMF (32 mL) and cooled to 0° C. Sodium hydride (60% w/w in mineral oil, 1.30 mg, 32.5 mmol, 1.03 equiv.) was added in portions and the mixture was allowed to warm to room temperature overnight. After the reaction was complete (TLC), acetic acid (5 mL) was added and the solvent was evaporated. The residue was suspended in Et₂O (100 mL) and filtered. The filter cake was washed with dichloromethane (2×50 mL), the filtrate was evaporated and purified by column chromatography (silica gel, ethyl acetate/n-hexane 1/4, v/v as eluent) to yield 2-(2-methoxyethoxy)-5-nitropyridine (3.96 g, 63%) as a yellow solid.

Step 2: 2-(2-methoxyethoxy)-5-nitropyridine (3.95 g, 19.9 mmol, 1 equiv.) was dissolved in ethanol (180 mL) and hydrogenated on an H-cube using 10% Pd on charcoal. The mixture was evaporated to yield 6-(2-methoxyethoxy)pyridin-3-amine (3.30 mg, 98%) as a colorless solid which was used without further purification.

Step 3: To a stirred solution of 6-(2-methoxyethoxy)pyridin-3-amine (501 mg, 2.98 mmol, 1 equiv.) in acetone (10 mL) pyridine (722 μL, 8.94 mmol, 3 equiv.) was added followed by phenyl chloroformate (489 μL, 3.87 mmol, 1.3 equiv.) at 0° C. and stirred at room temperature overnight. The reaction mixture was evaporated and purified by column chromatography to yield 2-(5-aminopyridin-2-yloxy)ethanol (silica gel, methyl tert-buthyl ether/methanol 1/1, v/v as eluent) to yield phenyl 6-(2-methoxyethoxy)pyridin-3-ylcarbamate (686 mg, 80%) as a colorless solid.

Step 4: To a stirred solution of (2-(4-methylpiperidin-1-yl)-6-(trifluoromethyl)pyridin-3-yl)methanamine (95 mg, 0.35 mmol, 1.0 eq) in acetonitrile (8 mL) was added triethylamine (0.193 mL, 1.39 mmol, 4.0 eq) followed by phenyl 6-(2-methoxyethoxy)pyridin-3-ylcarbamate (102 mg, 0.355 mmol, 1.02 eq) and stirred for 16 h at reflux. The reaction mixture was concentrated under vacuum and the residue was purified (column chromatography, silica gel, ethyl acetate/cyclohexane, 2/1, v/v as eluent) to yield 1-(6-(2-methoxyethoxy)pyridin-3-yl)-3-((2-(4-methylpiperidin-1-yl)-6-(trifluoromethyl)pyridin-3-yl)methyl)urea (example compound 93, 136 mg; 84%) as a colorless solid.

### Synthesis of Example 126

### 1-(5-(Hydroxymethyl)pyridin-3-yl)-3-((2-(4-methylpiperidin-1-yl)-6-(trifluoromethyl)pyridin-3-yl)methyl)urea

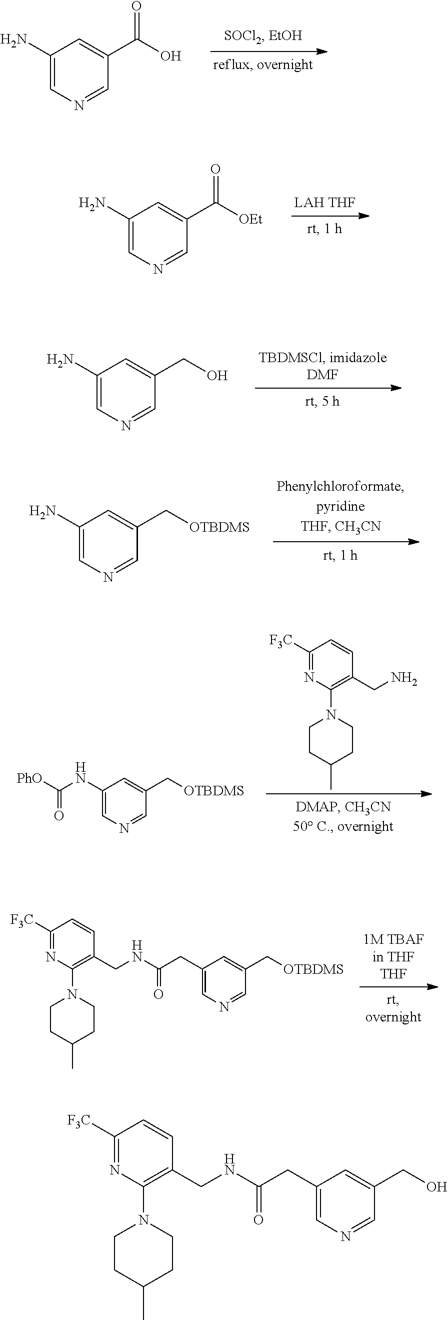

Step 1: To a stirred solution of 5-aminonicotinic acid (300 mg, 2.17 mmol) in ethanol was slowly added thionyl chloride at 0° C. The reaction mixture was stirred overnight under reflux. Then the mixture was cooled to room temperature and the solvent was removed in vacuo. Then it was dissolved in ethylacetate and washed with saturated sodium bicarbonate solution. The organic layer was dried over MgSO₄ and filtered. The filtrate was removed in vacuo. The crude condition of ethyl 5-aminonicotinate (315 mg, 89%) was obtained.

Step 2: To a stirred solution of lithium aluminium hydride (254 mg, 5.36 mmol) in tetrahydrofuran was slowly added solution of ethyl 5-aminonicotinate (223 mg, 1.34 mmol) in tetrahydrofuran at 0° C. under nitrogen atmosphere. The reaction mixture was stirred at 0° C. for 30 minutes then at room temperature for 3 h. The mixture was quenched at 0° C. with 1N HCl until pH is 3 then basified with sodium carbonate solution until pH is 7. Then the mixture was filtered using celite to remove LAH residue and it was dissolved in ethylacetate and washed with saturated sodium carbonate solution. The organic layer was dried over MgSO₄ and filtered. The filtrate was removed in vacuo. The crude condition of (5-aminopyridin-3-yl)methanol (111 mg, crude) was obtained in 54% yield.

Step 3: To a stirred solution of (5-aminopyridin-3-yl)methanol (87 mg, 0.89 mmol) in dimethylformamide were added imidazole (12 mg, 1.77 mmol) and tert-butyldimethylchlorosilane (134 mg, 0.89 mmol). The reaction mixture was stirred at room temperature for 5 h. The mixture dissolved in ethylacetate and washed with water several times. The organic layer was dried over MgSO₄ and filtered. The filtrate was removed in vacuo. The crude was purified by column chromatography. 5-((tert-Butyldimethylsilyloxy)methyl)pyridin-3-amine (132 mg) was obtained in 50% yield.

Step 4: To a stirred solution of 5-((tert-butyldimethylsilyloxy)methyl)pyridin-3-amine (132 mg, 0.55 mmol) in tetrahydrofuran and acetonitrile as co-solvent were added phenylchloroformate (0.073 mL, 0.58 mmol) and pyridine (0.054 mL, 0.66 mmol). The reaction mixture was stirred for 1 h at room temperature. The mixture dissolved in ethylacetate and washed with water and brine. The organic layer was dried over MgSO₄ and filtered. The filtrate removed in vacuo. The crude was purified by column chromatography. Phenyl 5-((tert-butyldimethylsilyloxy)methyl)pyridin-3-ylcarbamate (171 mg) was obtained in 86% yield.

Step 5: To a stirred solution of phenyl 5-((tert-butyldimethylsilyloxy)methyl)pyridin-3-ylcarbamate (100 mg, 0.28 mmol) and (2-(4-methylpiperidin-1-yl)-6-(trifluoromethyl)pyridin-3-yl)methanamine (61 mg, 0.28 mmol) in acetonitrile were added dimethylaminopyridine (27 mg, 0.28 mmol). The reaction mixture was stirred overnight at 50° C. The mixture dissolved in ethylacetate and washed with water and brine. The organic layer was dried over MgSO₄ and filtered. The filtrate removed in vacuo. The crude was purified by column chromatography. 2-(5-((tert-butyldimethylsilyloxy)methyl)pyridin-3-yl)-N-((2-(4-methylpiperidin-1-yl)-6-(trifluoromethyl)pyridin-3-yl)methyl)acetamide (107 mg) was obtained as 89% yield.

Step 6: To a stirred solution of 2-(5-((tert-butyldimethylsilyloxy)methyl)pyridin-3-yl)-N-((2-(4-methylpiperidin-1-yl)-6-(trifluoromethyl)pyridin-3-yl)methyl)acetamide (107 g, 0.20 mmol) in tetrahydrofuran was added 1M tetra-n-butylammoniumfluoride (0.22 mL, 0.22 mmol). The reaction mixture was stirred for 18 h at room temperature. Then another portion of 1M tetra-n-butylammoniumfluoride (0.78 mL, 0.78 mmol) was added and the mixture was stirred for another 4 h. The mixture was quenched with saturated sodium bicarbonate solution then dissolved in ethylacetate and washed with water. The organic layer was dried over MgSO₄ and filtered. The filtrate was removed in vacuo. The crude was purified by column chromatography. 2-(5-(Hydroxymethyl)pyridin-3-yl)-N-((2-(4-methylpiperidin-1-yl)-6-(trifluoromethyl)pyridin-3-yl)methyl)acetamide (77 mg) was obtained in 92% yield.

### Synthesis of Example 127

### 1-(5-(Hydroxymethyl)pyridin-2-yl)-3-((2-(4-methylpiperidin-1-yl)-6-(trifluoromethyl)pyridin-3-yl)methyl)urea

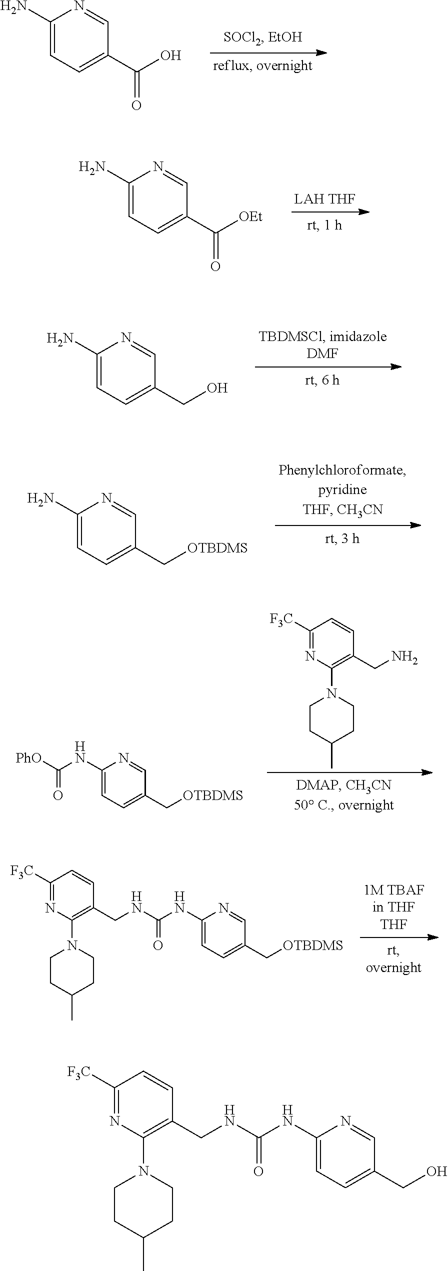

Step 1: To a stirred solution of 6-aminonicotinic acid (300 mg, 2.51 mmol) in ethanol was slowly added thionyl chloride (0.55 mL, 4.34 mmol) at 0° C. The reaction mixture was stirred overnight under reflux. Then the mixture was cooled to room temperature and the solvent was removed in vacuo. Then it was dissolved in ethylacetate and washed with saturated sodium bicarbonate solution. The organic layer was dried over MgSO₄ and filtered. The filtrate was removed in vacuo. The crude condition of ethyl 6-aminonicotinate (317 mg, crude) was obtained in 76% yield.

Step 2: To a stirred solution of lithium aluminium hydride (73 mg, 1.93 mmol) in tetrahydrofuran was slowly added solution of ethyl 6-aminonicotinate (80 mg, 0.48 mmol) in tetrahydrofuran at 0° C. under nitrogen. The reaction mixture was stirred at 0° C. for 30 minutes then at room temperature for 3 h. The mixture was quenched at 0° C. with 1N HCl until pH is 3 then basified with sodium carbonate solution until pH is 7. Then the mixture was filtered using celite to remove LAH residue and it was dissolved in ethylacetate and washed with saturated sodium carbonate solution. The organic layer was dried over MgSO₄ and filtered. The filtrate was removed in vacuo. The crude condition of (6-aminopyridin-3-yl)methanol (30 mg, crude) was obtained in 50% yield.

Step 3: To a stirred solution of (6-aminopyridin-3-yl)methanol (30 mg, 0.24 mmol) in dimethylformamide were added imidazole (33 mg, 0.48 mmol) and tert-butyldimethylchlorosilane (36 mg, 0.24 mmol). The reaction mixture was stirred at room temperature for 5 h. The mixture was dissolved in ethylacetate and washed with water several times to remove dimethylformamide residue. The organic layer was dried over MgSO₄ and filtered. The filtrate was removed in vacuo. The crude was purified by column chromatography. 5-((tert-butyldimethylsilyloxy)methyl)pyridin-2-amine (35 mg) was obtained in 35% yield.

Step 4: To a stirred solution of 5-((tert-butyldimethylsilyloxy)methyl)pyridin-2-amine (35 mg, 0.15 mmol) in tetrahydrofuran and acetonitrile as a co-solvent were added phenylchloroformate (0.018 mL, 0.15 mmol) and pyridine (0.015 mL, 0.18 mmol). The reaction mixture was stirred for 1 h at room temperature. The mixture was dissolved in ethylacetate and washed with water and brine. The organic layer was dried over MgSO₄ and filtered. The filtrate was removed in vacuo. The crude was purified by column chromatography. Phenyl 5-((tert-butyldimethylsilyloxy)methyl)pyridin-2-ylcarbamate (75 mg) was obtained in 99% yield.

Step 5: To a stirred solution of phenyl 5-((tert-butyldimethylsilyloxy)methyl)pyridin-2-ylcarbamate (75 mg, 0.21 mmol) and (2-(4-methylpiperidin-1-yl)-6-(trifluoromethyl)pyridin-3-yl)methanamine (58 mg, 0.21 mmol) in acetonitrile was added dimethylaminopyridine (24 mg, 0.21 mmol). The reaction mixture was stirred overnight at 50° C. The mixture was dissolved in ethylacetate and washed with water and brine. The organic layer was dried over MgSO₄ and filtered. The filtrate was removed in vacuo. The crude was purified by column chromatography. 1-(5-((tert-Butyldimethylsilyloxy)methyl)pyridin-2-yl)-3-((2-(4-methylpiperidin-1-yl)-6-(trifluoromethyl)pyridin-3-yl)methyl)urea (93 mg) was obtained in 82% yield.

Step 6: To a stirred solution of 1-(5-((tert-butyldimethylsilyloxy)methyl)pyridin-2-yl)-3-((2-(4-methylpiperidin-1-yl)-6-(trifluoromethyl)pyridin-3-yl)methyl)urea (93 g, 0.17 mmol) in tetrahydrofuran was added 1M tetra-n-butylammoniumfluoride (0.26 mL, 0.26 mmol). The reaction mixture was stirred for 18 h at room temperature. Then another portion of 1M tetra-n-butylammoniumfluoride (0.39 mL, 0.39 mmol) was added and the mixture was stirred for another 4 h. The mixture was quenched with saturated sodium bicarbonate solution then dissolved in ethylacetate and washed with water. The organic layer was dried over MgSO₄ and filtered. The filtrate was removed in vacuo. The crude was purified by column chromatography. 1-(5-(Hydroxymethyl)pyridin-2-yl)-3-((2-(4-methylpiperidin-1-yl)-6-(trifluoromethyl)pyridin-3-yl)methyl)urea (24 mg) was obtained in 33% yield.

### Synthesis of Example 128

### 1-(3-(Hydroxymethyl)pyridin-4-yl)-3-((2-(4-methylpiperidin-1-yl)-6-(trifluoromethyl)pyridin-3-yl)methyl)urea

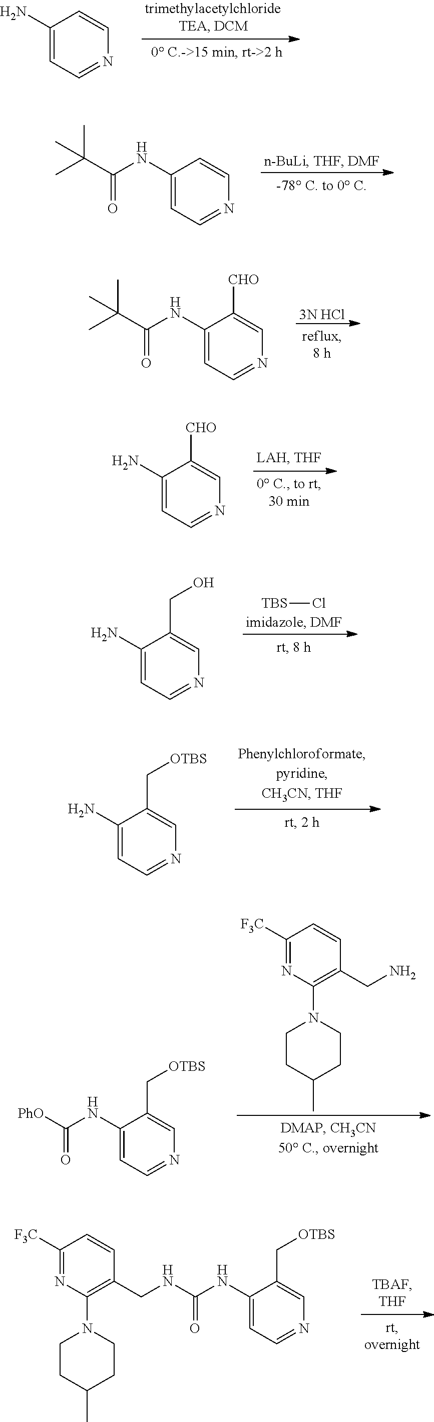

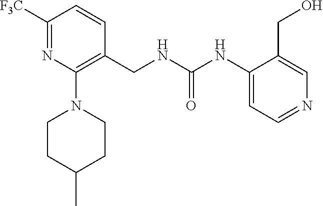 [SMILES: CC1CCN(c2nc(C(F)(F)F)ccc2CNC(=O)Nc2ccncc2CO)CC1]

Step 1: A solution of trimethylacetylcholride (423 mg, 3.51 mmol, 1.1 eq) in dichloromethane was slowly added to an ice cooled solution of pyridin-4-amine (300 mg, 3.19 mmol) and triethylamine (0.56 mL, 3.98 mmol, 1.25 eq) of dichloromethane. The resulting mixture was stirred in and ice bath for 15 min and then at room temperature for 2 h and poured into water. The reaction mixture was washed with dilute NaHCO₃ dried over Na₂SO₄, and evaporated. The crude was purified by column chromatography to give N-(pyridin-4-yl)pivalamide (377 mg, 66%).

Step 2: N-(Pyridin-4-yl)pivalamide (377 mg, 2.12 mmol) was dissolved in anhydrous tetrahydrofuran under inert atmosphere and cooled to −78° C. Within 1 h, a 1.6 M hexane solution of buthyl-lithium (3.3 mL, 5.29 mmol, 2.5 eq) was added drop wise. Then the reaction mixture was warmed to 0° C., stirred for 3 h, and anhydrous dimethylformamide (0.5 mL, 6.35 mmol, 3 eq) in anhydrous tetrahydrofuran (3 mL) was added. Subsequently, the solution was warmed to room temperature and stirred for an additional 45 min. The mixture was poured onto a mixture of 6 N HCl (5 mL) and ice (5 g). After stirring for 5 min, the solution was neutralized with K₂CO₃ (3.3 g) and extracted with diethylether. The organic layer was dried over MgSO₄ and the solvent was removed in vacuo. The residue was purified by columnchromatography to get N-(3-formylpyridin-4-yl)pivalamide (258 mg, 59%).

Step 3: N-(3-Formylpyridin-4-yl)pivalamide (245 mg, 1.20 mmol) was dissolved in 3 N HCl (2.47 mL) and heated to reflux for 8 h. TLC showed complete consumption of starting material. The mixture was extracted with diethylether. The aqueous phase was made alkali with K₂CO₃ and extracted with chloroform. The organic layer was dried over MgSO₄ and concentrated under reduced pressure. The crude was purified by column chromatography to give 4-aminonicotinaldehyde (57 mg, 40%).

Step 4: A solution of 4-aminonicotinaldehyde (57 mg, 0.47 mmol) in tetrahydrofuran was cooled in an ice bath and lithium aluminium hydride (27 mg, 0.70 mmol, 1.5 eq) was added. The ice bath was removed and the reaction mixture was sittred for 30 min. TLC showed complete consumption of starting material. The reaction mixture was quenched with water (1 mL) and 1 N HCl (2 mL) was added extracted with ethylacetate. The organic part was washed with water and brine. The organic layer was dried over MgSO₄ and concentrated under reduced pressure. The residue was used for the next reaction with in a crude state (60 mg, 99%).

Step 5: To a stirred solution of (4-aminopyridin-3-yl)methanol (200 mg, 1.61 mmol) in dimethylformamide were added imidazole (219 mg, 3.22 mmol, 2 eq) and tert-butyldimethylchlorosilane (267 mg, 1.77 mmol, 1.1 eq). The reaction mixture was stirred at room temperature for 5 h. The mixture was dissolved in ethylacetate and washed with water several times. The organic layer was dried over MgSO₄ and filtered. The filtrate was removed in vacuo. The crude was purified by column chromatography get 3-((tert-butyldimethylsilyloxy)methyl)pyridin-4-amine (325 mg, 85%).

Step 6: 3-((tert-Butyldimethylsilyloxy)methyl)pyridin-4-amine (325 mg, 1.36 mmol) was dissolved in acetonitrile (3 mL) and tetrahydrofuran (4 mL). The reaction mixture was added pyridine (0.13 mL, 1.64 mmol, 1.2 eq) and phenyl chloroformate (0.18 mL, 1.43 mmol, 1.05 eq) and stirred at room temperature for 3 h under nitrogen atmosphere. TLC showed complete consumption of starting material. The reaction mixture was diluted with water and extracted with ethylacetate. The organic part was washed with water and brine. The organic layer was dried over MgSO₄ and concentrated under reduced pressure. The crude was purified by column chromatography to give pure phenyl 3-((tert-butyldimethylsilyloxy)methyl)pyridin-4-ylcarbamate (151 mg, 46%).

Step 7: To a solution of phenyl 3-((tert-butyldimethylsilyloxy)methyl)pyridin-4-ylcarbamate (75 mg, 0.21 mmol) in acetonitrile (3 mL) was added dimethylaminopyridine (26 mg, 0.21 mmol, 1 eq) and (2-(4-methylpiperidin-1-yl)-6-(trifluoromethyl)pyridin-3-yl)methanamine (63 mg, 0.23 mmol, 1.1 eq) at room temperature. The reaction mixture was heated to 50° C. for overnight. TLC showed complete consumption of starting material. The reaction mixture was diluted with water and extracted with ethylacetate. The organic part was washed with water and brine. The organic layer was dried over MgSO₄ and concentrated under reduced pressure. The crude was purified by column chromatography to give pure 1-(3-((tert-butyldimethylsilyloxy)methyl)pyridin-4-yl)-3-((2-(4-methylpiperidin-1-yl)-6-(trifluoromethyl)pyridin-3-yl)methyl)urea (103 mg, 92%).

Step 8: To a stirred solution of 1-(3-((tert-butyldimethylsilyloxy)methyl)pyridin-4-yl)-3-((2-(4-methylpiperidin-1-yl)-6-(trifluoromethyl)pyridin-3-yl)methyl)urea (103 mg, 0.19 mmol) in tetrahydrofuran was added 1 M tetra-n-butylammoniumfluoride (0.38 mL, 0.38 mmol, 2 eq). The reaction mixture was stirred for 18 h at room temperature. The mixture was quenched with saturated sodium bicarbonate solution then dissolved in ethylacetate and washed with water. The organic layer was dried over MgSO₄ and filtered. The filtrate removed in vacuo. The crude was purified by column chromatography to get 1-(3-(hydroxymethyl)pyridin-4-yl)-3-((2-(4-methylpiperidin-1-yl)-6-(trifluoromethyl)pyridin-3-yl)methyl)urea (45 mg, 55%).

### Synthesis of Example 129

### 1-(6-(1,2-Dihydroxyethyl)pyridin-3-yl)-3-((2-(4-methylpiperidin-1-yl)-6-(trifluoromethyl)pyridin-3-yl)methyl)urea

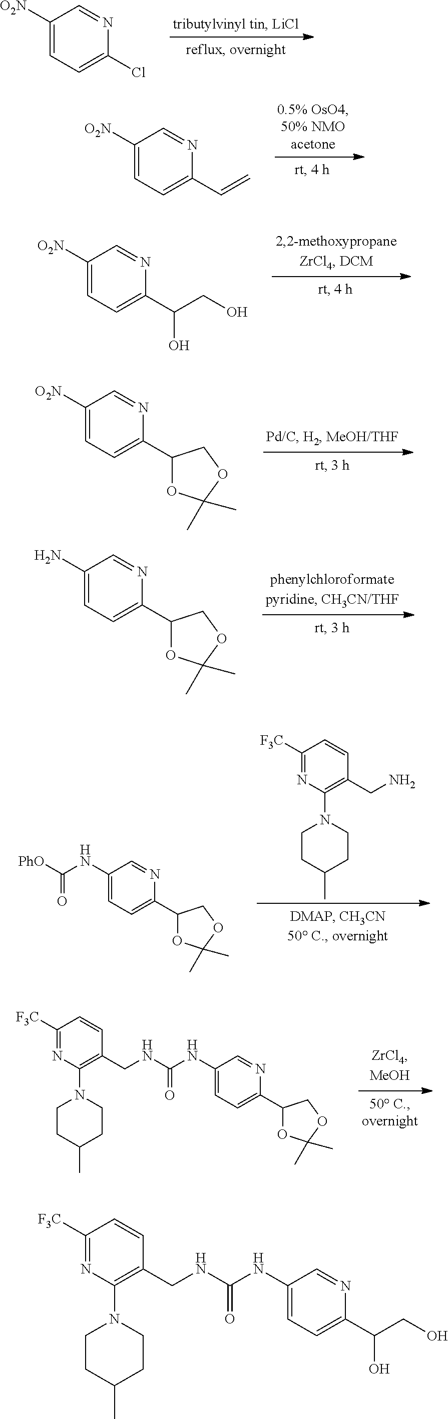

Step 1: To the solution 2-chloro-4-nitropyridine (500 mg, 3.15 mmol) in tetrahydrofuran was added lithium chloride (936 mg, 22.08 mmol, 7 eq), Pd(PPh₃)₄ (547 mg, 0.47 mmol, 0.15 eq) and tributyl vinyltin (1.84 mL, 6.31 mmol, 2 eq) at room temperature. The reaction mixture was refluxed for overnight under nitrogen atmosphere. TLC showed complete consumption of starting material. The reaction mixture was cooled to room temperature. The mixture was diluted with ethylacetate and the organic layer was washed with saturated potassium fluoride solution and then extracted with ethylacetate. The organic part was washed with brine. The organic layer was dried over MgSO₄ and concentrated under reduced pressure to afford crude product which was purified by column chromatography to afford 5-nitro-2-vinylpyridine (350 mg, 74%).

Step 2: To the solution of 5-nitro-2-vinylpyridine (350 mg, 2.33 mmol) in acetone under nitrogen atmosphere was added of 0.5% osmium tetroxide (in H₂O) (2.36 mL, 0.05 mmol, 0.02 eq) and 50% N-methylmorpholine-N-oxide (in H₂O) (1.66 mL, 6.99 mmol, 3 eq). Reaction mixture was stirred at room temperature for 4 h. TLC showed complete consumption of starting material. The reaction mixture was diluted with water and extracted with ethylacetate. The organic part was washed with brine. The organic layer was dried over MgSO₄ and concentrated under reduced pressure to afford crude product which was purified by column chromatography to afford 1-(5-nitropyridin-2-yl)ethane-1,2-diol (368 mg, 86%).

Step 3: A solution of 1-(5-nitropyridin-2-yl)ethane-1,2-diol (368 mg, 2.00 mmol) in dichloromethane was treated with zirconium tetrachloride (47 mg, 0.20 mmol, 0.1 eq) and 2,2-methoxypropane (0.3 mL, 2.40 mmol, 1.2 eq). The mixture was stirred for 4 h at room temperature. TLC showed complete consumption of starting material. The reaction mixture was diluted with water and extracted with ethylacetate. The organic part was washed with brine. The organic layer was dried over MgSO₄ and concentrated under reduced pressure to afford crude product which was purified by column chromatography to afford 2-(2,2-dimethyl-1,3-dioxolan-4-yl)-5-nitropyridine (311 mg, 69%).

Step 4: 2-(2,2-Dimethyl-1,3-dioxolan-4-yl)-5-nitropyridine (311 mg, 1.38 mmol) was dissolved in methanol and tetrahydrofuran (1:1, 15 mL). 10% Pd/C (31 mg, 10%) were added to it. The resulting mixture was stirred at room temperature for 3 h under H₂. TLC showed complete consumption of starting material. The mixture was filtered through celite bed and the filterate was concentrated under reduced pressure. The crude was purified by column chromatography to give 6-(2,2-dimethyl-1,3-dioxolan-4-yl)pyridin-3-amine (201 mg, 75%).

Step 5: 6-(2,2-Dimethyl-1,3-dioxolan-4-yl)pyridin-3-amine (201 mg, 1.04 mmol) was dissolved in acetonitrile (3 mL) and tetrahydrofuran (4 mL). To the reaction mixture was added pyridine (0.10 mL, 1.24 mmol, 1.2 eq) and phenyl chloroformate (0.14 mL, 1.09 mmol, 1.05 eq) and stirred at room temperature for 3 h under nitrogen atmosphere. TLC showed complete consumption of starting material. The reaction mixture was diluted with water and extracted with ethylacetate. The organic part was washed with water and brine. The organic layer was dried over MgSO₄ and concentrated under reduced pressure. The crude was purified by column chromatography to give phenyl 6-(2,2-dimethyl-1,3-dioxolan-4-yl)pyridin-3-ylcarbamate (321 mg, 99%).

Step 6: To a solution of phenyl 6-(2,2-dimethyl-1,3-dioxolan-4-yl)pyridin-3-ylcarbamate (105 mg, 0.33 mmol) in acetonitrile (3 mL) was added DMAP (41 mg, 0.33 mmol, 1 equiv)) and (2-(4-methylpiperidin-1-yl)-6-(trifluoromethyl)pyridin-3-yl)methanamine (100 mg, 0.37 mmol, 1.1 equiv) at room temperature. The reaction mixture was heated to 50° C. for overnight. TLC showed complete consumption of starting material. The reaction mixture was diluted with water and extracted with EA. The organic part was washed with water and brine. The organic layer was dried over MgSO₄ and concentrated under reduced pressure. The crude was purified by column chromatography to give 1-(6-(2,2-dimethyl-1,3-dioxolan-4-yl)pyridin-3-yl)-3-((2-(4-methylpiperidin-1-yl)-6-(trifluoromethyl)pyridin-3-yl)methyl)urea (149 mg, 90%).

Step 7: A solution of 1-(6-(2,2-dimethyl-1,3-dioxolan-4-yl)pyridin-3-yl)-3-((2-(4-methylpiperidin-1-yl)-6-(trifluoromethyl)pyridin-3-yl)methyl)urea (149 mg, 0.31 mmol) in Methanol was added ZrCl₄ (22 mg, 0.09 mmol, 0.3 eq) at room temperature. The reaction mixture was heated to 50° C. for overnight. TLC showed complete consumption of starting material. The reaction mixture was diluted with water and extracted with EA. The organic part was washed with water and brine. The organic layer was dried over MgSO₄ and concentrated under reduced pressure. The crude was purified by column chromatography to give 1-(6-(1,2-dihydroxyethyl)pyridin-3-yl)-3-((2-(4-methylpiperidin-1-yl)-6-(trifluoromethyl)pyridin-3-yl)methyl)urea (44 mg, 32%). Mass spectrometric data are cited hereinafter by way of example for the following exemplary compounds (Tables 1a and 1b):

TABLE 1a

| Exemplary | |
|---|---|
| compound | [M + H] |
| 5 | 406.1 |
| 6 | 424.1 |
| 7 | 386.1 |
| 8 | 402.1 |
| 9 | 367.2 |
| 10 | 364.4 |
| 13 | 393.1 |
| 14 | 407.1 |
| 19 | 393.4 |
| 22 | 381.2 |
| 24 | 384.2 |
| 31 | 393.3 |
| 32 | 407.2 |
| 38 | 393.2 |
| 39 | 394.2 |
| 40 | 394.4 |
| 41 | 394.2 |
| 42 | 395.1 |
| 47 | 394.2 |
| 49 | 427.0 |
| 67 | 517.1 |
| 74 | 450.1 |
| 75 | 525.6 |
| 76 | 527.4 |
| 77 | 491.2 |
| 78 | 517.2 |
| 79 | 531.2 |
| 80 | 533.4 |
| 81 | 538.2 |
| 84 | 500.4 |
| 92 | 500.3 |
| 95 | 488.0 |
| 96 | 517.5 |
| 97 | 529.6 |
| 98 | 526.0 |
| 99 | 560.0 |
| 104 | 505.2 |
| 105 | 449.4 |
| 108 | 465.2 |
| 114 | 463.4 |
| 120 | 437.3 |

TABLE 1b

| Exemplary | | Exemplary | | Exemplary | |
|---|---|---|---|---|---|
| compound | [M + H] | compound | [M + H] | compound | [M + H] |
| 55 | 423.9 | 67 | 517.1 | 85 | 466.3 |
| 86 | 453.2 | 87 | 480.3 | 88 | 467.2 |
| 89 | 480.1 | 90 | 467.2 | 91 | 481.3 |
| 104 | 505.2 | 105 | 449.4 | 107 | 478.9 |
| 114 | 463.4 | 116 | 493.4 | 117 | 479.1 |
| 118 | 479.1 | 123 | 454.2 | 124 | 468.2 |
| 126 | 424.1 | 127 | 424.1 | 128 | 424.1 |
| 129 | 454.2 | 130 | 450.2 | 131 | 467.3 |

### Pharmacological Methods

### I. Functional Testing Carried Out on the Vanilloid Receptor 1 (VR1/TRPV1 Receptor)

The agonistic or antagonistic effect of the substances to be tested on the rat-species vanilloid receptor 1 (VR1/TRPV1) can be determined using the following assay. In this assay, the influx of Ca²⁺ through the receptor channel is quantified with the aid of a Ca²⁺-sensitive dye (type Fluo-4, Molecular Probes Europe BV, Leiden, the Netherlands) in a fluorescent imaging plate reader (FLIPR, Molecular Devices, Sunnyvale, USA).

### Method:

Complete medium: 50 mL HAMS F12 nutrient mixture (Gibco Invitrogen GmbH, Karlsruhe, Germany) with 10% by volume of FCS (foetal calf serum, Gibco Invitrogen GmbH, Karlsruhe, Germany, heat-inactivated); 2_mM L-glutamine (Sigma, Munich, Germany); 1% by weight of AA solution (antibiotic/antimyotic solution, PAA, Pasching, Austria) and 25 ng/mL NGF medium (2.5 S, Gibco Invitrogen GmbH, Karlsruhe, Germany)

Cell culture plate: Poly-D-lysine-coated, black 96-well plates having a clear base (96-well black/clear plate, BD Biosciences, Heidelberg, Germany) are additionally coated with laminin (Gibco Invitrogen GmbH, Karlsruhe, Germany), the laminin being diluted with PBS (Ca—Mg-free PBS, Gibco Invitrogen GmbH, Karlsruhe, Germany) to a concentration of 100 μg/mL. Aliquots having a laminin concentration of 100 μg/mL are removed and stored at −20° C. The aliquots are diluted with PBS in a ratio of 1:10 to 10 μg/mL of laminin and respectively 50 μL of the solution are pipetted into a recess in the cell culture plate. The cell culture plates are incubated for at least two hours at 37° C., the excess solution is removed by suction and the recesses are each washed twice with PBS. The coated cell culture plates are stored with excess PBS which is not removed until just before the feeding of the cells.

### Preparation of the Cells:

The vertebral column is removed from decapitated rats and placed immediately into cold HBSS buffer (Hank's buffered saline solution, Gibco Invitrogen GmbH, Karlsruhe, Germany), i.e. buffer located in an ice bath, mixed with 1% by volume (percent by volume) of an AA solution (antibiotic/antimyotic solution, PAA, Pasching, Austria). The vertebral column is cut longitudinally and removed together with fasciae from the vertebral canal. Subsequently, the dorsal root ganglia (DRG) are removed and again stored in cold HBSS buffer mixed with 1% by volume of an AA solution. The DRG, from which all blood remnants and spinal nerves have been removed, are transferred in each case to 500 μL of cold type 2 collagenase (PAA, Pasching, Austria) and incubated for 35 minutes at 37° C. After the addition of 2.5% by volume of trypsin (PAA, Pasching, Austria), incubation is continued for 10 minutes at 37° C. After complete incubation, the enzyme solution is carefully pipetted off and 500 μL of complete medium are added to each of the remaining DRG. The DRG are respectively suspended several times, drawn through cannulae No. 1, No. 12 and No. 16 using a syringe and transferred to a 50 mL Falcon tube which is filled up to 15 mL with complete medium. The contents of each Falcon tube are respectively filtered through a 70 μm Falcon filter element and centrifuged for 10 minutes at 1,200 rpm and room temperature. The resulting pellet is respectively taken up in 250 μL of complete medium and the cell count is determined.

The number of cells in the suspension is set to 3×10⁵ per mL and 150 μL of this suspension are in each case introduced into a recess in the cell culture plates coated as described hereinbefore. In the incubator the plates are left for two to three days at 37° C., 5% by volume of CO₂ and 95% relative humidity. Subsequently, the cells are loaded with 2 μM of Fluo-4 and 0.01% by volume of Pluronic F127 (Molecular Probes Europe BV, Leiden, the Netherlands) in HBSS buffer (Hank's buffered saline solution, Gibco Invitrogen GmbH, Karlsruhe, Germany) for 30 min at 37° C., washed 3 times with HBSS buffer and after further incubation for 15 minutes at room temperature used for Ca²⁺measurement in a FLIPR assay. The Ca²⁺-dependent fluorescence is in this case measured before and after the addition of substances (λex=488 nm, λem=540 nm). Quantification is carried out by measuring the highest fluorescence intensity (FC, fluorescence counts) over time.

### FLIPR Assay:

The FLIPR protocol consists of 2 substance additions. First the compounds to be tested (10 μM) are pipetted onto the cells and the Ca²⁺ influx is compared with the control (capsaicin 10 μM). This provides the result in % activation based on the Ca²⁺ signal after the addition of 10 μM of capsaicin (CP). After 5 minutes' incubation, 100 nM of capsaicin are applied and the Ca²⁺ influx is also determined.

Desensitising agonists and antagonists lead to suppression of the Ca²⁺ influx. The % inhibition is calculated compared to the maximum achievable inhibition with 10 μM of capsazepine.

Triple analyses (n=3) are carried out and repeated in at least 3 independent experiments (N=4).

Starting from the percentage displacement caused by different concentrations of the compounds to be tested of general formula I, IC₅₀ inhibitory concentrations which cause a 50-percent displacement of capsaicin were calculated. K, values for the test substances were obtained by conversion by means of the Cheng-Prusoff equation (Cheng, Prusoff; Biochem. Pharmacol. 22, 3099-3108, 1973).

### Pharmacological Data:

The affinity of the compounds according to the invention for the vanilloid receptor 1 (VR1/TRPV1 receptor) was determined as described hereinbefore.

The compounds according to the invention display affinity to the VR1/TRPV1 receptor as shown in Tables 2 and 3 given below. In said table Cap denotes capsaicin and AG denotes agonist.

The value after the “@” symbol indicates the concentration at which the inhibition (as a percentage) was respectively determined.

TABLE 2

| Exemplary | TRPV1 human |
|---|---|
| compound | (f) Ki [nM] CAP |
| 5 | 91 |
| 6 | 48% @ 5 μM |
| 7 | 18% @ 5 μM |
| 8 | 39% @ 5 μM |
| 9 | 23% @ 5 μM |
| 10 | 73 |
| 13 | 7 |
| 14 | 45 |
| 19 | 35 |
| 22 | 32% @ 5 μM |
| 24 | 13% @ 5 μM |
| 31 | 6 |
| 32 | 10 |
| 38 | AG |
| 39 | 11 |
| 40 | 107 |
| 41 | 39% @ 5 μM |
| 42 | 9 |
| 47 | 38 |
| 49 | AG |
| 67 | 6 |
| 74 | AG |
| 75 | 57 |
| 76 | 77 |
| 77 | 80 |
| 78 | 42 |
| 79 | 11 |
| 80 | 60 |
| 81 | 5 |
| 84 | 50 |
| 92 | 9 |
| 95 | 34% @ 5 μM |
| 96 | 1 |
| 97 | 2 |
| 98 | 63 |
| 99 | 9 |
| 104 | 9 |
| 105 | 27 |
| 108 | 12 |
| 114 | 12 |
| 120 | 44 |

TABLE 3

| Exem- | TRPV1 | Exem- | TRPV1 | Exem- | TRPV1 |
|---|---|---|---|---|---|
| plary | human | plary | human | plary | human |
| com- | (f) Ki | com- | (f) Ki | com- | (f) Ki |
| pound | [nM] CAP | pound | [nM] CAP | pound | [nM] CAP |
| 55 | 11 | 67 | 6 | 85 | 2 |
| 86 | 1 | 87 | 27 | 88 | 16 |
| 89 | 53 | 90 | 19 | 91 | 12% @ 1 μM |
| 96 | 1 | 97 | 2 | 104 | 9 |
| 105 | 27 | 107 | 81 | 114 | 12 |
| 116 | 78 | 117 | 9 | 118 | 10 |
| 123 | 11 | 124 | 4 | 126 | 26 |
| 127 | 61 | 128 | 69 | 129 | 39 |
| 130 | 15 | 131 | 29 | | |

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A compound of formula (I) 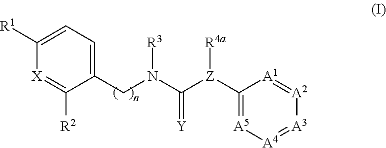
wherein
n represents 1, 2, 3 or 4;
X represents N or CH;
Y represents O, S, or N—CN;
Z represents N or C—R^{4b};
A¹ represents N or CR⁵;
A² represents N or CR⁸;
A³ represents N or CR⁷;
A⁴ represents N or CR⁸;
A⁵ represents N or CR⁹;
with the proviso that 1, 2 or 3 of variables A¹, A², A³, A⁴ and A⁵ represent a nitrogen atom;
R⁰ represents a C₁₋₁₀ aliphatic residue, unsubstituted or mono- or polysubstituted; a C₃₋₁₀ cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; aryl or heteroaryl, in each case unsubstituted or mono- or polysubstituted and in each case optionally bridged via a C₁₋₈ aliphatic group, which in turn may be unsubstituted or mono- or polysubstituted;
R¹ represents a C₁₋₄ aliphatic residue, unsubstituted or mono- or polysubstituted, a C₃₋₆ cycloaliphatic residue or a 3 to 6 membered heterocycloaliphatic residue, in each case unsubstituted or mono- or polysubstituted;
R² represents R⁰; OR⁰; SR⁰; NH₂; NHR⁰ or N(R⁰)₂;
R³ represents H or a C₁ aliphatic residue, unsubstituted or mono- or polysubstituted;
R^{4a} represents H; a C₁₋₄ aliphatic residue, unsubstituted or mono- or polysubstituted; a C₃₋₆ cycloaliphatic residue, unsubstituted or mono- or polysubstituted; or aryl, unsubstituted or mono- or polysubstituted;
R^{4b} represents H; or a C₁ aliphatic residue, unsubstituted, mono- or polysubstituted; or
R^{4a} and R^{4b} together with the carbon atom connecting them form a C₃₋₆ cycloaliphatic residue, unsubstituted or mono- or polysubstituted;
R⁵, R⁶, R⁷, R⁸, and R⁹ each independently represent H; F; Cl; Br; I; CN; CF₃; CF₂H; CFH₂; CF₂Cl; CFCl₂; NO₂; R⁰; C(═O)—H; C(═O)—R⁰; C(═O)—OH; C(═O)—OR⁰; C(═O)—NH₂; C(═O)—NHR⁰; C(═O)—N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NHR⁰; O—C(═O)—N(R⁰)₂; O—S(═O)₂—R⁰; O—S(═O)₂—OH; O—S(═O)₂—OR⁰; O—S(═O)₂—NH₂; O—S(═O)₂—NHR⁰; O—S(═O)₂—N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NHR⁰; NR⁰—C(═O)—N(R⁰)₂; NH—S(═O)₂—OH; NH—S(═O)₂—R⁰; NH—S(═O)₂—OR⁰; NH—S(═O)₂—NH₂; NH—S(═O)₂—NHR⁰; NH—S(═O)₂—N(R⁰)₂; NR⁰—S(═O)₂—OH; NR⁰—S(═O)₂—R⁰; NR⁰—S(═O)₂—OR⁰; NR⁰—S(═O)₂—NH₂; NR⁰—S(═O)₂—NHR⁰; NR⁰—S(═O)₂—N(R⁰)₂; SH; SCF₃; SCF₂H; SCFH₂; SCF₂Cl; SCFCl₂; SR⁰; S(═O)—R⁰; S(═O)₂—R⁰; S(═O)₂—OH; S(═O)₂—OR⁰; S(═O)₂—NH₂; S(═O)₂—NHR⁰; or S(═O)₂—N(R⁰)₂;
in which an “aliphatic group” and an “aliphatic residue” can in each case, independently of one another, be branched or unbranched, saturated or unsaturated;
in which a “cycloaliphatic residue” and a “heterocycloaliphatic residue” can in each case, independently of one another, be saturated or unsaturated;
in which “mono- or polysubstituted” with respect to an “aliphatic group”, an “aliphatic residue”, a “cycloaliphatic residue” and a “heterocycloaliphatic residue” relates in each case independently of one another, with respect to the corresponding residues or groups, to the substitution of one or more hydrogen atoms each independently of one another by at least one substituent selected from the group consisting of F; Cl; Br; I; NO₂; CN; ═O; ═NH; ═N(OH); ═C(NH₂)₂; CF₃; CF₂H; CFH₂; CF₂Cl; CFCl₂; R⁰; C(═O)—H; C(═O)—R⁰; C(═O)—OH; C(═O)—OR⁰; CO—NH₂; C(═O)—NHR⁰; C(═O)—N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂—OH; O—S(═O)₂—OR⁰; O—S(═O)₂—NH₂; O—S(═O)₂—NHR⁰; O—S(═O)₂—N(R⁰)₂; NH₂; NH—R⁰; N(R⁰)₂; NH—C(═O)—R⁰; NH—C(═O)—O—R⁶; NH—C(═O)—NH₂; NH—C(═O)—NHR⁰; NH—C(═O)—N(R)₂; NR⁰—C(═O)—R⁰; NR⁰—C(═O)—O—R⁰; NR⁰—C(═O)—NH₂; NR⁰—C(═O)—NHR⁰; NR⁰—C(═O)—N(R⁰)₂; NH—S(═O)₂—OH; NH—S(═O)₂—R⁰; NH—S(═O)₂—OR⁰; NH—S(═O)₂—NH₂; NH—S(═O)₂—NHR⁰; NH—S(═O)₂—N(R⁰)₂; NR⁰—S(═O)₂—OH; NR⁰—S(═O)₂—R⁰; NR⁰—S(═O)₂—OR⁰; NR⁰—S(═O)₂—NH₂; NR⁰—S(═O)₂—NHR⁰; NR⁰—S(═O)₂—N(R⁰)₂; SH; SCF₃; SCF₂H; SCFH₂; SCF₂Cl; SCFCl₂; SR⁰; S(═O)—R⁰; S(═O)₂—R⁰; S(═O)₂—OH; S(═O)₂—OR⁰; S(═O)₂—NH₂; S(═O)₂—NHR⁰; and S(═O)₂—N(R⁰)₂; and
in which “mono- or polysubstituted” with respect to “aryl” and a “heteroaryl” relates, with respect to the corresponding residues, in each case independently of one another, to the substitution of one or more hydrogen atoms each independently of one another by at least one substituent selected from the group consisting of F; Cl; Br; I; NO₂; CN; CF₃; CF₂H; CFH₂; CF₂Cl; CFCl₂; R⁰; C(═O)—H; C(═O)—R⁰; C(═O)—OH; C(═O)—OR⁶; CO—NH₂; C(═O)—NHR⁰; C(═O)—N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂—OH; O—S(═O)₂—OR⁰; O—S(═O)₂—NH₂; O—S(═O)₂—NHR⁰; O—S(═O)₂—N(R⁰)₂; NH₂; NHR⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂—OH; NH—S(═O)₂—R⁰; NH—S(═O)₂—OR⁰; NH—S(═O)₂—NH₂; NH—S(═O)₂—NHR⁰; NH—S(═O)₂—N(R⁰)₂; NR⁰—S(═O)₂—OH; NR⁰—S(═O)₂R⁰; NR⁰—S(═O)₂—OR⁰; NR⁰—S(═O)₂—NH₂; NR⁰—S(═O)₂—NHR⁰; NR⁰—S(═O)₂—N(R⁰)₂; SH; SCF₃; SCF₂H; SCFH₂; SCF₂Cl; SCFCl₂; SR⁰; S(═O)—R⁰; S(═O)₂—R⁰; S(═O)₂—OH; S(═O)₂—OR⁰; S(═O)₂—NH₂; S(═O)₂—NHR⁰; and S(═O)₂—N(R⁰)₂;
optionally in the form of a single stereoisomer or a mixture of stereoisomers, in the form of the free compound and/or a physiologically acceptable salt thereof.

2. A compound according to claim 1, wherein n represents 1.

3. A compound according to claim 1, wherein Y represents O.

4. A compound according to claim 1, wherein R¹ is selected from:
the group consisting of CF₃, methyl, ethyl, n-propyl, isopropyl, n-butyl, sec.-butyl, and tert.-butyl, or
the group consisting of cyclopropyl, cyclobutyl, cyclopentyl, and cyclohexyl.

5. A compound according to claim 1, wherein
R² represents a substructure (T1) 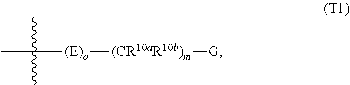
wherein
E represents O, S, or NR¹¹, wherein
R¹¹ represents H or a C₁₋₄ aliphatic residue, unsubstituted or mono- or polysubstituted with one or more substituents each independently selected from the group consisting of F, Cl, Br, I, OH, O—C₁₋₄ alkyl, OCF₃, NH₂, NH—C₁₋₄ alkyl and N(C₁₋₄ alkyl)₂;
o represents 0 or 1;
R^{10a} and R^{10b} each independently represent H; F; Cl; Br; I; or a C₁₋₄ aliphatic residue, unsubstituted or mono- or polysubstituted with one or more substituents independently selected from the group consisting of F, Cl, Br, I, OH, O—C₁₋₄ alkyl, OCF₃, NH₂, NH—C₁₋₄ alkyl and N(C₁₋₄ alkyl)₂;
m represents 0, 1, 2, 3 or 4; and
G represents:
a C₁₋₄ aliphatic residue, unsubstituted or mono- or polysubstituted with one or more substituents each independently selected from the group consisting of F, Cl, Br, I, NO₂, CN, OH, ═O, O—C₁₋₄ alkyl, O—C₁₋₄ alkylen-O—C₁₋₄ alkyl, OCF₃, CF₃, NH₂, NH(C₁₋₄ alkyl), N(C₁₋₄ alkyl)₂, SH, S—C₁₋₄ alkyl, SCF₃, phenyl and pyridyl, wherein phenyl or pyridyl are respectively unsubstituted or mono- or polysubstituted with one or more substituents each independently selected from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH; or
a C₃₋₁₀ cycloaliphatic residue or a 3 to 10 membered heterocyclo-aliphatic residue, in each case unsubstituted or mono- or polysubstituted with one or more substituents each independently selected from the group consisting of F, Cl, Br, I, NO₂, CN, OH, ═O, O—C₁₋₄ alkyl, OCF₃, C₁₋₄ alkyl, CF₃, SH, S—C₁₋₄ alkyl, SCF₃, NH₂, NH(C₁₋₄ alkyl), N(C₁₋₄ alkyl)₂, phenyl and pyridyl, wherein phenyl or pyridyl are respectively unsubstituted or mono- or polysubstituted with one or more substituents each independently selected from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH; or
an aryl or heteroaryl, unsubstituted or mono- or polysubstituted with one or more substituents each independently selected from the group consisting of F, Cl, Br, I, NO₂, CN, OH, O—C₁₋₄ alkyl, OCF₃, C₁₋₄ alkyl, CF₃, SH, S—C₁₋₄ alkyl, SCF₃, NH₂, NH(C₁₋₄ alkyl), N(C₁₋₄ alkyl)₂, phenyl and pyridyl, wherein phenyl or pyridyl are respectively unsubstituted or mono- or polysubstituted with one or more substituents each independently selected from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH.

6. A compound according to claim 5, wherein m represents 0, 1 or 2.

7. A compound according to claim 6, wherein m represents 0 or 1.

8. A compound according to claim 1, wherein R³ is selected from the group consisting of H, methyl and ethyl.

9. A compound according to claim 1, wherein:
R^{4a} represents H, methyl, ethyl, cyclopropyl, cyclobutyl, cyclopentyl, cyclohexyl, or phenyl, wherein phenyl may be unsubstituted or substituted with 1, 2, 3, 4 or 5 substituents independently selected from the group consisting of F, Cl, Br, I, NO₂, CN, CF₃, CF₂H, CFH₂, CF₂Cl, CFCl₂, OH, NH₂, NH(C₁₋₄ alkyl) and N(C₁₋₄ alkyl)(C₁₋₄ alkyl), C₁₋₄ alkyl, and O—C₁₋₄-alkyl; and
R^{4b} represents H, methyl, or ethyl, or
R^{4a} and R^{4b} together with the carbon atom connecting them form a cyclopropyl, cyclobutyl, cyclopentyl, or cyclohexyl ring.

10. A compound according to claim 1, wherein the partial structure 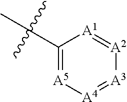
represents a moiety selected from the group consisting of

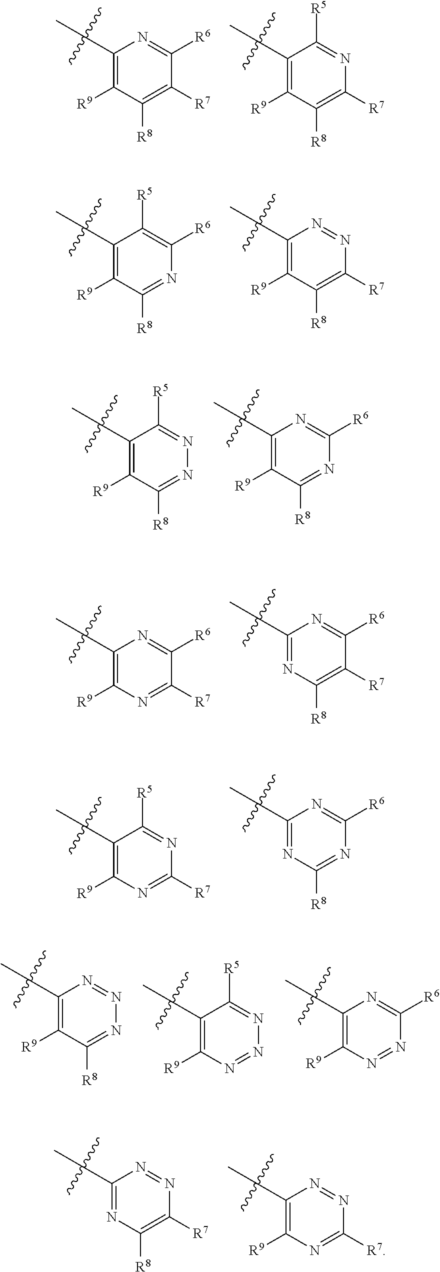

11. A compound according to claim 1, wherein R⁵, R⁶, R⁷, R⁸, and R⁹ each independently represent:
H; F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; NH₂; C(═O)—NH₂; C(═O)—H; C(═O)—OH; S(═O)₂—OH; S(═O)₂—NH₂; or
a C₁₋₁₀ aliphatic residue, (C₁₋₈ aliphatic group)-OH, (C₁₋₈ aliphatic group)-O—C₁₋₁₀ aliphatic residue, (C₁₋₈ aliphatic group)-O—(C₁₋₈ aliphatic group)-OH, (C₁₋₈ aliphatic group)-O—(C₁₋₈ aliphatic group)-O—C₁₋₁₀ aliphatic residue, a (C₁₋₈ aliphatic group)-NH—C₁₋₁₀ aliphatic residue, a (C₁₋₈ aliphatic group)-NH—(C₁₋₈ aliphatic residue)-OH, a (C₁₋₈ aliphatic group)-N(C₁₋₁₀ aliphatic residue)-(C₁₋₈ aliphatic residue)-OH, a (C₁₋₈ aliphatic group)-NH—S(═O)₂—C₁₋₁₀ aliphatic residue, a (C₁₋₈ aliphatic group)-NH—S(═O)₂—NH₂, a (C₁₋₈ aliphatic group)-S(═O)₂—C₁₋₁₀ aliphatic residue, a C(═O)—C₁₋₁₀ aliphatic residue, a C(═O)—NH—C₁₋₁₀ aliphatic residue, or
an O—C₁₋₁₀ aliphatic residue, a O—(C₁₋₈ aliphatic group)-O—C₁₋₁₀ aliphatic residue, O—(C₁₋₈ aliphatic group)-OH, or
an NH—C₁₋₁₀ aliphatic residue, a N(C₁₋₁₀ aliphatic residue)₂, a NH—(C₁₋₈ aliphatic group)-O—C₁₋₁₀ aliphatic residue, a NH—(C₁₋₈ aliphatic group)-OH, a N(C₁₋₁₀ aliphatic residue)[(C₁₋₈ aliphatic group)-OH], a N(C₁₋₁₀ aliphatic residue)[(C₁₋₈ aliphatic group)-O—C₁₋₁₀ aliphatic residue], a NH—C(═O)—C₁₋₁₀ aliphatic residue, a N(C₁₋₁₀ aliphatic residue)[(C(═O)—C₁₋₁₀ aliphatic residue)], a N(C₁₋₁₀ aliphatic residue)[(C₁₋₈ aliphatic group)-O—C₁₋₁₀ aliphatic residue], a N(C₁₋₁₀ aliphatic residue)[(C₁₋₈ aliphatic group)-OH], a NH—S(═O)₂—C₁₋₁₀ aliphatic residue, a N(C₁₋₁₀ aliphatic residue)[S(═O)₂—C₁₋₁₀ aliphatic residue], or
an S(═O)₂—C₁₋₁₀ aliphatic residue, a S(═O)₂—NH—C₁₋₁₀ aliphatic residue, a S(═O)₂—N(C₁₋₁₀ aliphatic residue)₂, a S—C₁₋₁₀ aliphatic residue, wherein each of the C₁₋₁₀ aliphatic residue and C₁₋₈ aliphatic groups are unsubstituted, or
a C₃₋₁₀ cycloaliphatic residue, a C(═O)—C₃₋₁₀ cycloaliphatic residue, a C(═O)NH—C₃₋₁₀ cycloaliphatic residue a O—C₃₋₁₀ cycloaliphatic residue, a O—(C₁₋₈ aliphatic group)-C₃₋₁₀ cycloaliphatic residue, a S—C₃₋₁₀ cycloaliphatic residue, a S—(C₁₋₈ aliphatic group)-C₃₋₁₀ cycloaliphatic residue, a NH—C₃₋₁₀ cycloaliphatic residue, a NH—C(═O)—C₃₋₁₀ cycloaliphatic residue, a NH—(C₁₋₈ aliphatic group)-C₃₋₁₀ cycloaliphatic residue, a N(C₁₋₁₀ aliphatic residue)(C₃₋₁₀ cycloaliphatic residue), a 3 to 10 membered heterocycloaliphatic residue, a C(═O)-(3 to 10 membered heterocycloaliphatic residue), a C(═O)—NH-(3 to 10 membered heterocycloaliphatic residue), a O-(3 to 10 membered heterocycloaliphatic residue), a O—(C₁₋₈ aliphatic group)-(3 to 10 membered heterocycloaliphatic residue), a S-(3 to 10 membered heterocycloaliphatic residue), a S—(C₁₋₈ aliphatic group)-(3 to 10 membered heterocyclo-aliphatic residue), a NH-(3 to 10 membered heterocycloaliphatic residue), a NH—C(═O)-(3 to 10 membered heterocycloaliphatic residue), NH—(C₁₋₈ aliphatic group)-(3 to 10 membered heterocycloaliphatic residue), a N(C₁₋₁₀ aliphatic residue) (3 to 10 membered heterocycloaliphatic residue), wherein:
each of said cycloaliphatic residues may optionally be bridged by a C₁₋₈ aliphatic group,
each of the C₁₋₁₀ aliphatic residue and the C₁₋₈ aliphatic group are unsubstituted, and
each of the C₃₋₁₀ cycloaliphatic residue and the 3 to 10 membered heterocycloaliphatic residue, respectively, can independently be unsubstituted or mono- or polysubstituted with one or more substituents each independently selected from the group consisting of F, Cl, Br, I, C₁₋₄ alkyl, C₁₋₄ alkyl-OH, CF₃, C(═O)—C₁₋₄ alkyl, O—C₁₋₄ alkyl, O—C₁₋₄ alkyl-OH, O—C₁₋₄ alkyl-O—C₁₋₄ alkyl, ═O, OCF₃, OH, SH, S—C₁₋₄ alkyl, SCF₃, SO₂—C₁₋₄ alkyl, NH₂, ═NH, ═N(OH), NH—C₁₋₄ alkyl, N(C₁₋₄ alkyl)₂, NH—SO₂—C₁₋₄ alkyl, NH—C(═O)—C₁₋₄ alkyl, or
aryl, C(═O)-aryl, C(═O)—NH-aryl, O-aryl, a O—(C₁₋₈ aliphatic group)-aryl, S-aryl, a S—(C₁₋₈ aliphatic group)-aryl, a NH-aryl, NH—C(═O)-aryl, NH—S(═O)₂-aryl a NH—(C₁₋₈ aliphatic group)-aryl, a N(C₁₋₁₀ aliphatic residue)(aryl), heteroaryl, C(═O)-heteroaryl, C(═O)—NH-heteroaryl, O-heteroaryl, O—(C₁₋₈ aliphatic group)-heteroaryl, S-(heteroaryl), S—(C₁₋₈ aliphatic group)-(heteroaryl), NH-(heteroaryl), NH—C(═O)-heteroaryl, NH—S(═O)₂-heteroaryl, NH—(C₁₋₈ aliphatic group)(heteroaryl), N(C₁₋₁₀ aliphatic residue)(heteroaryl), wherein
each of the aforementioned residues can optionally be bridged by a C₁₋₈ aliphatic group,
each of the aryl and heteroaryl of the aforementioned residues, respectively, can independently be unsubstituted or mono- or polysubstituted with one or more substituents each independently selected from the group consisting of F, Cl, Br, I, C₁₋₄ alkyl, C₁₋₄ alkyl-OH, CF₃, C(═O)—C₁₋₄ alkyl, O—C₁₋₄ alkyl, O—C₁₋₄ alkyl-OH, O—C₁₋₄ alkyl-O—C₁₋₄ alkyl, OCF₃, OH, SH, S—C₁₋₄ alkyl, SCF₃, SO₂—C₁₋₄ alkyl, NH₂, NH—C₁₋₄ alkyl, N(C₁₋₄ alkyl)₂, NH—SO₂—C₁₋₄ alkyl, NH—C(═O)—C₁₋₄ alkyl, phenyl and pyridyl, wherein phenyl or pyridyl are respectively unsubstituted or mono- or polysubstituted with one or more substituents each independently selected from the group consisting of F, Cl, Br, I, NO₂, CN, OH, O—C₁₋₄ alkyl, O—C₁₋₄ alkylene-O—C₁₋₄ alkyl OCF₃, C₁₋₄ alkyl, C₁₋₄ alkylene-O—C₁₋₄-alkyl, C(═O)—OH, CF₃, CF₂H, CHF₂, NH₂, NH(C₁₋₄ alkyl), N(C₁₋₄ alkyl)₂, SH, S—C₁₋₄ alkyl, SCF₃ and S(═O)₂OH, and
each of the C₁₋₁₀ aliphatic residues and the C₁₋₈ aliphatic groups of the aforementioned residues is unsubstituted.

12. A compound according to claim 1, wherein
n represents 1;
X represents N or CH;
Y represents O;
Z represents N or C—R^{4b};
A¹ represents N or CR⁵;
A² represents N or CR⁶;
A³ represents N or CR⁷;
A⁴ represents N or CR⁸;
A⁵ represents N or CR⁹;
with the proviso that 1, 2 or 3 of variables A¹, A², A³, A⁴ and A⁵ represent a nitrogen atom;
R¹ is selected from the group consisting of tert-butyl, CF₃, cyclopropyl, cyclobutyl, cyclopentyl, and cyclohexyl;
R² represents the substructure (T1) 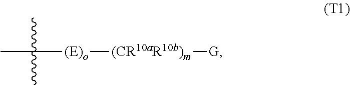
wherein
E represents O, S, or NR¹¹, wherein
R¹¹ represents H or a C₁₋₄ aliphatic residue, unsubstituted or mono- or polysubstituted with one or more substituents each independently selected from the group consisting of F, Cl, Br, I, OH, O—C₁₋₄ alkyl, OCF₃, NH₂, NH—C₁₋₄ alkyl and N(C₁₋₄ alkyl)₂;
o represents 0 or 1;
R^{10a} and R^{10b} each independently represent H; F; Cl; Br; I; or a C₁₋₄ aliphatic residue, unsubstituted or mono- or polysubstituted with one or more substituents each independently selected from the group consisting of F, Cl, Br, I, OH, O—C₁₋₄ alkyl, OCF₃, NH₂, NH—C₁₋₄ alkyl and N(C₁₋₄ alkyl)₂;
m represents 0, 1, 2, 3 or 4; and
G represents
a C₁₋₄ aliphatic residue, unsubstituted or mono- or polysubstituted with one or more substituents each independently selected from the group consisting of F, Cl, Br, I, NO₂, CN, OH, ═O, O—C₁₋₄ alkyl, O—C₁₋₄ alkylen-O—C₁₋₄ alkyl, OCF₃, CF₃, NH₂, NH(C₁₋₄ alkyl), N(C₁₋₄ alkyl)₂, SH, S—C₁₋₄ alkyl, SCF₃, phenyl and pyridyl, wherein phenyl or pyridyl are respectively unsubstituted or mono- or polysubstituted with one or more substituents each independently selected from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH; or
a C₃₋₁₀ cycloaliphatic residue or a 3 to 10 membered heterocyclo-aliphatic residue, in each case unsubstituted or mono- or polysubstituted with one or more substituents each independently selected from the group consisting of F, Cl, Br, I, NO₂, CN, OH, ═O, O—C₁₋₄ alkyl, OCF₃, C₁₋₄ alkyl, CF₃, SH, S—C₁₋₄ alkyl, SCF₃, NH₂, NH(C₁₋₄ alkyl), N(C₁₋₄ alkyl)₂, phenyl and pyridyl, wherein phenyl or pyridyl are respectively unsubstituted or mono- or polysubstituted with one or more substituents each independently selected from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH; or
an aryl or heteroaryl, unsubstituted or mono- or polysubstituted with one or more substituents each independently selected from the group consisting of F, Cl, Br, I, NO₂, CN, OH, O—C₁₋₄ alkyl, OCF₃, C₁₋₄ alkyl, CF₃, SH, S—C₁₋₄ alkyl, SCF₃, NH₂, NH(C₁₋₄ alkyl), N(C₁₋₄ alkyl)₂, phenyl and pyridyl, wherein phenyl or pyridyl are respectively unsubstituted or mono- or polysubstituted with one or more substituents each independently selected from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH;
R³ is selected from the group consisting of H, methyl, and ethyl.
R^{4a} represents H; methyl, ethyl, cyclopropyl, cyclobutyl, cyclopentyl, cyclohexyl, or phenyl, wherein phenyl is unsubstituted or substituted with 1, 2, 3, 4 or 5 substituents independently selected from the group consisting of F; Cl; Br; I; NO₂; CN; CF₃; CF₂H; CFH₂; CF₂Cl; CFCl₂; OH, NH₂, NH(C₁₋₄ alkyl) and N(C₁₋₄ alkyl)(C₁₋₄ alkyl), C₁₋₄ alkyl, and O—C₁₋₄-alkyl; and
R^{4b} represents H; methyl, or ethyl, or
R^{4a} and R^{4b} together with the carbon atom connecting them form cyclopropyl, cyclobutyl, cyclopentyl, or cyclohexyl ring;
R⁵, R⁶, R⁷, R⁹ and R⁹ are each independently selected from the group consisting of:
H; F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; NH₂; C(═O)—NH₂; C(═O)—H; C(═O)—OH; S(═O)₂—OH; S(═O)₂—NH₂;
a C₁₋₁₀ aliphatic residue, (C₁₋₈ aliphatic group)-OH, (C₁₋₈ aliphatic group)-O—C₁₋₁₀ aliphatic residue, (C₁₋₈ aliphatic group)-O—(C₁₋₈ aliphatic group)-OH, (C₁₋₈ aliphatic group)-O—(C₁₋₈ aliphatic group)-O—C₁₋₁₀ aliphatic residue, a (C₁₋₈ aliphatic group)-NH—C₁₋₁₀ aliphatic residue, a (C₁₋₈ aliphatic group)-NH—(C₁₋₈ aliphatic residue)-OH, a (C₁₋₈ aliphatic group)-N(C₁₋₁₀ aliphatic residue)-(C₁₋₈ aliphatic residue)-OH, a (C₁₋₈ aliphatic group)-NH—S(═O)₂—C₁₋₁₀ aliphatic residue, a (C₁₋₈ aliphatic group)-NH—S(═O)₂—NH₂, a (C₁₋₈ aliphatic group)-S(═O)₂—C₁₋₁₀ aliphatic residue,
a O—C₁₋₁₀ aliphatic residue, a O—(C₁₋₈ aliphatic group)-O—C₁₋₁₀ aliphatic residue, O—(C₁₋₈ aliphatic group)-OH,
a NH—C₁₋₁₀ aliphatic residue, a N(C₁₋₁₀ aliphatic residue)₂, a NH—(C₁₋₈ aliphatic group)-O—C₁₋₁₀ aliphatic residue, a NH—(C₁₋₈ aliphatic group)-OH, a N(C₁₋₁₈ aliphatic residue)[(C₁₋₈ aliphatic group)-O—C₁₋₁₀ aliphatic residue], a N(C₁₋₁₀ aliphatic residue)[(C₁₋₈ aliphatic group)-OH], a NH—S(═O)₂—C₁₋₁₀ aliphatic residue,
wherein each of the aforementioned C₁₋₁₀ aliphatic residue and C₁₋₈ aliphatic groups can in each case be unsubstituted or monosubstituted with OH;
a C₃₋₁₀ cycloaliphatic residue, a C(═O)—C₃₋₁₀ cycloaliphatic residue, a C(═O)NH—C₃₋₁₀ cycloaliphatic residue, a O—C₃₋₁₀ cycloaliphatic residue, a NH—C₃₋₁₀ cycloaliphatic residue, a NH—C(═O)—C₃₋₁₀ cycloaliphatic residue, a 3 to 10 membered heterocycloaliphatic residue, a C(═O)-(3 to 10 membered heterocycloaliphatic residue), a C(═O)—NH-(3 to 10 membered heterocycloaliphatic residue), a O-(3 to 10 membered heterocycloaliphatic residue), a NH-(3 to 10 membered heterocycloaliphatic residue), a NH—C(═O)-(3 to 10 membered heterocycloaliphatic residue),
wherein each of the C₃₋₁₀ cycloaliphatic residue and the 3 to 10 membered heterocycloaliphatic residue, respectively, can independently be unsubstituted or mono- or polysubstituted with one or more substituents each independently selected from the group consisting of F, Cl, Br, I, C₁₋₄ alkyl, C₁₋₄ alkylene-OH, C₁₋₄ alkylene-O—C₁₋₄ alkyl, CF₃, C(═O)—C₁₋₄ alkyl, O—C₁₋₄ alkyl, O—C₁₋₄ alkylene-OH, O—C₁₋₄ alkylene-O—C₁₋₄ alkyl, OCF₃, OH, SH, S—C₁₋₄ alkyl, SCF₃, SO₂—C₁₋₄ alkyl, NH₂, NH—C₁₋₄ alkyl, N(C₁₋₄ alkyl)₂, NH—SO₂—C₁₋₄ alkyl, NH—C(═O)—C₁₋₄ alkyl; and
aryl, C(═O)-aryl, C(═O)—NH-aryl, NH—C(═O)-aryl, heteroaryl, C(═O)-heteroaryl, C(═O)—NH-heteroaryl, NH—C(═O)-heteroaryl,
wherein each aryl and heteroaryl of the aforementioned residues, respectively, can independently be unsubstituted or mono- or polysubstituted with one or more substituents each independently selected from the group consisting of F, Cl, Br, I, NO₂, CN, OH, O—C₁₋₄ alkyl, O—C₁₋₄ alkylene-O—C₁₋₄ alkyl, O—C₁₋₄ alkylene-OH, OCF₃, C₁₋₄ alkyl, C₁₋₄ alkylene-O—C₁₋₄-alkyl, C₁₋₄ alkylene-OH, C(═O)—C₁₋₄ alkyl, CF₃, CF₂H, CHF₂, SH, S—C₁₋₄ alkyl, SCF₃, SO₂—C₁₋₄ alkyl, NH₂, NH(C₁₋₄ alkyl), N(C₁₋₄ alkyl)₂, NH—SO₂—C₁₋₄ alkyl, NH—C(═O)—C₁₋₄ alkyl, phenyl and pyridyl, wherein phenyl or pyridyl are respectively unsubstituted or mono- or polysubstituted with one or more substituents each independently selected from the group consisting of F, Cl, Br, I, NO₂, CN, OH, O—C₁₋₄ alkyl, O—C₁₋₄ alkylene-O—C₁₋₄ alkyl OCF₃, C₁₋₄ alkyl, C₁₋₄ alkylene-O—C₁₋₄-alkyl, C(═O)—OH, CF₃, CF₂H, CHF₂, NH₂, NH(C₁₋₄ alkyl), N(C₁₋₄ alkyl)₂, SH, S—C₁₋₄ alkyl, SCF₃ and S(═O)₂OH.

13. A compound according to claim 12, wherein m represents 0, 1 or 2.

14. A compound according to claim 13, wherein m represents 0 or 1.

15. A compound according to claim 1, wherein
R⁵ and R⁹ are each independently selected from the group consisting of H; F; Cl; Br; I; CF₃; OH; CH₂OH; methyl; O-methyl,
R⁶ and R⁸ are each independently of one another selected from the group consisting of H; F; Cl; Br; I; CF₃; OH; CH₂OH; methyl; O-methyl; and
R⁷ is selected from the group consisting of:
H; F; Cl; Br; I; CN; CF₃; CF₂H; CFH₂; OH; OCF₃; SH; SCF₃; NH₂; C(═O)—NH₂; S(═O)₂—OH; S(═O)₂—NH₂;
C₁₋₄ alkyl, C₁₋₄ alkylene-OH, C₁₋₄ alkylene-O—C₁₋₄ alkyl, C₁₋₄ alkylene-O—C₁₋₄ alkylene-OH, C₁₋₄ alkylene-O—C₁₋₄ alkylene-O—C₁₋₄ alkyl, C₁₋₄ alkylene-S(═O)₂—C₁₋₄ alkyl, C₁₋₄ alkylene-NH—S(═O)₂—C₁₋₄ alkyl, C₁₋₄ alkylene-NH—S(═O)₂—NH₂, C₁₋₄ alkylene-NH—C₁₋₄ alkylene-OH, C₁₋₄ alkylene-NH—C₁₋₄ alkylene-O—C₁₋₄ alkyl, C₁₋₄ alkylene-N(C₁₋₄ alkyl)-C₁₋₄ alkylene-OH, C₁₋₄ alkylene-N(C₁₋₄ alkyl)-C₁₋₄ alkylene-O—C₁₋₄ alkyl, O—C₁₋₄ alkyl, O—C₁₋₄ alkylene-OH, O—C₁₋₄ alkylene-O—C₁₋₄ alkyl, NH—C₁₋₄ alkyl, N(C₁₋₄ alkyl)₂, NH—C₁₋₄ alkylene-OH, NH—C₁₋₄ alkylene-O—C₁₋₄ alkyl, N(C₁₋₄ alkyl)-[C₁₋₄ alkylene-OH], N(C₁₋₄ alkyl)-[C₁₋₄ alkylene-O—C₁₋₄ alkyl], NH—S(═O)₂—C₁₋₄ alkyl, wherein each C₁₋₄ alkylene can be unsubstituted or monosubstituted with OH,
a C₃₋₆ cycloaliphatic residue, O—C₃₋₆ cycloaliphatic residue, a 3 to 6 membered heterocycloaliphatic residue, wherein said C₃₋₆ cycloaliphatic residue and the 3 to 6 membered heterocycloaliphatic residue, respectively, can be unsubstituted or mono- or polysubstituted with one or more substituents each independently selected from the group consisting of F, Cl, Br, I, OH, O—C₁₋₄ alkyl, NH₂, NH(C₁₋₄ alkyl), and N(C₁₋₄ alkyl)₂, and C₁₋₄ alkyl, and
phenyl, C(═O)—NH-phenyl, NH—C(═O)-phenyl, heteroaryl, C(═O)—NH-heteroaryl, NH—C(═O)-heteroaryl,
wherein each phenyl and heteroaryl of the aforementioned residues, respectively, can independently be unsubstituted or mono- or polysubstituted with one or more substituents each independently selected from the group consisting of F, Cl, Br, I, OH, O—C₁₋₄ alkyl, C₁₋₄ alkyl, and CF₃.

16. A compound according to claim 15, wherein R⁷ represents:
a C₃₋₆ cycloaliphatic residue or O—C₃₋₆ cycloaliphatic residue, wherein the C₃₋₆ cycloaliphatic residue is selected from the group consisting of cyclopropyl, cyclobutyl, cyclopentyl, cyclohexyl, or
a 3 to 6 membered heterocycloaliphatic residue selected from the group consisting of tetrahydropyranyl, azetidinyl, piperidinyl, morpholinyl and pyrrolidinyl.

17. A compound according to claim 1, selected from the group consisting of:
1. N-((2-Pentyl-6-(trifluoromethyl)pyridin-3-yl)methyl)-2-(pyridin-2-yl)acetamide;
2. N-((2-Cyclopentyl-6-(trifluoromethyl)pyridin-3-yl)methyl)-2-(pyridin-2-yl)acetamide;
3. 1-(Pyridin-2-yl)-3-((2-(tetrahydro-2H-pyran-4-yl)-6-(trifluoromethyl)pyridin-3-yl)methyl)urea;
4. N-((2-(Cyclohexyl methyl)-6-(trifluoromethyl)pyridin-3-yl)methyl)-2-(pyridin-2-yl)acetamide;
5. N-((2-(3-Chlorophenyl)-6-(trifluoromethyl)pyridin-3-yl)methyl)-2-(pyridin-2-yl)acetamide;
6. N-((2-(3-Chloro-4-fluorophenyl)-6-(trifluoromethyl)pyridin-3-yl)methyl)-2-(pyridin-2-yl)acetamide;
7. 2-(Pyridin-2-yl)-N-((2-m-tolyl-6-(trifluoromethyl)pyridin-3-yl)methyl)acetamide;
8. N-((2-(3-Methoxyphenyl)-6-(trifluoromethyl)pyridin-3-yl)methyl)-2-(pyridin-2-yl)acetamide;
9. N-((2-(Butylamino)-6-(trifluoromethyl)pyridin-3-yl)methyl)-2-(pyridin-2-yl)acetamide;
10. 2-(Pyridin-2-yl)-N-((2-(pyrrolidin-1-yl)-6-(trifluoromethyl)pyridin-3-yl)methyl)acetamide;
11. N-(2-(4-Methylpiperidin-1-yl)-4-(trifluoromethyl)benzyl)-2-(pyridin-2-yl)acetamide;
12. N-((6-tert-Butyl-2-(4-methylpiperidin-1-yl)pyridin-3-yl)methyl)-2-(pyridin-2-yl)acetamide;
13. N-((2-(4-Methylpiperidin-1-yl)-6-(trifluoromethyl)pyridin-3-yl)methyl)-2-(pyridin-2-yl)acetamide;
14. N-((2-(4-Methylpiperidin-1-yl)-6-(trifluoromethyl)pyridin-3-yl)methyl)-2-(pyridin-2-yl)propanamide;
15. 2-Methyl-N-((2-(4-methylpiperidin-1-yl)-6-(trifluoromethyl)pyridin-3-yl)methyl)-2-(pyridin-2-yl)propanamide;
16. N-((2-(4-Methylpiperidin-1-yl)-6-(trifluoromethyl)pyridin-3-yl)methyl)-1-(pyridin-2-yl)cyclopropanecarboxamide;
17. 2-Cyclohexyl-N-((2-(4-methylpiperidin-1-yl)-6-(trifluoromethyl)pyridin-3-yl)methyl)-2-(pyridin-2-yl)acetamide;
18. N-((2-(4-Methylpiperidin-1-yl)-6-(trifluoromethyl)pyridin-3-yl)methyl)-2-(pyridin-2-yl)-2-m-tolylacetamide;
19. 1-((2-(4-Methylpiperidin-1-yl)-6-(trifluoromethyl)pyridin-3-yl)methyl)-3-(pyridin-2-yl)urea;
20. 1-Methyl-3-((2-(4-methylpiperidin-1-yl)-6-(trifluoromethyl)pyridin-3-yl)methyl)-1-(pyridin-2-yl)urea;
21. 1-Methyl-1-((2-(4-methylpiperidin-1-yl)-6-(trifluoromethyl)pyridin-3-yl)methyl)-3-(pyridin-2-yl)urea;
22. N-((2-Morpholino-6-(trifluoromethyl)pyridin-3-yl)methyl)-2-(pyridin-2-yl)acetamide;
23. 1-((2-(4-(Dimethylamino)piperidin-1-yl)-6-(trifluoromethyl)pyridin-3-yl)methyl)-3-(pyridin-2-yl)urea;
24. N-((2-((2-Methoxyethoxy)methyl)-6-(trifluoromethyl)pyridin-3-yl)methyl)-2-(pyridin-2-yl)acetamide;
25. N-((2-Butoxy-6-(trifluoromethyl)pyridin-3-yl)methyl)-2-(pyridin-2-yl)acetamide;
26. N-((2-(Cyclobutylmethoxy)-6-(trifluoromethyl)pyridin-3-yl)methyl)-2-(pyridin-2-yl)acetamide;
27. N-((2-(Cyclohexyloxy)-6-(trifluoromethyl)pyridin-3-yl)methyl)-2-(pyridin-2-yl)acetamide;
28. N-(4-tert-Butyl-2-(cyclohexylthio)benzyl)-2-(pyridin-2-yl)acetamide;
29. N-(2-(Cyclohexylthio)-4-(trifluoromethyl)benzyl)-2-(pyridin-2-yl)acetamide;
30. N-((6-Cyclopropyl-2-(4-methylpiperidin-1-yl)pyridin-3-yl)methyl)-2-(pyridin-2-yl)acetamide;
31. N-((2-(4-Methylpiperidin-1-yl)-6-(trifluoromethyl)pyridin-3-yl)methyl)-2-(pyridin-3-yl)acetamide;
32. N-((2-(4-Methylpiperidin-1-yl)-6-(trifluoromethyl)pyridin-3-yl)methyl)-2-(pyridin-3-yl)propanamide;
33. N-(4-tert-Butyl-2-(4-methylpiperidin-1-yl)benzyl)-2-(pyridin-3-yl)acetamide;
34. N-((2-(Cyclohexylthio)-6-(trifluoromethyl)pyridin-3-yl)methyl)-2-(pyridin-3-yl)acetamide;
35. 1-((2-(3-Chlorophenyl)-6-(trifluoromethyl)pyridin-3-yl)methyl)-3-(pyridin-3-yl)urea;
36. 1-(Pyridin-3-yl)-3-((2-m-tolyl-6-(trifluoromethyl)pyridin-3-yl)methyl)urea;
37. 1-((2-(3-Methoxyphenyl)-6-(trifluoromethyl)pyridin-3-yl)methyl)-3-(pyridin-3-yl)urea;
38. N-((2-(4-Methylpiperidin-1-yl)-6-(trifluoromethyl)pyridin-3-yl)methyl)-2-(pyridin-4-yl)acetamide;
39. N-((2-(4-Methylpiperidin-1-yl)-6-(trifluoromethyl)pyridin-3-yl)methyl)-2-(pyrimidin-4-yl)acetamide;
40. N-((2-(4-Methylpiperidin-1-yl)-6-(trifluoromethyl)pyridin-3-yl)methyl)-2-(pyrazin-2-yl)acetamide;
41. N-((2-(4-Methylpiperidin-1-yl)-6-(trifluoromethyl)pyridin-3-yl)methyl)-2-(pyrimidin-2-yl)acetamide;
42. 1-((2-(4-Methylpiperidin-1-yl)-6-(trifluoromethyl)pyridin-3-yl)methyl)-3-(pyridazin-4-yl)urea;
43. 1-(2-(4-Methylpiperidin-1-yl)-4-(trifluoromethyl)benzyl)-3-(pyridazin-4-yl)urea;
44. 1-(4-tert-Butyl-2-(cyclohexylthio)benzyl)-3-(pyridazin-4-yl)urea;
45. 1-((2-(3-Fluorophenyl)-6-(trifluoromethyl)pyridin-3-yl)methyl)-3-(pyridazin-4-yl)urea;
46. 1-((2-(3-Chloro-4-fluorophenyl)-6-(trifluoromethyl)pyridin-3-yl)methyl)-3-(pyridazin-4-yl)urea;
47. N-((2-(4-Methylpiperidin-1-yl)-6-(trifluoromethyl)pyridin-3-yl)methyl)-2-(pyrimidin-5-yl)acetamide;
48. 1-((2-(4-Methylpiperidin-1-yl)-6-(trifluoromethyl)pyridin-3-yl)methyl)-3-(1,3,5-triazin-2-yl)urea;
49. 2-(6-Chloropyridin-3-yl)-N-((2-(4-methylpiperidin-1-yl)-6-(trifluoromethyl)pyridin-3-yl)methyl)propanamide;
50. 2-(5-Fluoropyridin-3-yl)-N-((2-(4-methylpiperidin-1-yl)-6-(trifluoromethyl)pyridin-3-yl)methyl)acetamide;
51. 1-(5-Fluoropyridin-3-yl)-3-((2-(4-methylpiperidin-1-yl)-6-(trifluoromethyl)pyridin-3-yl)methyl)urea;
52. 1-((2-(4-Methylpiperidin-1-yl)-6-(trifluoromethyl)pyridin-3-yl)methyl)-3-(2-methylpyrimidin-5-yl)urea;
53. 2-(6-(Hydroxymethyl)pyridin-3-yl)-N-((2-(4-methylpiperidin-1-yl)-6-(trifluoromethyl)pyridin-3-yl)methyl)propanamide;
54. N-((2-(3-Fluorophenyl)-6-(trifluoromethyl)pyridin-3-yl)methyl)-2-(6-(hydroxymethyl)pyridin-3-yl)propanamide;
55. 1-(6-(Hydroxymethyl)pyridin-3-yl)-3-((2-(4-methylpiperidin-1-yl)-6-(trifluoromethyl)pyridin-3-yl)methyl)urea;
56. 1-(6-(Hydroxymethyl)pyridin-3-yl)-3-((2-pentyl-6-(trifluoromethyl)pyridin-3-yl)methyl)urea;
57. 1-((2-(3-Fluorphenyl)-6-(trifluormethyl)pyridin-3-yl)methyl)-3-(6-(hydroxymethyl)pyridin-3-yl)urea;
58. 1-(6-(Hydroxymethyl)pyridin-3-yl)-3-((2-m-tolyl-6-(trifluoromethyl)pyridin-3-yl)methyl)urea;
59. 1-(6-(Hydroxymethyl)pyridin-3-yl)-3-((2-(3-isopropylphenyl)-6-(trifluoromethyl)pyridin-3-yl)methyl)urea;
60. 1-((2-(3-(Dimethylamino)phenyl)-6-(trifluoromethyl)pyridin-3-yl)methyl)-3-(6-(hydroxymethyl)pyridin-3-yl)urea;
61. 1-(5-Fluoro-6-(hydroxymethyl)pyridin-3-yl)-3-((2-(4-methylpiperidin-1-yl)-6-(trifluoromethyl)pyridin-3-yl)methyl)urea;
62. 2-(6-(2-Hydroxyethyl)pyridin-3-yl)-N-((2-(4-methylpiperidin-1-yl)-6-(trifluoromethyl)pyridin-3-yl)methyl)propanamide;
63. 1-(6-(2-Hydroxyethyl)pyridin-3-yl)-3-((2-(4-methylpiperidin-1-yl)-6-(trifluoromethyl)pyridin-3-yl)methyl)urea;
64. 2-(6-((2-Hydroxyethoxy)methyl)pyridin-3-yl)-N-((2-(4-methylpiperidin-1-yl)-6-(trifluoromethyl)pyridin-3-yl)methyl)propanamide;
65. 1-((2-(4-Methylpiperidin-1-yl)-6-(trifluoromethyl)pyridin-3-yl)methyl)-3-(6-(methylsulfonylmethyl)pyridin-3-yl)urea;
66. 1-((2-(4-Methylpiperidin-1-yl)-6-(trifluoromethyl)pyridin-3-yl)methyl)-3-(6-(2-(methylsulfonyl)ethyl)pyridin-3-yl)urea;
67. 1-(5-Fluoro-6-(2-(methylsulfonyl)ethyl)pyridin-3-yl)-3-((2-(4-methylpiperidin-1-yl)-6-(trifluoromethyl)pyridin-3-yl)methyl)urea;
68. 1-((6-Cyclopropyl-2-(4-methylpiperidin-1-yl)pyridin-3-yl)methyl)-3-(5-fluoro-6-(2-(methylsulfonyl)ethyl)pyridin-3-yl)urea;
69. 1-(5-Fluoro-6-(2-(methylsulfonyl)ethyl)pyridin-3-yl)-3-((2-(3-fluorophenyl)-6-(trifluoromethyl)pyridin-3-yl)methyl)urea;
70. N-((5-(3-((2-(4-Methylpiperidin-1-yl)-6-(trifluoromethyl)pyridin-3-yl)methyl)ureido)pyridin-2-yl)methyl)methanesulfonamide;
71. N-((5-(3-((2-(4-Methylpiperidin-1-yl)-6-(trifluoromethyl)pyridin-3-yl)methyl)ureido)pyridin-2-yl)methyl)sulfuric diamide;
72. N-((5-(3-(2-(Cyclohexyloxy)-4-(trifluoromethyl)benzyl)ureido)pyridin-2-yl)methyl)sulfuric diamide;
73. N-((5-(3-((2-m-Tolyl-6-(trifluoromethyl)pyridin-3-yl)methyl)ureido)pyridin-2-yl)methyl)sulfuric diamide;
74. 5-(1-((2-(4-Methylpiperidin-1-yl)-6-(trifluoromethyl)pyridin-3-yl)methylamino)-1-oxopropan-2-yl)picolinamide;
75. 5-(1-((2-(4-Methylpiperidin-1-yl)-6-(trifluoromethyl)pyridin-3-yl)methylamino)-1-oxopropan-2-yl)-N-phenylpicolinamide;
76. 5-(1-((2-(4-Methylpiperidin-1-yl)-6-(trifluoromethyl)pyridin-3-yl)methylamino)-1-oxopropan-2-yl)-N-phenylpyrimidine-2-carboxamide;
77. 5-(1-((2-(Ethylamino)-6-(trifluoromethyl)pyridin-3-yl)methylamino)-1-oxopropan-2-yl)-N-(4-fluorophenyl)pyrimidine-2-carboxamide;
78. N-(4-Fluorophenyl)-5-(1-oxo-1-((2-(pyrrolidin-1-yl)-6-(trifluoromethyl)pyridin-3-yl)methylamino)propan-2-yl)pyrimidine-2-carboxamide;
79. N-(4-Fluorophenyl)-5-(1-oxo-1-((2-(piperidin-1-yl)-6-(trifluoromethyl)pyridin-3-yl)methylamino)propan-2-yl)pyrimidine-2-carboxamide;
80. N-(4-Fluorophenyl)-5-(1-((2-morpholino-6-(trifluoromethyl)pyridin-3-yl)methylamino)-1-oxopropan-2-yl)pyrimidine-2-carboxamide;
81. N-(4-Fluorophenyl)-5-(1-oxo-1-((2-m-tolyl-6-(trifluoromethyl)pyridin-3-yl)methylamino)propan-2-yl)pyrimidine-2-carboxamide;
82. 5-(1-oxo-1-((2-(piperidin-1-ylmethyl)-6-(trifluoromethyl)pyridin-3-yl)methylamino)propan-2-yl)-N-(4-(trifluoromethyl)phenyl)pyrimidine-2-carboxamide;
83. 1-((2-(4-Methylpiperidin-1-yl)-6-(trifluoromethyl)pyridin-3-yl)methyl)-3-(6-(tetrahydro-2H-pyran-4-yl)pyridin-3-yl)urea;
84. 2-(5-Amino-6-bromopyridin-2-yl)-N-((2-(4-methylpiperidin-1-yl)-6-(trifluoromethyl)pyridin-3-yl)methyl)propanamide;
85. 2-(6-(2-Hydroxyethylamino)pyridin-3-yl)-N-((2-(4-methylpiperidin-1-yl)-6-(trifluoromethyl)pyridin-3-yl)methyl)propanamide;
86. 1-(6-(2-Hydroxyethylamino)pyridin-3-yl)-3-((2-(4-methylpiperidin-1-yl)-6-(trifluoromethyl)pyridin-3-yl)methyl)urea;
87. 2-(6-(2-Methoxyethylamino)pyridin-3-yl)-N-((2-(4-methylpiperidin-1-yl)-6-(trifluoromethyl)pyridin-3-yl)methyl)propanamide;
88. 1-(6-(2-Methoxyethylamino)pyridin-3-yl)-3-((2-(4-methylpiperidin-1-yl)-6-(trifluoromethyl)pyridin-3-yl)methyl)urea;
89. 2-(6-((2-Hydroxyethyl)(methyl)amino)pyridin-3-yl)-N-((2-(4-methylpiperidin-1-yl)-6-(trifluoromethyl)pyridin-3-yl)methyl)propanamide;
90. 1-(6-((2-Hydroxyethyl)(methyl)amino)pyridin-3-yl)-3-((2-(4-methylpiperidin-1-yl)-6-(trifluoromethyl)pyridin-3-yl)methyl)urea;
91. 1-(6-((2-Methoxyethyl)(methyl)amino)pyridin-3-yl)-3-((2-(4-methylpiperidin-1-yl)-6-(trifluoromethyl)pyridin-3-yl)methyl)urea;
92. N-((2-(4-Methylpiperidin-1-yl)-6-(trifluormethyl)pyridin-3-yl)methyl)-2-(6-(methylsulfonamido)pyridin-3-yl)propanamide;
93. N-(5-(3-((2-(4-Methylpiperidin-1-yl)-6-(trifluoromethyl)pyridin-3-yl)methyl)ureido)pyridin-2-yl)methanesulfonamide;
94. N-(5-(3-((6-Cyclopropyl-2-(4-methylpiperidin-1-yl)pyridin-3-yl)methyl)ureido)pyridin-2-yl)methanesulfonamide;
95. 2-(6-(Methylsulfonamido)pyridin-3-yl)-N-((2-morpholino-6-(trifluoromethyl)pyridin-3-yl)methyl)propanamide;
96. 2-(5-Fluoro-6-(methylsulfonamido)pyridin-3-yl)-N-((2-(4-methylpiperidin-1-yl)-6-(trifluoromethyl)pyridin-3-yl)methyl)propanamide;
97. 2-(5-Methoxy-6-(methylsulfonamido)pyridin-3-yl)-N-((2-(4-methylpiperidin-1-yl)-6-(trifluoromethyl)pyridin-3-yl)methyl)propanamide;
98. N-(5-(1-((2-(4-Methylpiperidin-1-yl)-6-(trifluoromethyl)pyridin-3-yl)methylamino)-1-oxopropan-2-yl)pyridin-2-yl)benzamide;
99. 4-Chloro-N-(5-(1-((2-(4-methylpiperidin-1-yl)-6-(trifluoromethyl)pyridin-3-yl)methylamino)-1-oxopropan-2-yl)pyridin-2-yl)benzamide;
100. 4-Chloro-N-(5-(1-(2-(4-methylpiperidin-1-yl)-4-(trifluoromethyl)benzylamino)-1-oxopropan-2-yl)pyridin-2-yl)benzamide;
101. 4-Chloro-N-(5-(1-(2-(cyclohexylthio)-4-(trifluoromethyl)benzylamino)-1-oxopropan-2-yl)pyridin-2-yl)benzamide;
102. N-(5-(1-(4-tert-Butyl-2-(cyclohexylthio)benzylamino)-1-oxopropan-2-yl)pyridin-2-yl)-4-chlorobenzamide;
103. 4-Chloro-N-(5-(1-(2-(cyclopentyloxy)-4-(trifluoromethyl)benzylamino)-1-oxopropan-2-yl)pyridin-2-yl)benzamide;
104. 1-(6-(Dimethylamino)-5-(trifluoromethyl)pyridin-3-yl)-3-((2-(4-methylpiperidin-1-yl)-6-(trifluoromethyl)pyridin-3-yl)methyl)urea;
105. 1-(6-(Azetidin-1-yl)pyridin-3-yl)-3-((2-(4-methylpiperidin-1-yl)-6-(trifluoromethyl)pyridin-3-yl)methyl)urea;
106. 1-(6-(Azetidin-1-yl)-5-fluoropyridin-3-yl)-3-((2-(4-methylpiperidin-1-yl)-6-(trifluoromethyl)pyridin-3-yl)methyl)urea;
107. 1-(6-(Azetidin-1-yl)-5-methoxypyridin-3-yl)-3-((2-(4-methylpiperidin-1-yl)-6-(trifluoromethyl)pyridin-3-yl)methyl)urea;
108. 1-(6-(3-Hydroxyazetidin-1-yl)pyridin-3-yl)-3-((2-(4-methylpiperidin-1-yl)-6-(trifluoromethyl)pyridin-3-yl)methyl)urea;
109. 1-(6-(3-Hydroxyazetidin-1-yl)pyridin-3-yl)-3-((2-pentyl-6-(trifluoromethyl)pyridin-3-yl)methyl)urea;
110. 1-(6-(3-Hydroxyazetidin-1-yl)pyridin-3-yl)-3-((2-m-tolyl-6-(trifluoromethyl)pyridin-3-yl)methyl)urea;
111. 1-(6-(3-Hydroxyazetidin-1-yl)pyridin-3-yl)-3-((2-methoxy-6-(trifluoromethyl)pyridin-3-yl)methyl)urea;
112. 1-(6-(3-Hydroxyazetidin-1-yl)pyridin-3-yl)-3-((2-isobutoxy-6-(trifluoromethyl)pyridin-3-yl)methyl)urea;
113. 1-((2-(Cyclobutylmethoxy)-6-(trifluoromethyl)pyridin-3-yl)methyl)-3-(6-(3-hydroxyazetidin-1-yl)pyridin-3-yl)urea;
114. 1-((2-(4-Methylpiperidin-1-yl)-6-(trifluoromethyl)pyridin-3-yl)methyl)-3-(6-(pyrrolidin-1-yl)pyridin-3-yl)urea;
115. 1-(5-Fluoro-6-(pyrrolidin-1-yl)pyridin-3-yl)-3-((2-(4-methylpiperidin-1-yl)-6-(trifluoromethyl)pyridin-3-yl)methyl)urea;
116. 1-(5-Methoxy-6-(pyrrolidin-1-yl)pyridin-3-yl)-3-((2-(4-methylpiperidin-1-yl)-6-(trifluoromethyl)pyridin-3-yl)methyl)urea;
117. (S)-1-(6-(3-Hydroxypyrrolidin-1-yl)pyridin-3-yl)-3-((2-(4-methylpiperidin-1-yl)-6-(trifluoromethyl)pyridin-3-yl)methyl)urea;
118. (R)-1-(6-(3-Hydroxypyrrolidin-1-yl)pyridin-3-yl)-3-((2-(4-methylpiperidin-1-yl)-6-(trifluoromethyl)pyridin-3-yl)methyl)urea;
119. 1-(6-Hydroxypyridin-3-yl)-3-((2-(4-methylpiperidin-1-yl)-6-(trifluoromethyl)pyridin-3-yl)methyl)urea;
120. 2-(6-Methoxypyridin-3-yl)-N-((2-(4-methylpiperidin-1-yl)-6-(trifluoromethyl)pyridin-3-yl)methyl)propanamide;
121. 1-(2-Methoxypyrimidin-5-yl)-3-((2-(4-methylpiperidin-1-yl)-6-(trifluoromethyl)pyridin-3-yl)methyl)urea;
122. 1-(2-Cyclobutoxypyrimidin-5-yl)-3-((2-(4-methylpiperidin-1-yl)-6-(trifluoromethyl)pyridin-3-yl)methyl)urea;
123. 1-(6-(2-Hydroxyethoxy)pyridin-3-yl)-3-((2-(4-methylpiperidin-1-yl)-6-(trifluoromethyl)pyridin-3-yl)methyl)urea;
124. 1-(6-(2-Methoxyethoxy)pyridin-3-yl)-3-((2-(4-methylpiperidin-1-yl)-6-(trifluoromethyl)pyridin-3-yl)methyl)urea;
125. 1-(6-(2-Hydroxyethoxy)pyridin-3-yl)-3-((2-m-tolyl-6-(trifluoromethyl)pyridin-3-yl)methyl)urea;
126. 1-(5-(Hydroxymethyl)pyridin-3-yl)-3-((2-(4-methylpiperidin-1-yl)-6-(trifluoromethyl)pyridin-3-yl)methyl)urea;
127. 1-(5-(Hydroxymethyl)pyridin-2-yl)-3-((2-(4-methylpiperidin-1-yl)-6-(trifluoromethyl)pyridin-3-yl)methyl)urea;
128. 1-(3-(Hydroxymethyl)pyridin-4-yl)-3-((2-(4-methylpiperidin-1-yl)-6-(trifluoromethyl)pyridin-3-yl)methyl)urea;
129. 1-(6-(1,2-Dihydroxyethyl)pyridin-3-yl)-3-((2-(4-methylpiperidin-1-yl)-6-(trifluoromethyl)pyridin-3-yl)methyl)urea;
130. 1-((2-(3-Fluorophenyl)-6-(trifluoromethyl)pyridin-3-yl)methyl)-3-(6-(2-hydroxyethylamino)pyridin-3-yl)urea, and
131. 1-((5′-Chloro-6-(trifluoromethyl)-2,3′-bipyridin-3-yl)methyl)-3-(6-(2-hydroxyethylamino)pyridin-3-yl)urea,
optionally in the form of a single stereoisomer or a mixture of stereoisomers, in the form of the free compound and/or a physiologically acceptable salt thereof.

18. A pharmaceutical composition comprising a compound according to claim 1 and at least one pharmaceutically acceptable carrier or auxiliary substance.

19. A method of treating or inhibiting a disorder or disease selected from the group consisting of pain; hyperalgesia; allodynia; causalgia; migraine; depression; nervous affection; axonal injuries; neurodegenerative diseases; cognitive dysfunctions; epilepsy; respiratory diseases; coughs; urinary incontinence; overactive bladder; disorders and injuries of the gastrointestinal tract; duodenal ulcers; gastric ulcers; irritable bowel syndrome; strokes; eye irritations; skin irritations; neurotic skin diseases; allergic skin diseases; psoriasis; vitiligo; herpes simplex; inflammations; diarrhoea; pruritus; osteoporosis; arthritis; osteoarthritis; rheumatic diseases; eating disorders; medication dependency; misuse of medication; withdrawal symptoms in medication dependency; development of tolerance to medication; drug dependency; misuse of drugs; withdrawal symptoms in drug dependency; alcohol dependency; misuse of alcohol and withdrawal symptoms in alcohol dependency; or of effecting diuresis; antinatriuresis; influencing the cardiovascular system; increasing vigilance; treating wounds and/or burns; treating severed nerves; increasing libido; modulating movement activity; effecting anxiolysis; local anaesthesia or for inhibiting undesirable side effects triggered by the administration of vanilloid receptor 1 agonists in a mammal, said method comprising administering an effective amount of a compound according to claim 1 to said mammal.

20. A method according to claim 19,
wherein said disorder or disease is pain selected from the group consisting of acute pain, chronic pain, neuropathic pain, visceral pain and joint pain; a neurodegenerative disease selected from the group consisting of multiple sclerosis, Alzheimer's disease, Parkinson's disease and Huntington's disease; a memory disorder; a respiratory disease selected from the group consisting of asthma, bronchitis and pulmonary inflammation; an inflammation of the intestine, the eyes, the bladder, the skin or the nasal mucous membrane; an eating disorder selected from the group consisting of bulimia, cachexia, anorexia and obesity; or development of tolerance to natural or synthetic opioids; or
of inhibiting undesirable side effects selected from the group consisting of hyperthermia, hypertension and bronchoconstriction triggered by the administration of a vanilloid receptor 1 agonist selected from the group consisting of capsaicin, resiniferatoxin, olvanil, arvanil, SDZ-249665, SDZ-249482, nuvanil and capsavanil.
